SEQUENCE LISTING (8 sequences)

SEQ ID NO: 1 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 2 - protein, 164 aa, Homo sapiens
EQKLISEEDLNKELQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 3 - protein, 250 aa, Homo sapiens [sequence omitted]

SEQ ID NO: 4 - protein, 14 aa, Homo sapiens
EQKLISEEDLNKEL

SEQ ID NO: 5 - protein, 152 aa, Unknown Organism
  Description of Unknown Organism Illustrative peptide TNFa
RTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLLFAESGQVYFGIIAL

SEQ ID NO: 6 - protein, 147 aa, Unknown Organism
  Description of Unknown Organism Illustrative peptide LTa
TLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFALT

SEQ ID NO: 7 - protein, 146 aa, Unknown Organism
  Description of Unknown Organism Illustrative peptide CD40L
GDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 8 - protein, 163 aa, Unknown Organism
  Description of Unknown Organism Illustrative peptide Apo2L/Trail
PQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

(19) United States
(12) Patent Application Publication
(10) Pub. No.: US 2006/0136136 A1
(43) Pub. Date: Jun. 22, 2006

(54) Crystal structure of baff, and use thereof in drug design

(76) Inventor: Michael Karpusas, Upper Darby, PA (US)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 10/794,755

(22) Filed: Mar. 5, 2004

Related U.S. Application Data
(63) Continuation of application No. PCT/US02/28579, filed on Sep. 6, 2002.
(60) Provisional application No. 60/317,524, filed on Sep. 6, 2001.

(51) Int. Cl. G06F 19/00; C07K 14/535

(52) U.S. Cl. 702/19; 530/351

(57) ABSTRACT

The present invention relates to crystallizable compositions and crystals of BAFF. In addition, this invention relates to the high resolution structure of a BAFF polypeptide as obtained by X-ray crystallography. This invention also relates to a computer (machine) comprising a machine-readable data storage medium comprising a data storage material encoded with machine-readable data comprising the structure coordinates provided by this invention. This invention also relates to methods of using the structure coordinates of BAFF to solve the structure of similar or homologous molecules or molecular complexes and methods of determining the homology model of a similar or homologous molecule, such as APRIL. This invention also provides a computer capable of producing a three-dimensional representation of APRIL based on the homology model structure coordinates. This invention also relates to methods using the structure coordinates of BAFF to design chemical entities or compounds, including agonists or antagonists of BAFF, that specifically bind BAFF, as well as to design variants of BAFF agonists or antagonists, with improved properties (such as increased or decreased binding affinity for BAFF). This invention also provides variants of BAFF. This invention also relates to compositions comprising said chemical entities, compounds, including agonists or antagonists of BAFF, or variants.

This application claims benefit of U.S. provisional application No. 60/317,524, filed Sep. 6, 2001, the disclosure of which is incorporated by reference herein.

### TECHNICAL FIELD OF THE INVENTION

The present invention relates to crystallizable compositions and crystals of BAFF. In addition, this invention relates to the high resolution structure of a BAFF polypeptide as obtained by X-ray crystallography. This invention also relates to a computer (machine) comprising a machine-readable data storage medium comprising a data storage material encoded with machine-readable data comprising the structure coordinates provided by this invention. This invention also relates to methods of using the structure coordinates of BAFF to solve the structure of similar or homologous molecules or molecular complexes and methods of determining the homology model of a similar or homologous molecule, such as APRIL. This invention also provides a computer capable of producing a three-dimensional representation of APRIL based on the homology model structure coordinates. This invention also relates to methods using the structure coordinates of BAFF to design chemical entities or compounds, including agonists or antagonists of BAFF, that specifically bind BAFF, as well as to design variants of BAFF agonists or antagonists, with improved properties (such as increased or decreased binding affinity for BAFF). This invention also provides variants of BAFF. This invention also relates to compositions comprising said chemical entities, compounds, including agonists or antagonists of BAFF, or variants.

### BACKGROUND OF THE INVENTION

BAFF (which stands for B-cell activating factor belonging to the tumor necrosis factor (TNF) family) is a recently identified member of the TNF family of ligands (Shu, H. B., et al., J Leukoc Biol, 65(5): p. 680-3 (1999); Moore, P. A., et al., Science, 285(5425): p. 260-3 (1999); Schneider, P., et al., J Exp Med, 189(11): p. 1747-56 (1999)). BAFF is also known by other names, such as BlyS, TALL-1, THANK and zTNF4.

BAFF is a type II membrane protein that is expressed in different cell types, including T-cells and dendritic cells. BAFF can also exist as a soluble form; and the soluble form can induce proliferation of peripheral blood lymphocytes, just like the full-length protein (Schneider, P., et al., J Exp Med, 189(11): p. 1747-56 (1999), the disclosure of which is incorporated by reference herein). The full-length BAFF is a trimeric molecule that includes a globular extracellular TNF-homologous domain, an extracellular stalk, a short transmembrane segment and a small cytoplasmic domain. Soluble BAFF also forms trimers. Several studies indicate that BAFF is a key regulator of the peripheral B-cell population (Moore, P. A., et al., Science, 285(5425): p. 260-3 (1999); Schneider, P., et al., J Exp. Med, 189(11): p. 1747-56 (1999); Batten, M., et al., J Exp Med, 192(10): p. 1453-66 (2000)). BAFF has also been shown to play an essential role in the normal development of B-cells (Schiemann et al., An Essential Role for BAFF in the Normal Development of B-cells Through a BCMA-Independent Pathway, Sciencexpress (Aug. 16, 2001), at http://www.sciencexpress.org and Schiemann et al., Science Sep. 14, 2001; 293(5537): 2111-2114; the disclosures of both are incorporated by reference herein).

BAFF acts by binding to receptors expressed on B-cells and inducing B-cell proliferation and survival. So far, three receptors of BAFF have been identified: B-cell maturation antigen (“BCMA”) (Madry, C., et al., Int Immunol, 10(11): p. 1693-702 (1998); Thompson, J. S., et al., J Exp Med, 192(1): p. 129-35 (2000)), transmembrane activator and CAML interactor (“TACI”) (Xia, X. Z., et al., J Exp Med, 192(1): p. 137-43 (2000); Yan, M., et al., Science, 290(5491): p. 523-7 (2000))-and BAFF receptor (“BAFF-R”) (Thompson et al., BAFF-R, a Novel TNF Receptor That Specifically Interacts with BAFF, Sciencexpress (Aug. 16, 2001, at http://www.sciencexpress.org) and Thompson, J. S. et al., Science (Sep. 14, 2001); 293 (5537): 2108-2111, the disclosures of both of which are incorporated by reference herein). These receptors lack signal sequences and thus are classified as type III membrane proteins, a fact that is uncommon for TNF family receptors. Two of the receptors (BCMA and TACI) also bind to APRIL (which stands for a proliferation-inducing ligand), another TNF family member that is closely related to BAFF both structurally and functionally (Hahne, M., et al., J Exp Med, 188(6): p. 1185-90 (1998); Xia, X. Z., et al., J Exp Med, 192(1): p. 137-43 (2000); Gross, J. A., et al., Nature, 404(6781): p. 995-9 (2000); Wu, Y., et al., J Biol Chem, 275(45): p. 35478-85 (2000); Marsters, S. A., et al., Curr Biol. 10(13): p. 785-8 (2000)). It appears that BAFF signaling is primarily through BAFF-R receptor (Thompson et al., BAFF-R, a Novel TNF Receptor That Specifically Interacts with BAFF, Sciencexpress (Aug. 16, 2001), at http://www.sciencexpress.org and Thompson, J. S. et al., Science (Sep. 14, 2001); 293 (5537): 2108-2111). The extracellular domain of BAFF is involved in the interaction with one or more of the BAFF receptors. The extracellular domain of APRIL is involved in the interaction with one or more of the APRIL receptors.

The TNF family of ligands includes, among others, TNF-α, lymphotoxin-α (LT-α), lymphotoxin-β (LT-β), CD40 ligand (CD40L), OX40 ligand (OX40L), Fas ligand, and Apo2L/TRAIL (Locksley, R. M., et al., Cell, 104(4): p. 487-501 (2001)). Crystal structures have been determined for the TNF-homologous domains of TNF-α (Jones, E. Y., D. I. Stuart, and N. P. Walker, Nature, 338(6212): p. 225-8 (1989); Eck, M. J. and S. R. Sprang, J Biol Chem, 264(29): p. 17595-605 (1989)), LT-α(Eck, M. J., et al., J Biol Chem, 267(4): p. 2119-22 (1992)), CD40L (Karpusas, M., et al., Structure, 3(12): p. 1426 (1995) and Karpusas, M., et al., Structure, 3: p. 1031-1039 (1995)) and Apo2L/TRAIL (Cha, S. S., et al., Immunity, 11(2): p. 253-61 (1999); Hymowitz, S. G., et al., Biochemistry, 39(4): p. 633-40 (2000)). The structures show that these domains adopt the jellyroll, or β-sheet sandwich motif.

The TNF ligands induce a signal by binding to the corresponding TNF family receptors expressed on the surface of the target cell. Each receptor is an elongated molecule that consists of tandem repeats, termed cysteine-rich domains (“CRD”), due to the high number of disulfide bridges contained in each repeat. A more precise description of the modular structure of the TNF family receptors suggests that the basic building block is roughly half a CRD in terms of size, and can exist in different types, types A, B, C, each containing one or two disulfide bridges (Naismith, J. H. and S. R. Sprang, Trends Biochem Sci, 23(2): p. 74-9 (1998)).

Crystal structures of LT-α and TRAIL complexed with their receptors provided insights into the common aspects of TNF family ligand-receptor recognition (Banner, D. W., et al., Cell, 73(3): p. 431-45 (1993); Hymowitz, S. G., et al., Mol Cell, 4(4): p. 563-71 (1999); Mongkolsapaya, J., et al., Nat Struct Biol, 6(11): p. 1048-1053 (1999); Cha, S. S., et al., J Biol Chem, 275(40): p. 31171-7 (2000)). The structures show that TNF ligands induce a signal by forming a trimeric complex with the receptors.

The structural basis of the interaction of BAFF with its receptors is of special interest due to a very unusual characteristic: BCMA and BAFF-R are the only known TNF receptors that appear to contain only one CRD. In addition, sequence comparison indicates that BCMA, TACI and BAFF-R are rather distantly related to other members of the TNF receptor family (Madry, C., et al., Int Immunol, 10(11): p. 1693-702 (1998); Thompson et al., BAFF-R, a Novel TNF Receptor That Specifically Interacts with BAFF, Sciencexpress (Aug. 16, 2001), at http://www.sciencexpress.org and Thompson, J. S. et al., Science (Sep. 14, 2001); 293 (5537): 2108-2111). This suggests the presence of different folding motifs in the receptor extracellular domains and possibly new associated modes of binding.

Recent data indicate that BAFF and APRIL are key players in autoimmune disease, while APRIL is also implicated in cancer (Ware, C. F., J Exp Med, 192(11): p. F35-8 (2000); Yu, G., et al., Nat Immunol, 1(3): p. 252-6 (2000); Khare, S. D. and H. Hsu, Trends Immunol, 22(2): p. 61-63 (2001)) (mice over-expressing BAFF also display mature B-cell hyperplasia (Mackay, F., et al., J Exp Med, 190(11): p. 1697-710 (1999)). Transgenic mice that overexpressed BAFF exhibited increased numbers of peripheral B lymphocytes and developed an autoimmune condition similar to systemic lupus erythematosis (“SLE”) (Mackay, F., et al., J Exp Med, 190(11): p. 1697-710 (1999)). Moreover, the amount of BAFF in the serum of SLE patients is found to be elevated compared to healthy individuals (Zhang, J., et al., J Immunol, 166(1): p. 6-10 (2001)). These results indicate the involvement of BAFF in antibody-mediated autoimmune diseases. BAFF, therefore, is an attractive candidate target for diagnosis and treatment of autoimmune diseases, and possibly other immune system disorders. Indeed, when transgenic mice that overexpressed BAFF were treated with a soluble receptor of BAFF, B-cell population and severity of SLE-like disease were significantly reduced (Gross, J. A., et al., Nature, 404(6781): p. 995-9 (2000)). In addition, expression of APRIL is upregulated in many tumors and a soluble form of BCMA was found to inhibit tumor growth (Rennert, P., et al., J Exp Med, 192(11): p. 1677-84 (2000)).

Agents that bind to BAFF and interrupt its interaction with one or more of its receptors can be used to treat autoimmune diseases and other immune or non-immune disorders associated with inappropriate or abnormal BAFF expression. There is currently a need for agents that can serve as agonists or antagonists of BAFF. Further development of novel agents to serve as human therapeutic agents, which are effective in interrupting BAFF and its interaction with one or more receptors of BAFF, is hampered by the lack of structural information of BAFF. That information is provided for the first time by the present invention.

### SUMMARY OF THE INVENTION

Applicants have solved the above-identified problem by providing compositions, which can be crystallizable, and crystals of BAFF and methods for using such compositions and crystals.

This invention also provides the structure coordinates of BAFF.

This invention also provides methods for determining at least a portion of the three-dimensional structures of molecules or molecular complexes which contain at least some structurally similar features to BAFF. This invention also provides methods for determining at least a portion of the homology model structure of molecules or molecular complexes which contain at least some structurally similar features to BAFF. In a preferred embodiment, this invention provides methods for determining a homology model of APRIL. This invention also provides homology model coordinates of APRIL.

This invention also provides methods for designing chemical entities, compounds, such as agonists and antagonists of BAFF, and variants of agonists or antagonists of BAFF. This invention further relates to compositions comprising the chemical entities, the compounds, such as agonists and antagonists of BAFF, and variants of agonists or antagonists of BAFF, wherein such chemical entities, compounds, including agonists or antagonists of BAFF, and variants are rationally designed by means of the structure coordinates of BAFF, or portions thereof. The invention further relates to use of the above-identified chemical entities, compounds, such as agonists and antagonists of BAFF, and variants of an agonist or antagonist of BAFF, to treat conditions associated with inappropriate or abnormal BAFF activation or inactivation in a subject.

This invention also provides a computer, which comprises a storage medium comprising a data storage material, for producing three-dimensional representations of molecules or molecular complexes of BAFF and methods for using these three-dimensional representations to design: 1) chemical entities and compounds that associate with BAFF, 2) compounds, such as potential agonists or antagonists of BAFF, and 3) variants of an agonist or antagonist of BAFF, by using computational means to perform a fitting operation between chemical entities, compounds, such as agonists and antagonists of BAFF, and variants of an agonist or antagonist of BAFF and the molecules or molecular complexes of this invention. This invention also provides the chemical entities, the compounds, such as agonists and antagonists of BAFF, and variants of an agonist or antagonist of BAFF, and compositions comprising the chemical entities, the compounds and the variants.

This invention also provides methods for designing variants of BAFF. In a preferred embodiment, the variants of BAFF designed by these methods bind to a subset of the receptors that bind to BAFF. In another preferred embodiment, the variants of BAFF bind one or more receptors of BAFF with higher or lower affinity than native BAFF. This invention also provides these BAFF variants.

The foregoing and other objects (such as methods of using a homology model of APRIL to design chemical entities, compounds, including agonists or antagonists of BAFF, and variants of agonists or antagonists of APRIL), features and advantages of the present invention, as well as the invention itself, will be more fully understood from the following description of preferred embodiments.

### BRIEF DESCRIPTION OF THE DRAWINGS

The following abbreviations are used in FIGS. 8 and 10:

“Atom type” refers to the element whose coordinates are measured. The first letter in the column defines the element. “Resid” refers to the amino acid residue identity. Amino acid residue numbers for BAFF or APRIL correspond to the numbering system in full-length human BAFF (FIG. 9a; SEQ ID NO: 1) or full-length APRIL (FIG. 9c; SEQ ID NO:3), respectively.

“X, Y, Z” define the atomic position of the element measured.

“B” is a thermal factor that measures movement of the atom around its atomic center.

“Occ” is an occupancy factor that refers to the fraction of the molecules in which each atom occupies the position specified by the coordinates. A value of “1” indicates that each atom has the same conformation, i.e., the same position, in all molecules of the crystal.

“Mol” refers to the molecules in the asymmetric unit.

FIG. 1 depicts a view of a representative region of the final 2Fo-Fc electron density map contoured at 1.0 σ.

FIG. 2a depicts a ribbon diagram of BAFF trimer. The front monomer has β-strands labeled while the back monomers are unlabelled in dark and light grey. The figure was made with RIBBONS (Carson, J. Appl. Cryst., 24, pp. 958-961 (1991)).

FIG. 2b depicts a space filling model of BAFF trimer viewed along the 3-fold axis. The arrows point to the putative receptor binding sites. The figure was made with RIBBONS (Carson, J. Appl. Cryst., 24, pp. 958-961 (1991)).

FIG. 3a shows a sequence alignment of TNF family members based on structural superimpositions. The secondary structure assignment and numbering correspond to BAFF.

FIG. 3b shows superimposed Cα backbones of BAFF and TNF-α structures in stereo. BAFF is shown in dark grey and TNF-α in light grey. The β-strands of the structures superimpose well while the loops connecting the β-strands differ in the two structures.

FIG. 4 shows the solvent accessible surface of BAFF and APRIL trimers shaded according to electrostatic potential calculated with GRASP. Darker shaded areas represent positively charged regions or negatively charged regions. A schematic of the possible BAFF-R secondary structure is also shown. The arrows point to the putative location of the BAFF-R binding sites. In the BAFF structure the putative BAFF-R binding site is negatively charged, and will therefore compliment the positively charged BAFF-R secondary structural element which is positively charged. But the putative BAFF-R binding site in the APRIL structure is positively charged, and will thus repel the positively charged BAFF-R secondary structural element. The short vertical lines indicate the appropriate orientation of the BAFF and APRIL 3-fold axes.

FIG. 5 shows a diagram of a system used to carry out the instructions encoded by the storage medium of FIGS. 6 and 7.

FIG. 6 shows a cross-section of a magnetic storage medium.

FIG. 7 shows a cross section of an optically-readable data storage medium.

FIG. 8 (8-1 to 8-104) lists the atomic structure coordinates for the extracellular domain of human BAFF, as derived by X-ray crystallography from crystals of that polypeptide in protein data bank (PDB) format. Molecules A, B, C, K, L and M represent two BAFF trimers in the asymmetric unit.

FIG. 9a shows the amino acid (aa) sequence of full-length human BAFF (SEQ ID NO: 1). The section of BAFF present in the construct crystallized is shown in bold.

FIG. 9b shows myc-tagged human BAFF amino acids 136 to 285 fusion protein (SEQ ID NO: 2). The myc tag is in bold.

FIG. 9c shows the amino acid (aa) sequence of human APRIL (SEQ ID NO: 3; Swiss-Prot entry 075888). Bracketed residues correspond to residues 114 to 250.

FIG. 10 (10-1 to 10-50) lists the homology model structure coordinates for the extracellular domain of APRIL in protein data bank (PDB) format, as derived by homology modeling based on the X-ray diffraction from crystals of a BAFF polypeptide.

### DETAILED DESCRIPTION OF THE INVENTION

The following discussion illustrates and exemplifies the variety of contexts and circumstances in which the invention can be practiced, as well as providing specific embodiments of the invention.

Throughout this specification and claims, the word “comprise,” or variations such as “comprises” or “comprising,” will be understood to imply the inclusion of a stated integer or group of integers but not the exclusion of any other integer or group of integers.

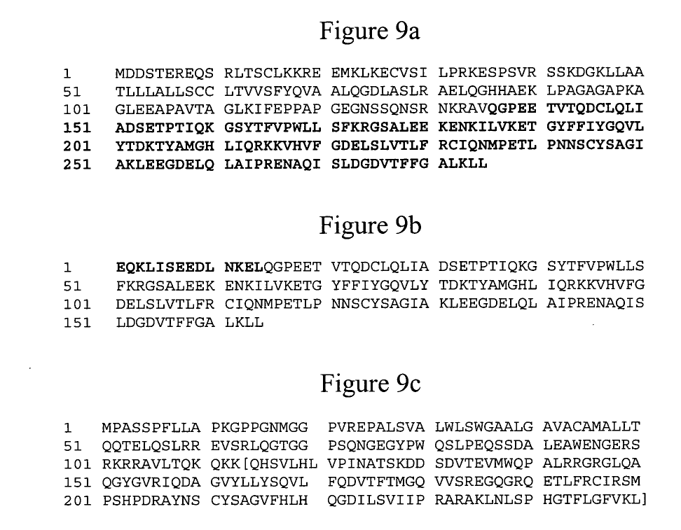

In addition, amino acid residue numbers given throughout the specification and claims for BAFF or APRIL correspond to the numbering system in full-length human BAFF (FIG. 9a; SEQ ID NO: 1) or full-length APRIL (FIG. 9c; SEQ ID NO:3), respectively.

AMINO ACIDS ABBREVIATIONS

| | | |
|---|---|---|
| A = | Ala = | Alanine |
| V = | Val = | Valine |
| L = | Leu = | Leucine |
| I = | Ile = | Isoleucine |
| P = | Pro = | Proline |
| F = | Phe = | Phenylalanine |
| W = | Trp = | Tryptophan |
| M = | Met = | Methionine |
| G = | Gly = | Glycine |
| S = | Ser = | Serine |
| T = | Thr = | Threonine |
| C = | Cys = | Cysteine |
| Y = | Tyr = | Tyrosine |
| N = | Asn = | Asparagine |
| Q = | Gln = | Glutamine |
| D = | Asp = | Aspartic Acid |
| E = | Glu = | Glutamic Acid |
| K = | Lys = | Lysine |
| R = | Arg = | Arginine |
| H = | His = | Histidine |

Compositions and Crystals

According to a preferred embodiment, the compositions of this invention are crystallizable. Those compositions comprise a BAFF polypeptide.

This invention also provides a crystal comprising a BAFF polypeptide.

The BAFF polypeptide is any BAFF polypeptide, preferably one that is capable of specifically binding to a receptor of BAFF. In a preferred embodiment, the BAFF polypeptide comprises the extracellular domain, or a portion thereof, of BAFF. In another preferred embodiment, the BAFF polypeptide comprises a polypeptide consisting of human BAFF (FIG. 9a; SEQ ID NO: 1) amino acid residues 136 to 285, which binds to a BCMA-Fc fusion protein. In a preferred embodiment, the BAFF is human BAFF (FIGS. 9a and 9b; SEQ ID NO: 1; SEQ ID NO: 2). In another preferred embodiment, the crystallizable composition comprises a trimer of BAFF polypeptides.

A BAFF polypeptide could be a fusion protein comprising BAFF, or a portion thereof, and one or more other proteins. The fusion protein could comprise BAFF, or a portion thereof, and one or more epitope tags, such as a myc tag.

Crystal Structures and Methods Using the Structure Coordinates That Define the Three-Dimensional Structure of BAFF

The crystallizable compositions provided by this invention are amenable to X-ray crystallography. Therefore, this invention also encompasses crystals of the crystallizable compositions. This invention also provides the three-dimensional structure as obtained by X-ray crystallography of a BAFF polypeptide at high resolution, such as at 2.8 Å resolution. See Example 1. In a preferred embodiment, the BAFF polypeptide is the extracellular domain of BAFF (for example, amino acids 136 to 285 of human BAFF (see FIG. 9b; SEQ ID NO: 2; or FIG. 9b; SEQ ID NO: 1)). In a preferred embodiment, the BAFF is human BAFF (see FIG. 9a; SEQ ID NO: 1). The BAFF polypeptide is preferably one that can bind to at least one receptor of BAFF, i.e., the BAFF polypeptide comprises a binding site for at least one receptor of BAFF.

In a preferred embodiment, the BAFF or the APRIL described herein is human BAFF or human APRIL.

The three-dimensional structures of other crystallizable compositions of this invention may also be determined by X-ray crystallography using X-ray crystallographic techniques routine in the art.

X-ray crystallography is a collection of techniques which allow the determination of the structure of a molecular entity. The techniques include crystallization of the entity, collection and processing of X-ray diffraction intensities, determination of phases (by, e.g., multiple isomorphous replacement, molecular replacement or difference Fourier techniques) and model building and refinement.

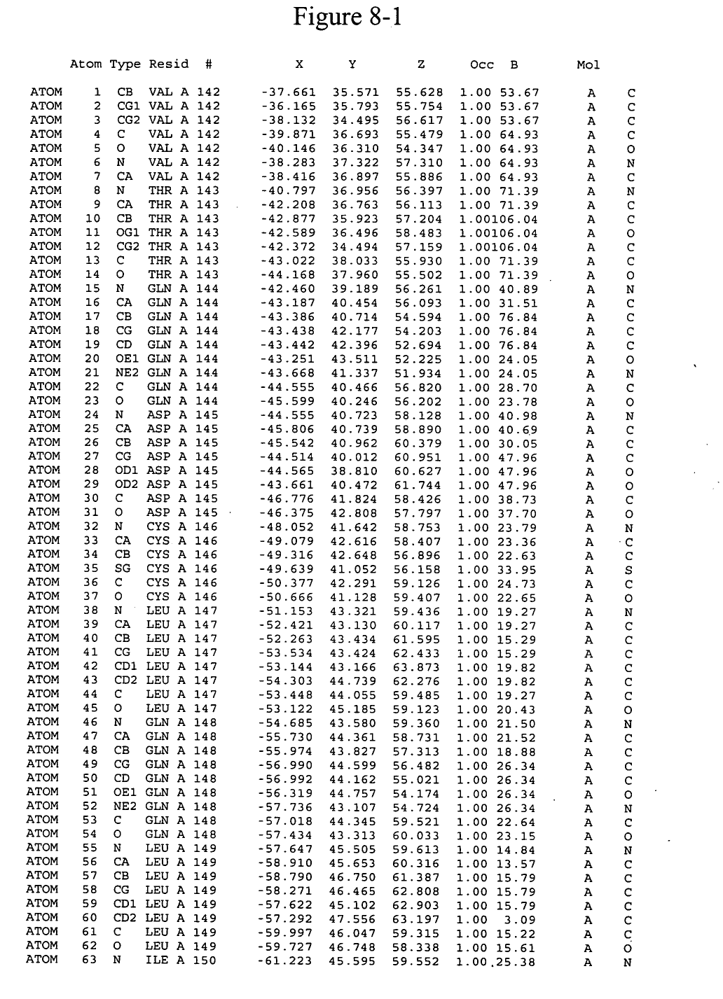

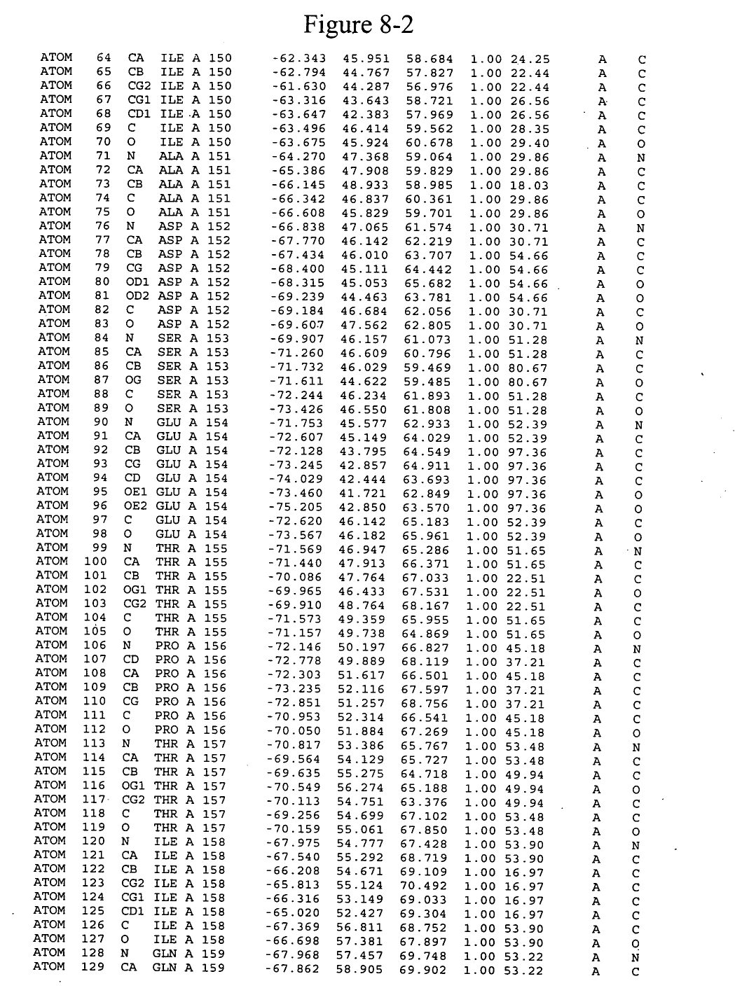

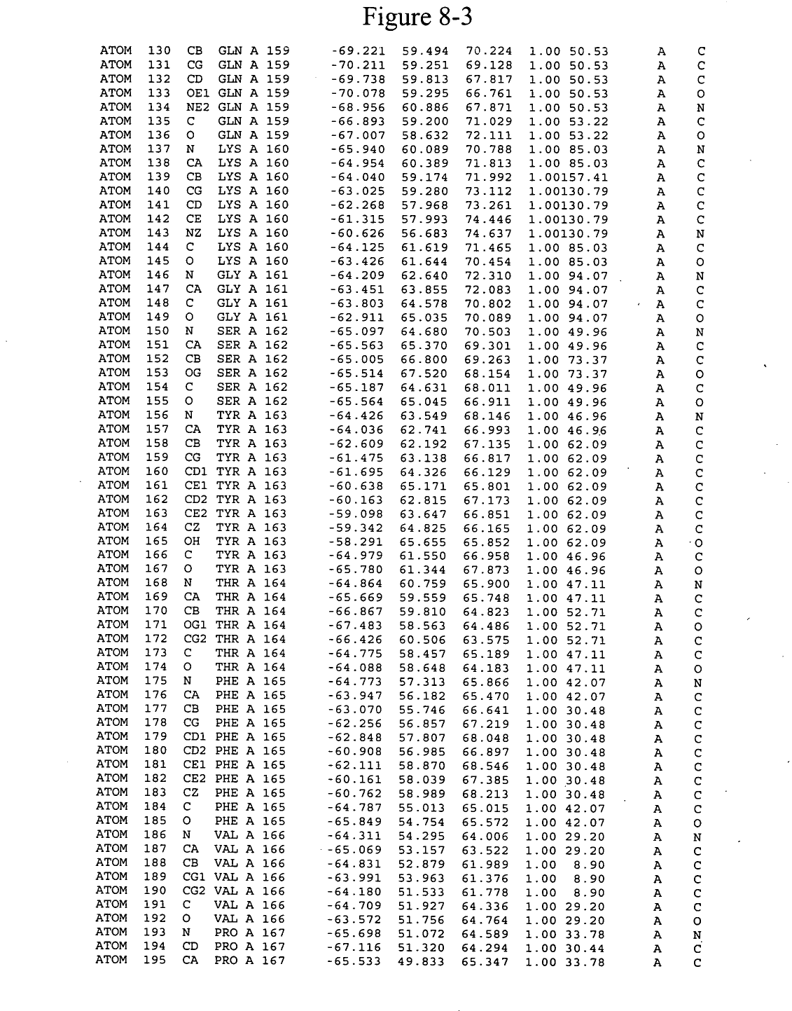

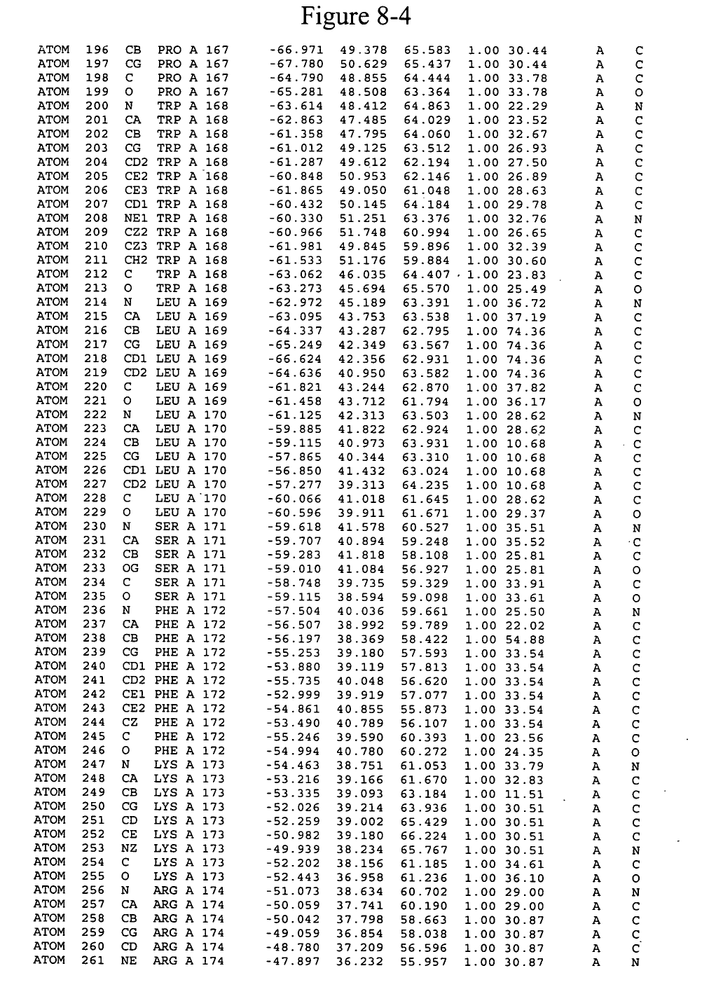

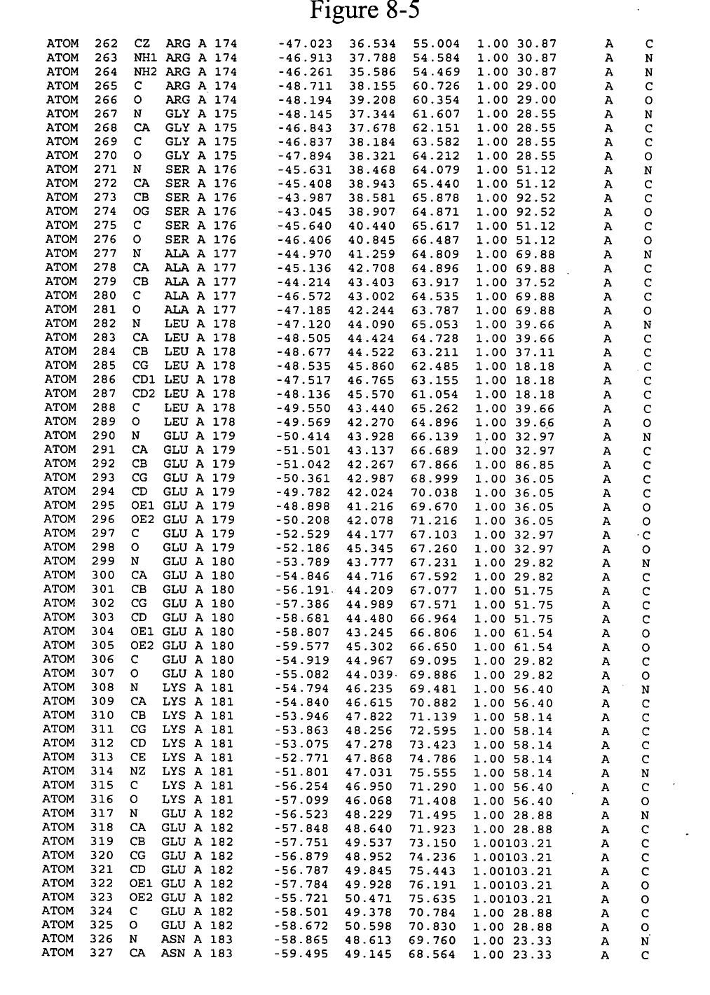

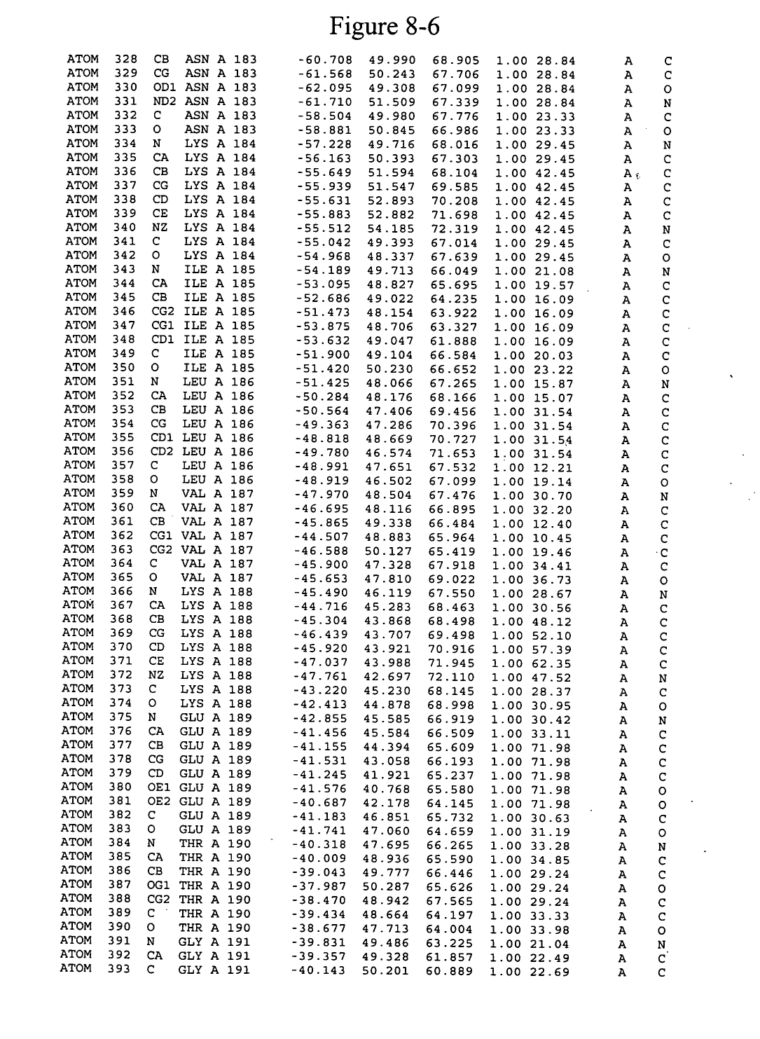

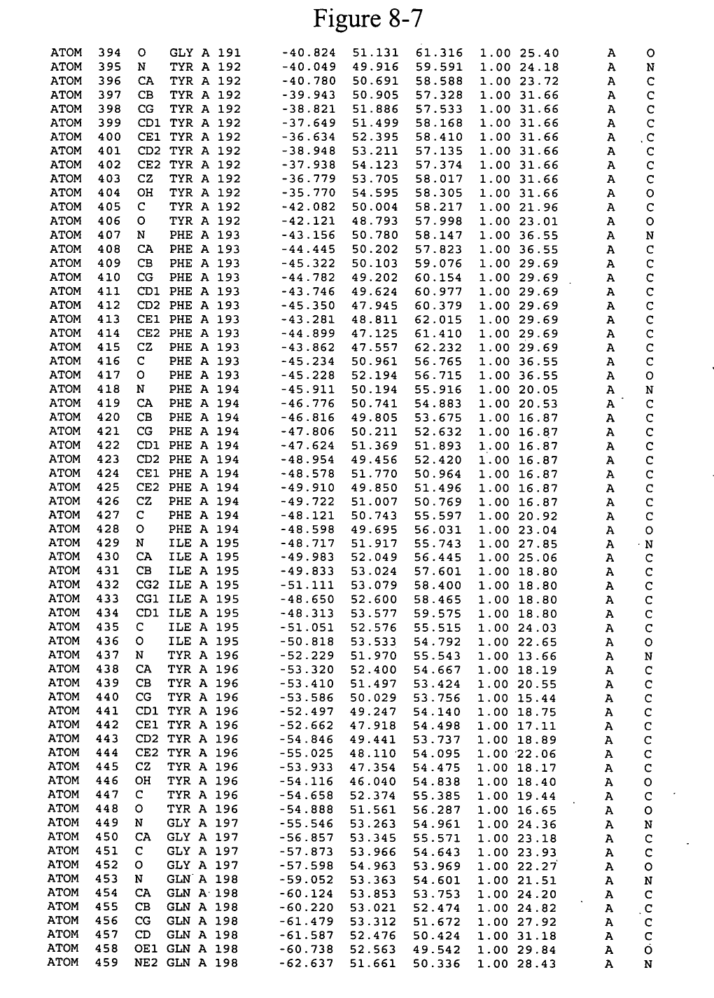

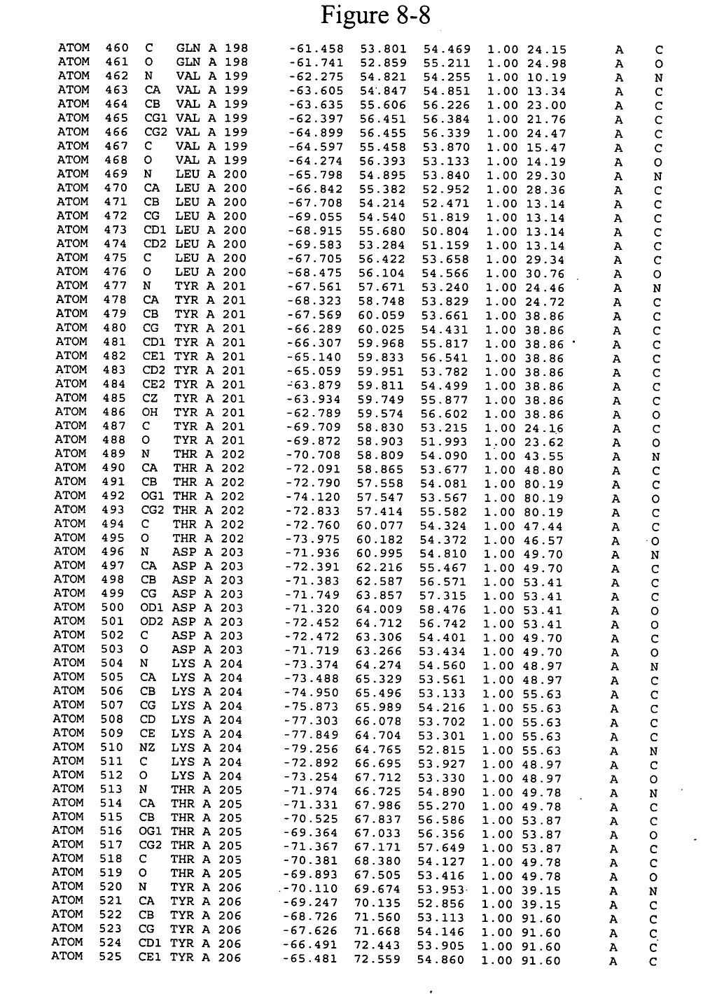

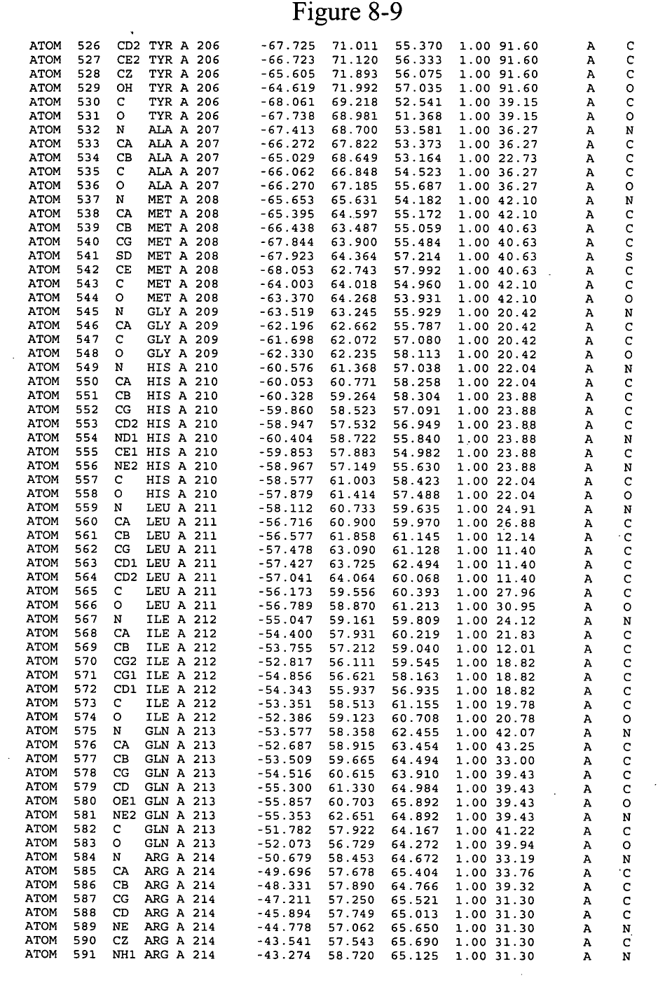

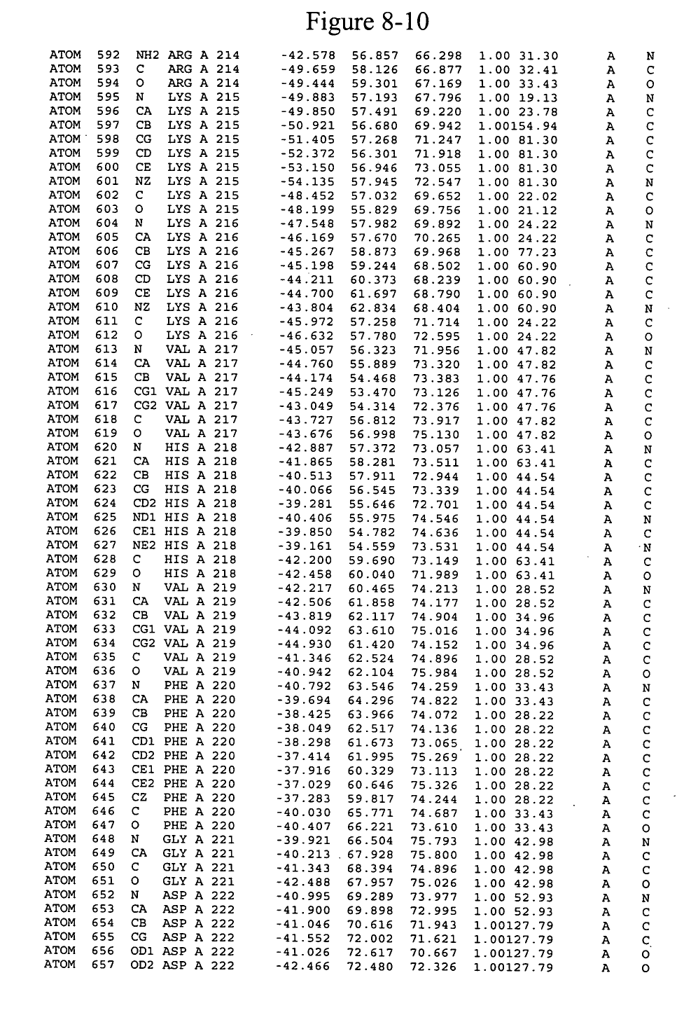

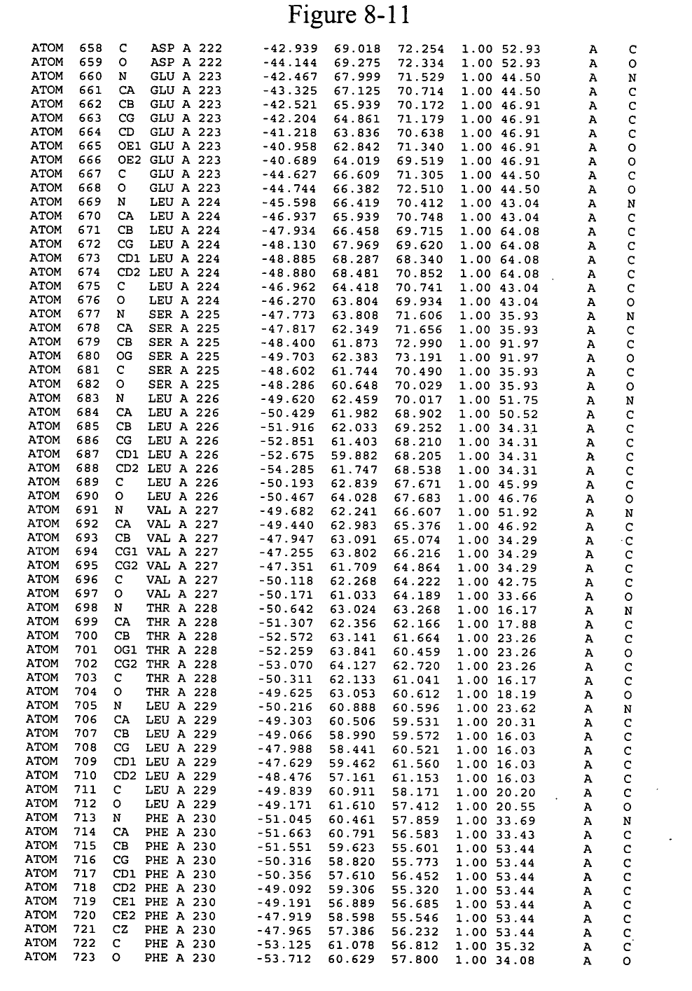

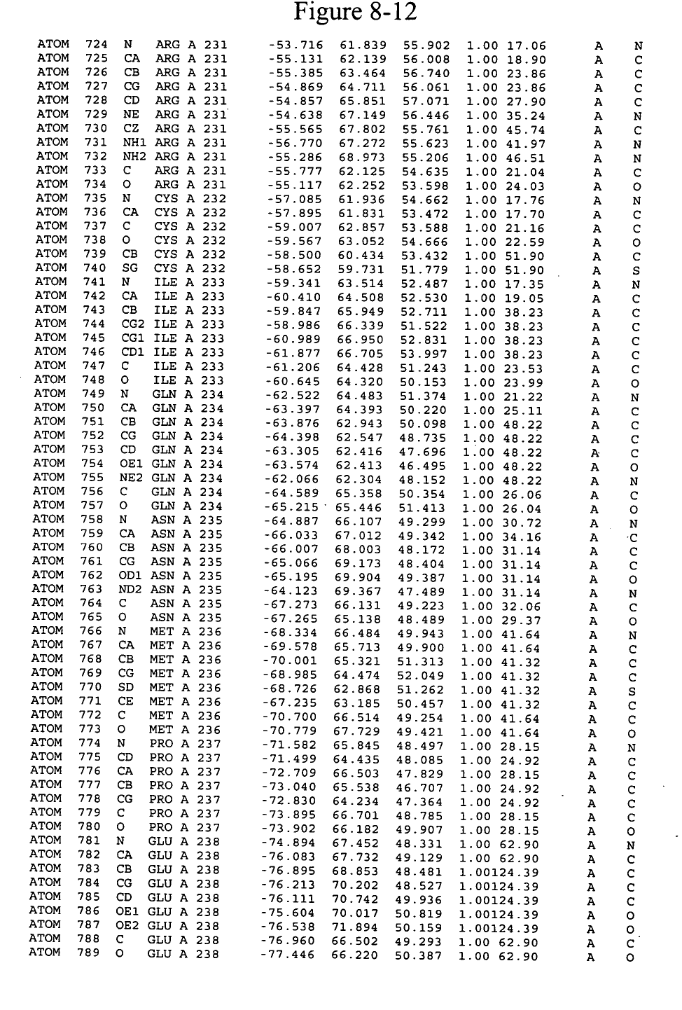

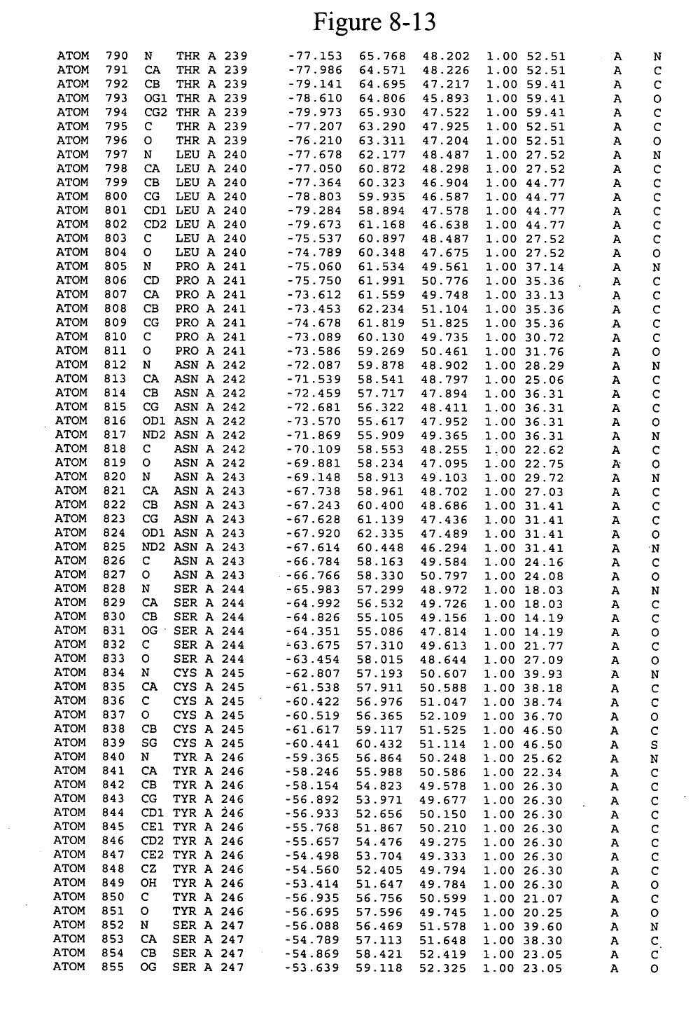

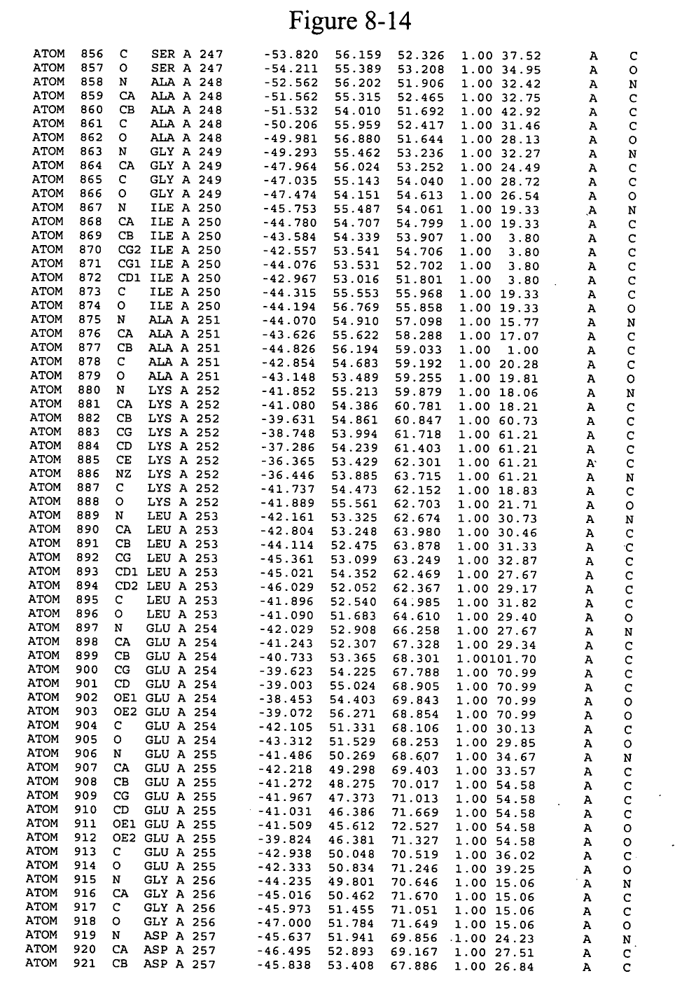

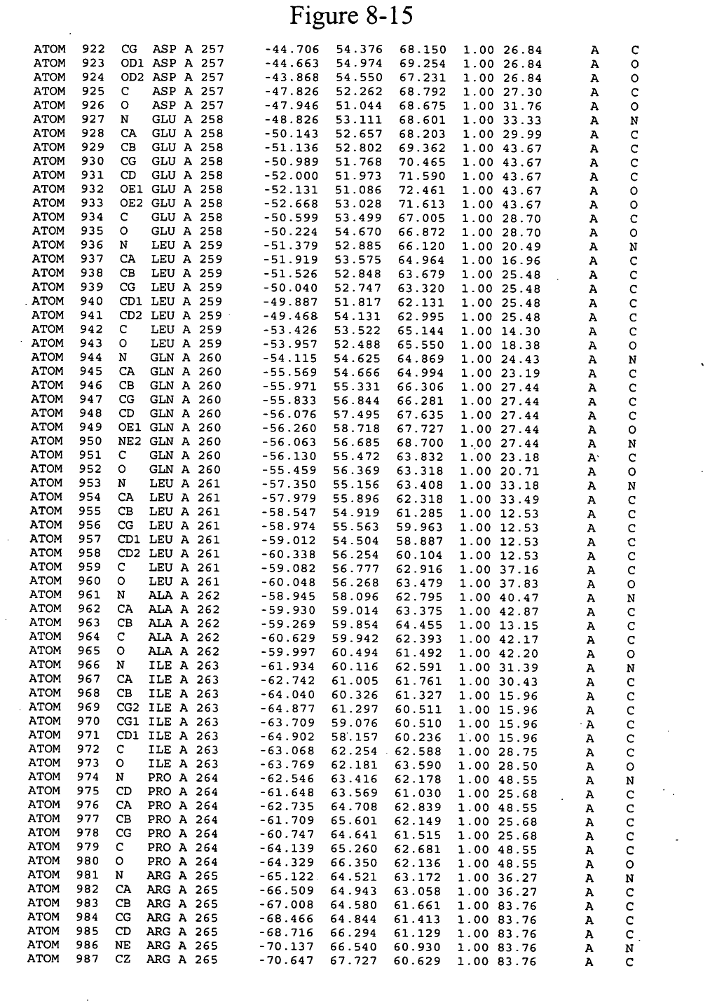

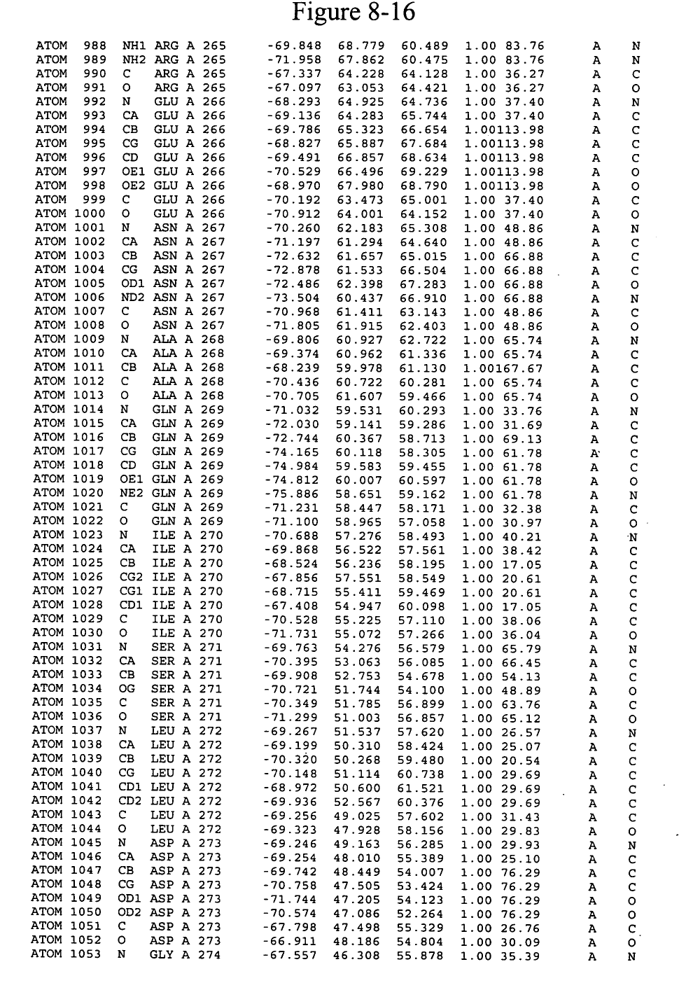

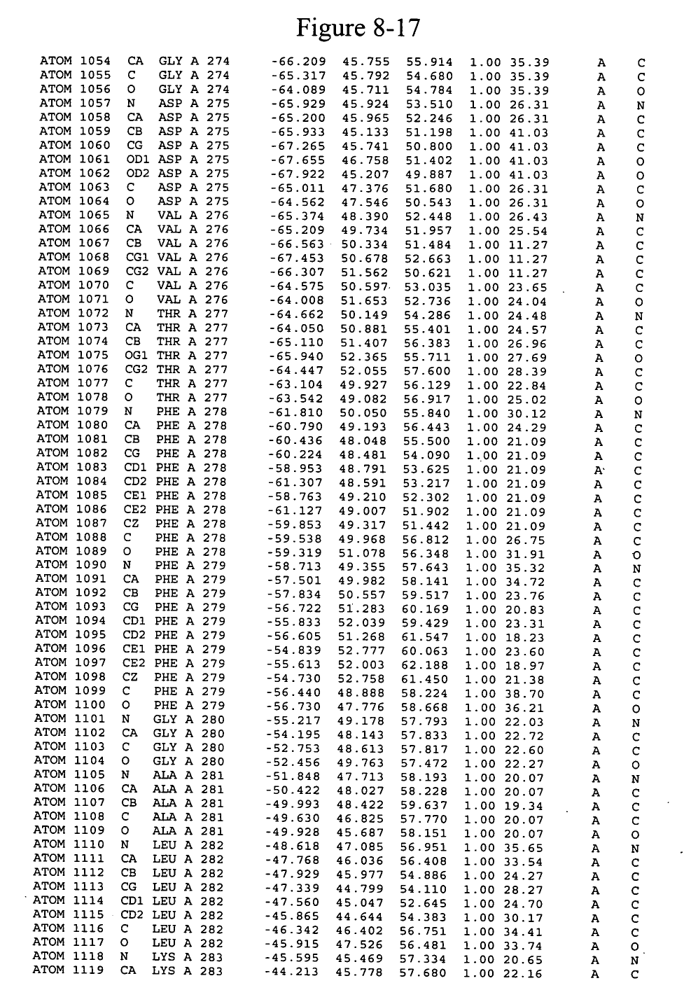

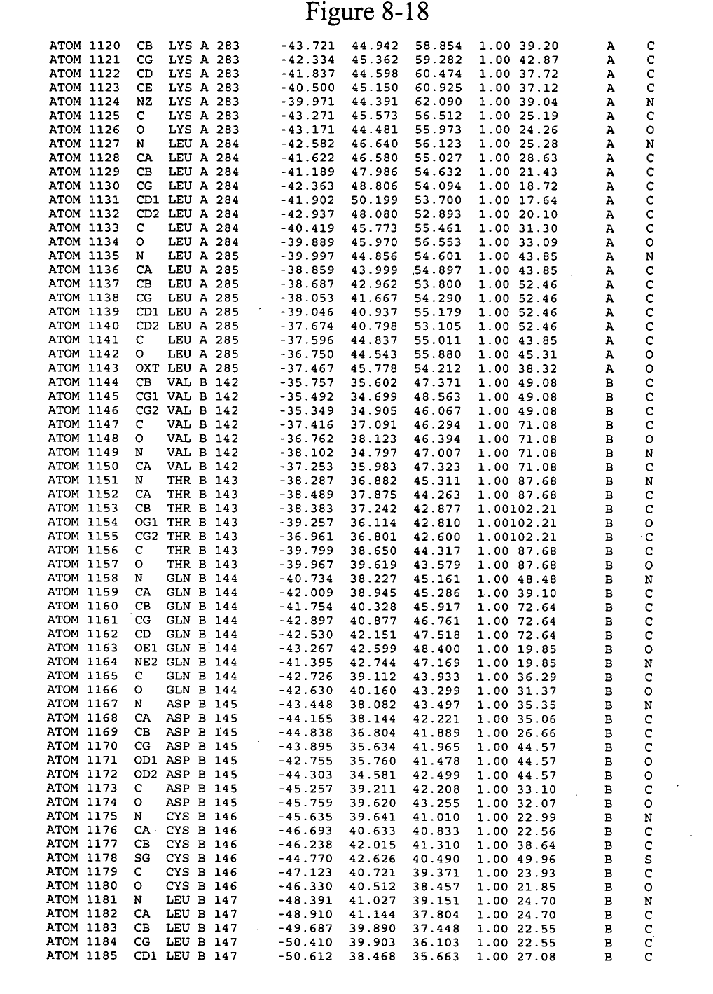

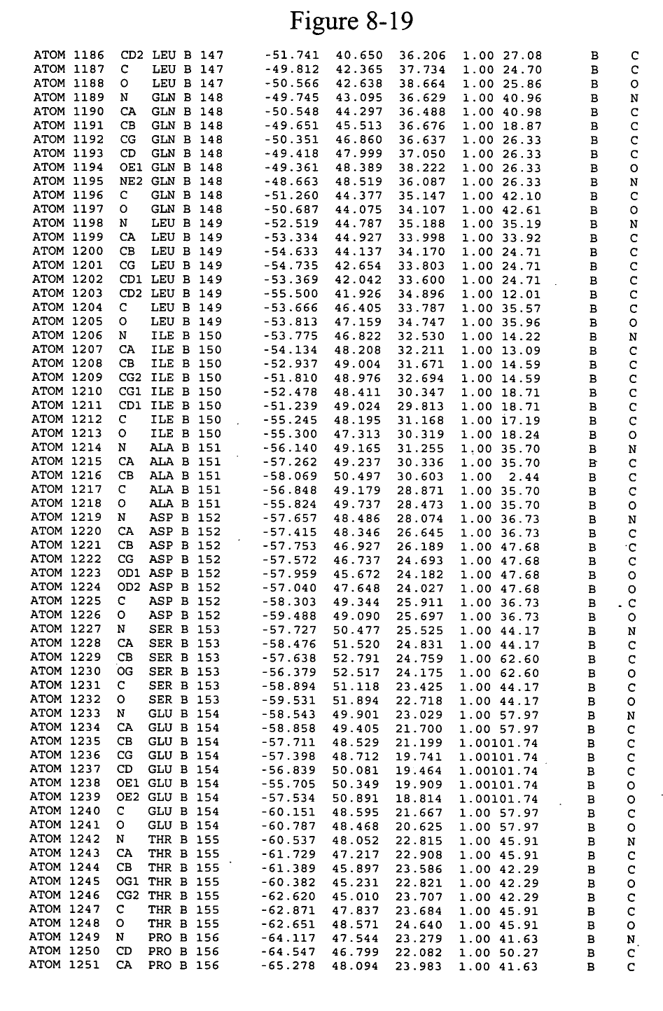

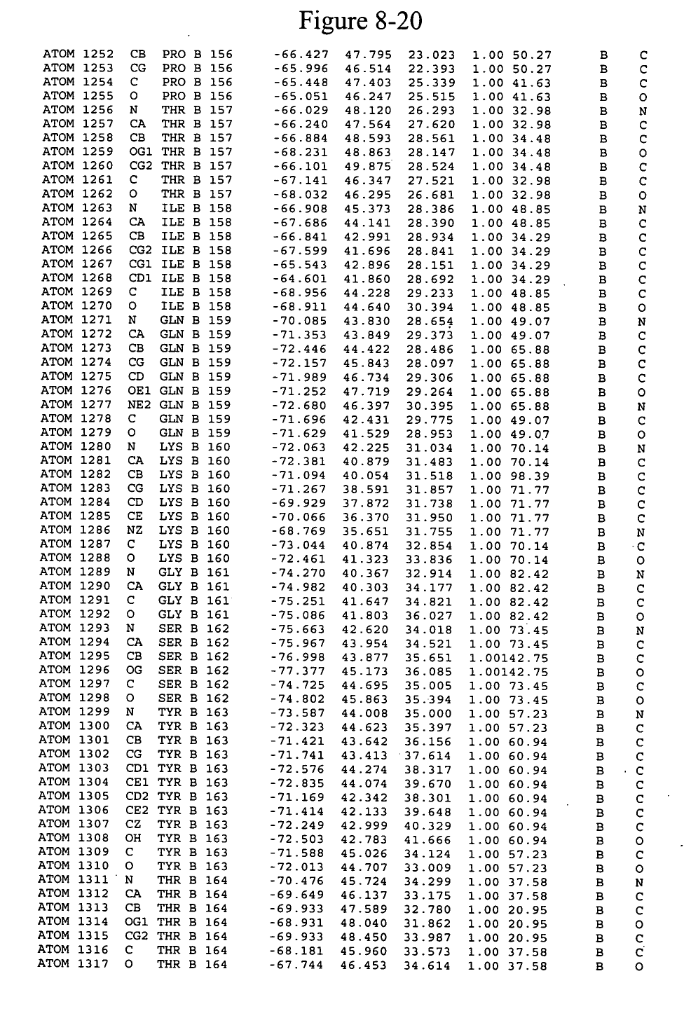

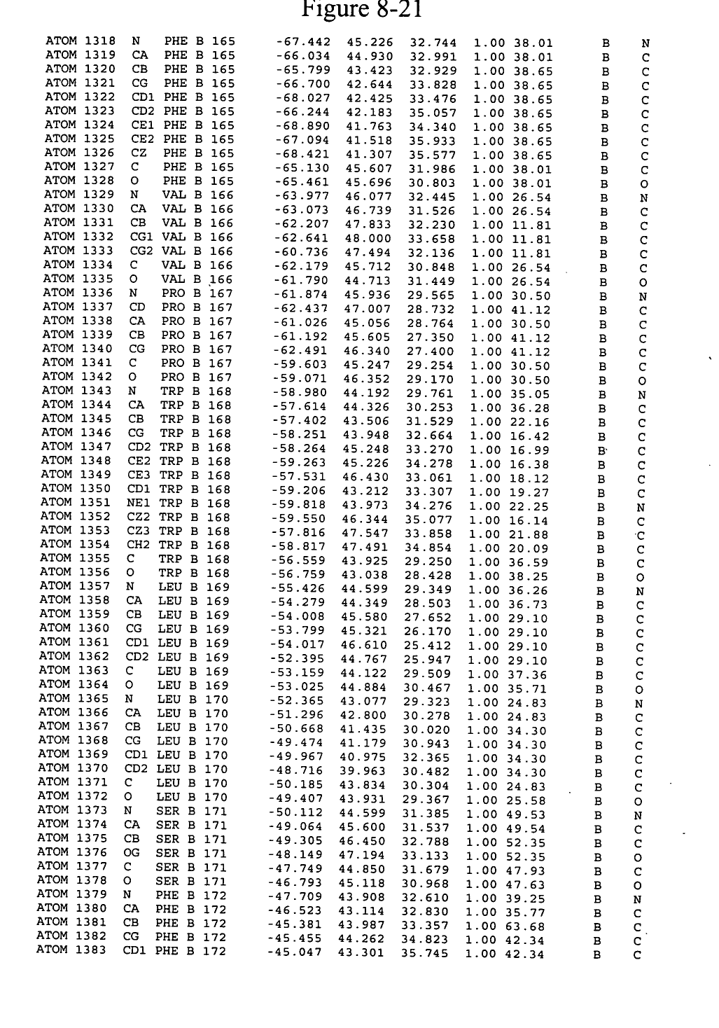

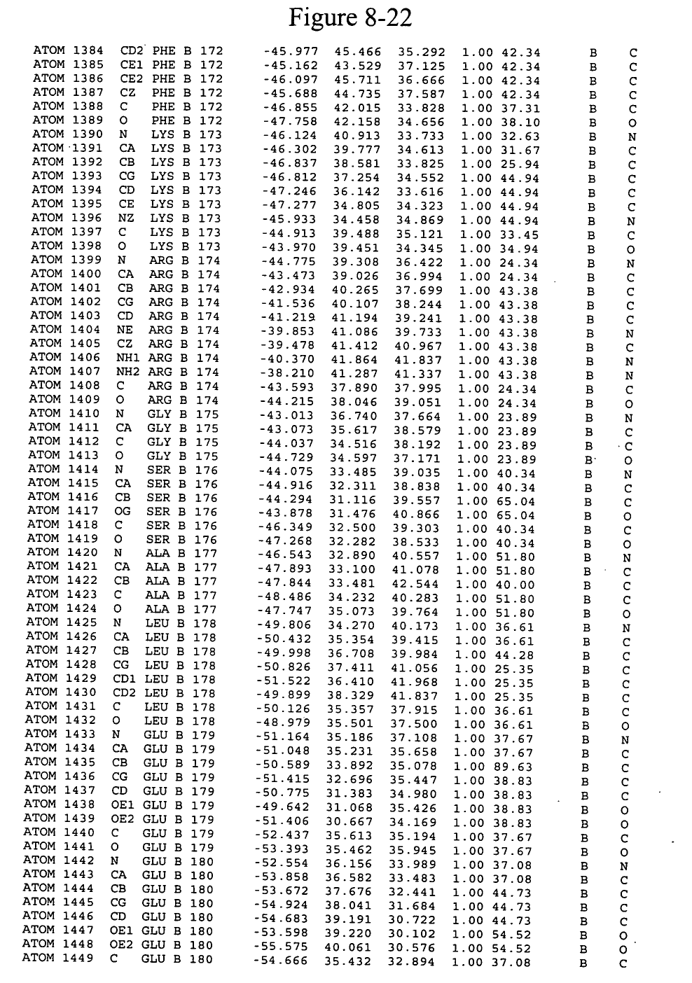

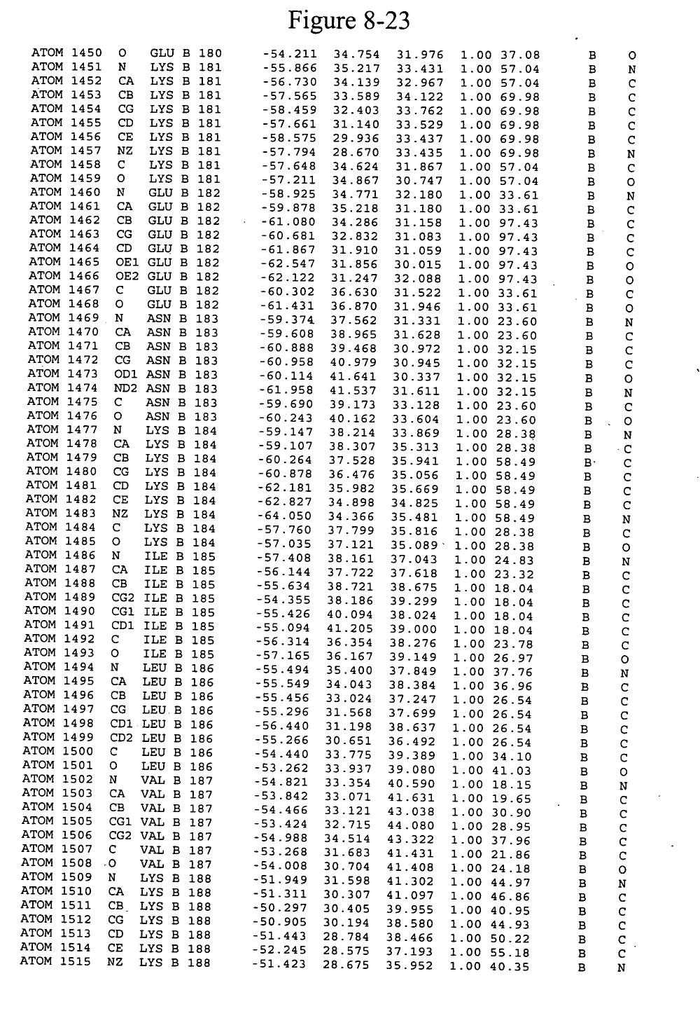

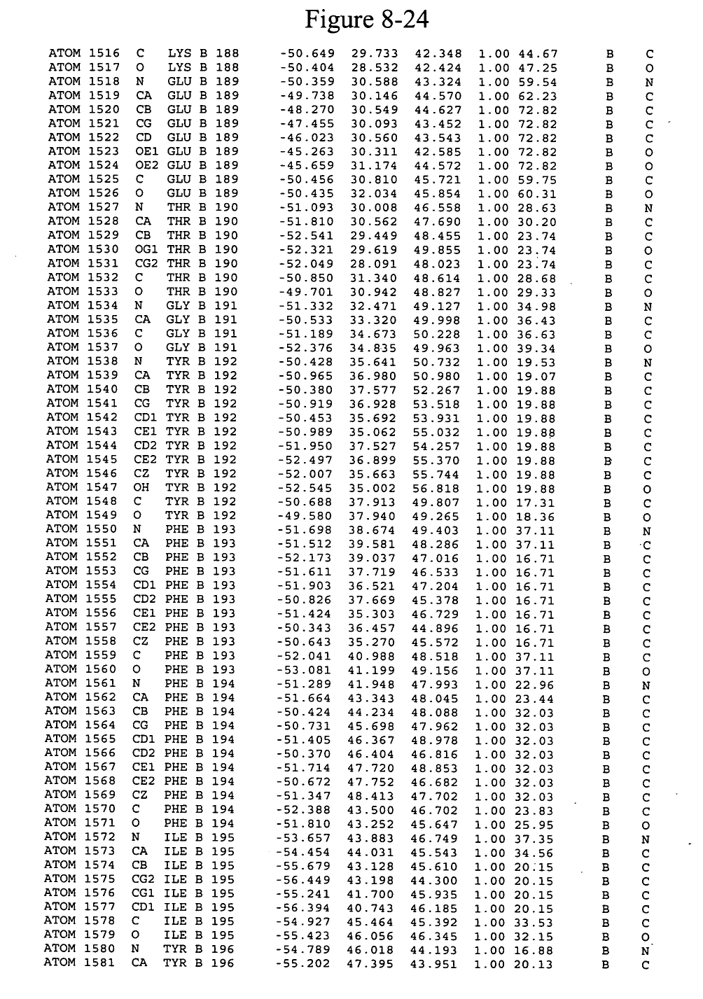

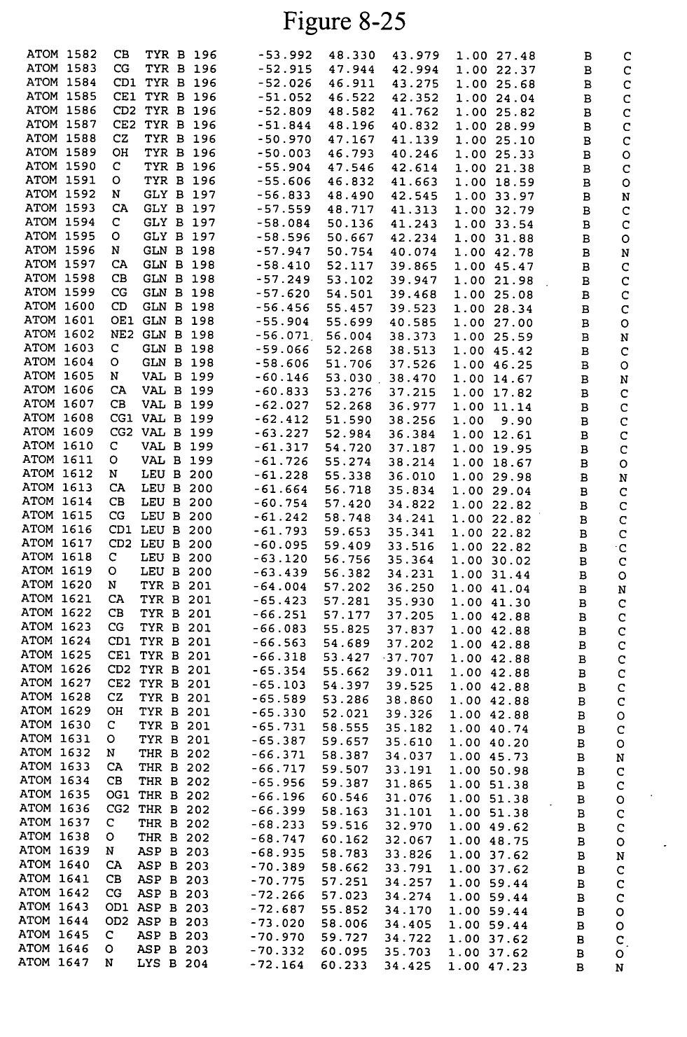

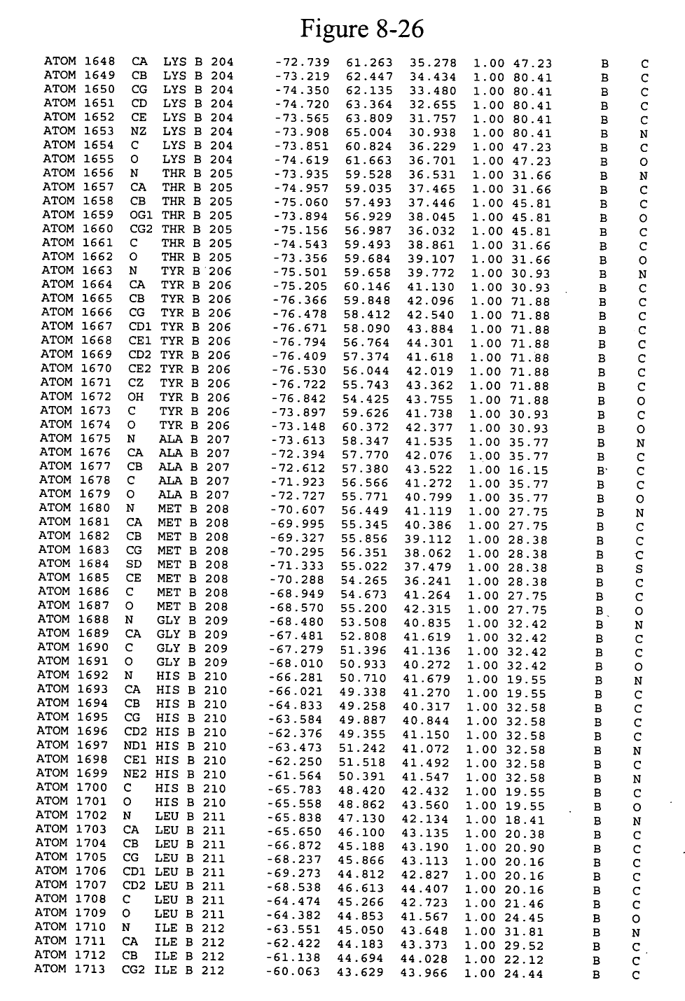

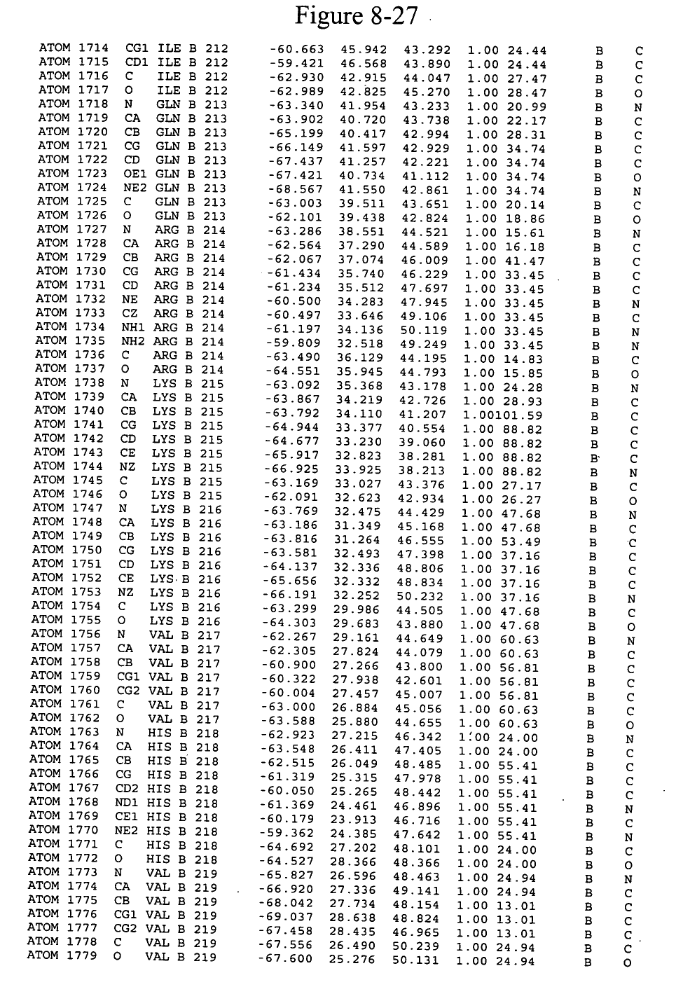

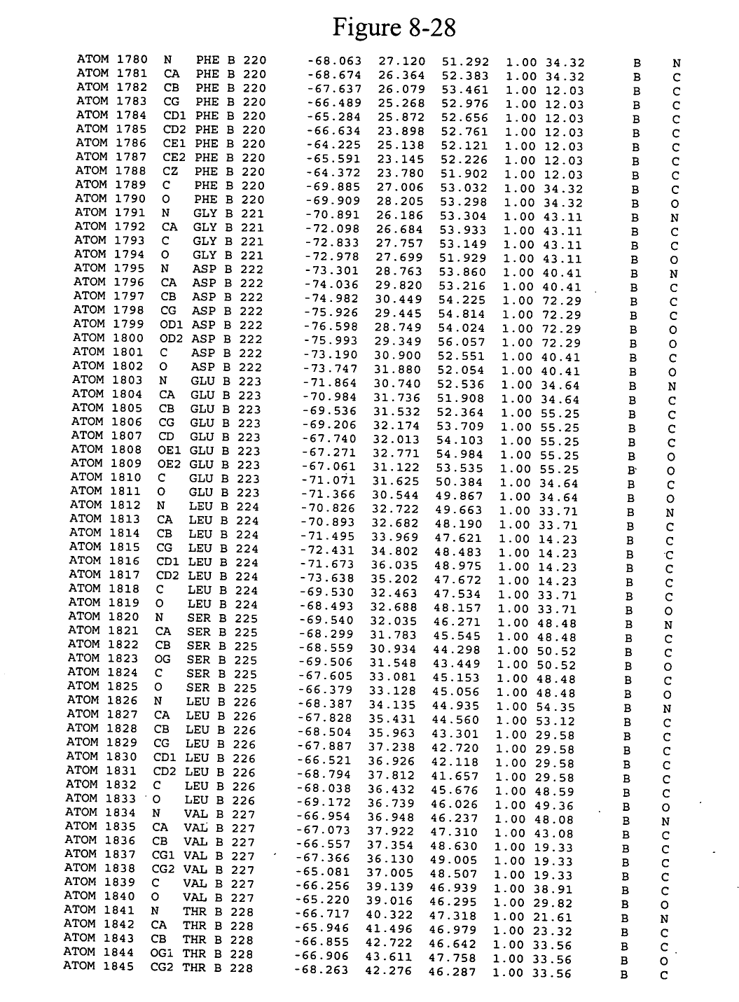

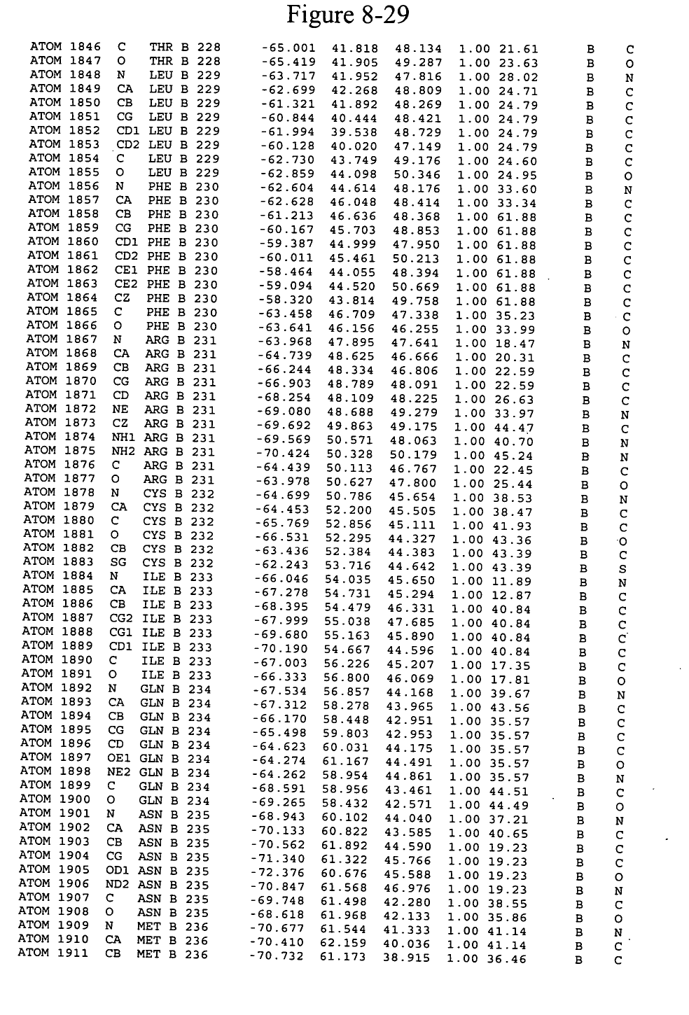

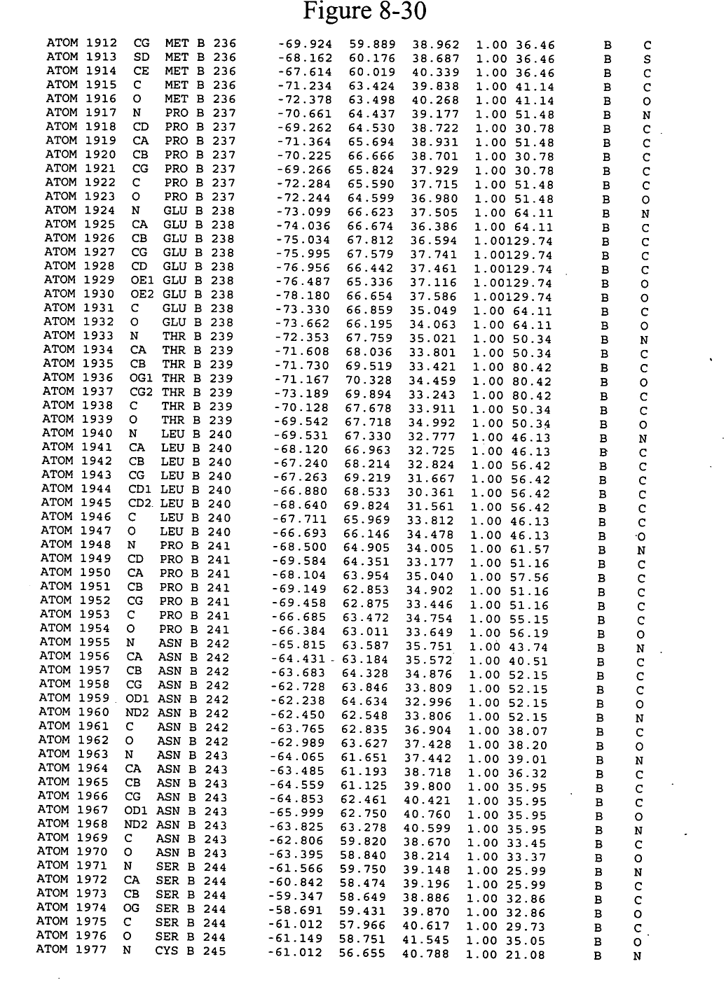

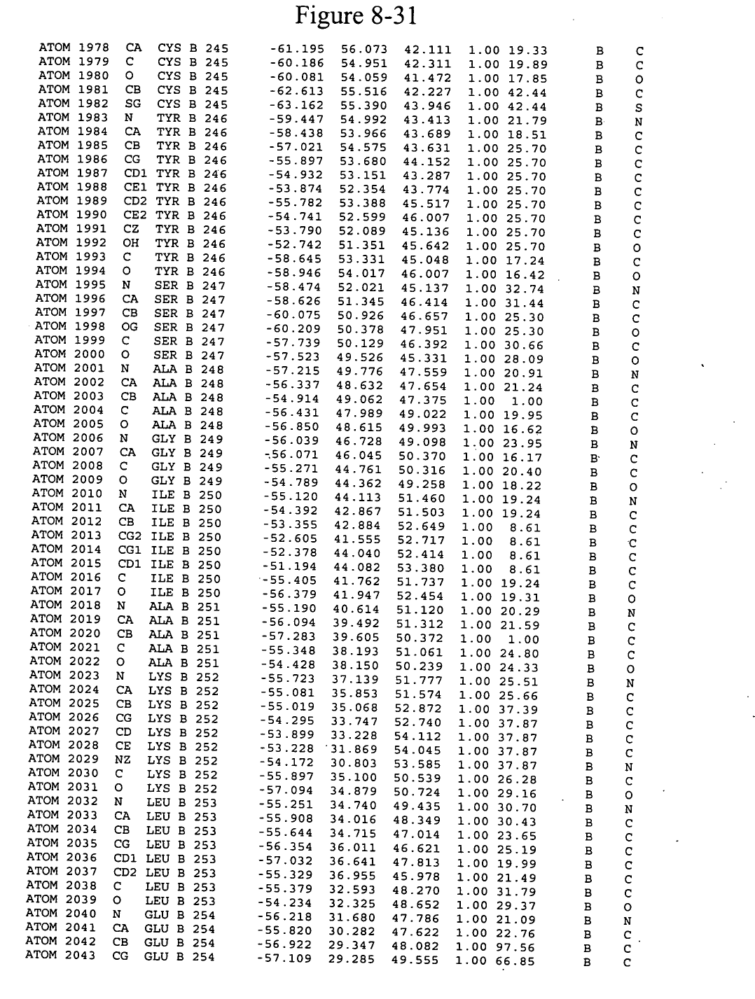

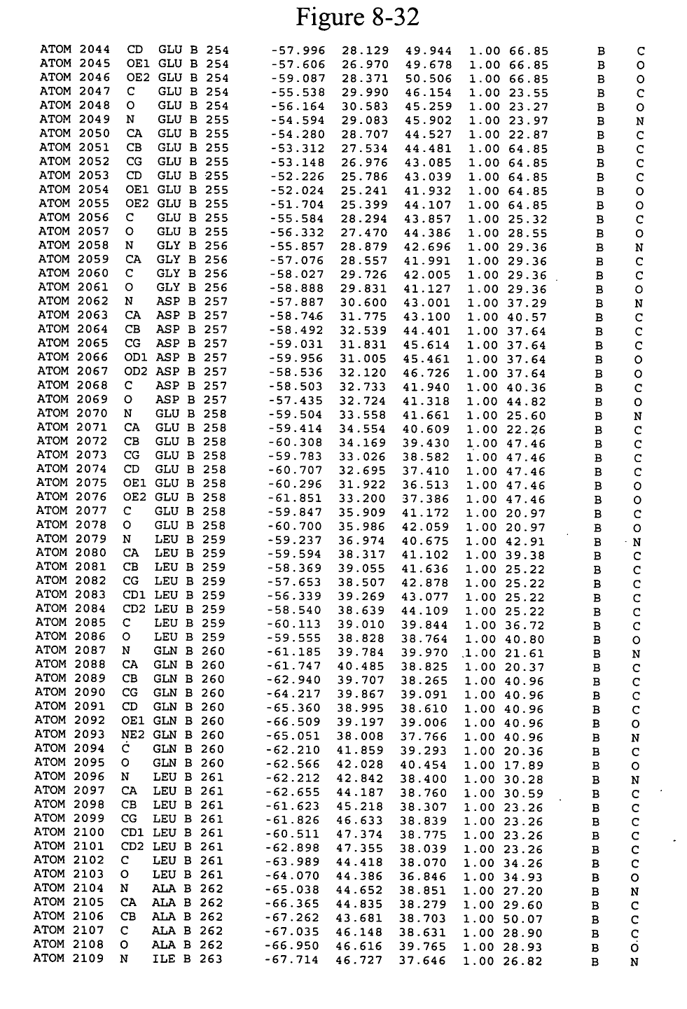

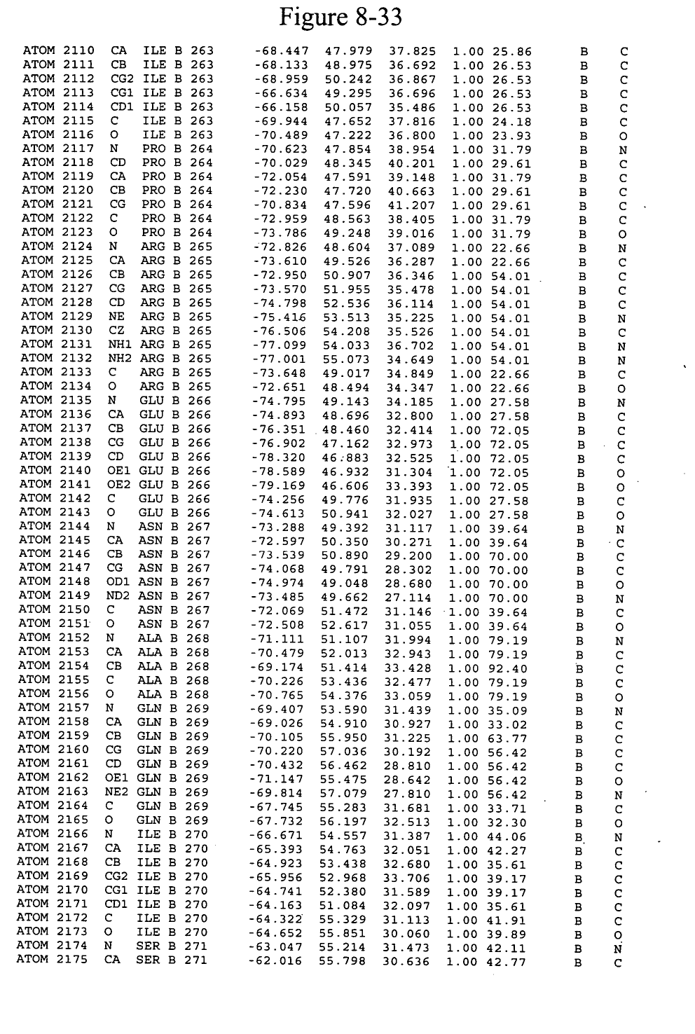

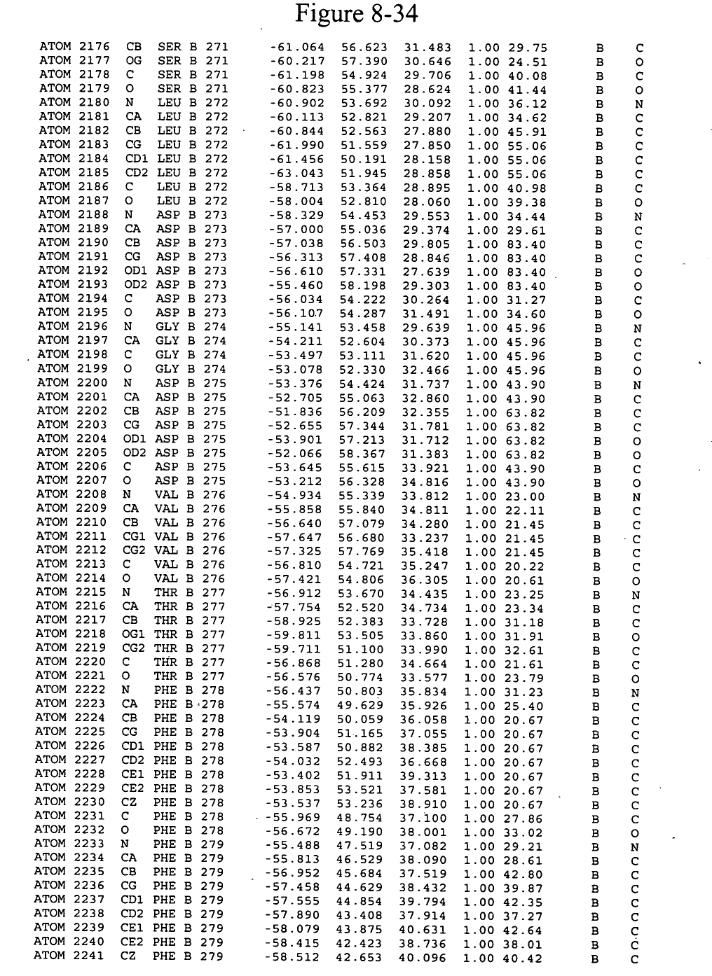

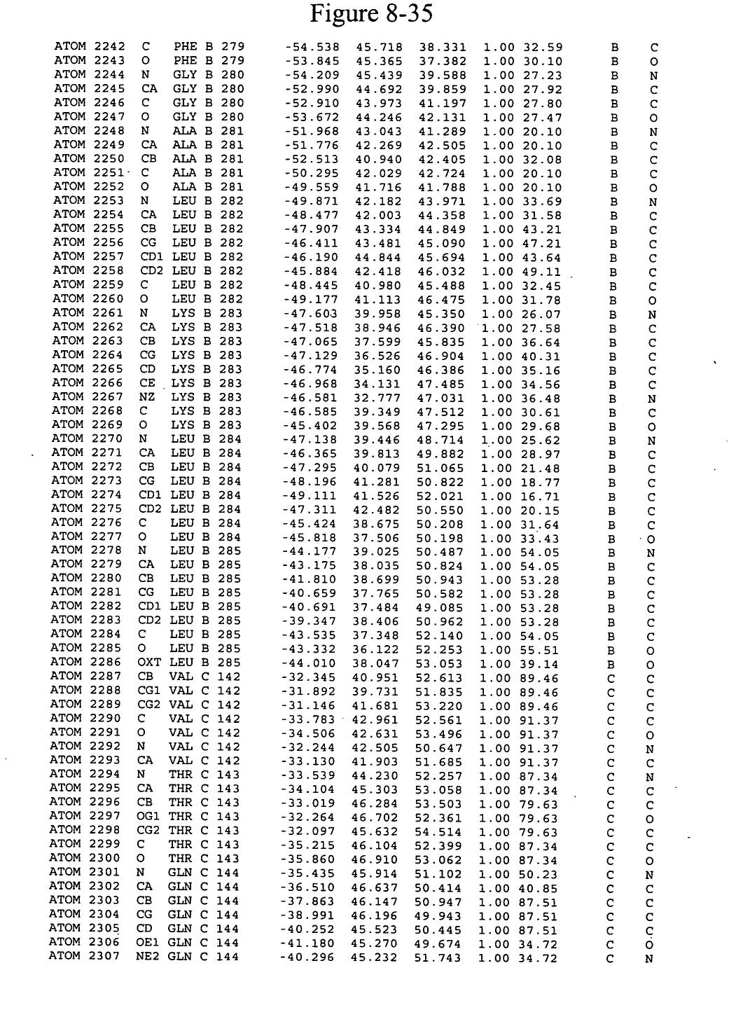

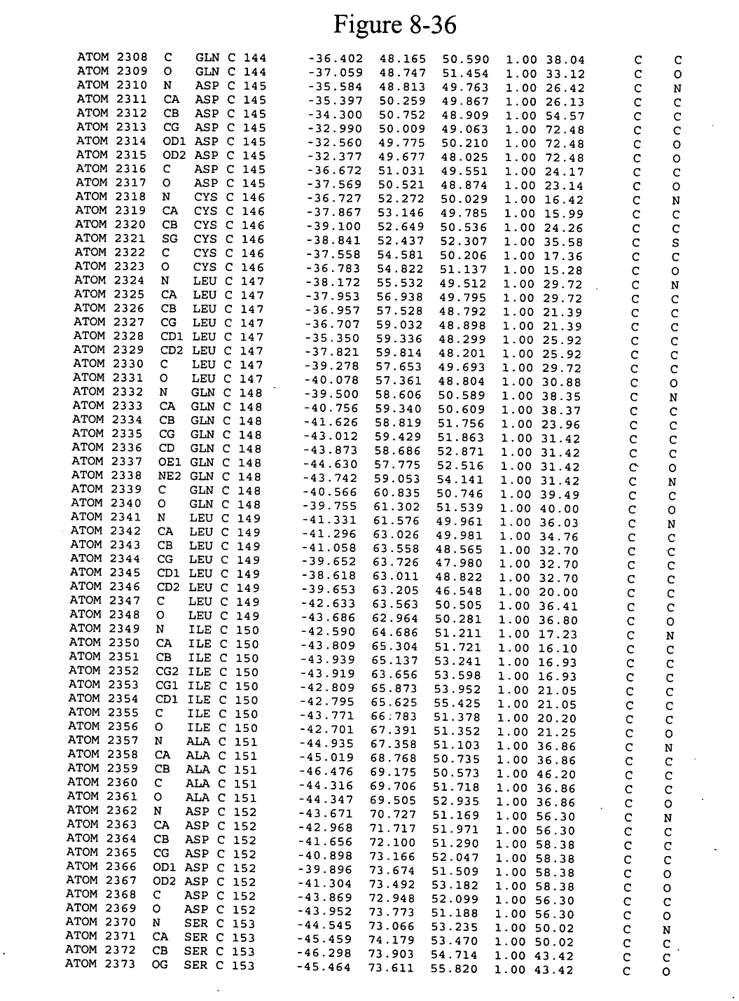

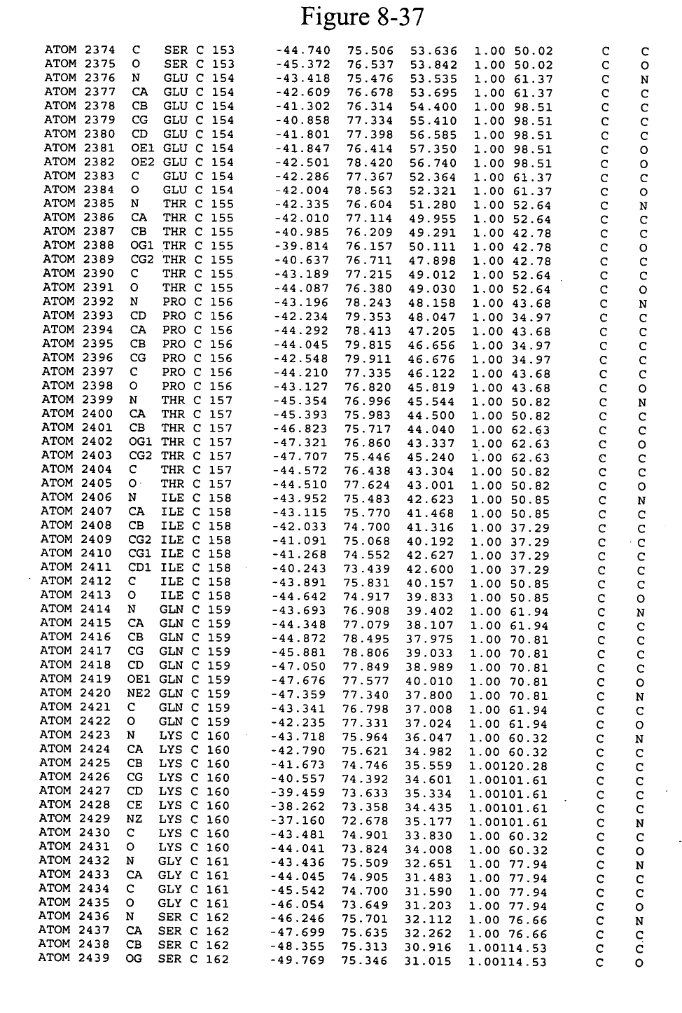

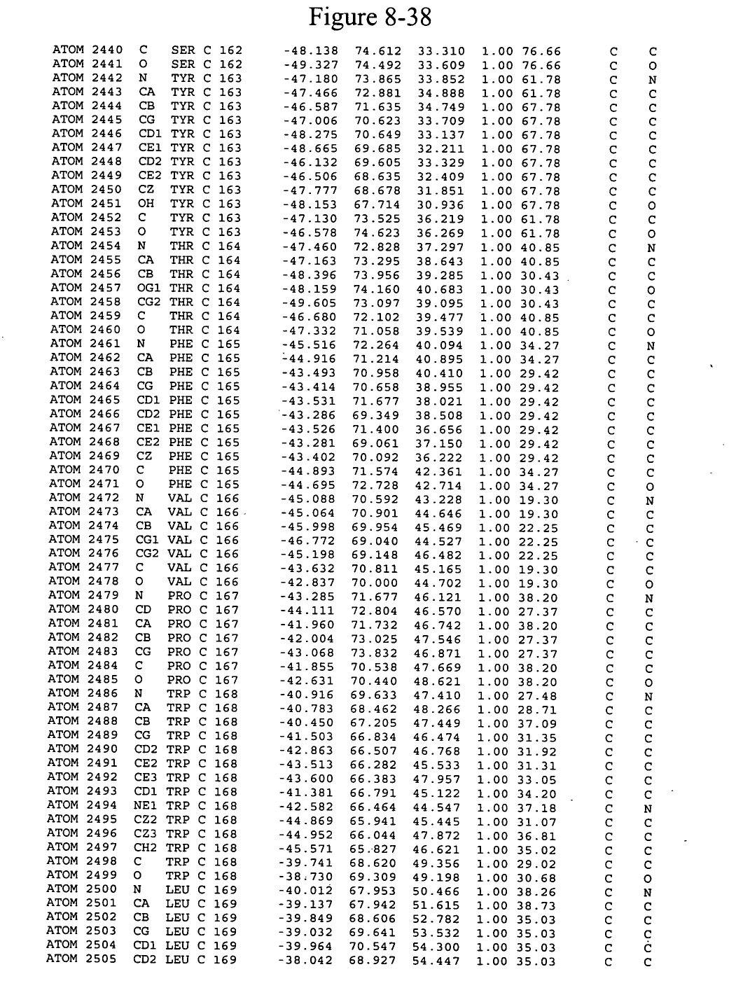

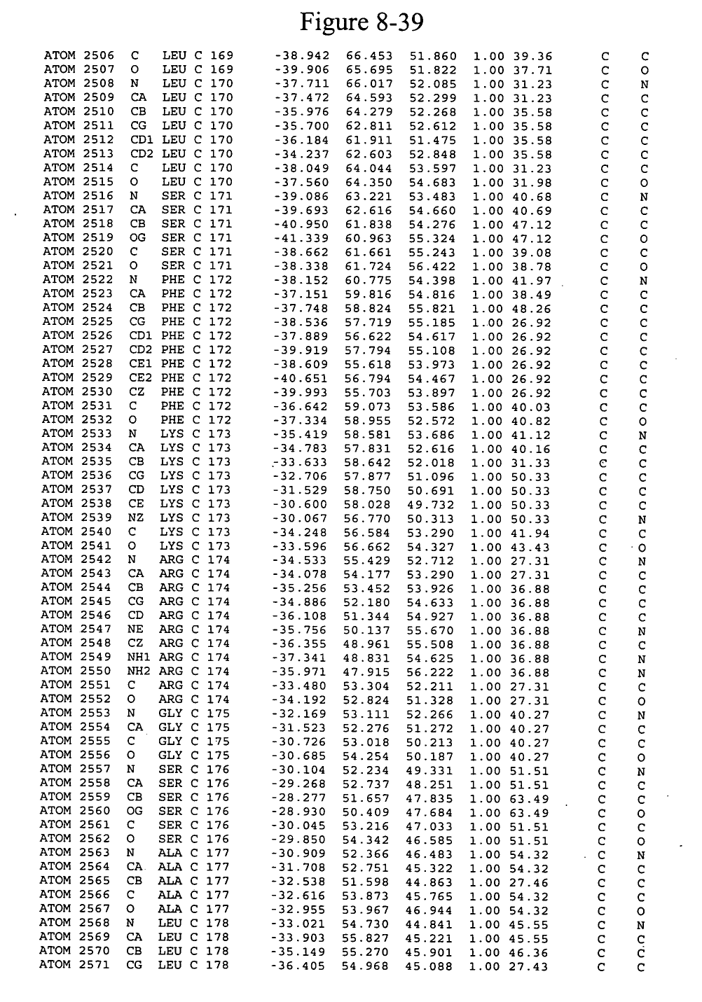

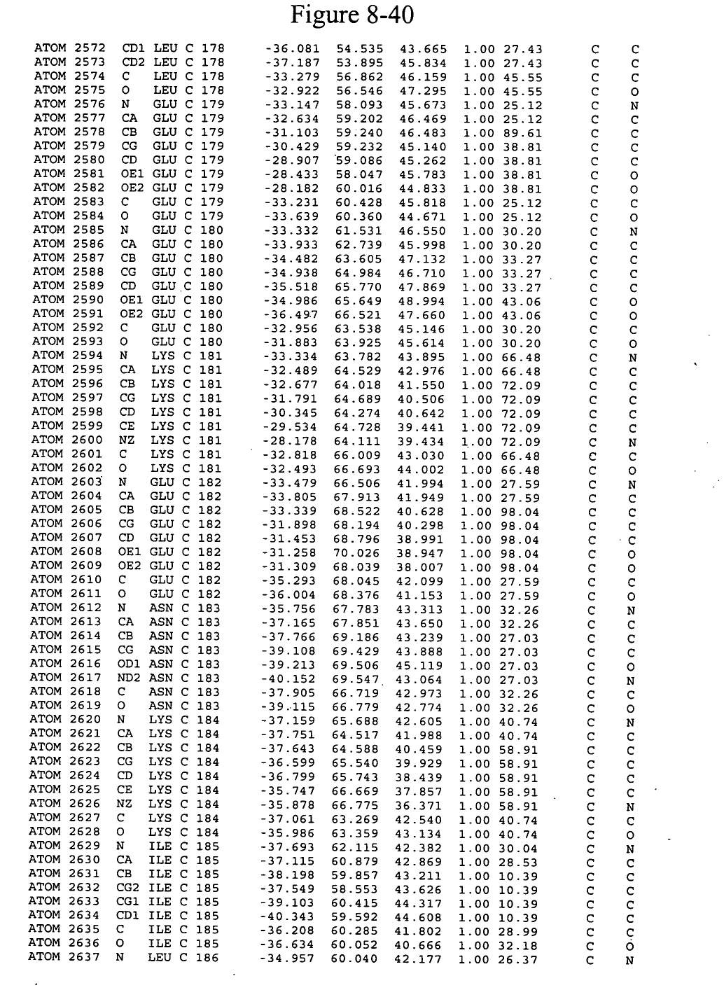

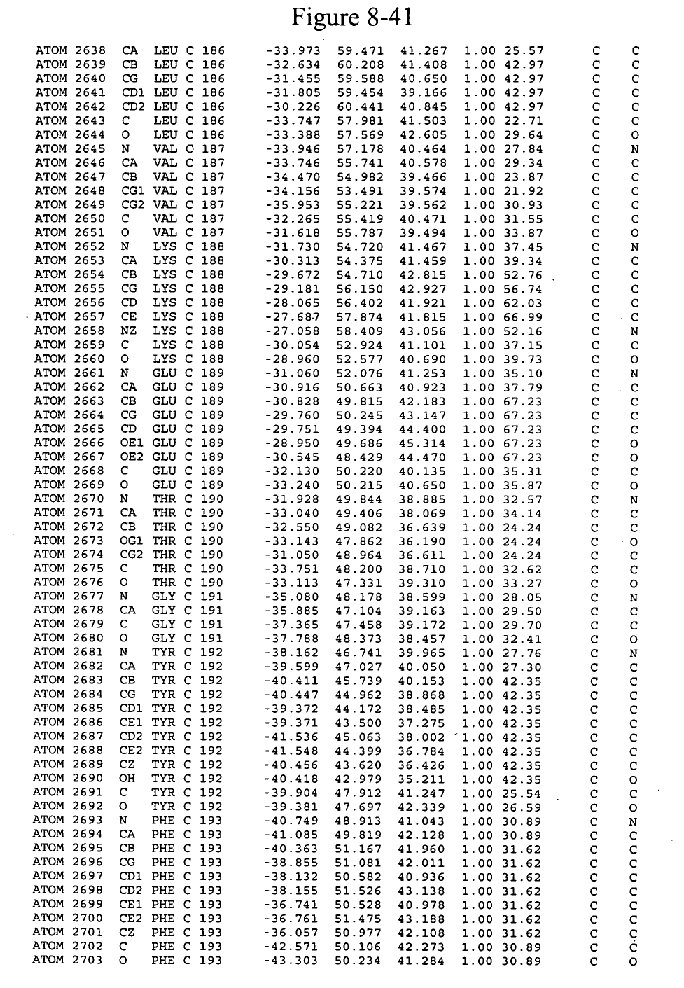

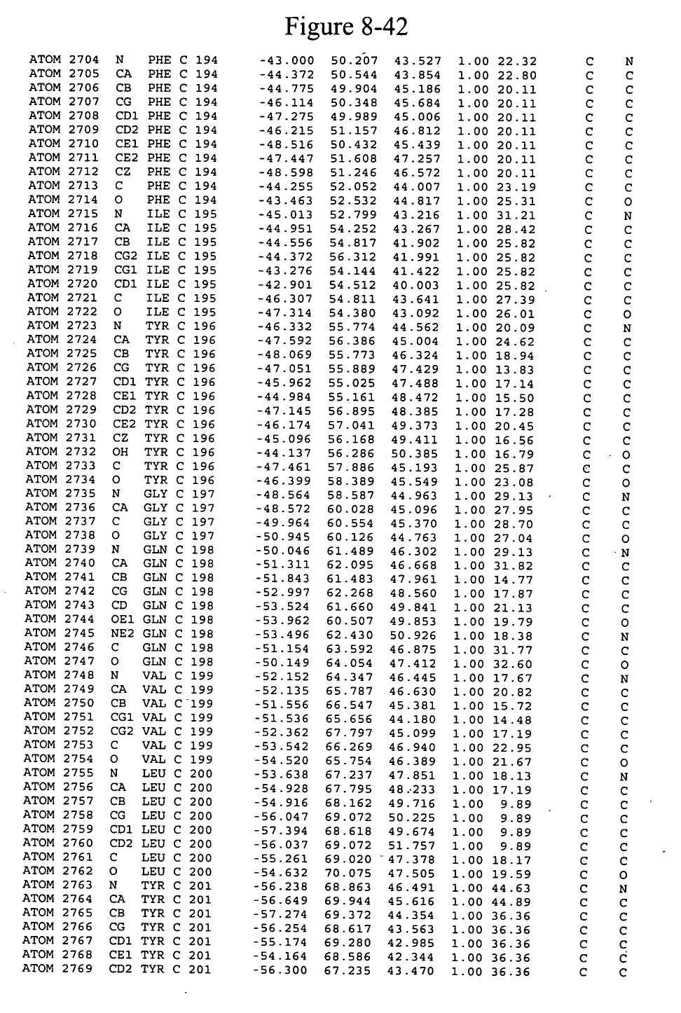

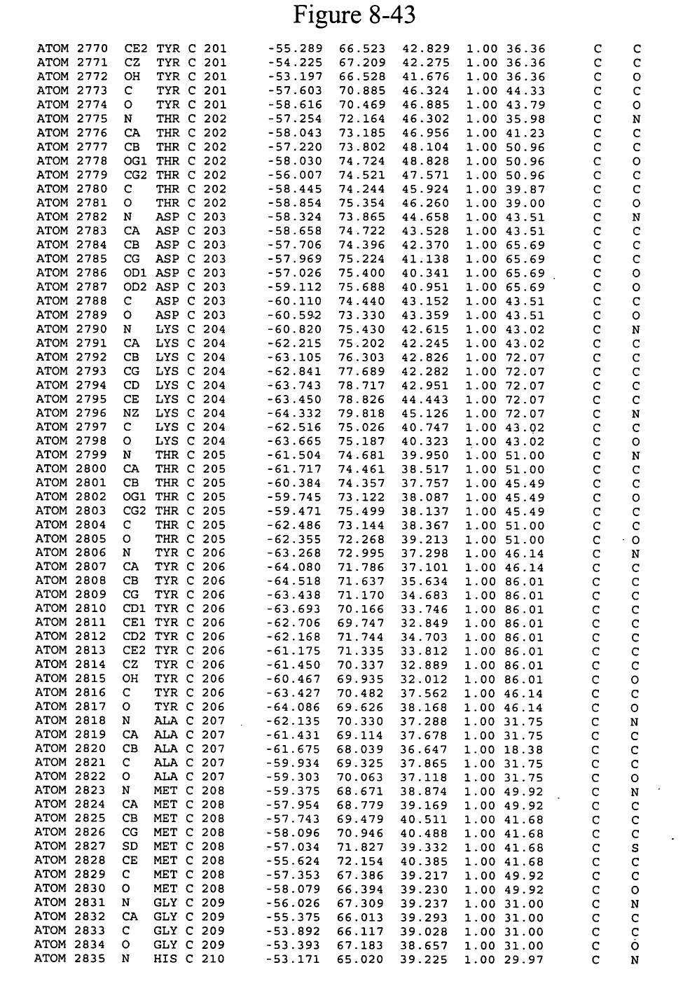

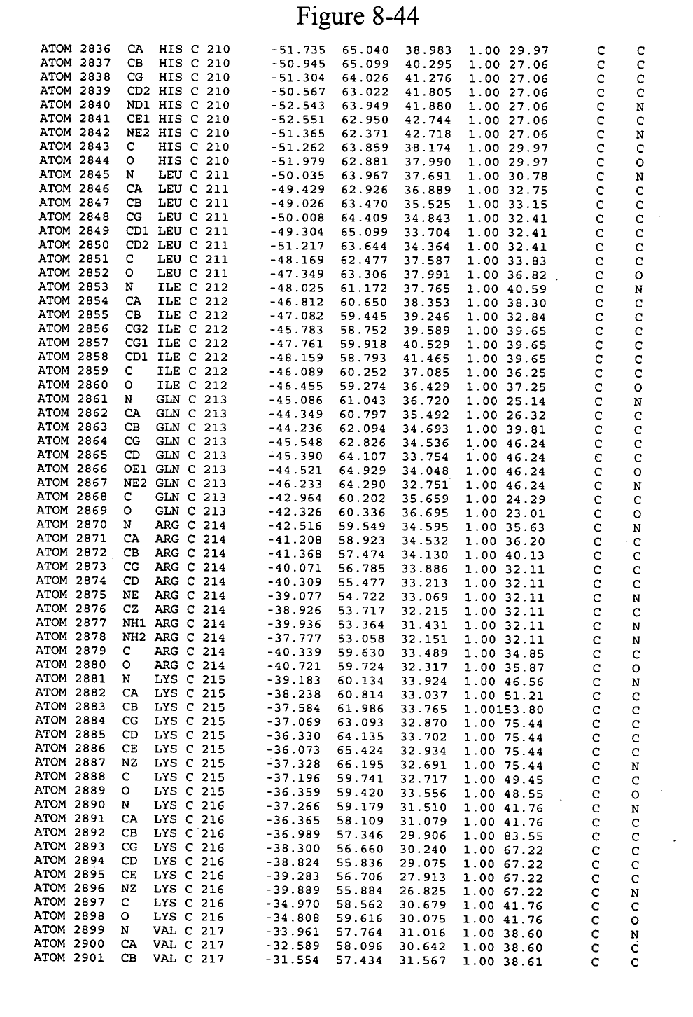

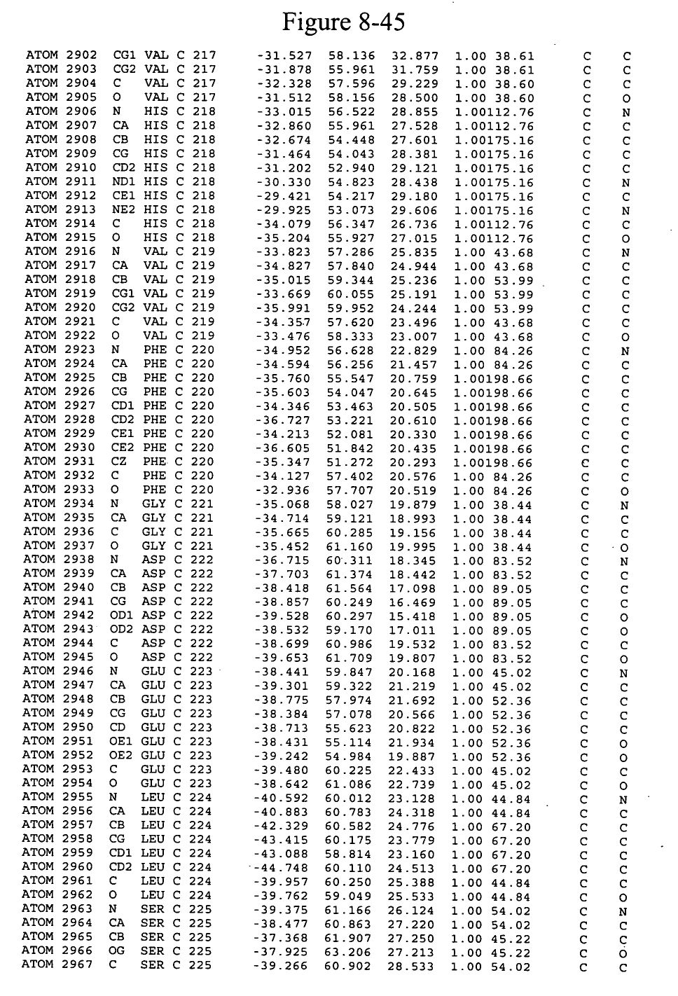

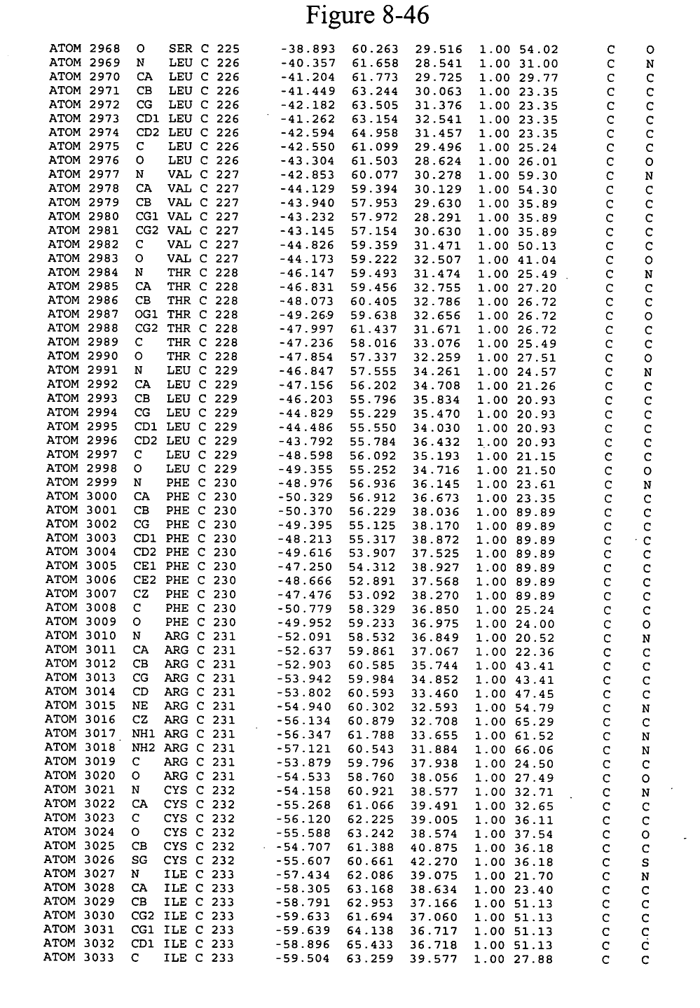

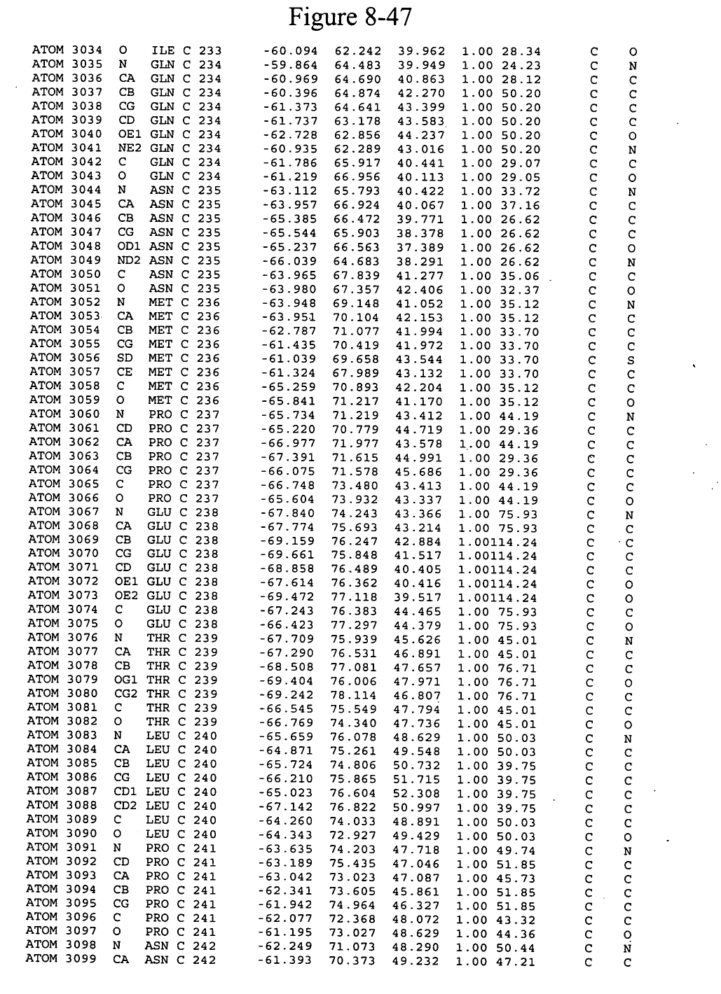

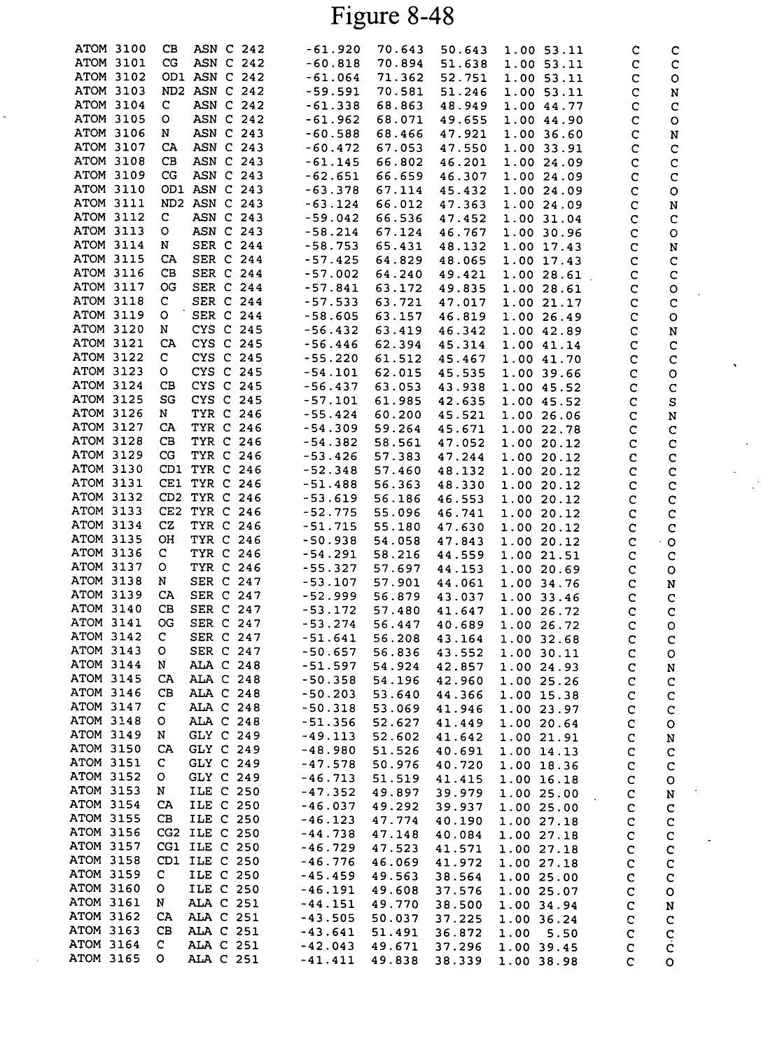

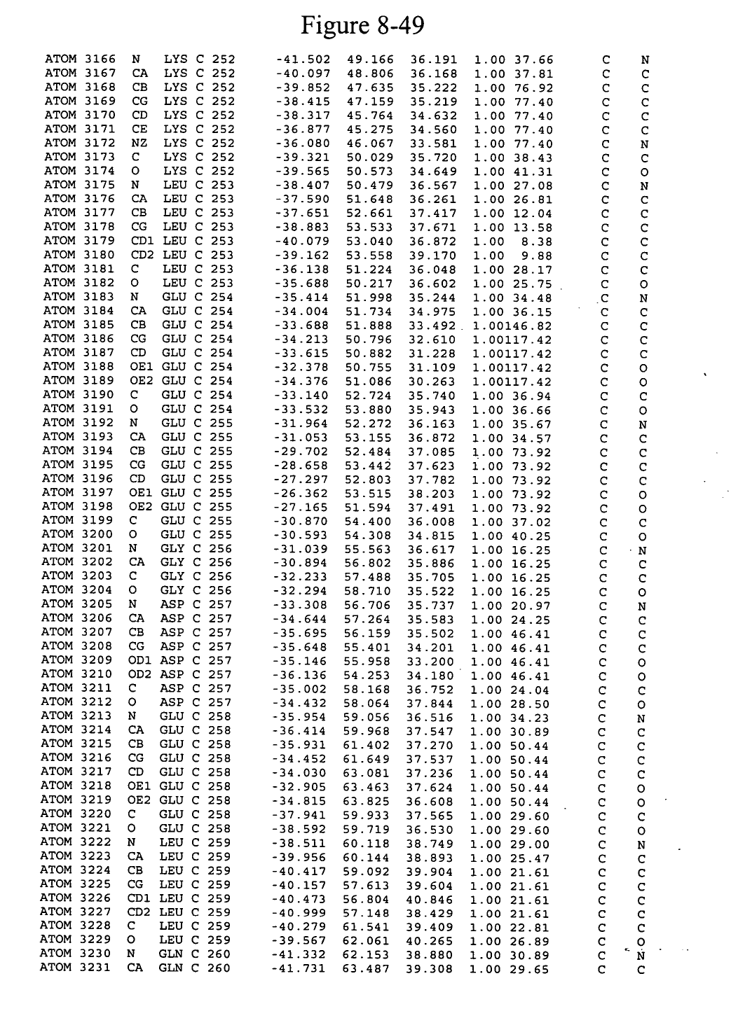

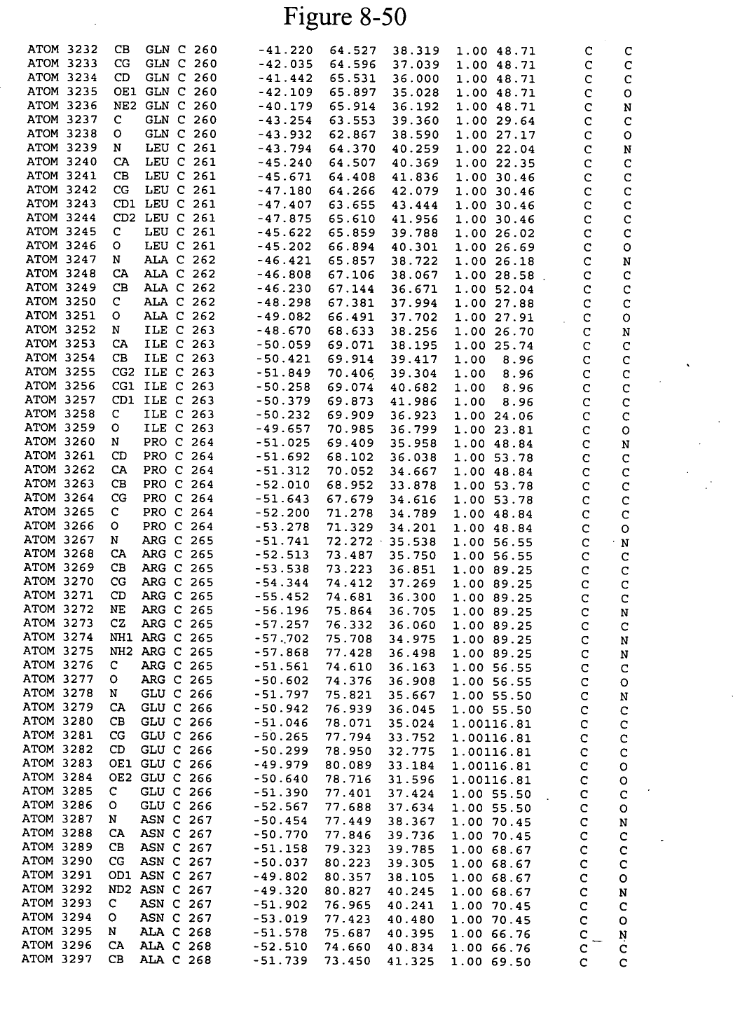

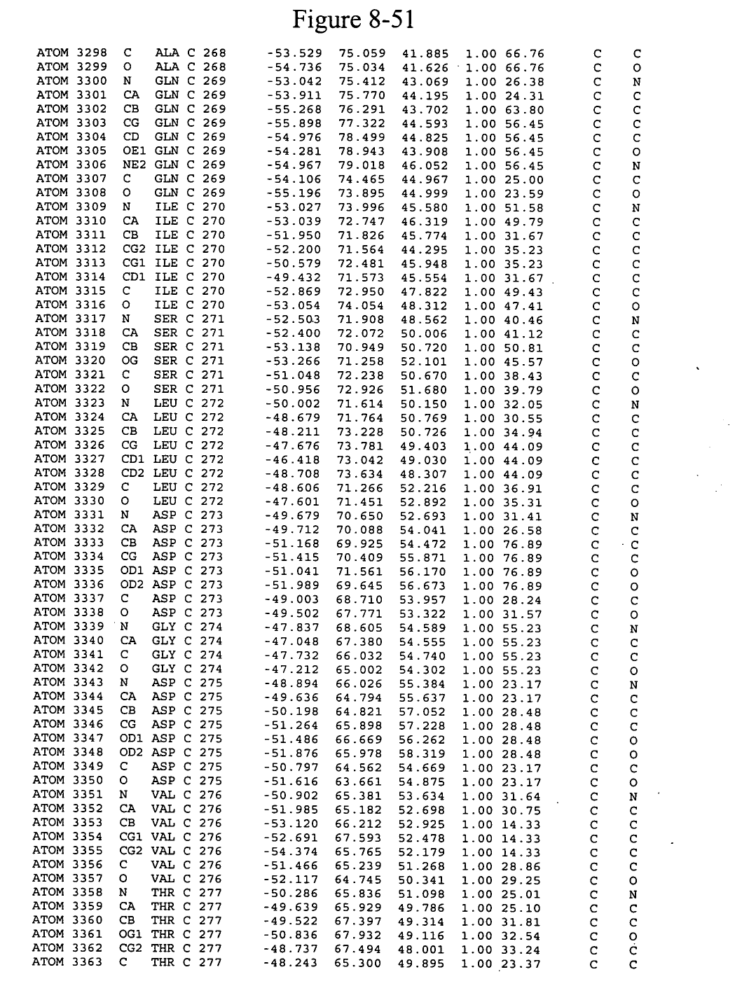

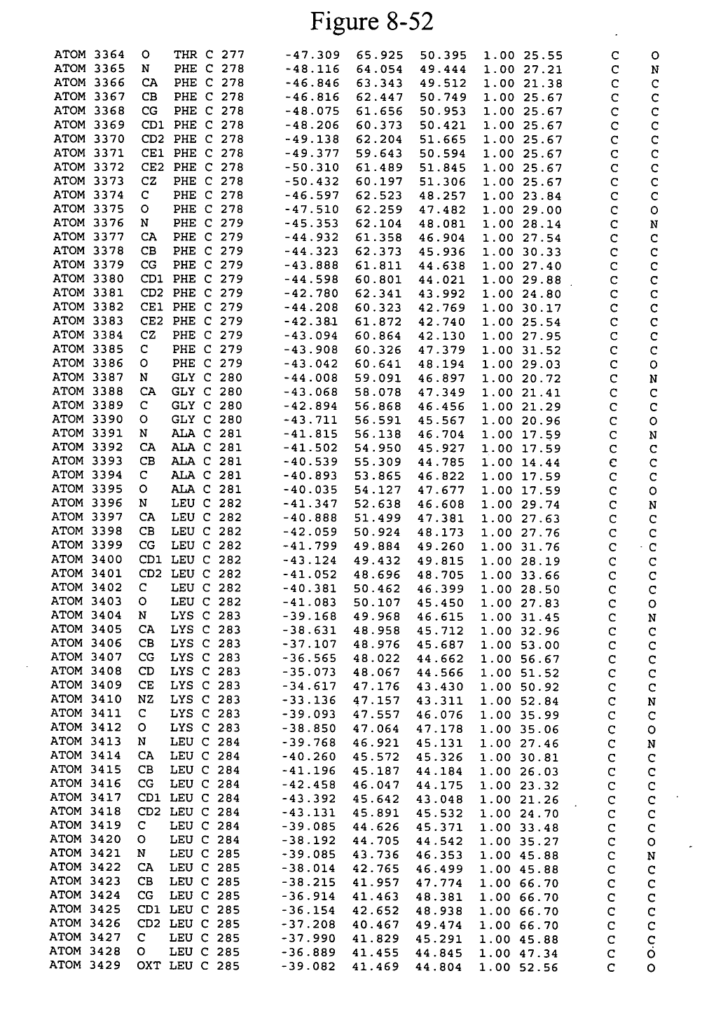

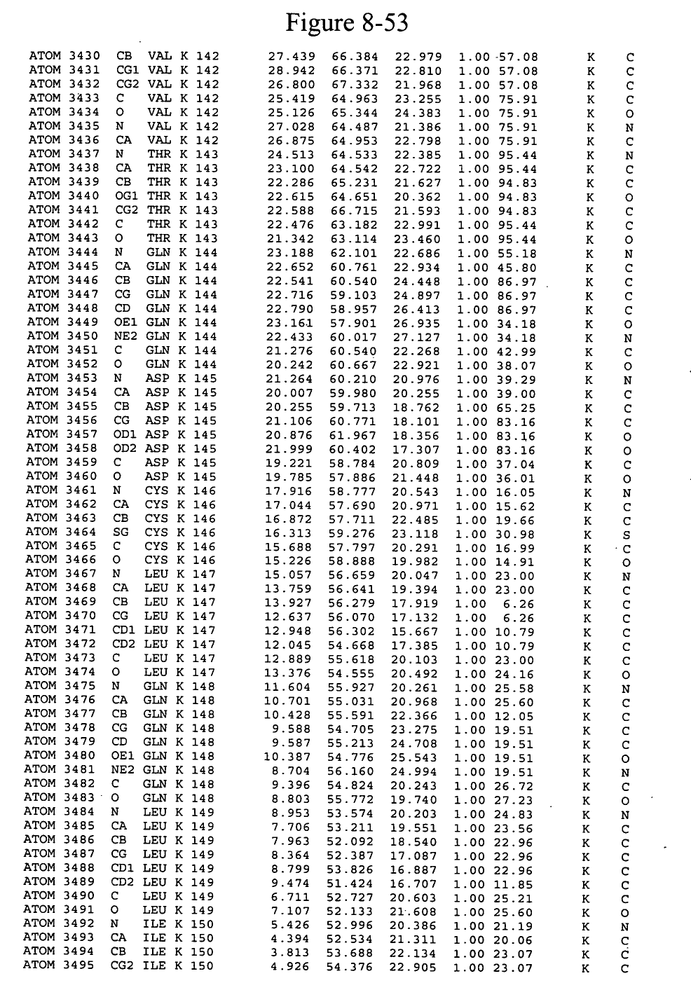

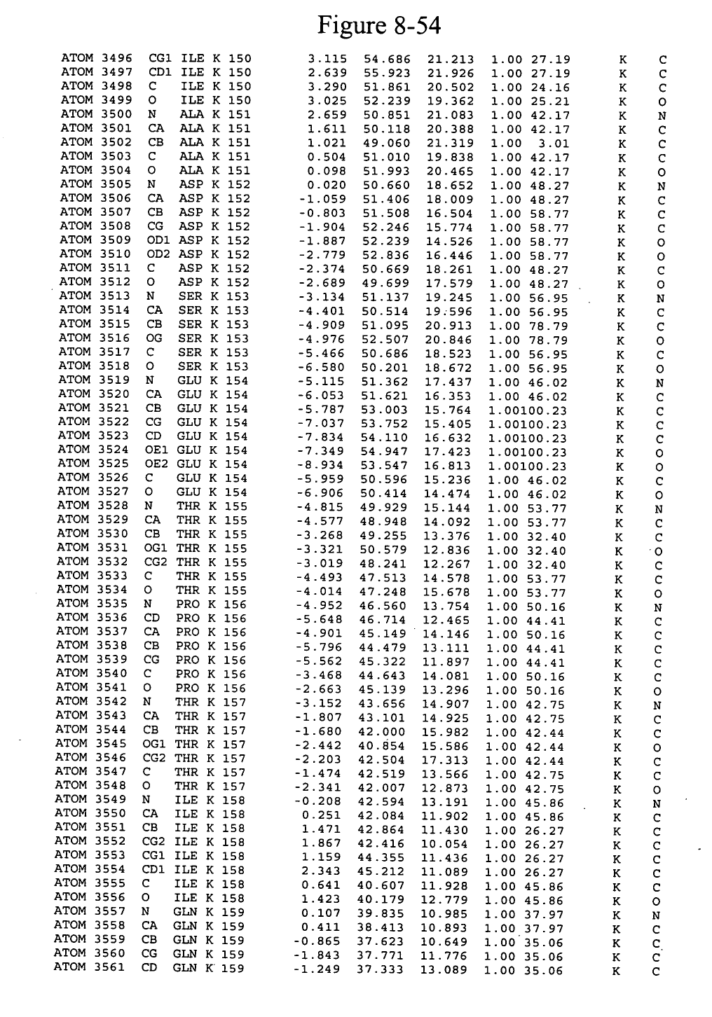

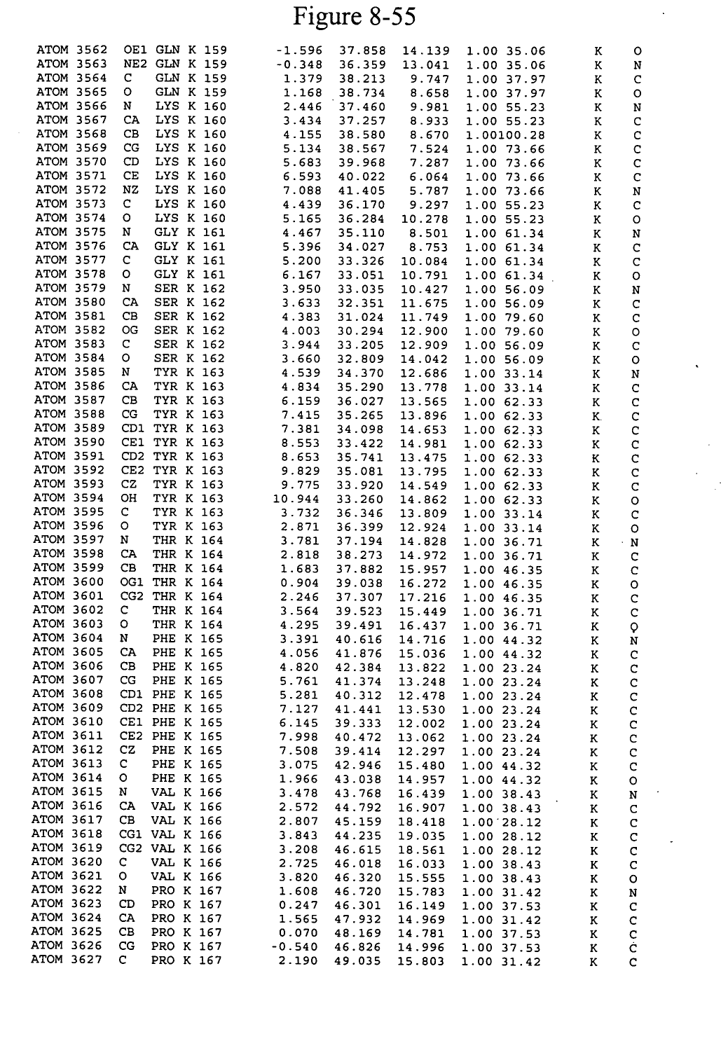

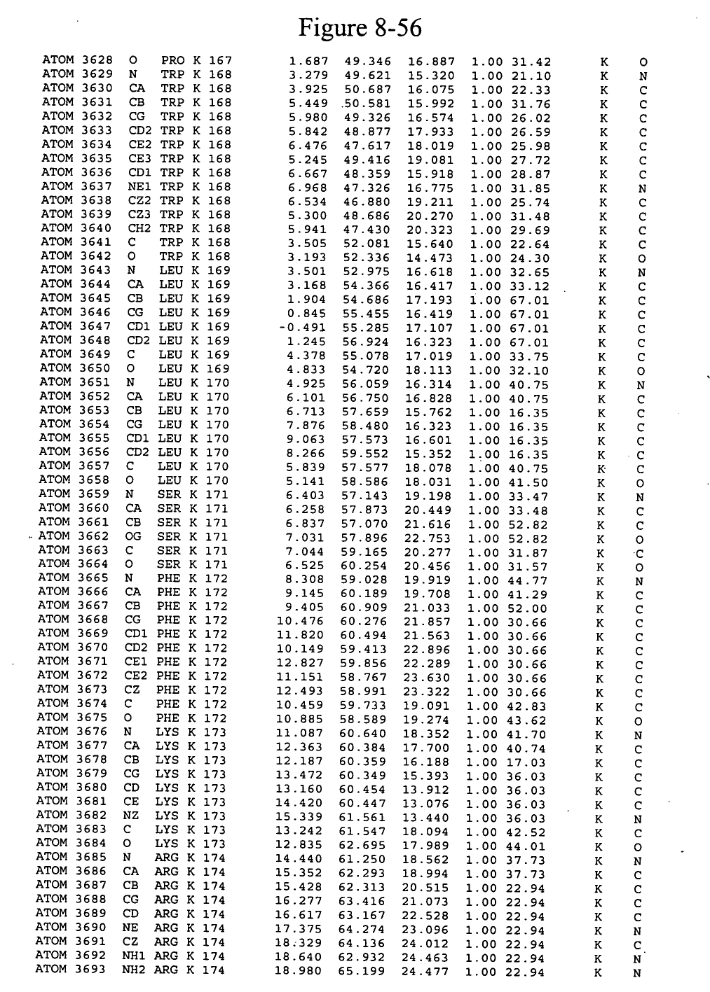

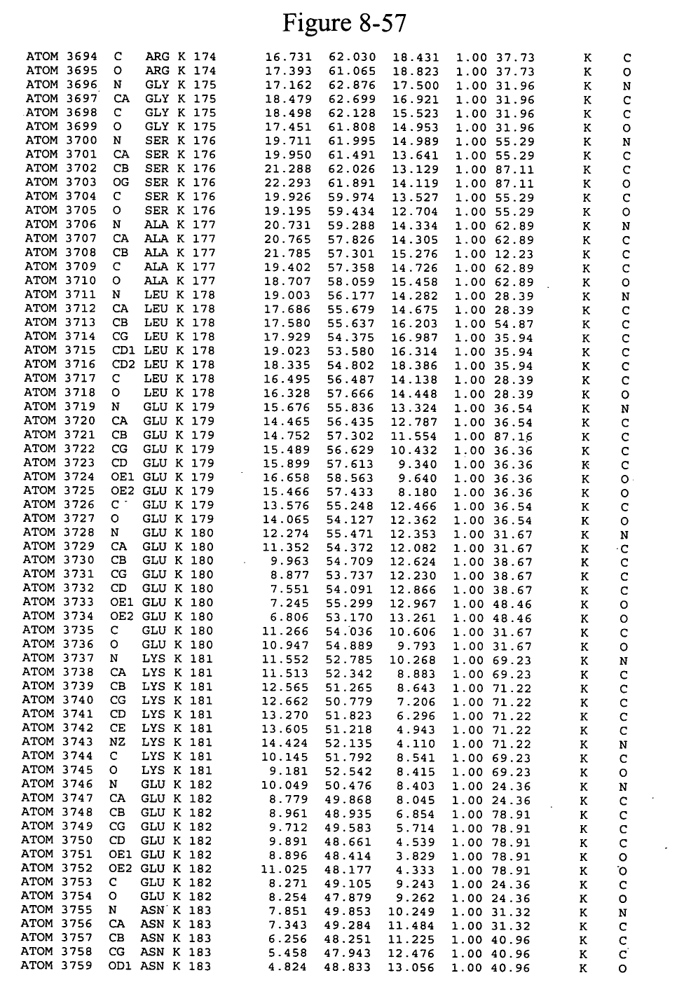

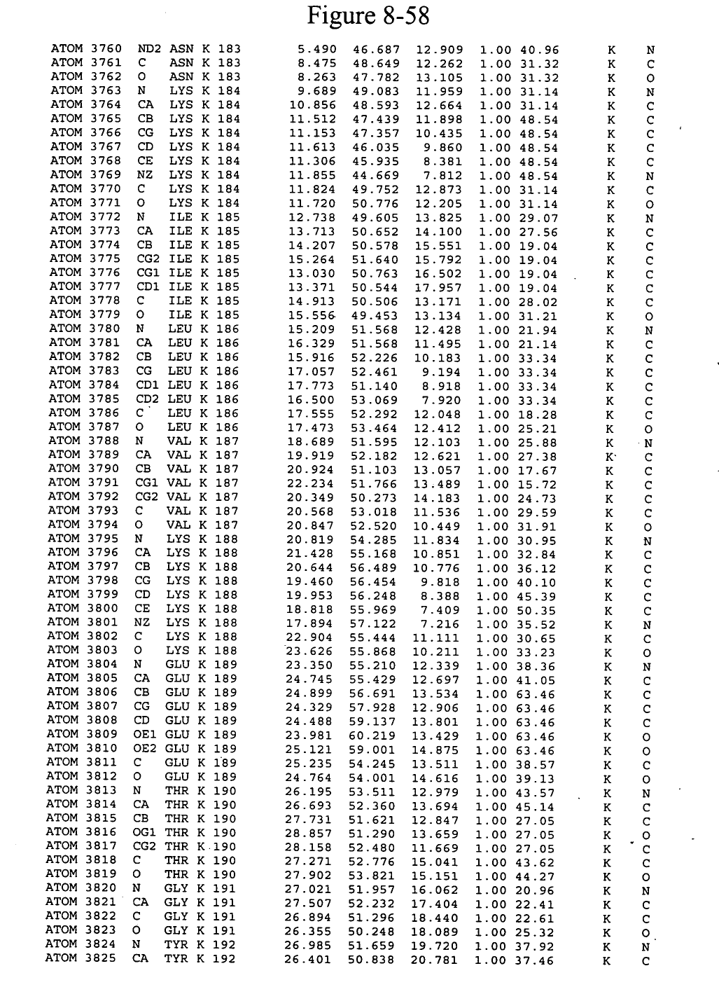

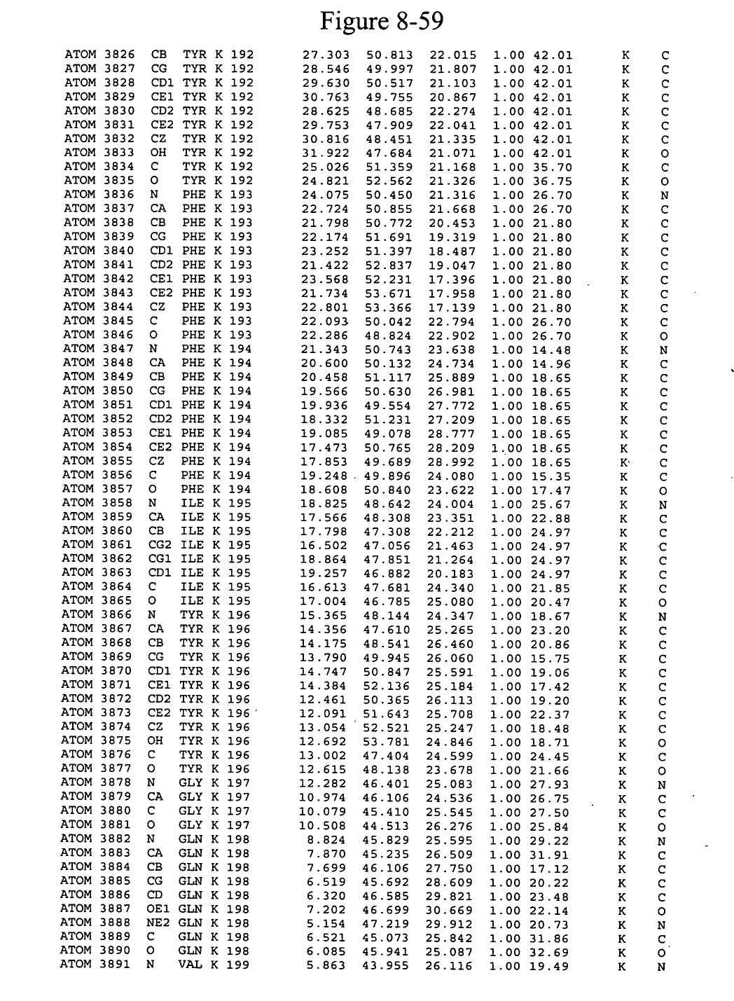

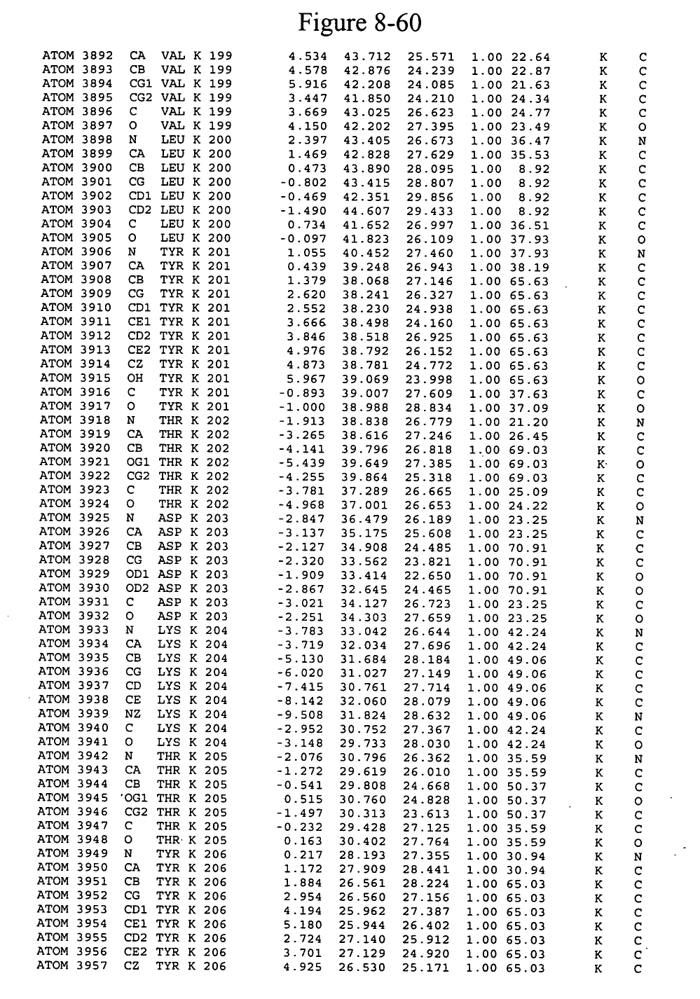

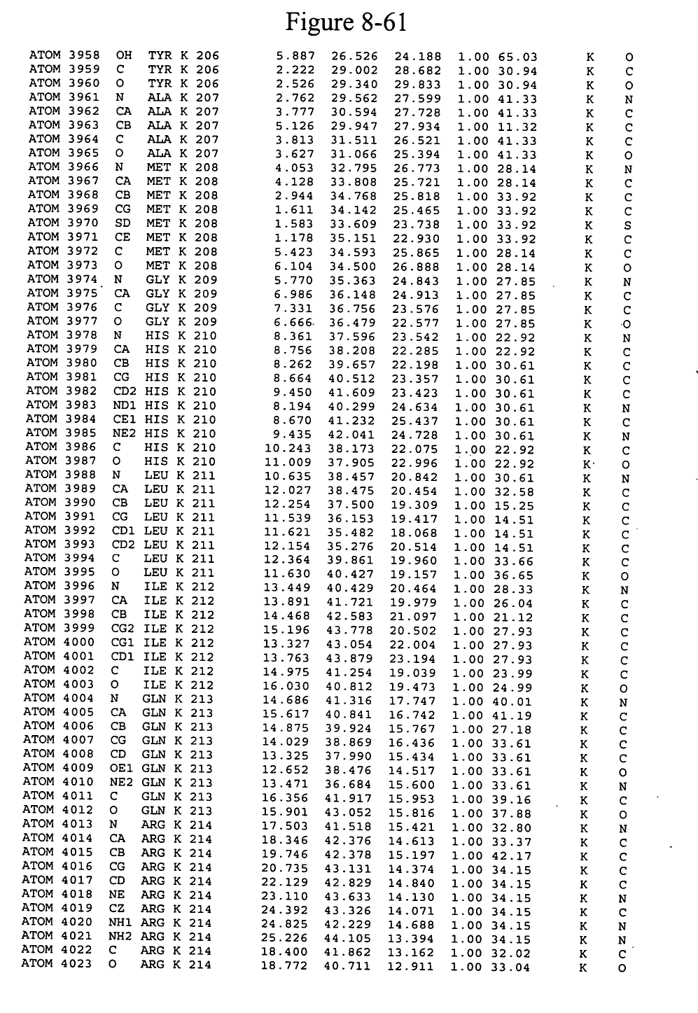

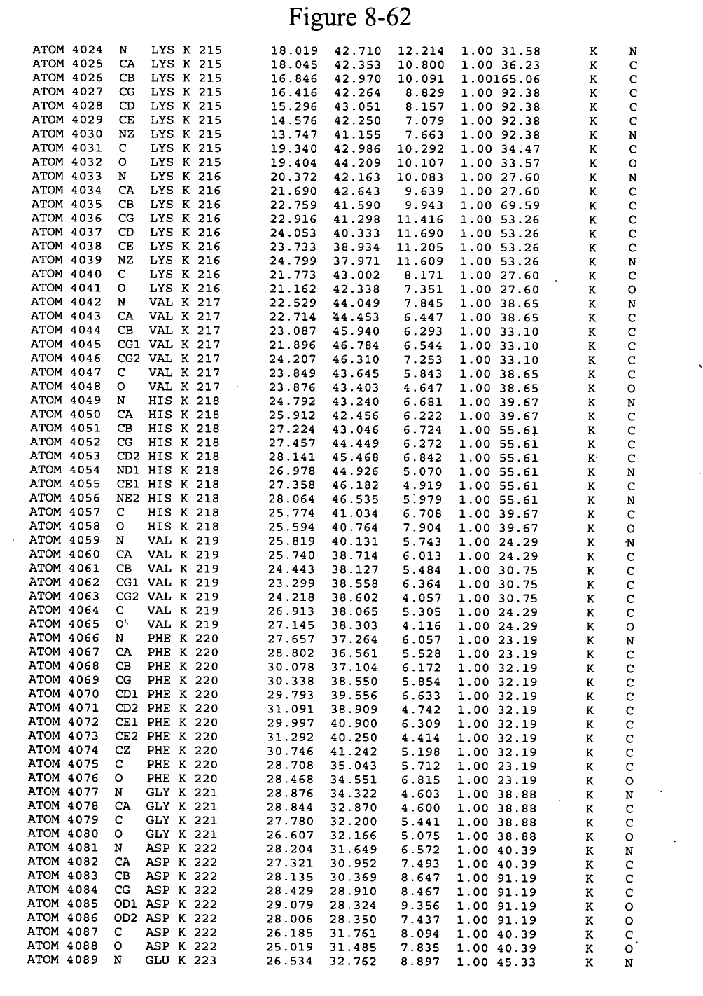

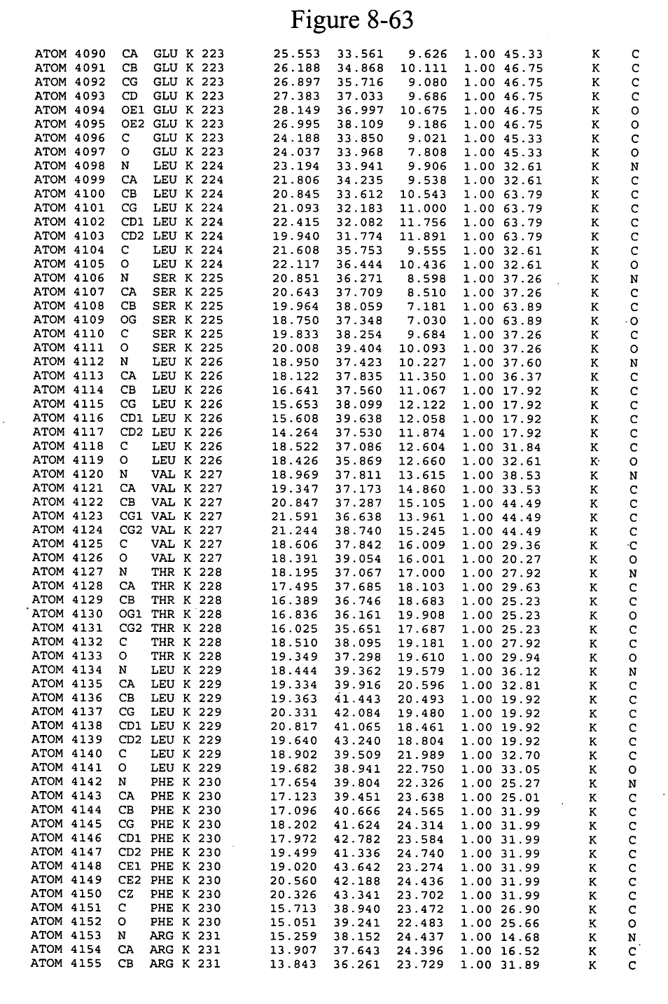

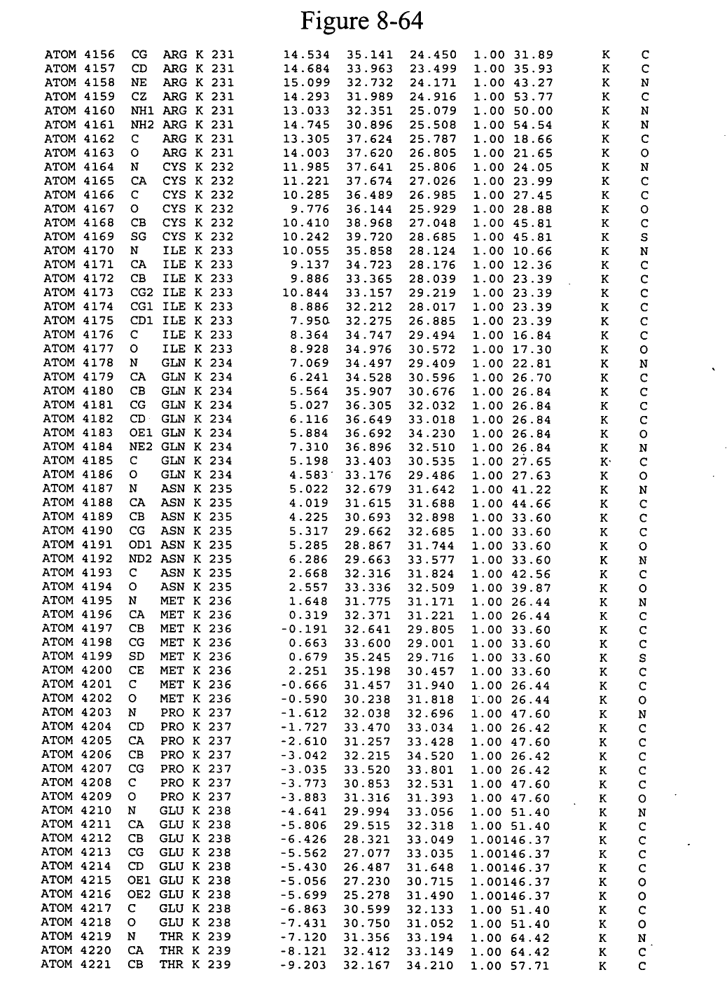

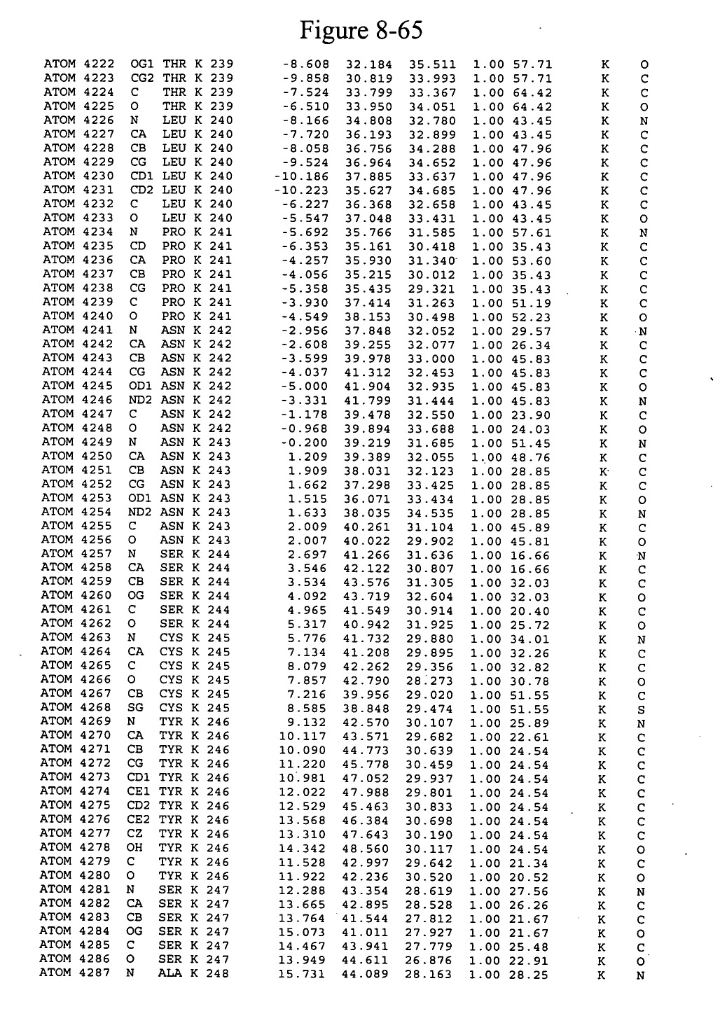

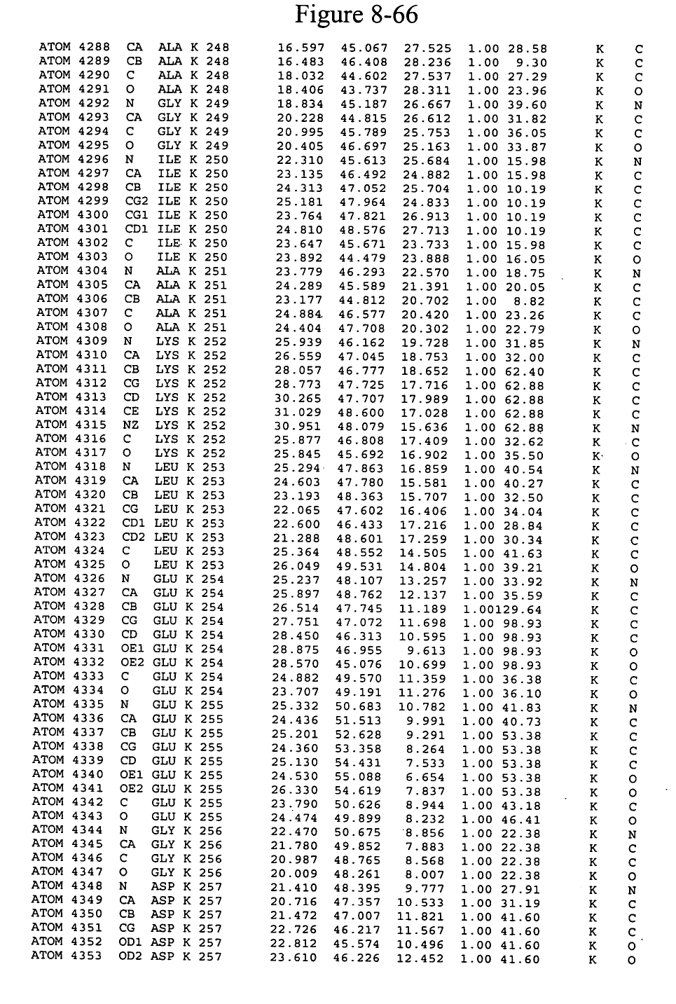

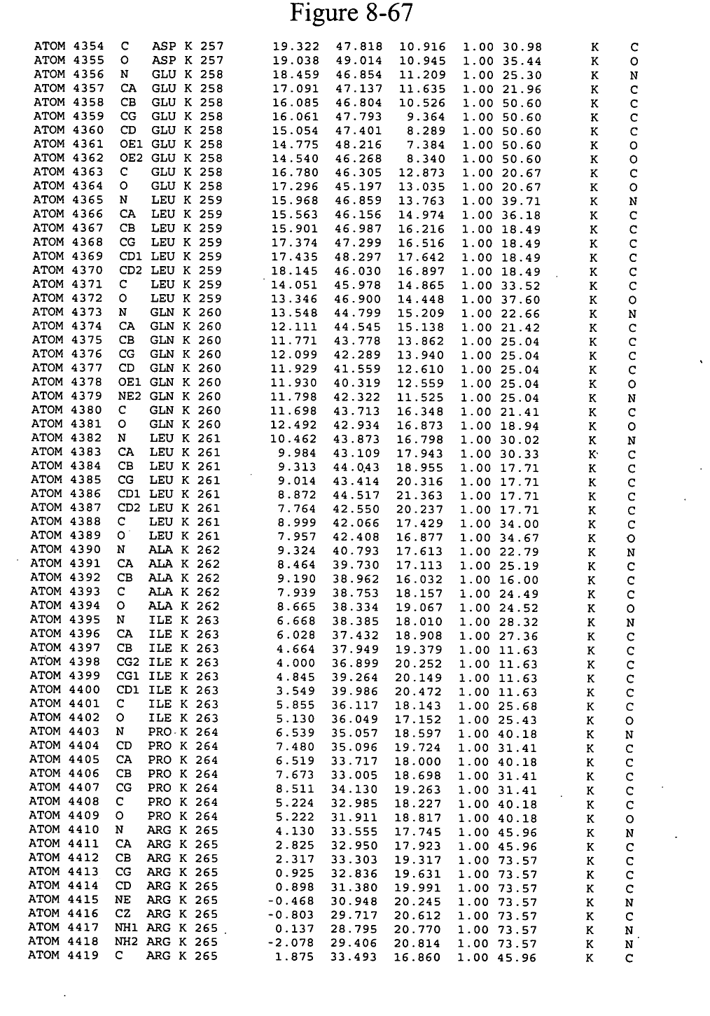

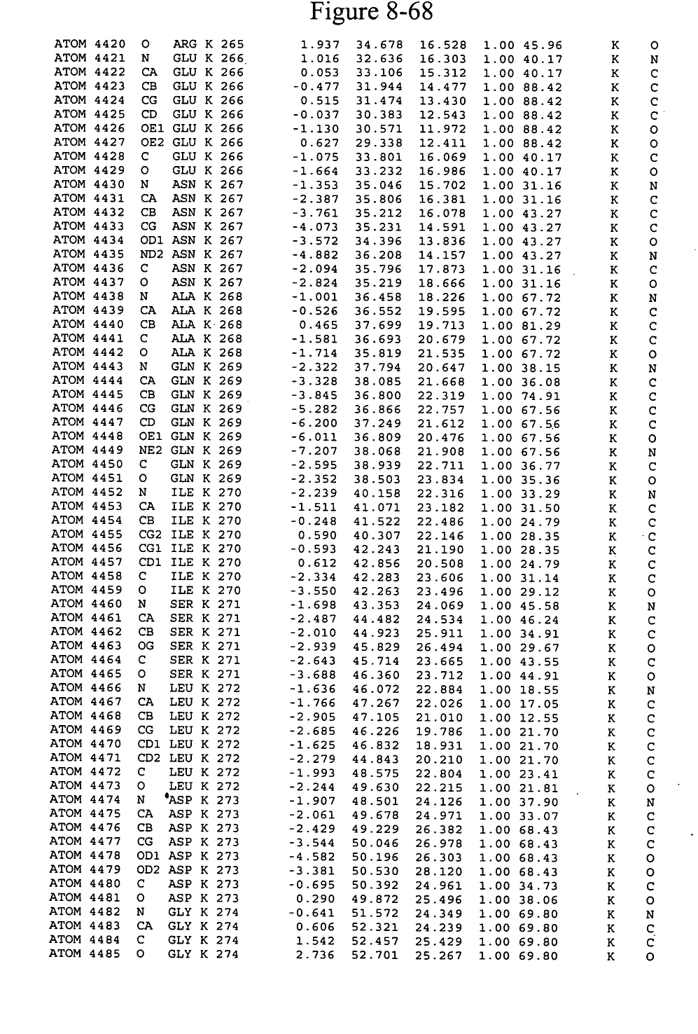

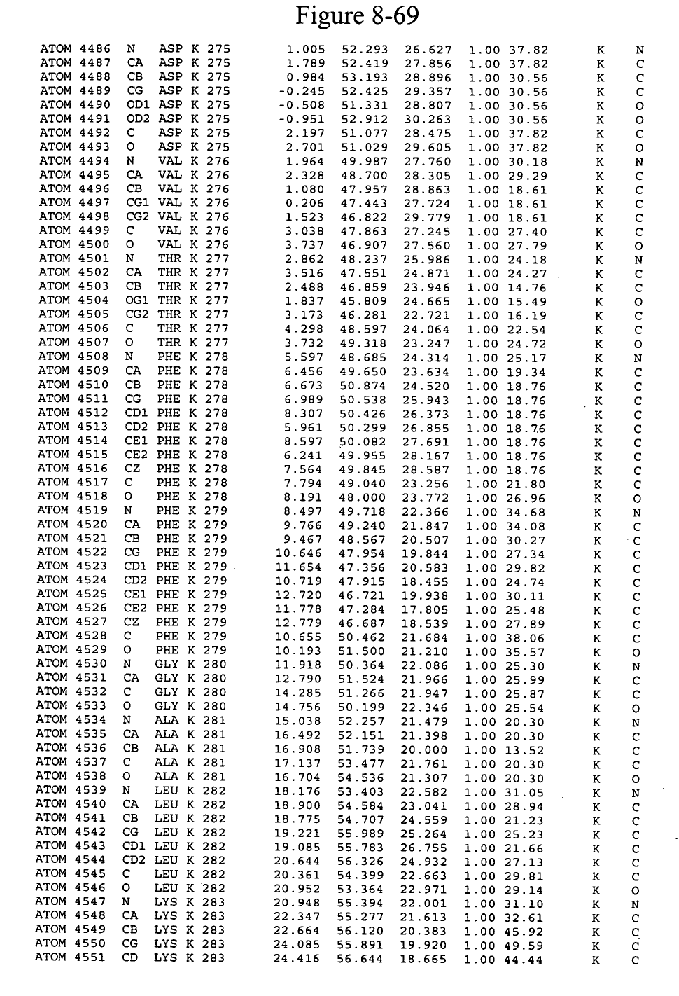

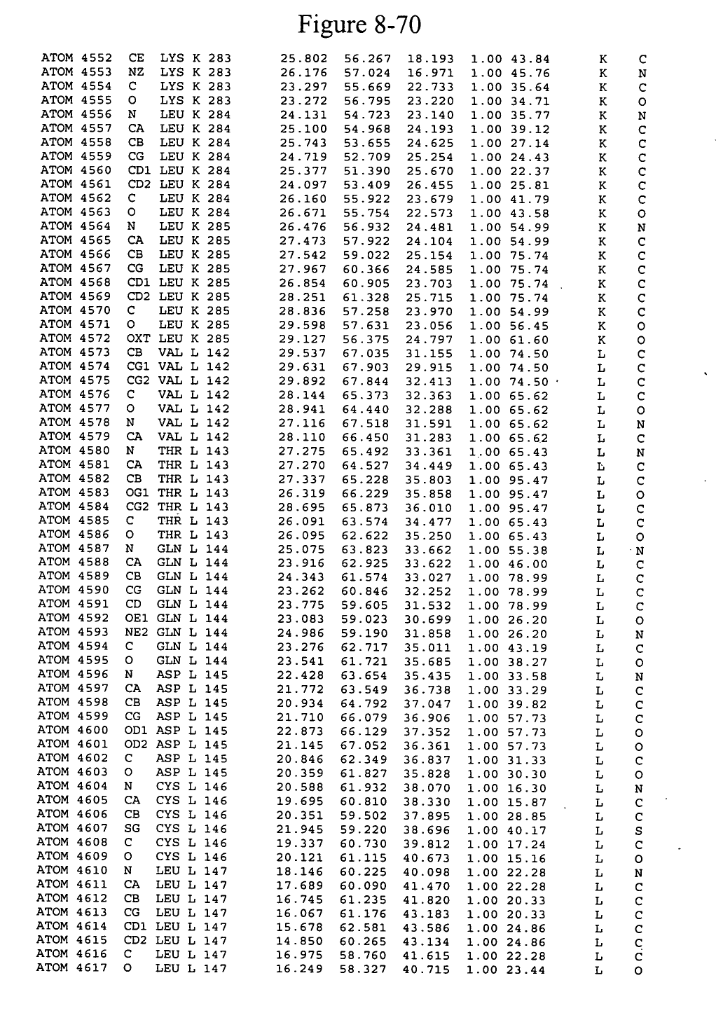

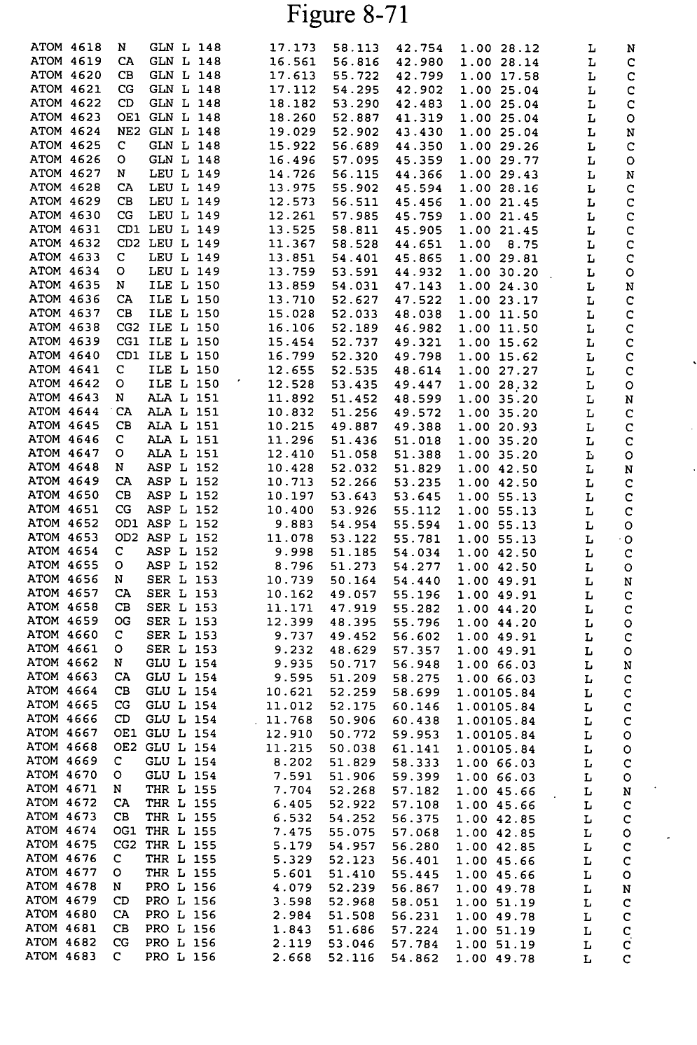

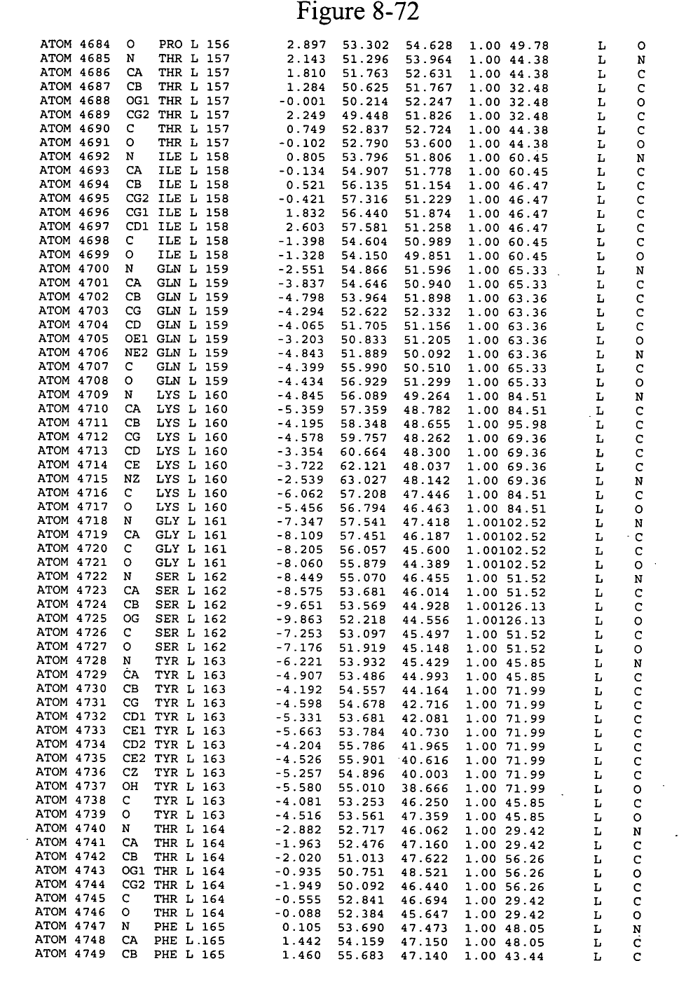

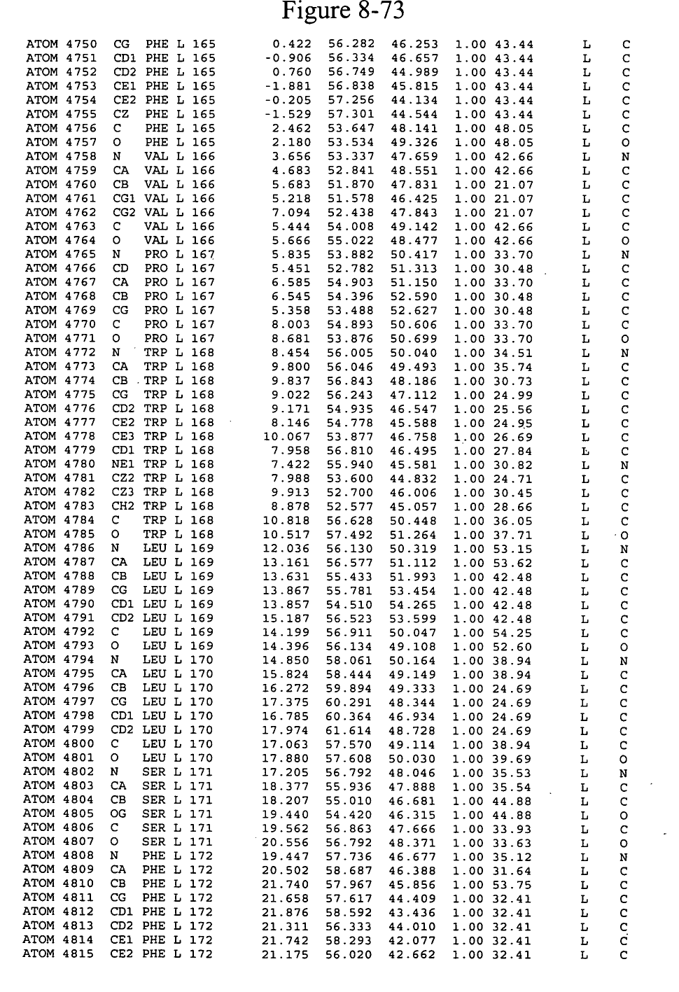

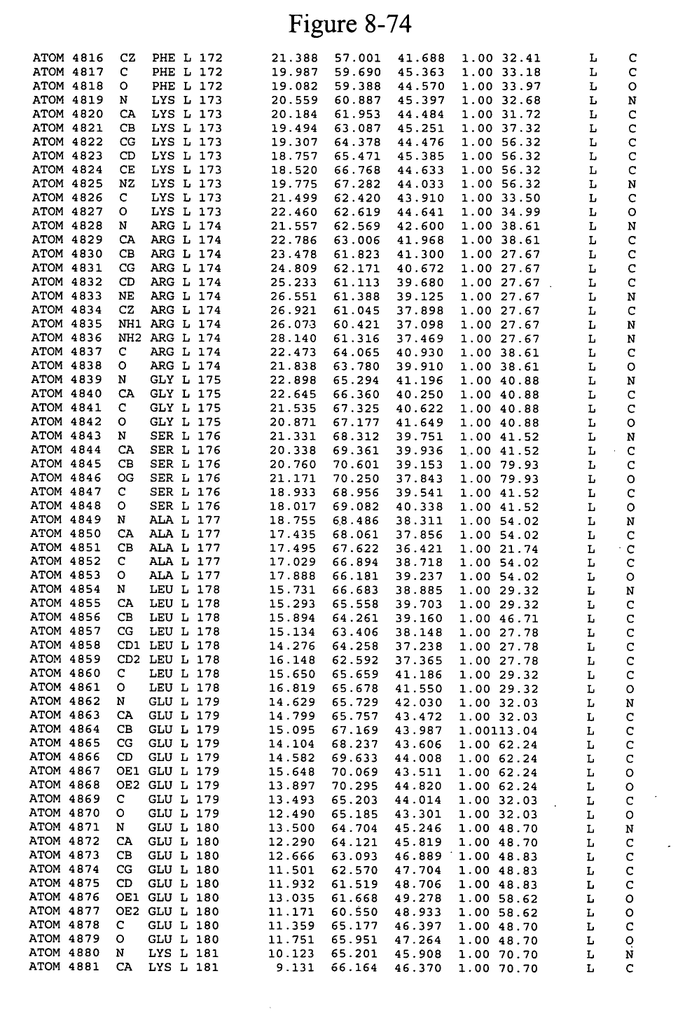

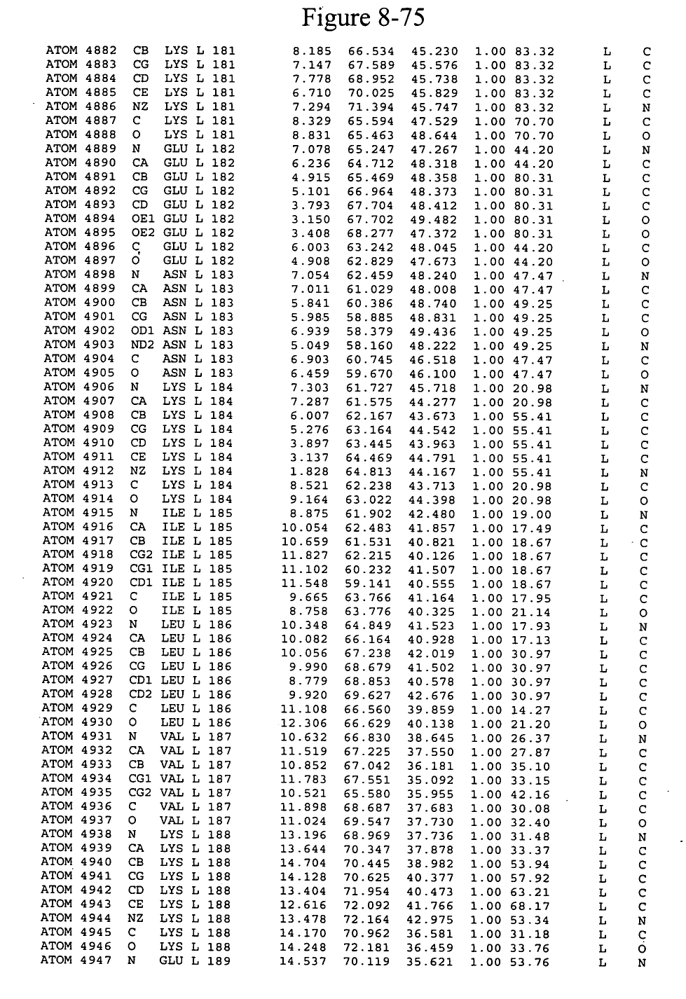

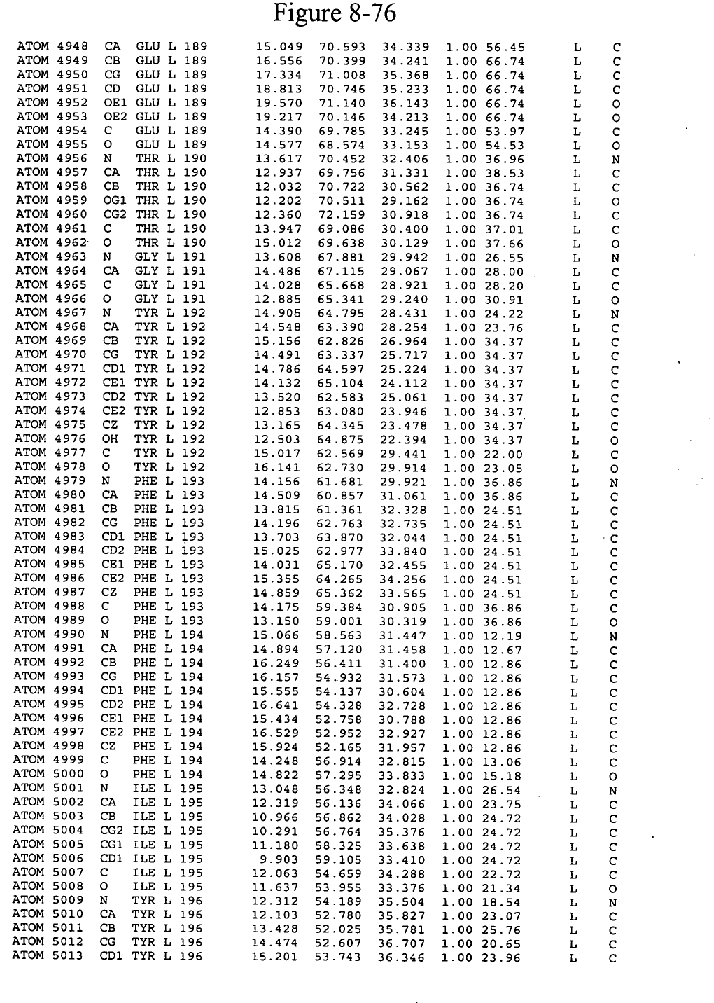

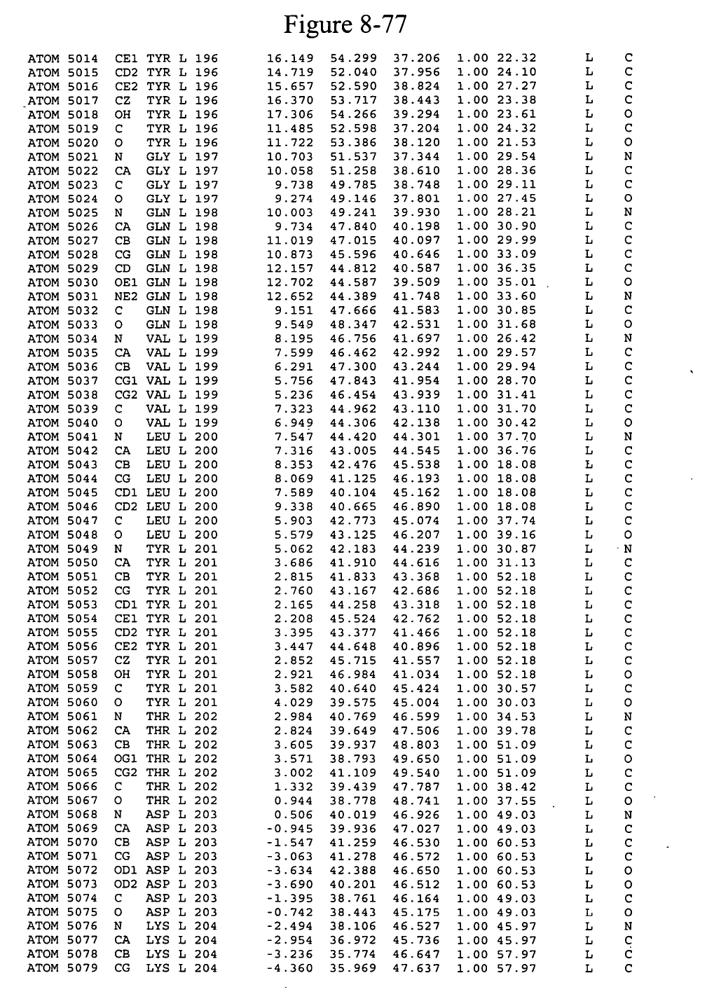

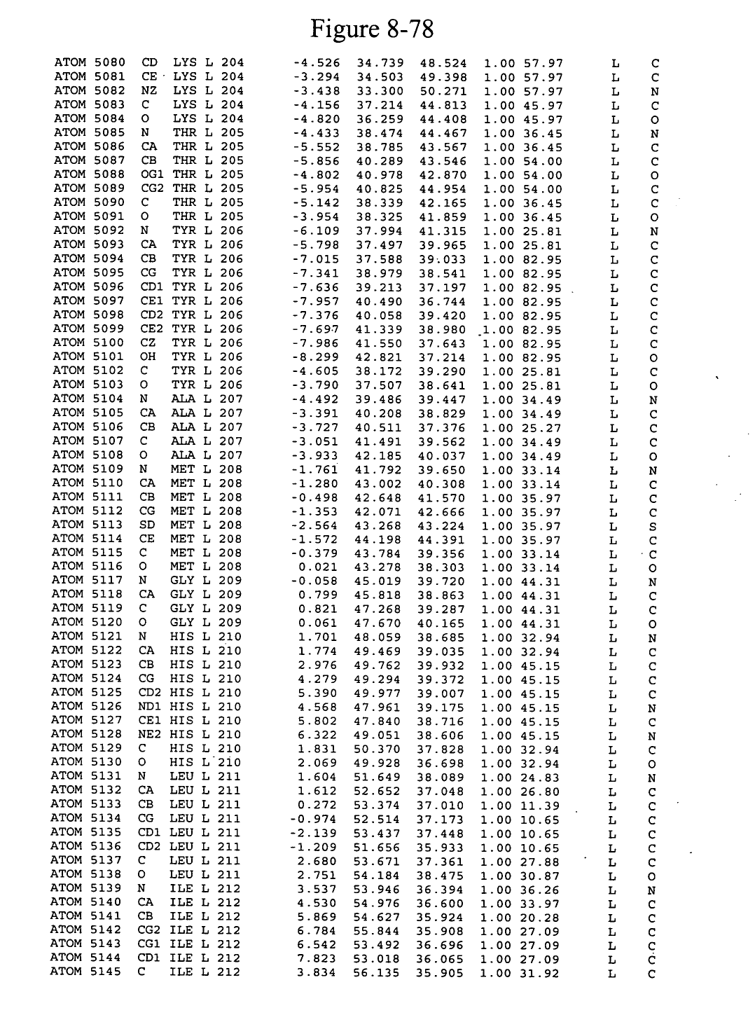

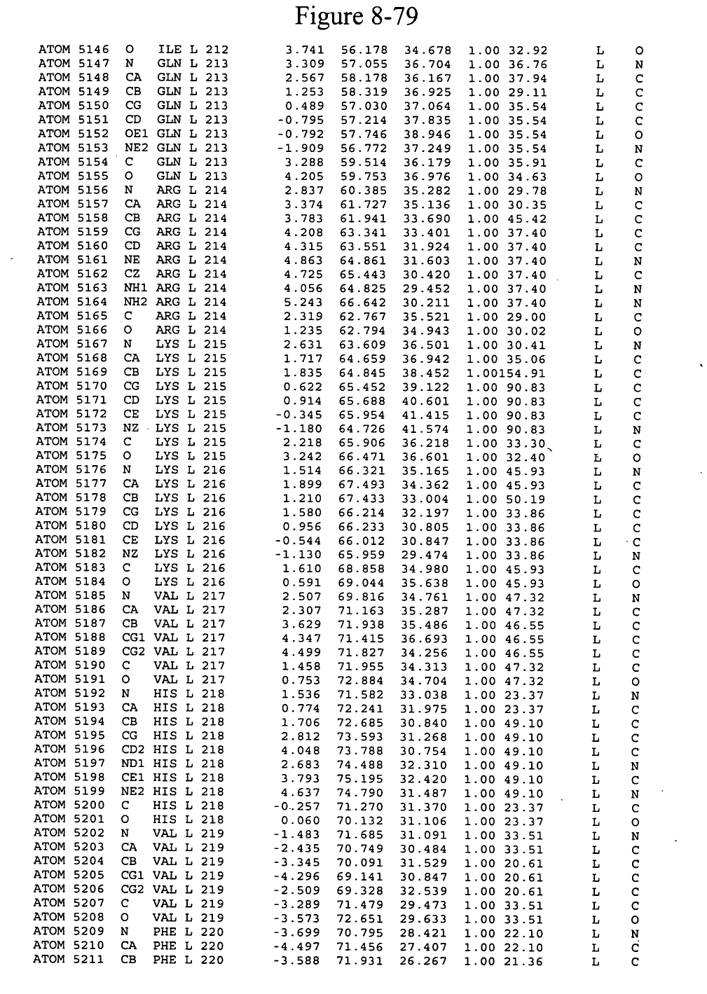

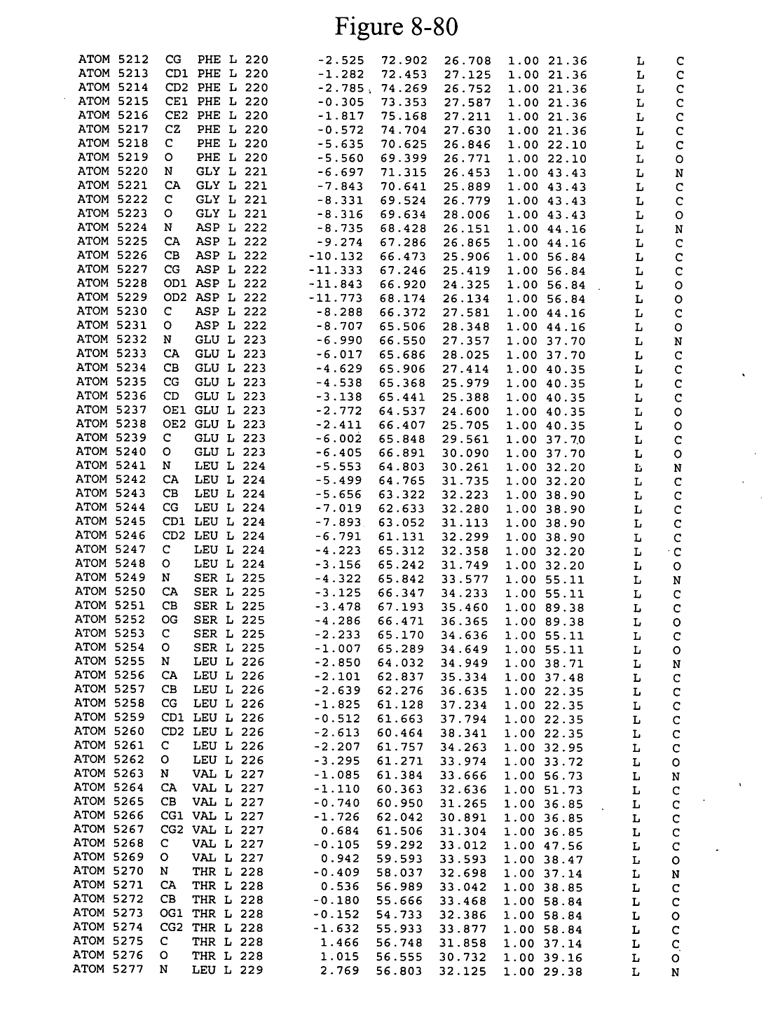

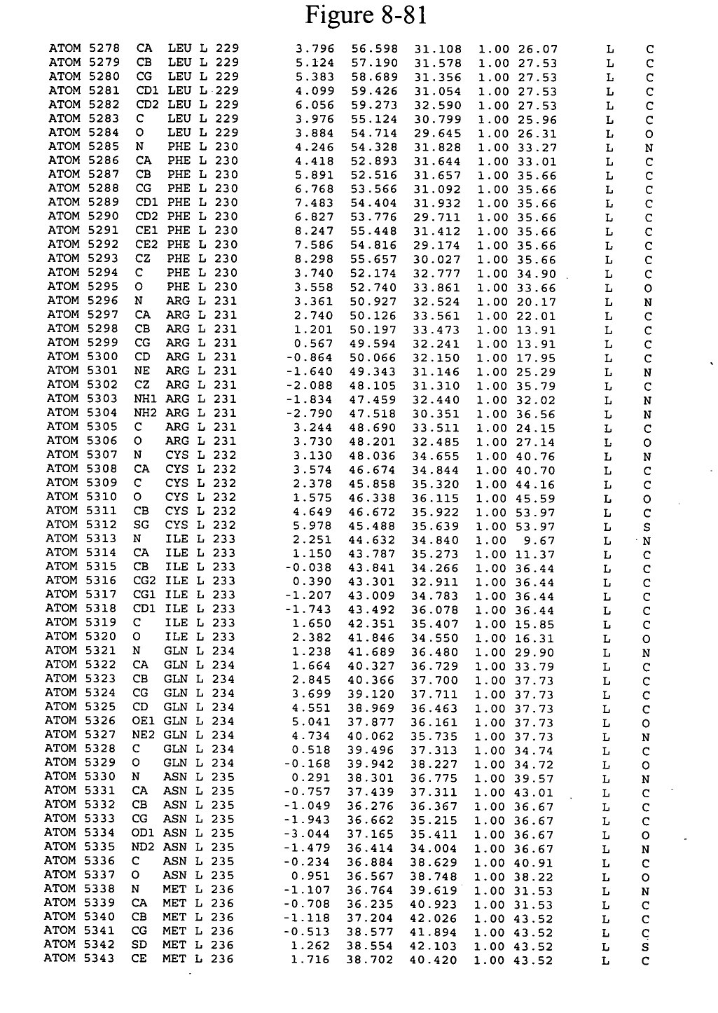

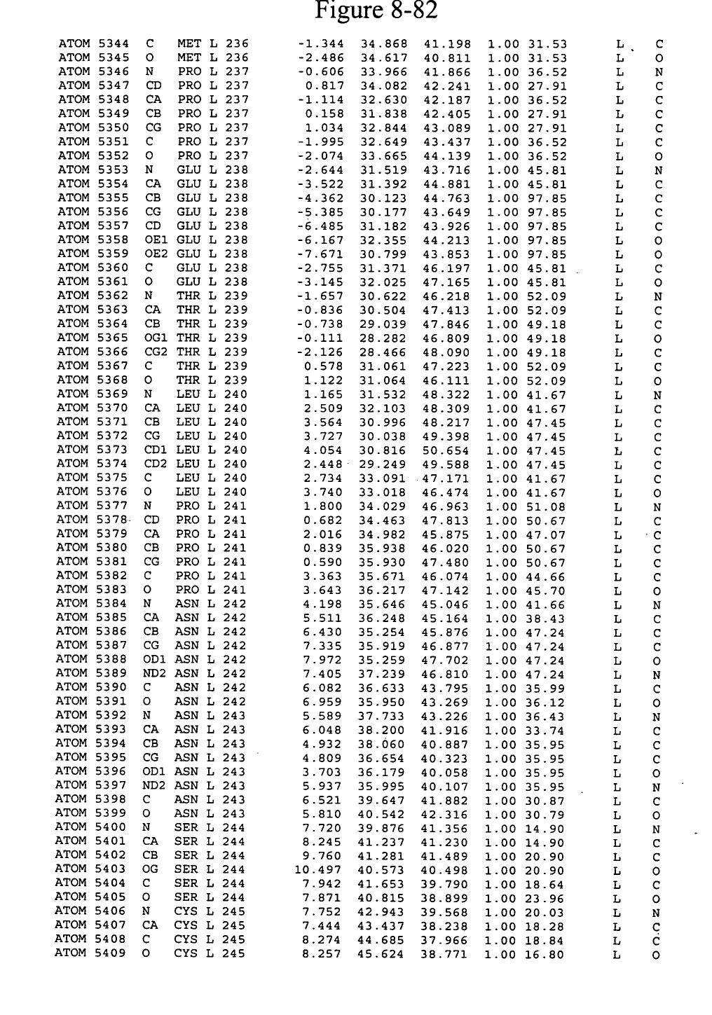

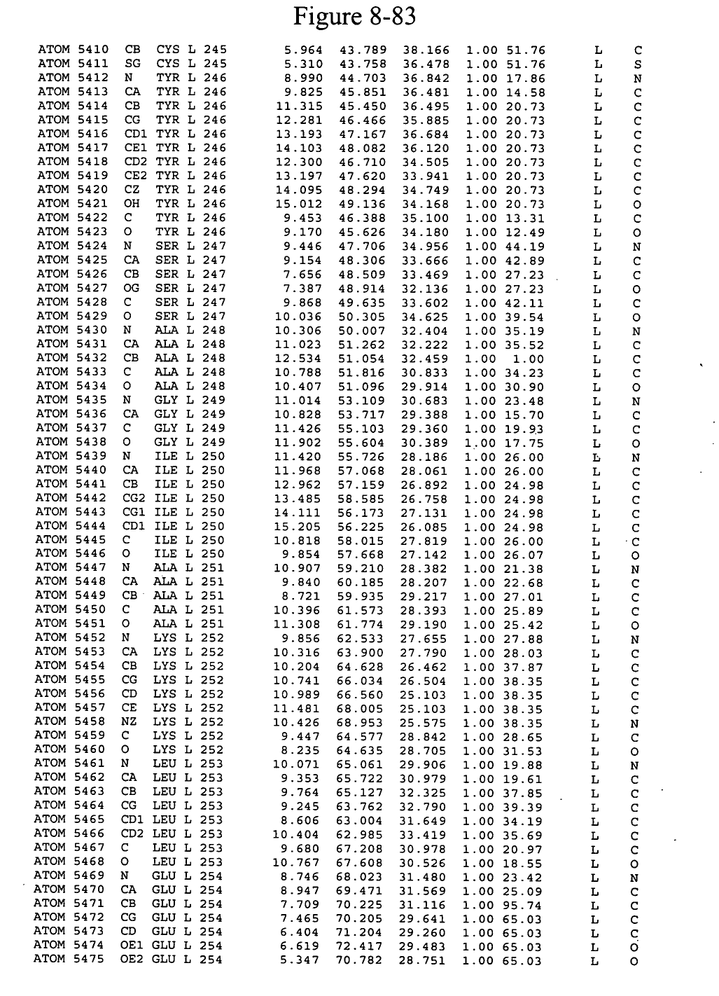

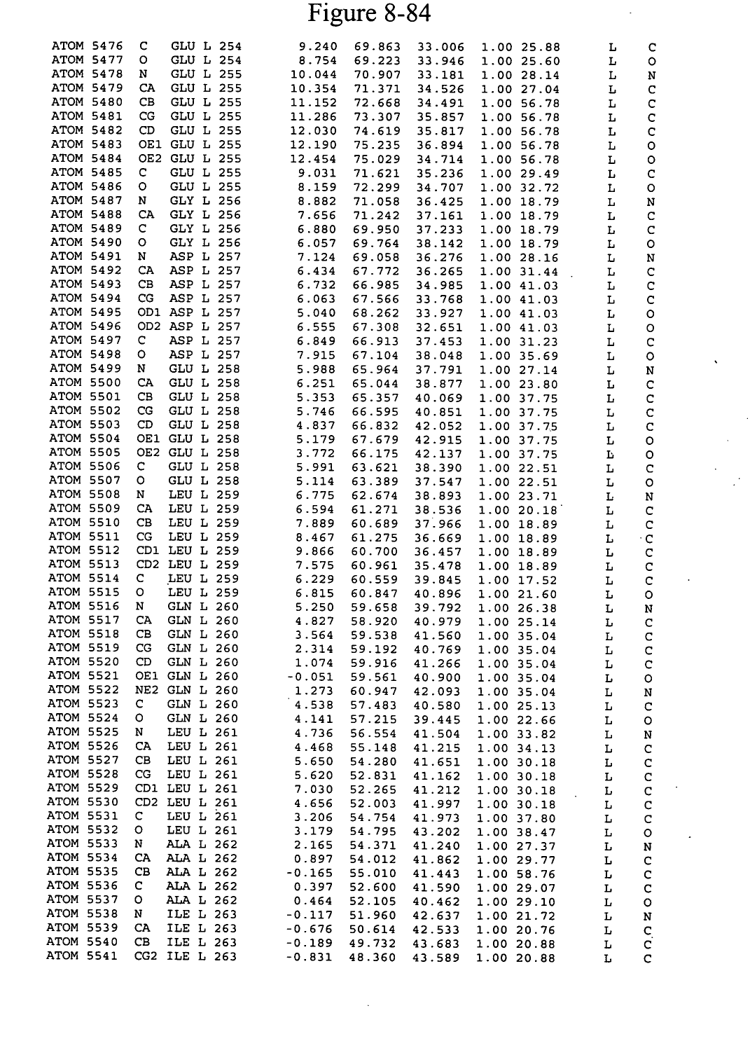

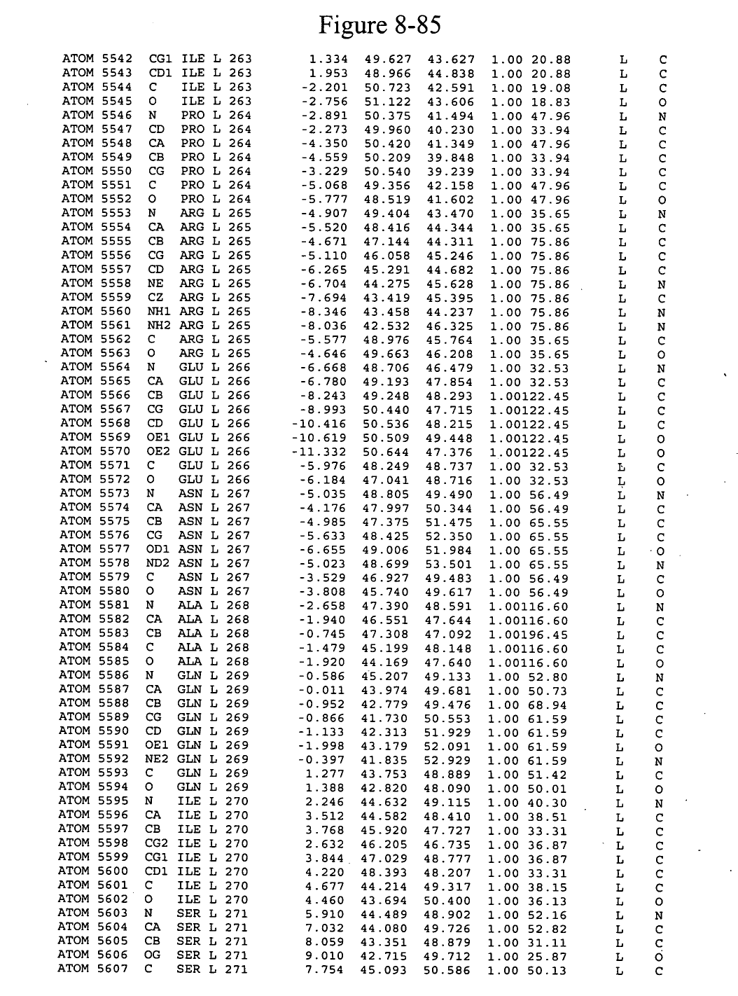

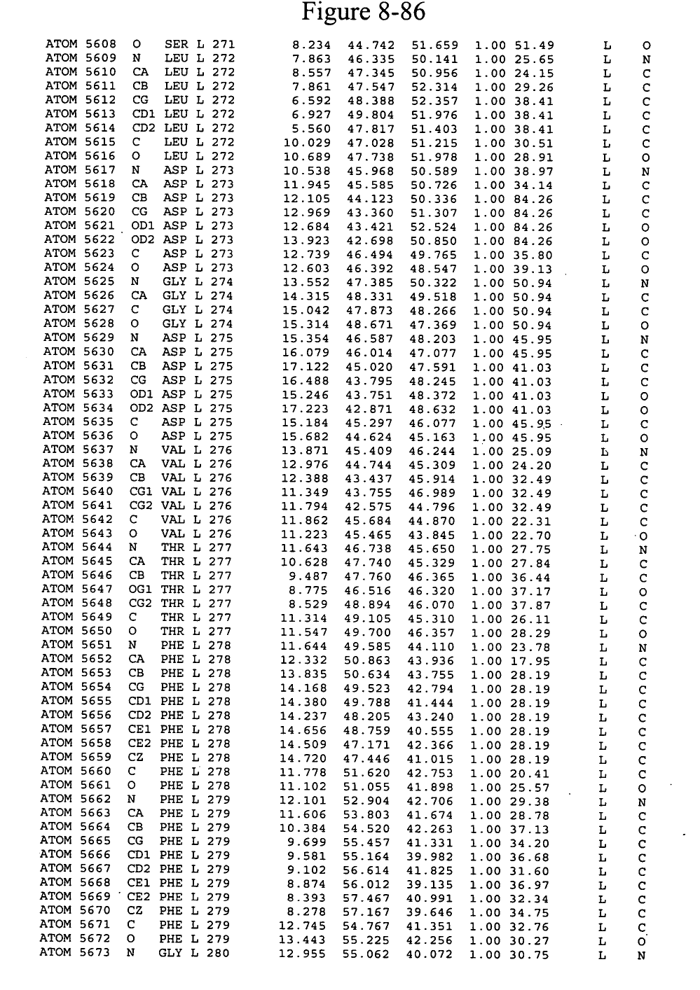

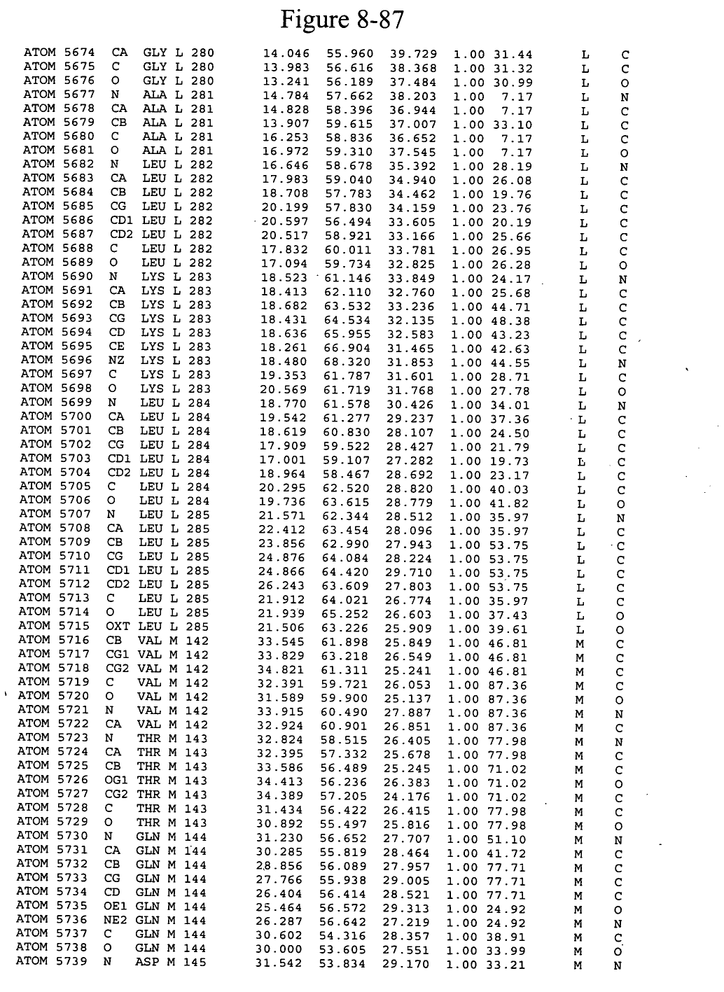

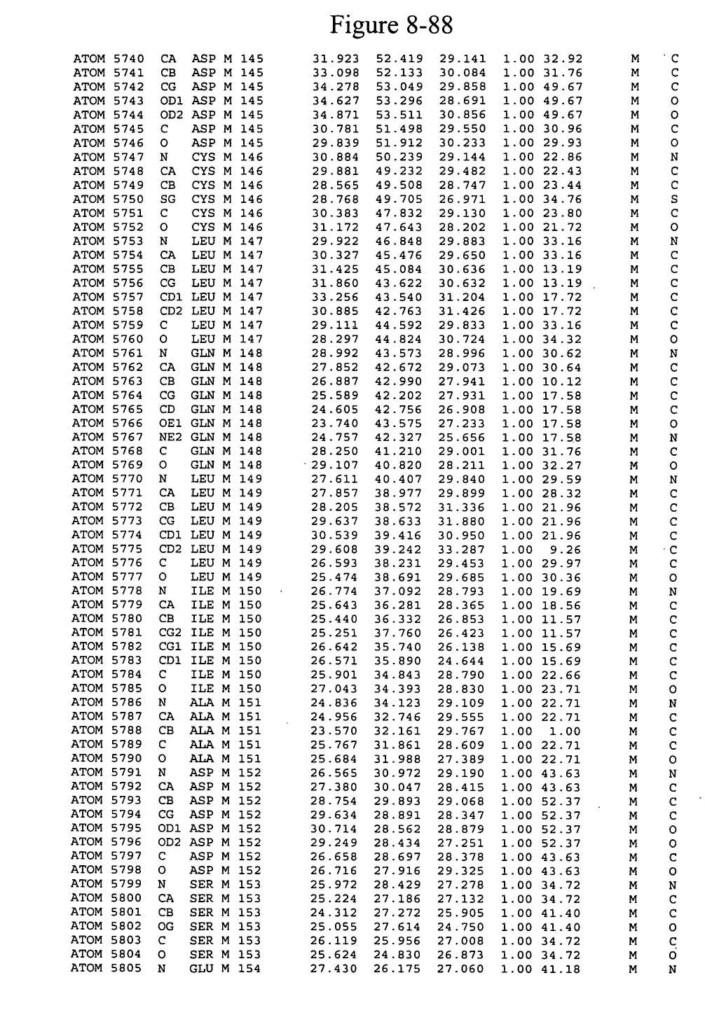

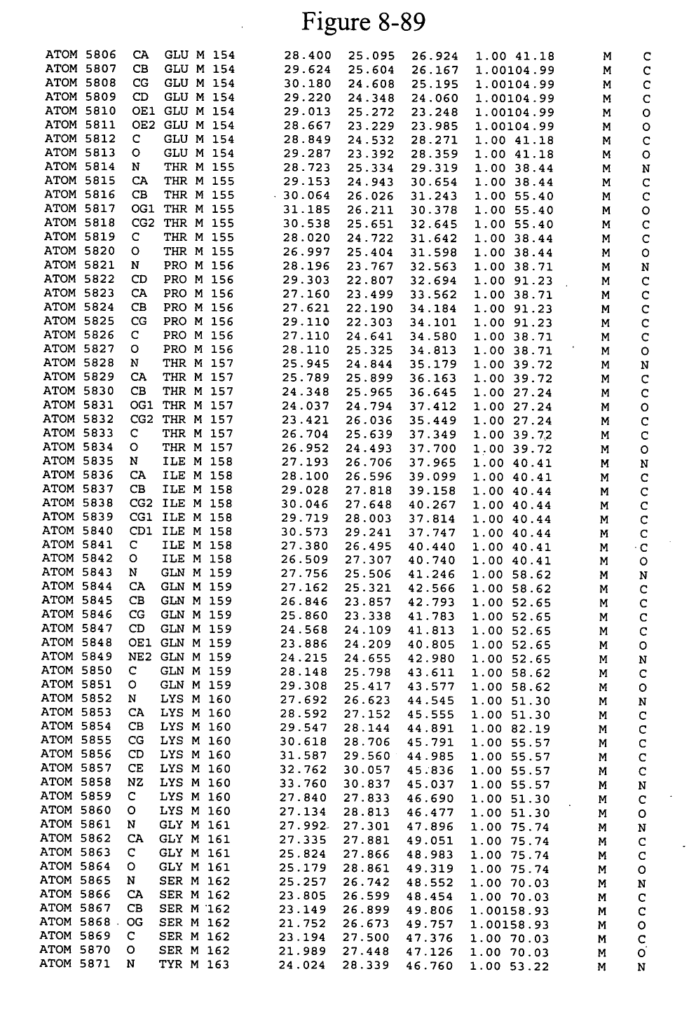

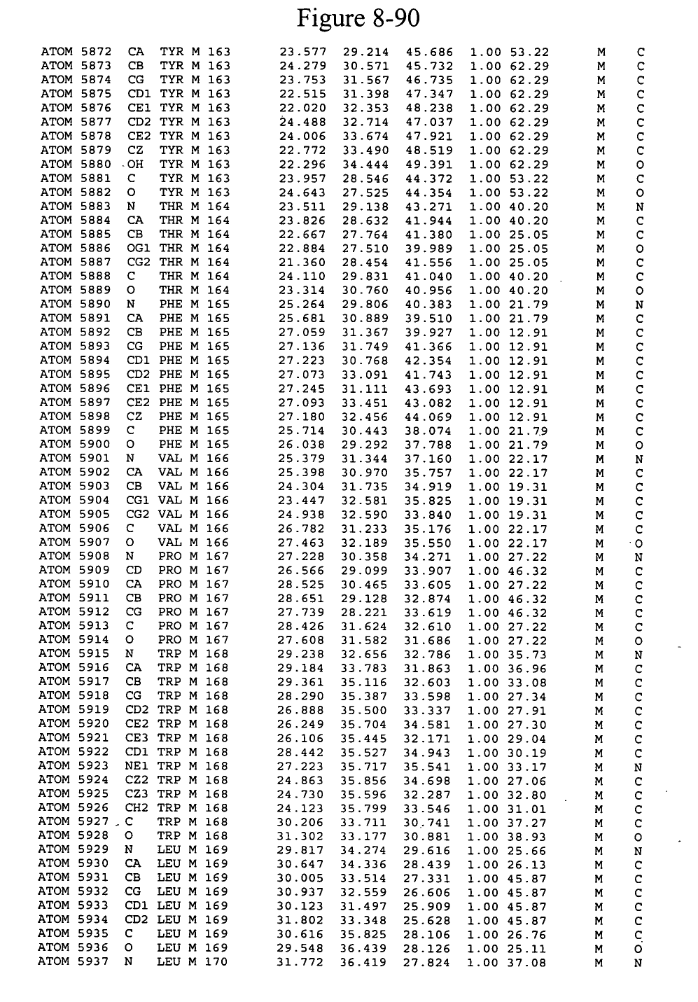

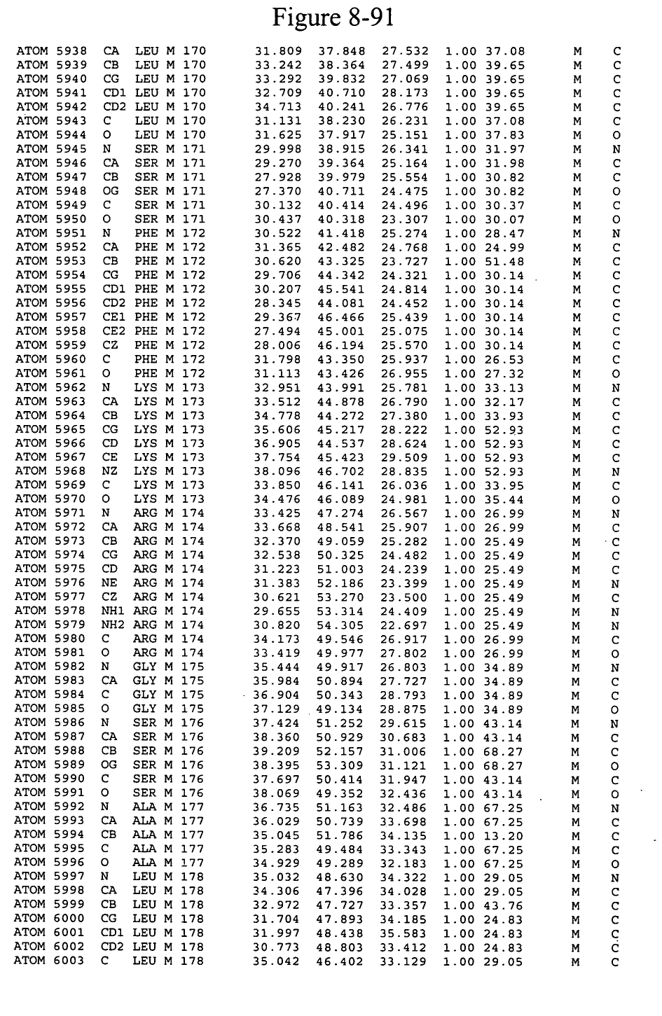

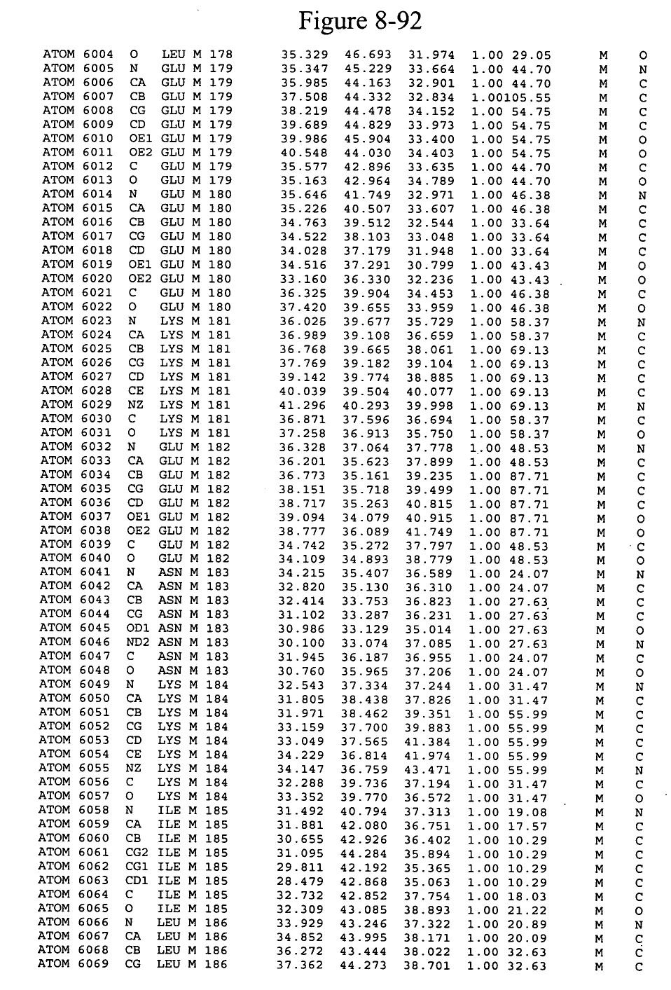

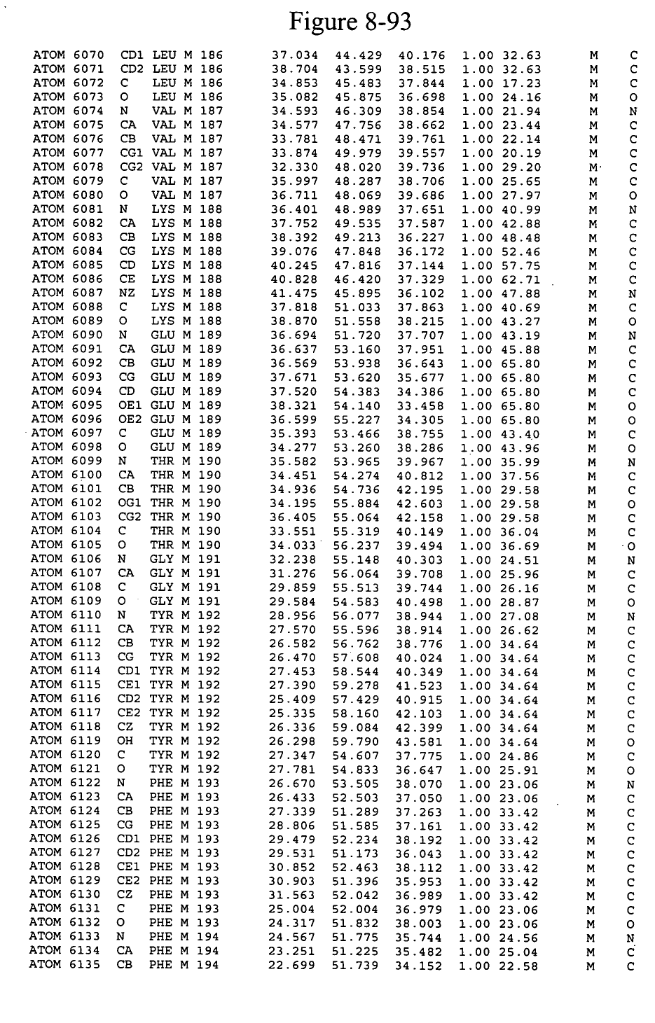

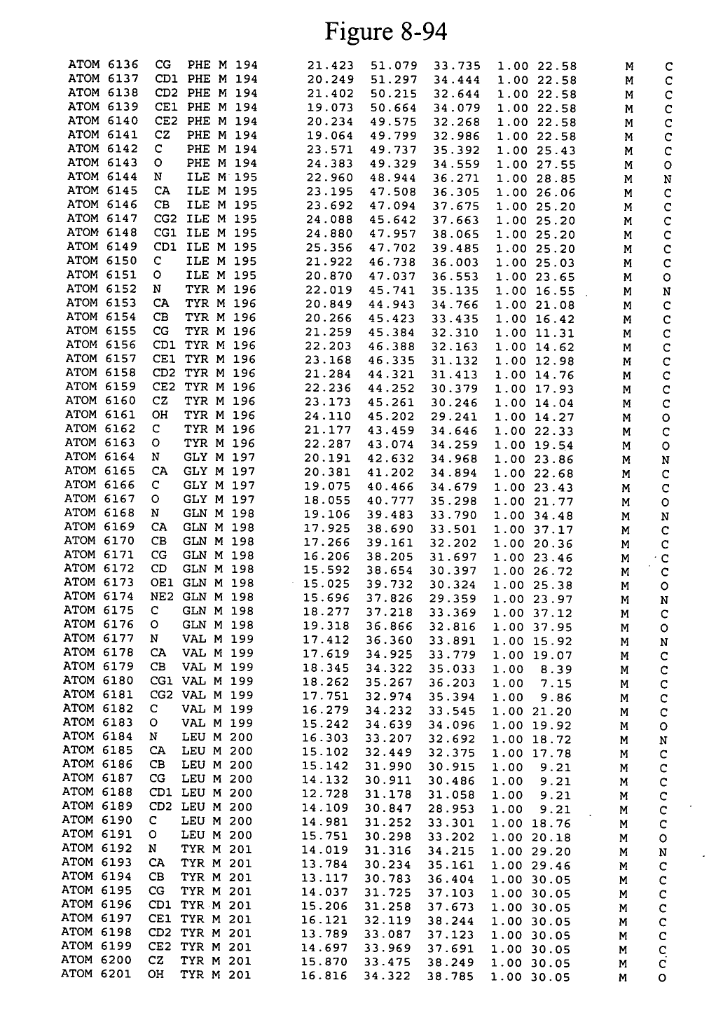

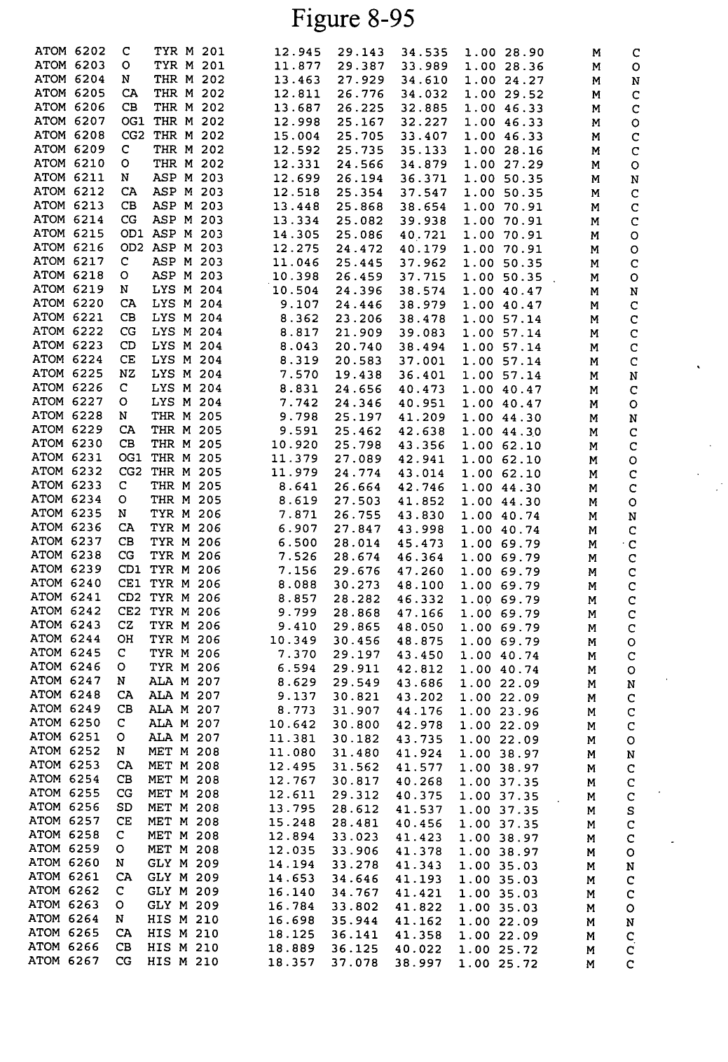

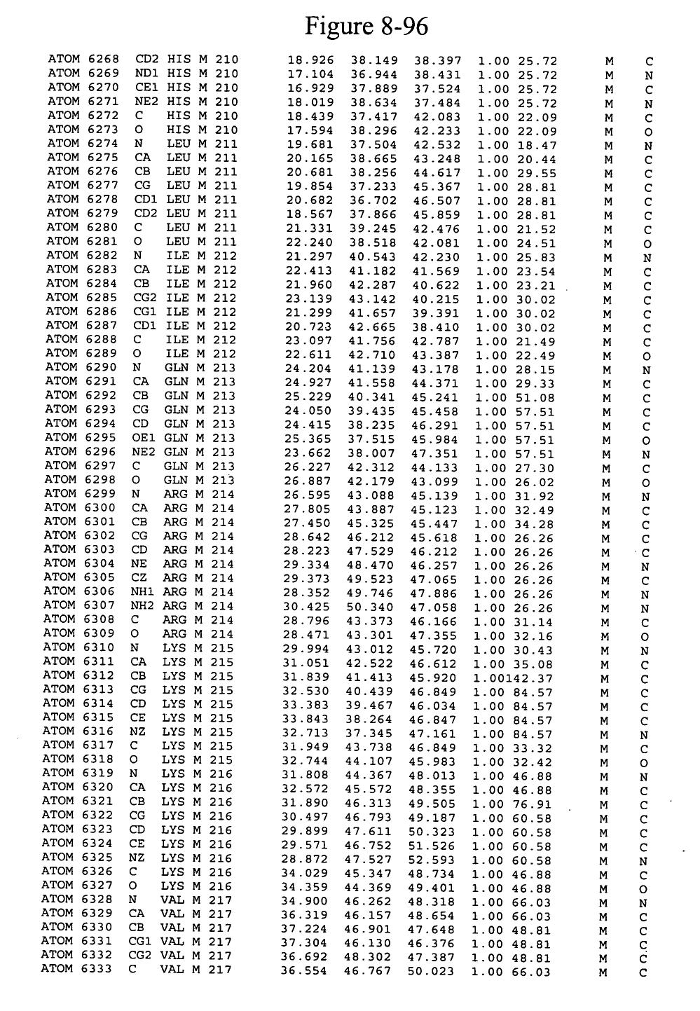

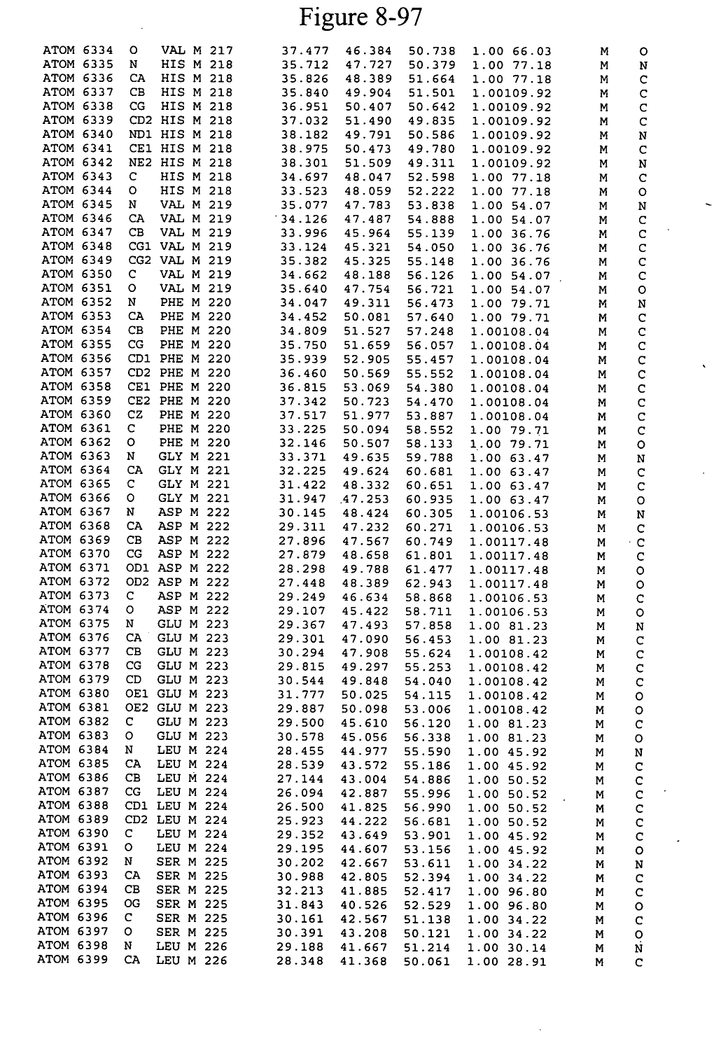

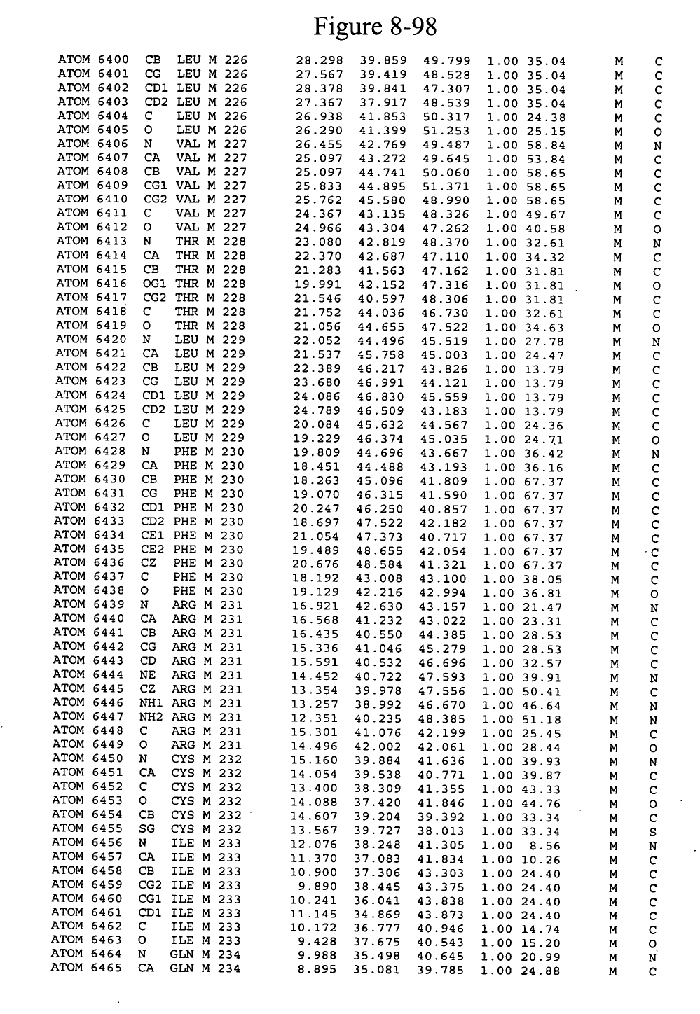

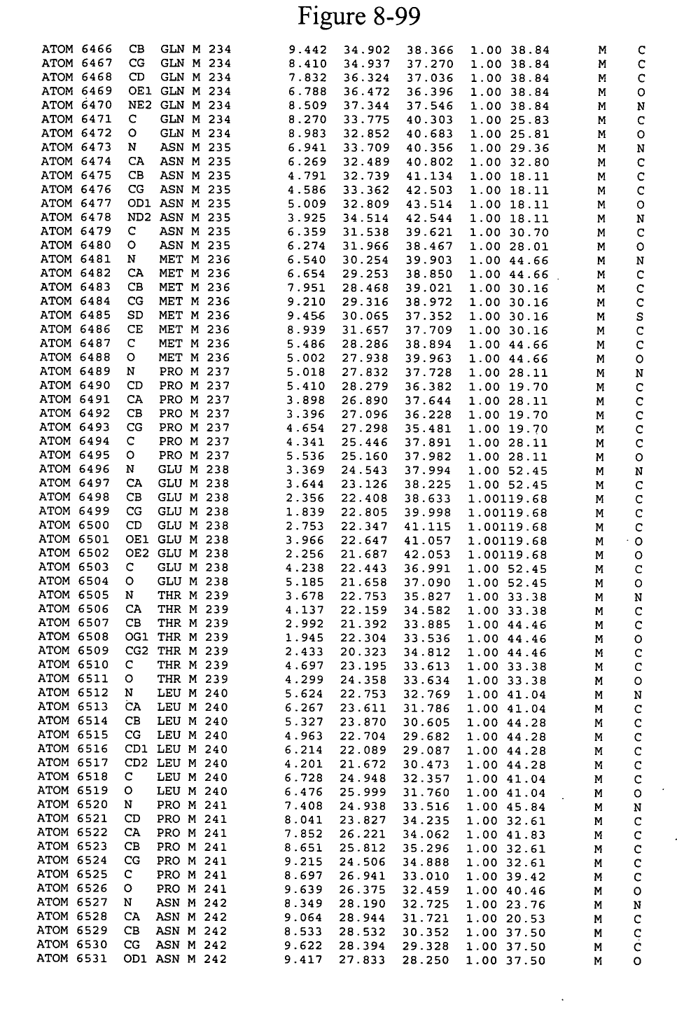

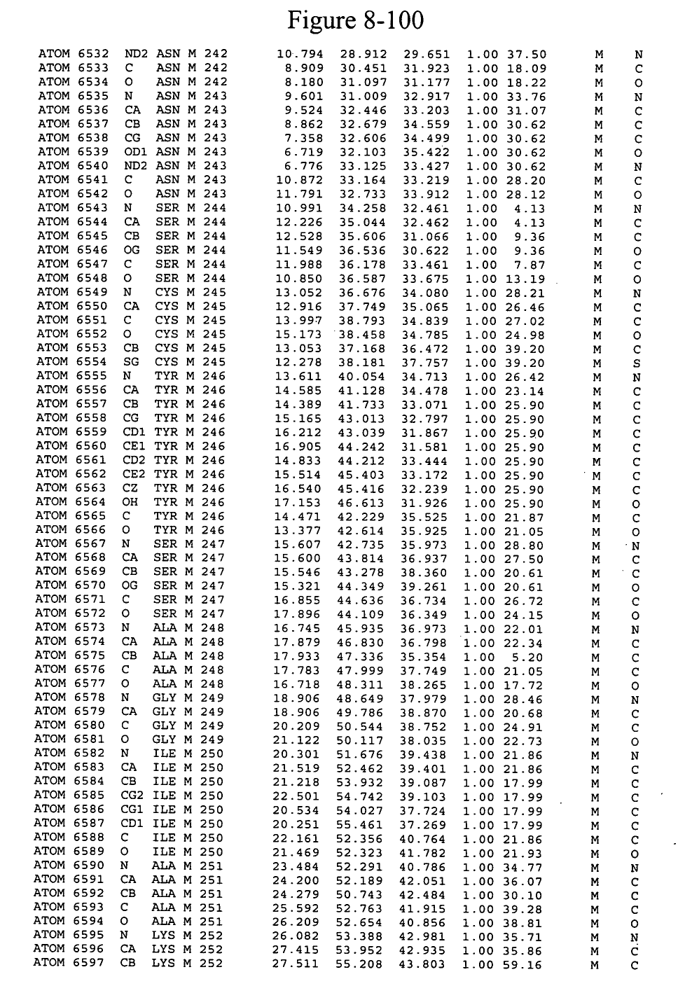

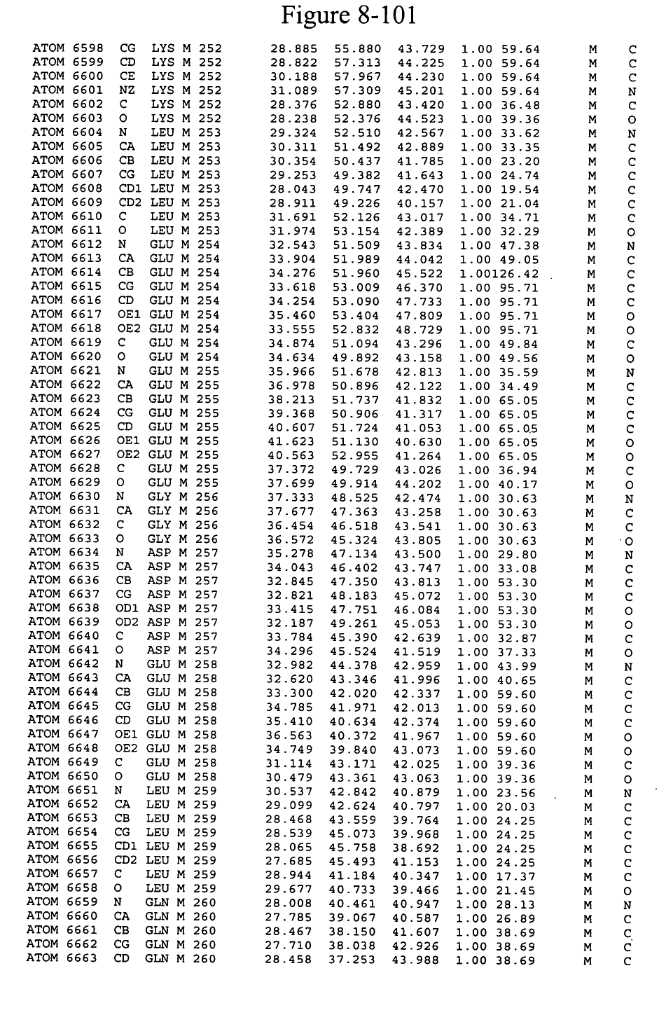

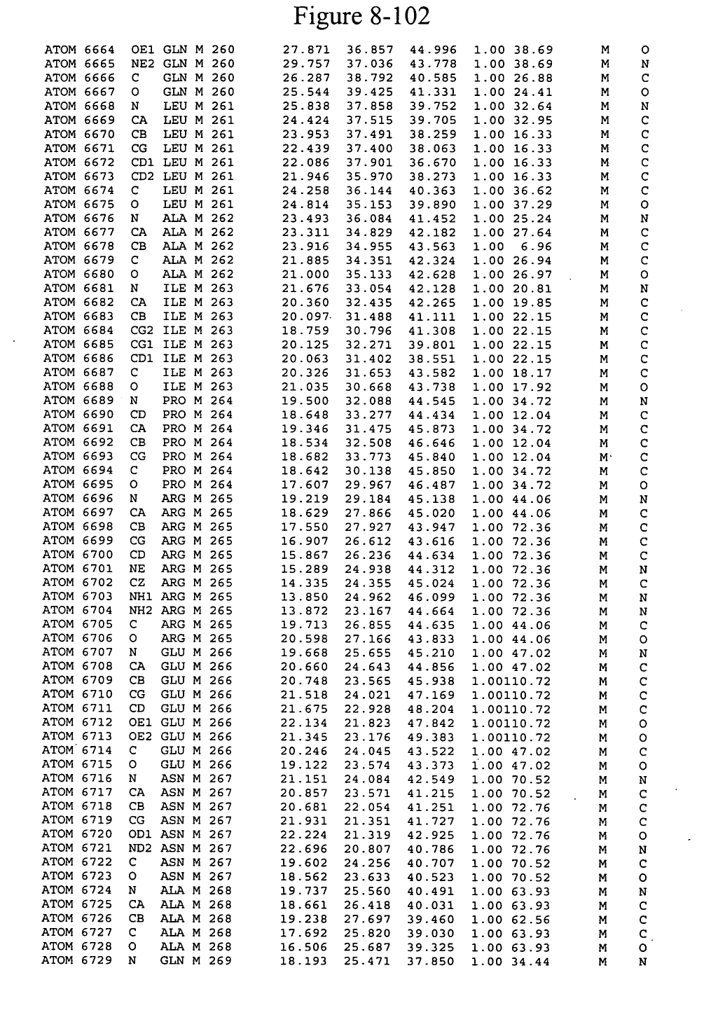

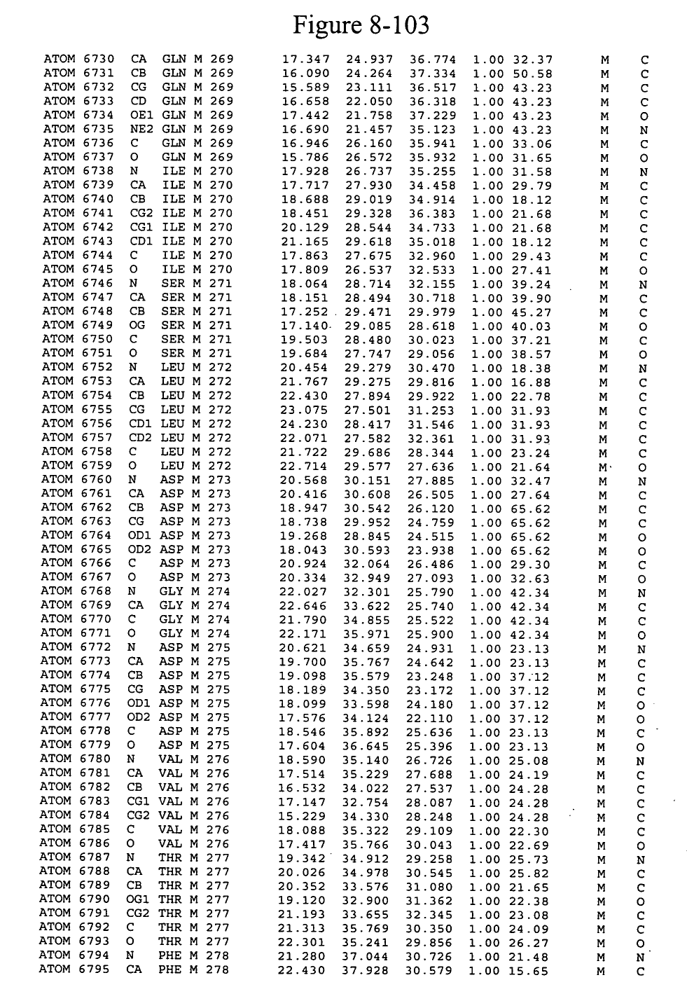

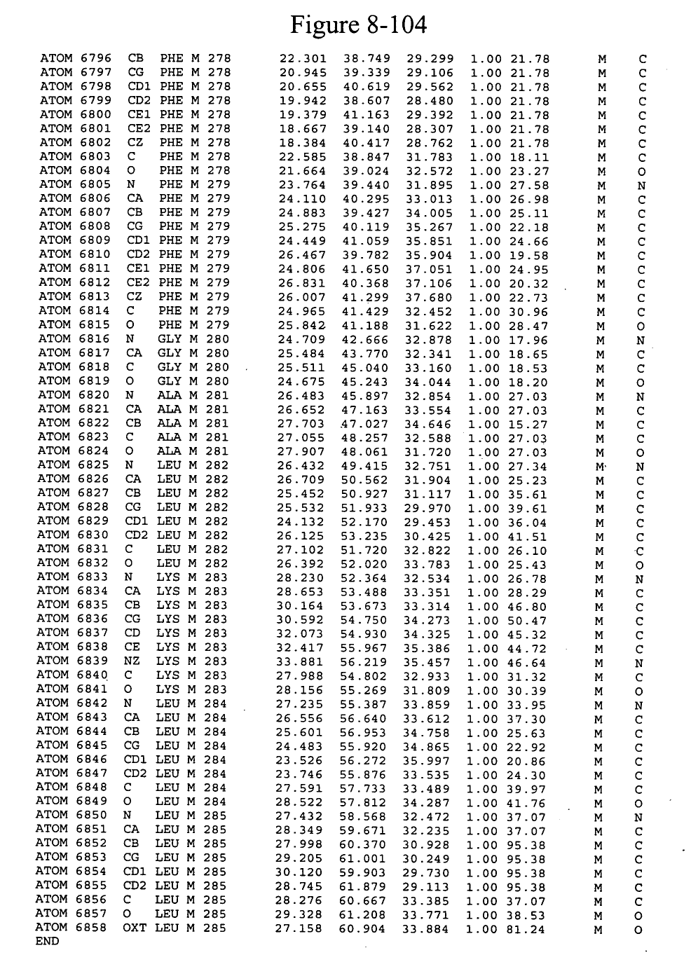

The three-dimensional structure of the extracellular domain of BAFF polypeptide is defined by a set of structure coordinates as set forth in FIG. 8. The term “structure coordinates” refers to Cartesian atomic coordinates derived from mathematical equations related to the patterns obtained on diffraction of a monochromatic beam of X-rays by the atoms (scattering centers) of an extracellular domain of a BAFF polypeptide in crystal form. The diffraction data are used to calculate an electron density map of the repeating unit of the crystal. The electron density maps are then used to establish the position of individual atoms of the extracellular domain of a BAFF polypeptide.

One embodiment of the present invention provides a molecule or a molecular complex defined by at least a portion or all of the structure coordinates of the BAFF polypeptide amino acids set forth in FIG. 8, or a homologue of said molecule or said molecular complex, wherein said homologue has a root mean square deviation from the backbone atoms of said amino acids between 0.00 Å and 1.50 Å, preferably between 0.00 Å and 1.00 Å, more preferably between 0.00 Å and 0.50 Å.

Another embodiment of the present invention provides a molecule or a molecular complex comprising a first binding site defined by structure coordinates of at least one or a plurality of BAFF amino acids selected from the group consisting of His218, Val219, Phe220, Gly221, Asp222, Glu223, Leu224, Ser225, Leu226, Val227, Pro264, Arg265, Glu266, Gly161, Ser162, Tyr163, Ala151, Asp152, Ser153, Glu154, Thr155, Pro156, Leu240, Pro241, Asn242, Ser171 and Phe172 according to FIG. 8; or a homologue of said molecule or molecular complex, wherein said homologue comprises a second binding site that has a root mean square deviation from the backbone atoms of said at least one or a plurality of BAFF amino acids between 0.00 Å and 1.50 Å, preferably between 0.00 Å and 1.00 Å, more preferably between 0.00 Å and 0.50 Å. The first or second binding site is preferably a binding site of BAFF for one or more receptors of BAFF.

Preferably, a certain embodiment of the present invention provides a molecule or a molecular complex comprising a first binding site defined by structure coordinates of at least four BAFF amino acids selected from the group consisting of His218, Val219, Phe220, Gly221, Asp222, Glu223, Leu224, Ser225, Leu226, Val227, Pro264, Arg265, Glu266, Gly161, Ser162, Tyr163, Ala151, Asp152, Ser153, Glu154, Thr155, Pro156, Leu240, Pro241, Asn242, Ser171 and Phe172 according to FIG. 8; or a homologue of said molecule or molecular complex, wherein said homologue comprises a second binding site that has a root mean square deviation from the backbone atoms of said at least four BAFF amino acids between 0.00 Å and 1.50Å, preferably between 0.00 Å and 1.00 Å, more preferably between 0.00 Å and 0.50 Å. The first or second binding site is preferably a binding site of BAFF for one or more receptors of BAFF.

Those of skill in the art will understand that a set of structure coordinates for a polypeptide is a relative set of points that define a shape in three dimensions. Thus, it is possible that an entirely different set of coordinates could define a similar or identical shape. Moreover, slight variations in the individual coordinates will have little effect on overall shape.

The variations in coordinates discussed above may be generated due to mathematical manipulations of the structure coordinates. For example, the structure coordinates set forth in FIG. 8 could be manipulated by crystallographic permutations of the structure coordinates, fractionalization of the structure coordinates, integer additions or subtractions to sets of the structure coordinates, inversion of the structure coordinates, or any combination thereof.

Alternatively, modification in the crystal structure due to mutations, additions, substitutions, and/or deletions of amino acids, or other changes in any of the components that make up the crystal, could also account for variations in structure coordinates. If such variations are within an acceptable standard error as compared to the original coordinates, the resulting three-dimensional shape is considered to be the same as that of the unmodified crystal.

Various computational analyses are therefore necessary to determine whether a molecule or molecular complex, or a portion thereof, is sufficiently similar to all or parts of the extracellular domain of BAFF polypeptide structure described herein as to be considered the same. Such analyses may be carried out in current software applications, such as the Molecular Similarity application of QUANTA (Molecular Simulations Inc., San Diego, Calif.) version 4.1, and as described in its accompanying User's Guide.

The Molecular Similarity application permits comparisons between different structures, different conformations of the same structure, and different parts of the same structure. The procedure used in Molecular Similarity to compare structures is divided into four steps: 1) load the structures to be compared; 2) define the atom equivalences in these structures; 3) perform a fitting operation; and 4) analyze the results.

Each structure is identified by a name. One structure is identified as the target (i.e., the fixed structure); all remaining structures are working structures (i.e., moving structures). Since atom equivalency within QUANTA is defined by user input, for the purpose of this invention, equivalent atoms such as protein backbone atoms (N, Cα, C and O) will be defined for all conserved residues between the two structures being compared. Also, only rigid fitting operations will be considered.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by QUANTA.

For the purpose of this invention, any molecule or molecular complex that has a root mean square deviation of conserved residue backbone atoms (N, Cα, C, O) between 0.00 Å and 1.50Å, preferably between 0.00 Å and 1.00 Å, more preferably between 0.00 Å and 0.05 Å, when superimposed on the relevant backbone atoms described by structure coordinates listed in FIG. 8 are considered identical.

The terms “root mean square deviation”, “r. m. s. deviation” or “r. m. s. positional deviation” mean the square root of the arithmetic mean of the squares of the deviations from the mean. It is a way to express the deviation or variation from a trend or object. For purposes of this invention, the “root mean square deviation” defines the variation in the backbone of a protein from the relevant portion of the backbone of the BAFF polypeptide as defined by the structure coordinates described herein.

Once the structure coordinates of a protein crystal have been determined, they are useful in solving the structures of other crystals.

In accordance with the present invention, the structure coordinates of a molecule or molecular complex comprising the extracellular domain of BAFF polypeptide is stored in a machine-readable storage medium. A machine could be a computer. Such data may be used for a variety of purposes, such as drug discovery, and X-ray crystallographic analysis of other protein crystals.

In order to use the structure coordinates generated for the BAFF molecule or molecular complex or one of its binding sites or homologues thereof, it is necessary to convert them into a three-dimensional shape. This is achieved through the use of commercially available software that is capable of generating a three-dimensional graphical representation of molecule or molecular complexes, or portions thereof, from a set of structure coordinates. Commercially available graphical software programs including, but not limited to, O (Jones et al., Acta Cryst. A47: p. 110-9 (1991)) and INSIGHTII (© Accelrys, Inc. and Molecular Simulations, Inc., San Diego, Calif.) are capable of generating three-dimensional representations of molecules or molecular complexes, or portions thereof, from a set of structure coordinates.

Accordingly, one embodiment of this invention provides a machine-readable data storage medium comprising a data storage material encoded with machine-readable data comprising a portion of or the entire set of the structure coordinates set forth in FIG. 8. A machine could be a computer. A computer which comprises the data storage medium is also provided by this invention. This invention also provides the computer with instructions to produce three-dimensional representations of the molecules or molecular complexes of BAFF by processing the machine-readable data of this invention. The computer of this invention further comprises a display for displaying the structure coordinates of this invention.

This invention also provides a computer for determining at least a portion, or all, of the structure coordinates corresponding to X-ray diffraction data obtained from a molecule or a molecular complex of BAFF, wherein said computer comprises:

a) a machine-readable data storage medium comprising a data storage material encoded with machine-readable data, wherein said data comprises at least a portion, or all, of the structure coordinates of BAFF polypeptide according to FIG. 8;

b) a machine-readable data storage medium comprising a data storage material encoded with machine-readable data, wherein said data comprises X-ray diffraction data obtained from said molecule or molecular complex; and

c) instructions for performing a Fourier transform of the machine readable data of (a) and for processing said machine readable data of (b) into structure coordinates.

The computer of this invention further comprises a display for displaying the structure coordinates of this invention.

This invention also provides a computer for determining at least a portion of the structure coordinates corresponding to X-ray diffraction data of a molecule or a molecular complex whose structure is unknown, wherein said computer comprises:

a) a machine-readable data storage medium comprising a data storage material encoded with machine-readable data, wherein said data comprises at least a portion, or all, of the structure coordinates according to FIG. 8;

b) a machine-readable data storage medium comprising a data storage material encoded with machine-readable data, wherein said data comprises an X-ray diffraction pattern of said molecule or molecular complex;

c) a working memory for storing instructions for processing said machine-readable data of a) and b);

d) a central processing unit coupled to said working memory and to said machine-readable data of a) and b) for performing a Fourier transform of the machine readable data of (a) and for processing said machine readable data of (b) into structure coordinates; and

e) preferably a display coupled to said central processing unit for displaying said structure coordinates of said molecule of molecular complex. In one embodiment, the unknown structure is at least a portion of BAFF. In another embodiment, the unknown structure comprises an APRIL polypeptide. In a preferred embodiment, the unknown structure comprises the extracellular domain of APRIL. In a more preferred embodiment, the unknown structure comprises a trimer of APRIL polypeptides.

This invention further provides a computer for producing a three-dimensional representation of:

a) a molecule or a molecular complex defined by at least a portion or all of the structure coordinates of the BAFF amino acids set forth in FIG. 8, or

b) a homologue of said molecule or molecular complex, wherein said homologue has a root mean square deviation from the backbone atoms of said amino acids of between 0.00 Å than 1.50Å, preferably between 0.00 Å and 1.00 Å, more preferably between 0.00 Å and 0.50 Å; and wherein said computer comprises:

(i) a machine-readable data storage medium comprising a data storage material encoded with machine-readable data, wherein said data comprises at least a portion or all of the structure coordinates of all of the BAFF amino acids set forth in FIG. 8; and

(ii) instructions for processing said machine-readable data into said three-dimensional representation.

This invention also provides a computer for producing a three-dimensional representation of:

a) a molecule or molecular complex comprising a first binding site defined by structure coordinates of at least one or a plurality of BAFF amino acids selected from the group consisting of His218, Val219, Phe220, Gly221, Asp222, Glu223, Leu224, Ser225, Leu226, Val227, Pro264, Arg265, Glu266, Gly161, Ser162, Tyr163, Ala151, Asp152, Ser153, Glu154, Thr155, Pro156, Leu240, Pro241, Asn242, Ser171 and Phe172 according to FIG. 8; or

b) a homologue of said molecule or molecular complex, wherein said homologue comprises a second binding site that has a root mean square deviation from the backbone atoms of said at least one or a plurality of BAFF amino acids of between 0.00 Å and 1.50Å, preferably between 0.00 Å and 1.00 Å, more preferably between 0.00 Å and 0.50A; wherein said computer comprises:

(i) a machine-readable data storage medium comprising a data storage material encoded with machine-readable data, wherein said data comprises the structure coordinates of at least one or a plurality of BAFF amino acids selected from the group consisting of His218, Val219, Phe220, Gly221, Asp222, Glu223, Leu224, Ser225, Leu226, Val227, Pro264, Arg265, Glu266, Gly161, Ser162, Tyr163, Ala151, Asp152, Ser153, Glu154, Thr155, Pro156, Leu240, Pro241, Asn242, Ser17l and Phe172 according to FIG. 8; and

(ii) instructions for processing said machine-readable data into said three-dimensional representation.

This invention also provides a computer for producing a three-dimensional representation of:

c) a molecule or molecular complex comprising a first binding site defined by structure coordinates of at least four BAFF amino acids selected from the group consisting of His218, Val219, Phe220, Gly221, Asp222, Glu223, Leu224, Ser225, Leu226, Val227, Pro264, Arg265, Glu266, Gly161, Ser162, Tyr163, Ala151, Asp152, Ser153, Glu154, Thr155, Pro156, Leu240, Pro241, Asn242, Ser171 and Phe172 according to FIG. 8; or

d) a homologue of said molecule or molecular complex, wherein said homologue comprises a second binding site that has a root mean square deviation from the backbone atoms of said at least four BAFF amino acids of between 0.00 Å and 1.50Å, preferably between 0.00 Å and 1.00 Å, more preferably between 0.00 Å and 0.50 Å;

wherein said computer comprises:

(i) a machine-readable data storage medium comprising a data storage material encoded with machine-readable data, wherein said data comprises the structure coordinates of at least four BAFF amino acids selected from the group consisting of His218, Val219, Phe220, Gly221, Asp222, Glu223, Leu224, Ser225, Leu226, Val227, Pro264, Arg265, Glu266, Gly161, Ser162, Tyr163, Ala151, Asp152, Ser153, Glu154, Thr155, Pro156, Leu240, Pro241, Asn242, Ser171 and Phe172 according to FIG. 8; and

(ii) instructions for processing said machine-readable data into said three-dimensional representation.

Preferably, the computer of this invention further comprises a display (which could be a computer screen) for displaying the structure coordinates of this invention. The first or the second binding site could be, inter alia, a binding site of BAFF for one or more receptors of BAFF.

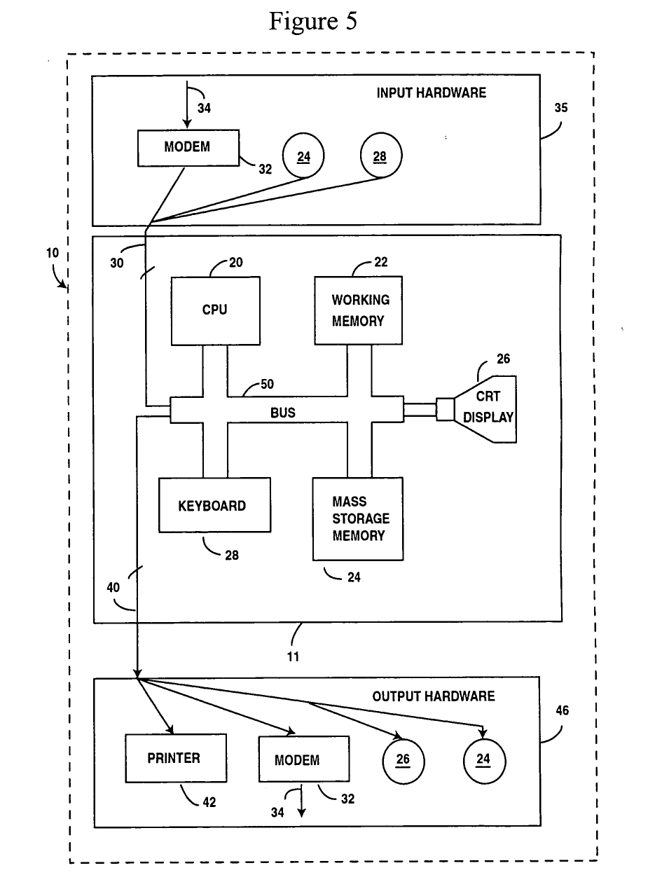

FIG. 5 demonstrates one version of these embodiments. System 10 includes a computer 11 comprising a central processing unit (“CPU”) 20, a working memory 22 which may be, e.g., RAM (random-access memory) or “core” memory, mass storage memory 24 (such as one or more disk drives or CD-ROM or DVD-ROM drives), one or more cathode-ray tube (“CRT”) display terminals 26, one or more keyboards 28, one or more input lines 30, and one or more output lines 40, all of which are interconnected by a conventional bidirectional system bus 50.

Input hardware 36, coupled to computer 11 by input lines 30, may be implemented in a variety of ways. Machine-readable data of this invention may be inputted via the use of a modem or modems 32 connected by a telephone line or dedicated data line 34. Alternatively or additionally, the input hardware 35 may comprise CD-ROM or DVD-ROM drives or disk drives 24. In conjunction with display terminal 26, keyboard 28 may also be used as an input device.

Output hardware 46, coupled to computer 11 by output lines 40, may similarly be implemented by conventional devices. By way of example, output hardware 46 may include CRT display terminal 26 for displaying a graphical representation of a binding site of this invention using a program such as QUANTA as described herein. Output hardware might also include a printer 42, so that hard copy output may be produced, or a disk drive 24, to store system output for later use.

In operation, CPU 20 coordinates the use of the various input and output devices 36, 46, coordinates data accesses from mass storage 24 and accesses to and from working memory 22, and determines the sequence of data processing steps. A number of programs may be used to process the machine-readable data of this invention. Such programs are discussed in reference to the computational methods of drug discovery as described herein. Specific references to components of the hardware system 10 are included as appropriate throughout the following description of the data storage medium.

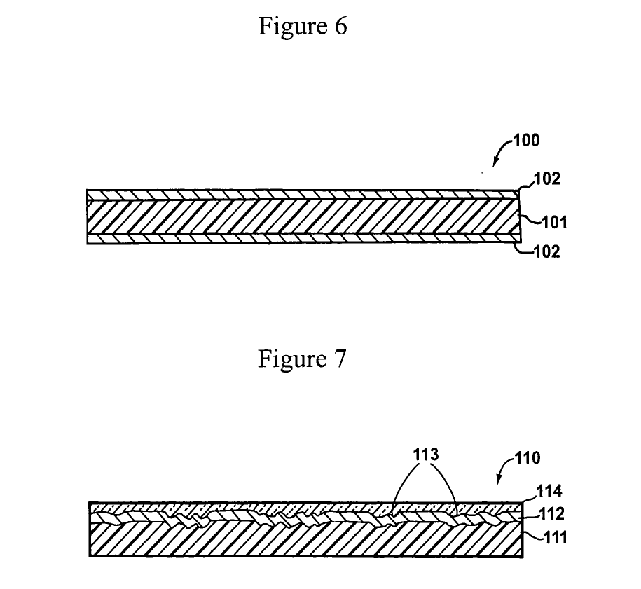

FIG. 6 shows a cross-section of a magnetic data storage medium 100 which can be encoded with machine-readable data that can be carried out by a system such as system 10 of FIG. 5. Medium 100 can be a conventional floppy diskette or hard disk, having a suitable substrate 101, which may be conventional, and a suitable coating 102, which may be conventional, on one or both sides, containing magnetic domains (not visible) whose polarity or orientation can be altered magnetically. Medium 100 may also have an opening (not shown) for receiving the spindle of a disk drive or other data storage device such as mass storage 24 of FIG. 5.

The magnetic domains of coating 102 of medium 100 are polarized or oriented so as to encode in a manner which may be conventional, machine readable data such as that described herein, for execution by a system such as system 10 of FIG. 5.

FIG. 7 shows a cross-section of an optically-readable data storage medium 110 which also can be encoded with such machine-readable data, or a set of instructions, which can be carried out by a system such as system 10 of FIG. 5. Medium 110 can be a conventional compact disk or DVD disk read only memory (CD-ROM or DVD-ROM) or a rewritable medium, such as a magneto-optical disk which is optically readable and magneto-optically writable. Medium 100 preferably has a suitable substrate 111, which may be conventional, and a suitable coating 112, which may be conventional, usually of one side of substrate 111.

In the case of CD-ROM, as is well known, coating 112 is reflective and is impressed with a plurality of pits 113 to encode the machine-readable data. The arrangement of pits is read by reflecting laser light off the surface of coating 112. A protective coating 114, which preferably is substantially transparent, is provided on top of coating 112.

In the case of a magneto-optical disk, as is well known, coating 112 has no pits 113, but has a plurality of magnetic domains whose polarity or orientation can be changed magnetically when heated above a certain temperature, as by a laser (not shown). The orientation of the domains can be read by measuring the polarization of laser light reflected from coating 112. The arrangement of the domains encodes the data as described above.

For the first time, the present invention permits the use of structure-based and rational drug design techniques to design, select, and synthesize chemical entities and compounds, such as agonists or antagonists of BAFF. Additionally, the present invention permits the use of structure-based or rational drug design techniques to make improvements of conventional BAFF agonists or antagonists, that are capable of binding to the extracellular domain of BAFF.

One particularly useful drug design technique enabled by this invention is iterative drug design. Iterative drug design is a method for optimizing associations between a protein and a compound (that compound includes an antibody) by determining and evaluating the three-dimensional structures of successive sets of protein/compound complexes.

Those of skill in the art will realize that association of natural receptors or substrates with the binding sites of their corresponding ligand (such as BAFF) or enzymes is the basis of many biological mechanisms of action. Similarly, many drugs (which include monoclonal antibodies) exert their biological effects through association with the binding sites of, for example, ligands (such as BAFF), receptors (such as one of the receptors of BAFF) and enzymes. Such associations may occur with all or any parts of the binding sites. An understanding of such associations will help lead to the design of drugs having more favorable associations with their target ligand (such as BAFF), receptor or enzyme, and thus, improved biological effects. Therefore, this information is valuable in designing potential chemical entities or synthetic compounds that bind the ligands (such as BAFF), receptors or enzymes. Such synthetic compounds could act as agonists or antagonists of the ligands (such as BAFF), receptors or enzymes.

The term “binding site”, as used herein, refers to a region of a protein, that, as a result of its shape, favorably associates with, inter alia, another protein, a chemical entity, a synthetic compound or an antibody, or an antigen binding fragment thereof. For example, BAFF has a binding site for each of its three known receptors, BCMA, TACI and BAFF-R.

This invention also provides a method of determining a binding site of BAFF for one or more receptors of BAFF. In one embodiment, the binding site of BAFF for one or more of its receptors is determined based on the location of the binding site of known, homologous TNF family members for their receptors. See, e.g., Example 1. In a preferred embodiment, the binding site of BAFF for one or more receptors of BAFF is determined by a method comprising the steps of:
a) generating by biochemical means biochemical structure-function data, said data comprising one or more amino acid residues of BAFF that when mutated results in a reduction in binding between BAFF and one or more of the receptors of BAFF (i.e., said data, for example, show that mutation of one or more particular amino acid residues of BAFF results in reduction in binding between BAFF and one or more receptors of BAFF); and
b) using said data to define amino acid residues of the BAFF structure coordinates according to FIG. 8 that interact with one or more receptors of BAFF.
   In a more preferred embodiment, the binding site of BAFF for one or more of its receptors is determined by determining the co-crystal structure of BAFF, or a portion thereof that binds to a receptor of BAFF, and one of its receptors, or a portion of that receptor that binds to BAFF. The binding site of BAFF for all of its receptors can be determined by determining the co-crystal structures of BAFF and each of its receptors.

In iterative drug design, crystals of a series of protein complexed with a compound, chemical entity or another protein that binds the protein are obtained and then the three-dimensional structure of each molecule or molecular complex is solved. Such an approach provides insight into the association between the proteins and compounds or chemical entities of each complex. This is accomplished by selecting proteins, compounds or chemical entities with agonistic or antagonistic activity, obtaining crystals of this new protein/protein, compound or chemical entity complex, solving the three-dimensional structure of the complex, and comparing the associations between the new protein/protein, compound or chemical entity complex and previously solved protein/protein, compound or chemical entity complexes. By observing how changes in the protein, compound or chemical entity affect the protein/protein, compound or chemical entity associations, these associations may be optimized.

In some cases, iterative drug design is carried out by forming successive protein/protein, protein/compound or protein/chemical entity complexes and then crystallizing each new complex. Alternatively, a pre-formed protein crystal is soaked in the presence of another protein, a compound or a chemical entity, thereby forming a protein/protein, protein/compound or protein/chemical entity complexes complex and obviating the need to crystallize each individual complex.

The structure coordinates of BAFF set forth in FIG. 8 can also be used to aid in obtaining structural information about another crystallized molecule or molecular complex. This may be achieved by any of a number of well-known techniques, including molecular replacement. This method is especially useful for determining the structures of BAFF mutants and homologues, one homologue being APRIL.

The structure coordinates set forth in FIG. 8 can also be used to generate homology models of proteins having 30% or higher homology thereto. A homology model of human APRIL, generated using the structure coordinates of BAFF shown in FIG. 8, is detailed in Example 2.

Accordingly, this invention provides a method of utilizing the structure coordinates of BAFF to obtain a homology model structure of a molecule (or molecular complex) whose structure is unknown and at least a portion of whose structure is similar to the structure of BAFF (one such molecule being APRIL), comprising the step of:

applying at least a portion (or all) of the structure coordinates set forth in FIG. 8 to generate a three-dimensional molecular model of at least a portion (or all) of the molecule whose structure is unknown, to generate a homology model structure of that molecule. In one embodiment, the unknown structure is at least a portion of BAFF. In another embodiment, the unknown structure comprises an APRIL polypeptide. In a preferred embodiment, the unknown structure comprises the extracellular domain of APRIL. In a more preferred embodiment, the unknown structure comprises a trimer of APRIL polypeptides.

This invention also provides a computer for producing a three-dimensional representation of:

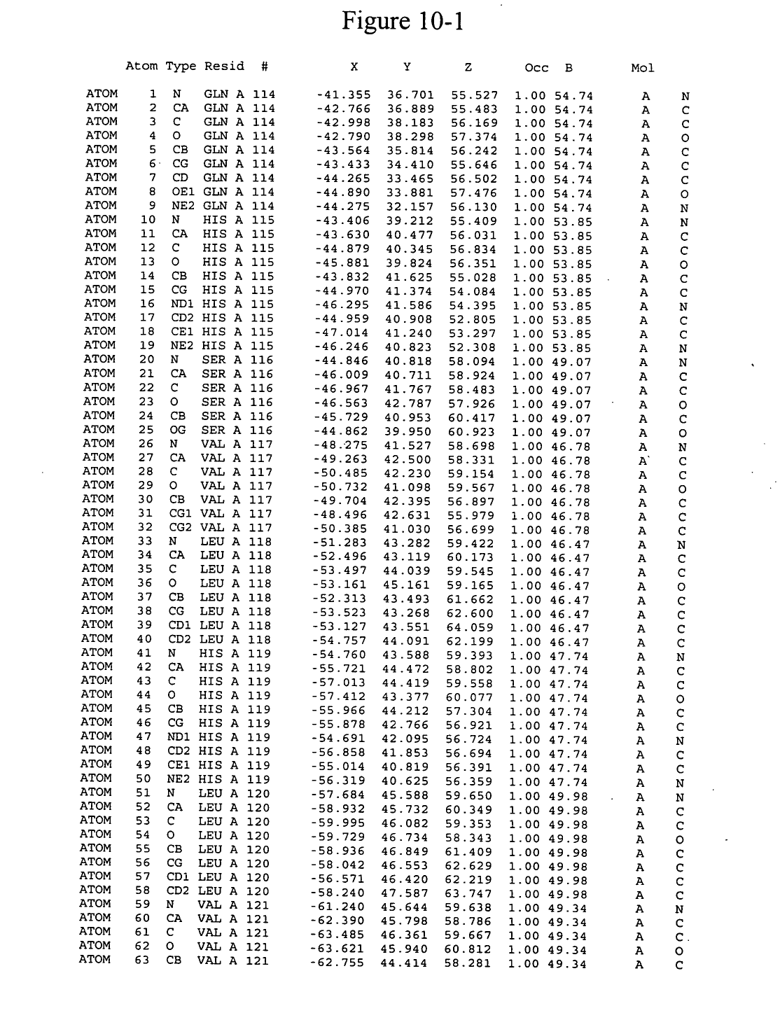

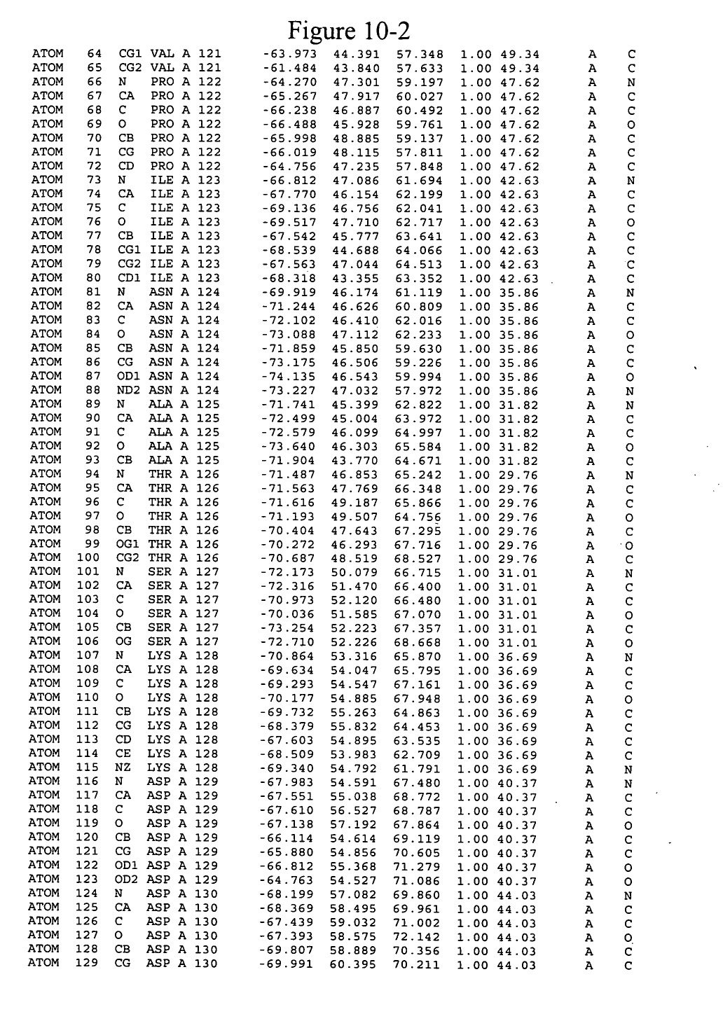

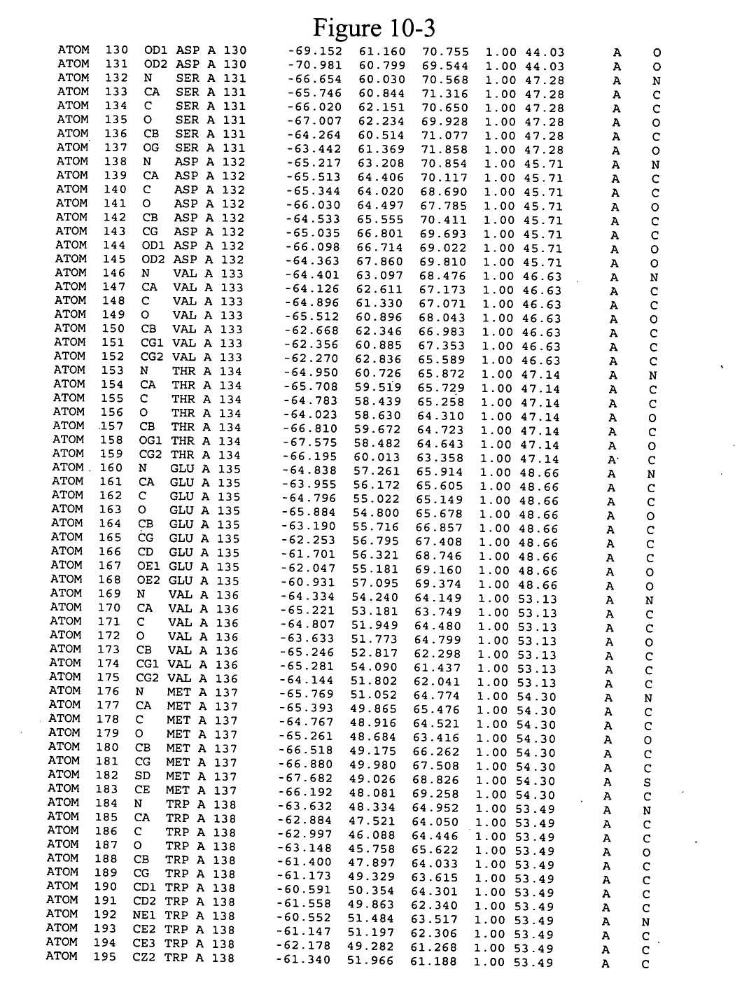

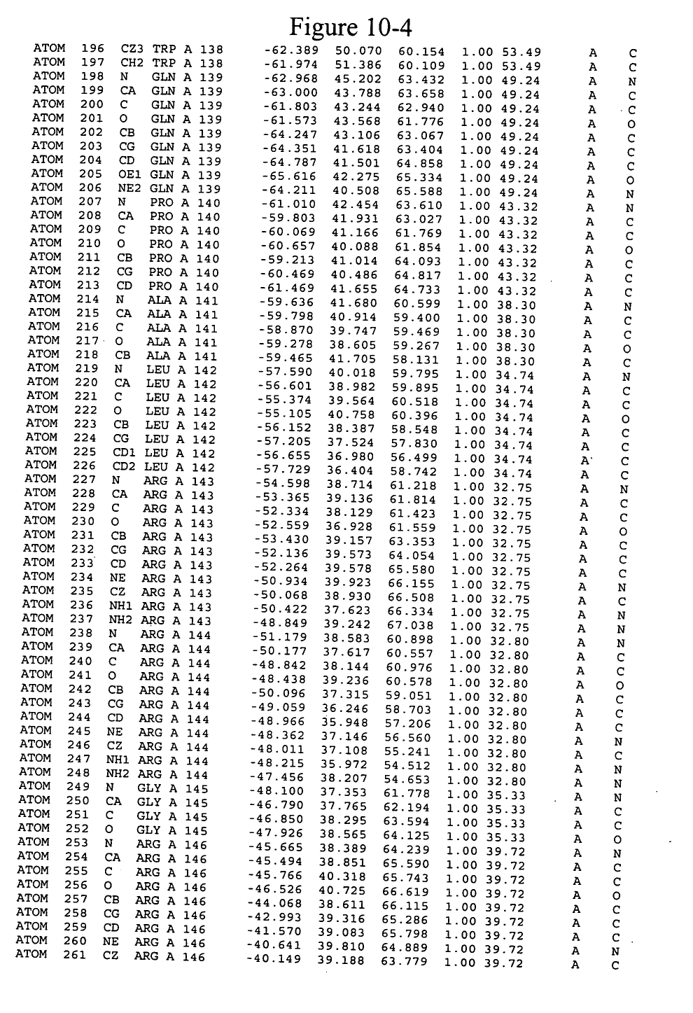

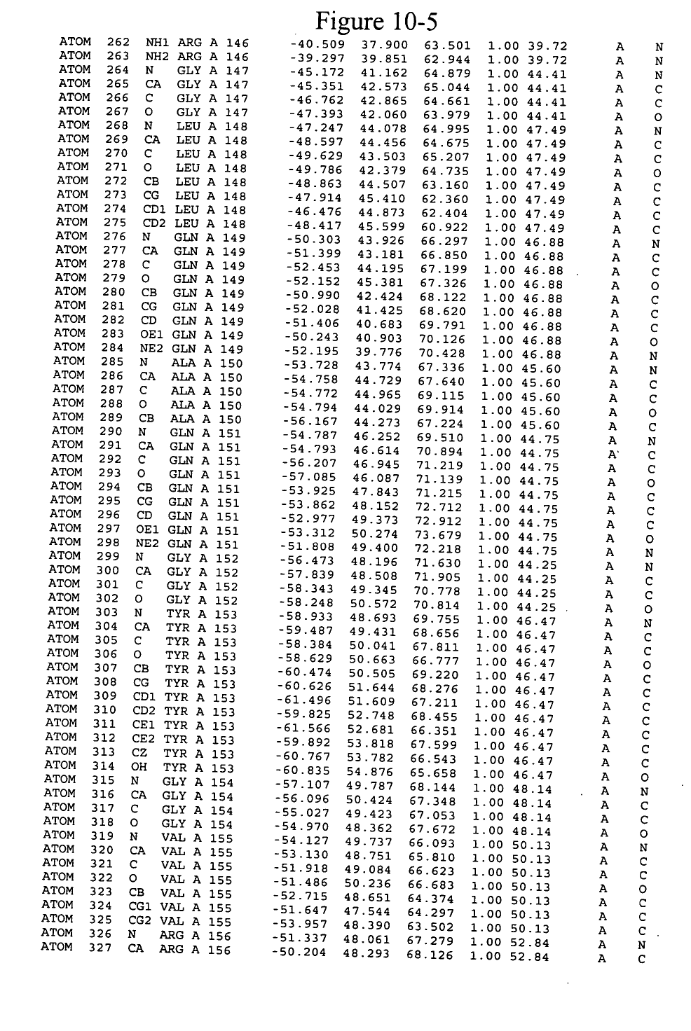

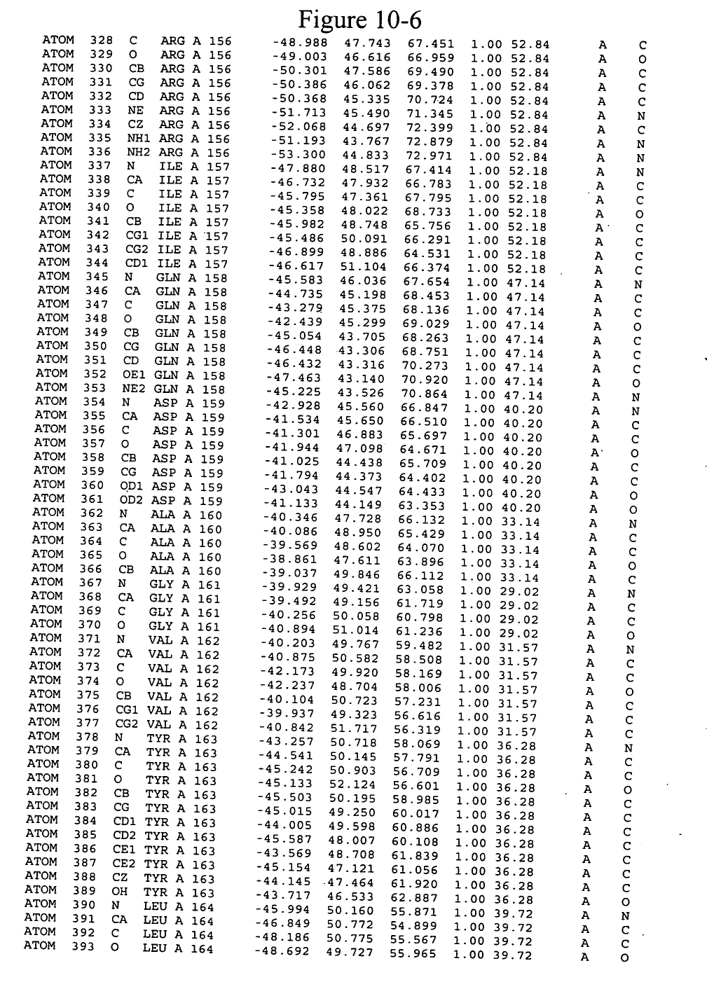

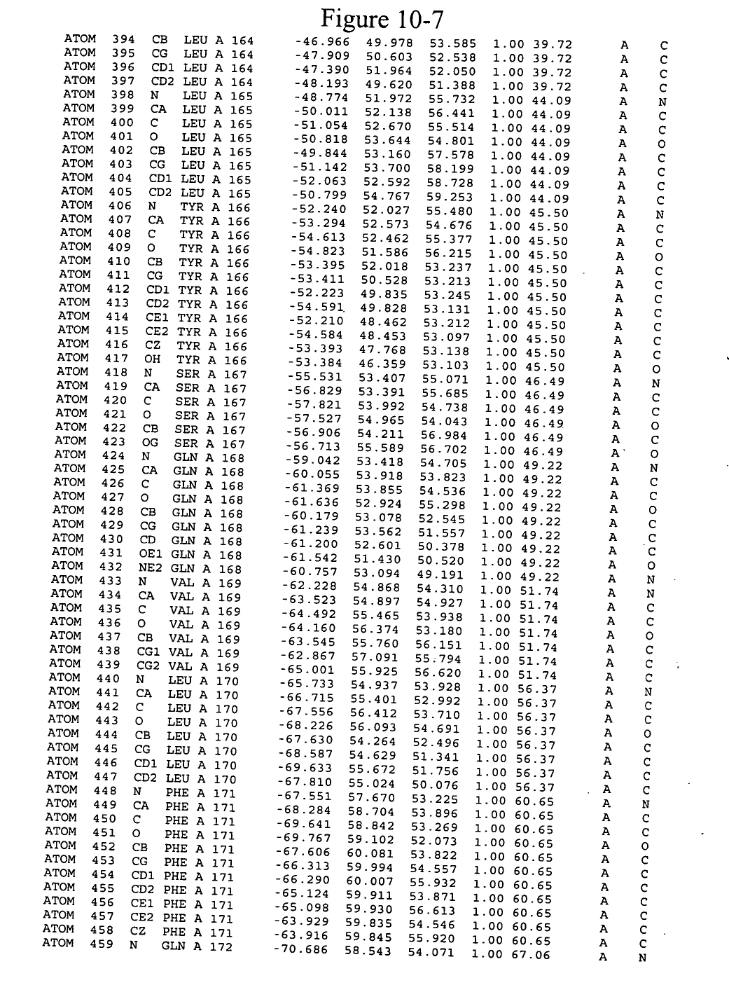

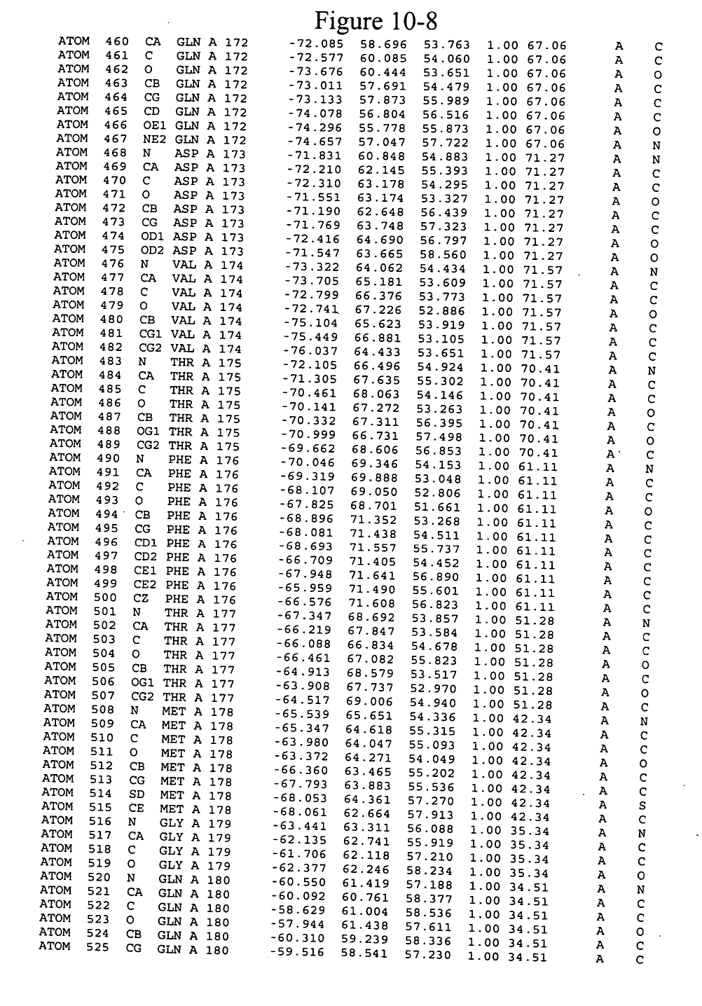

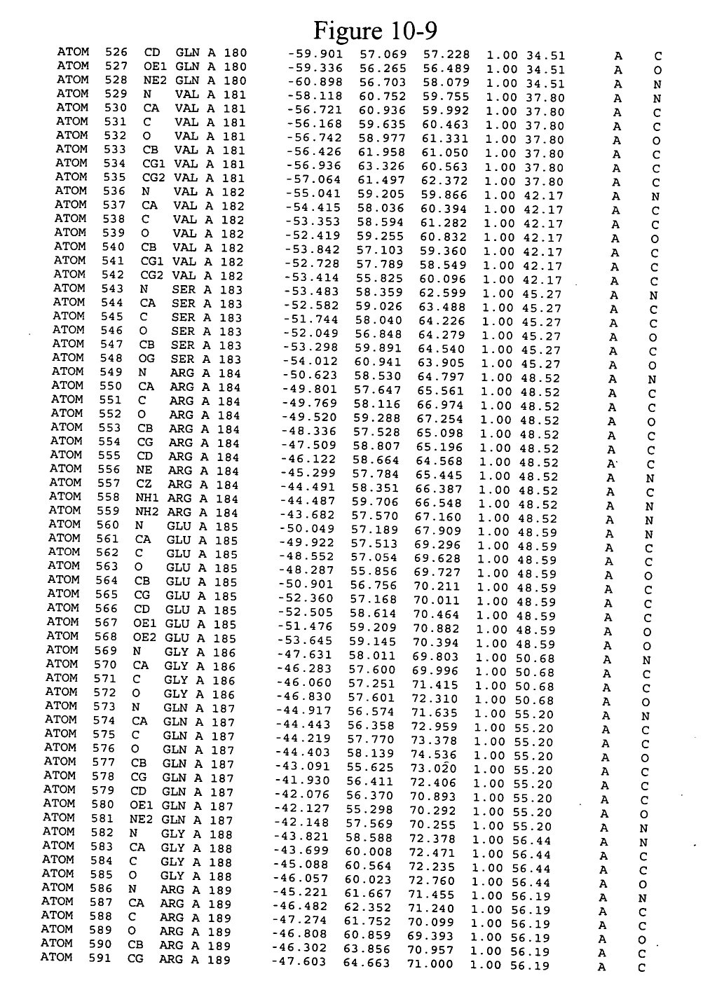

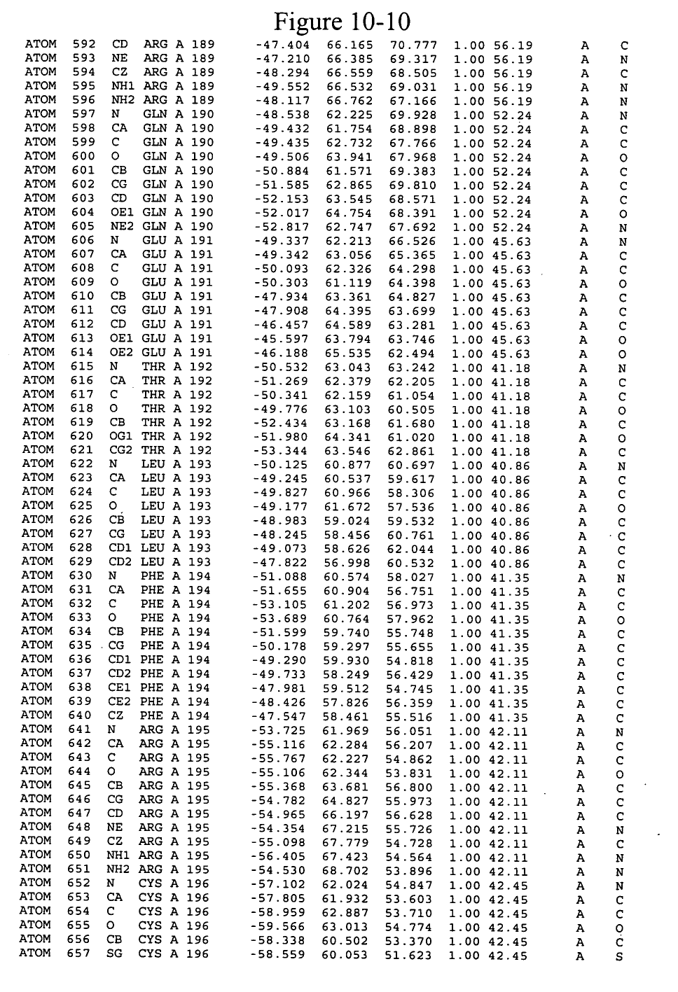

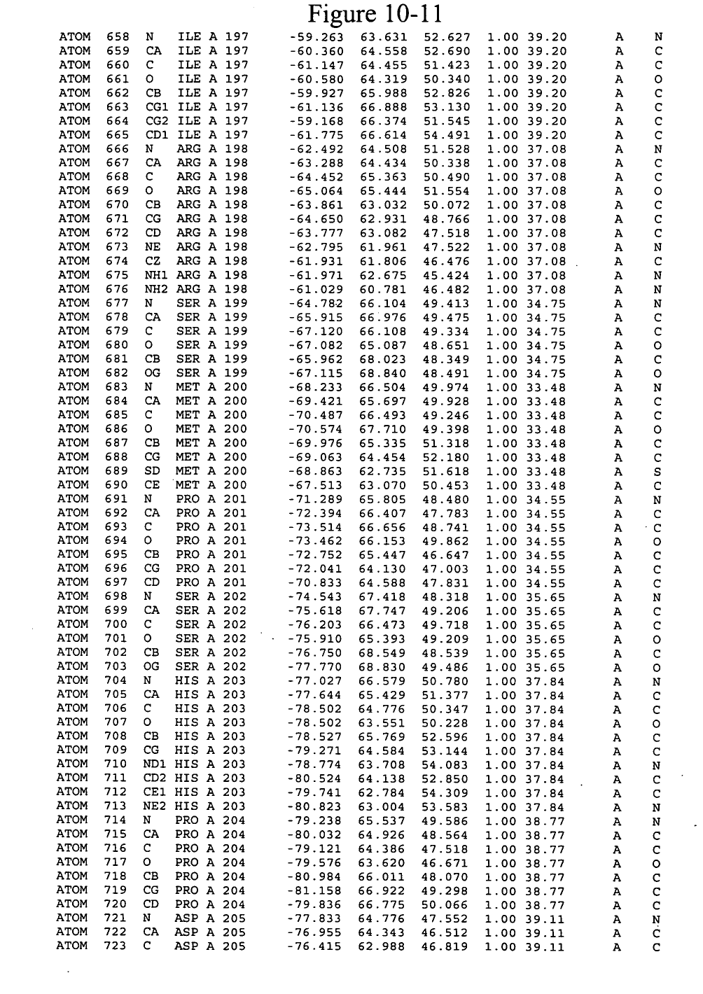

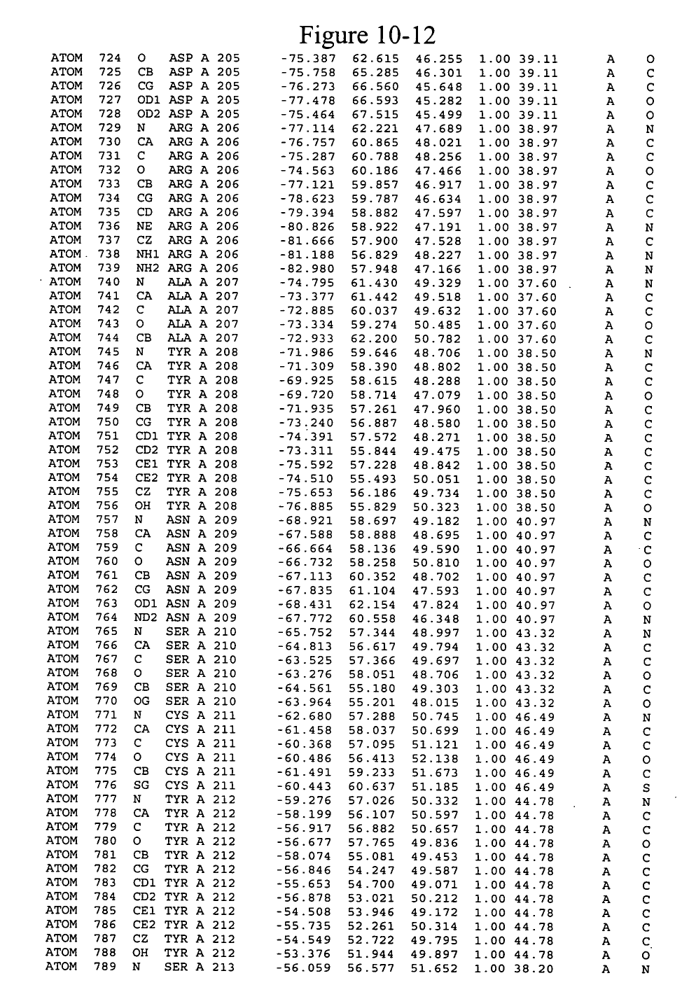

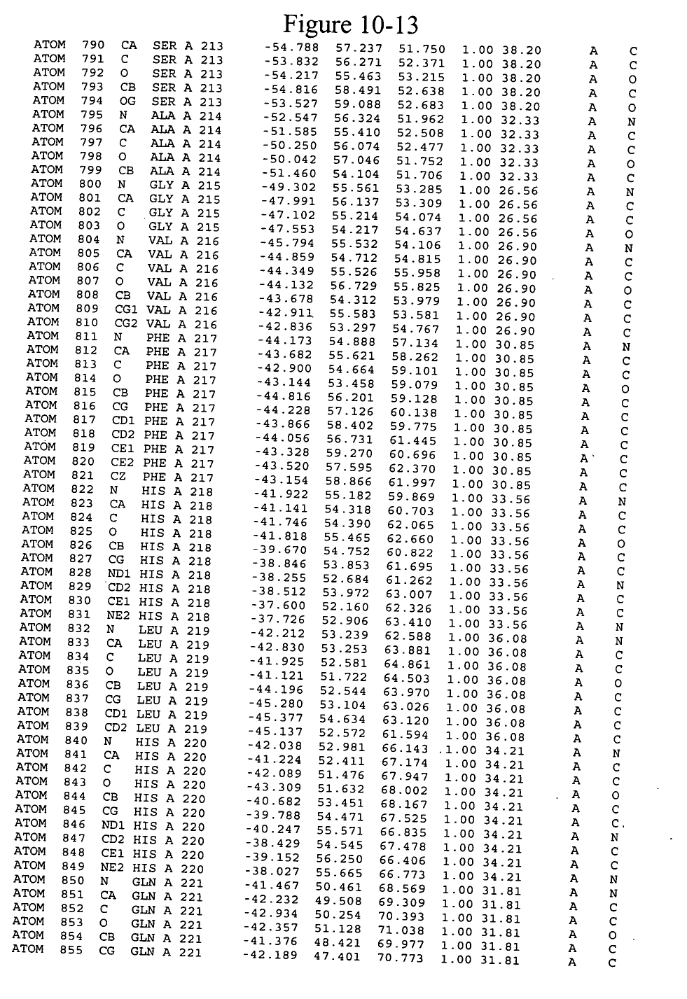

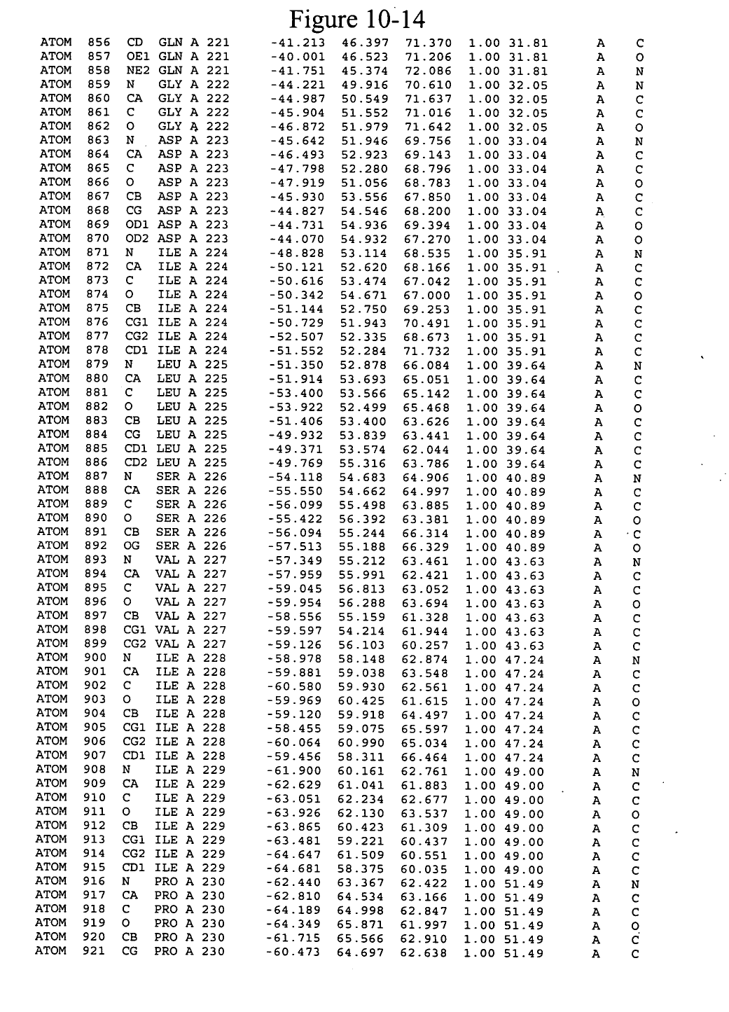

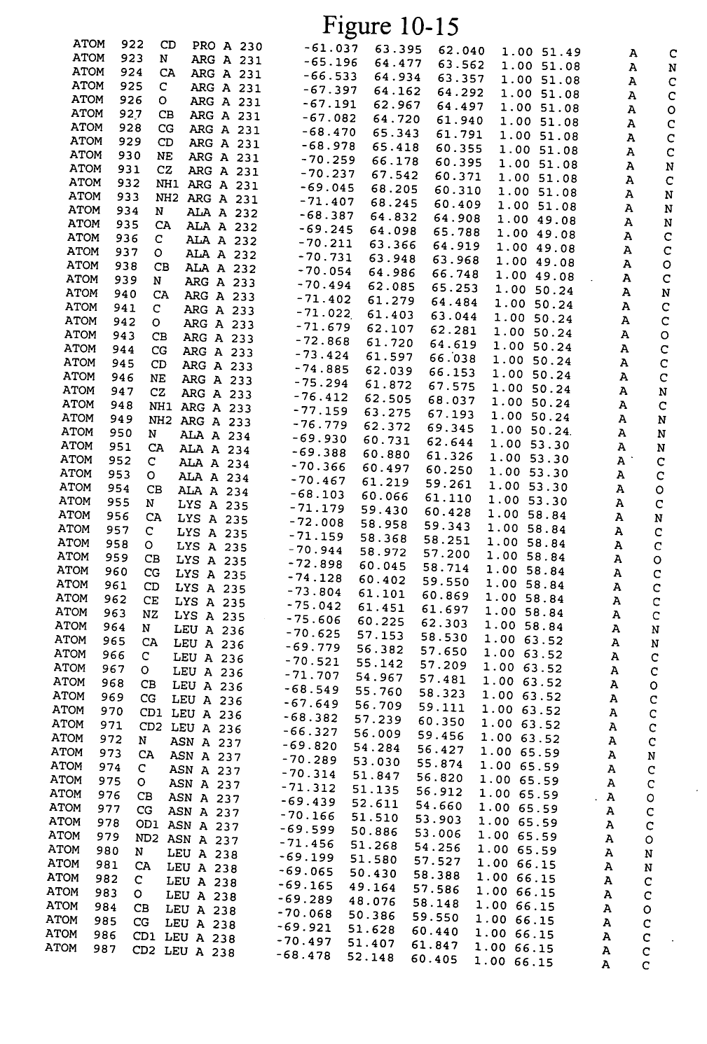

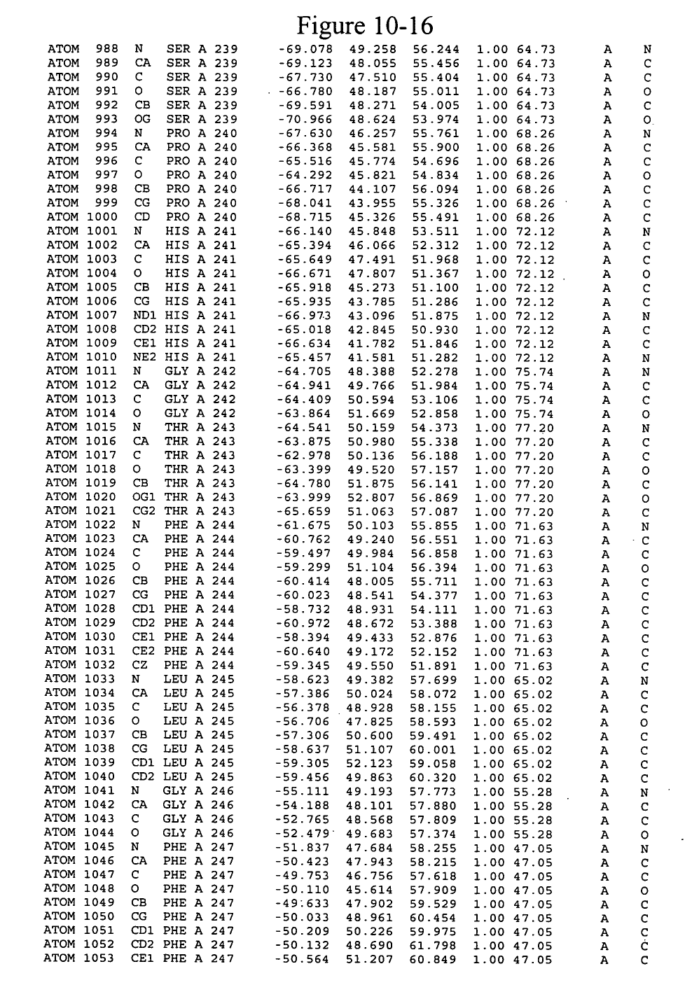

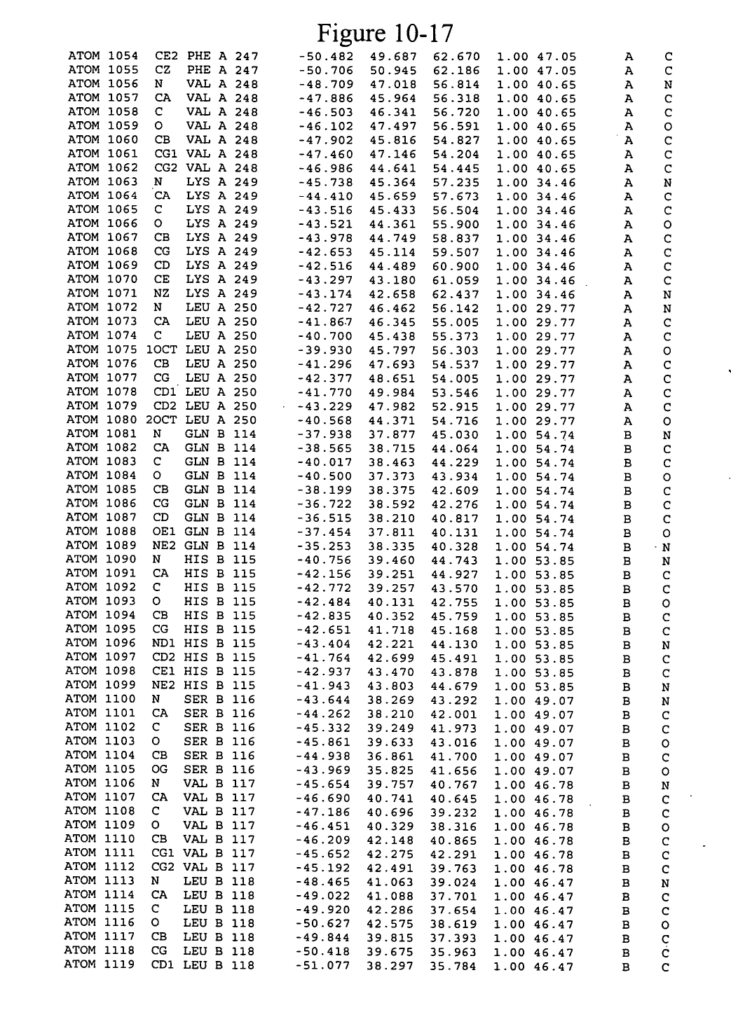

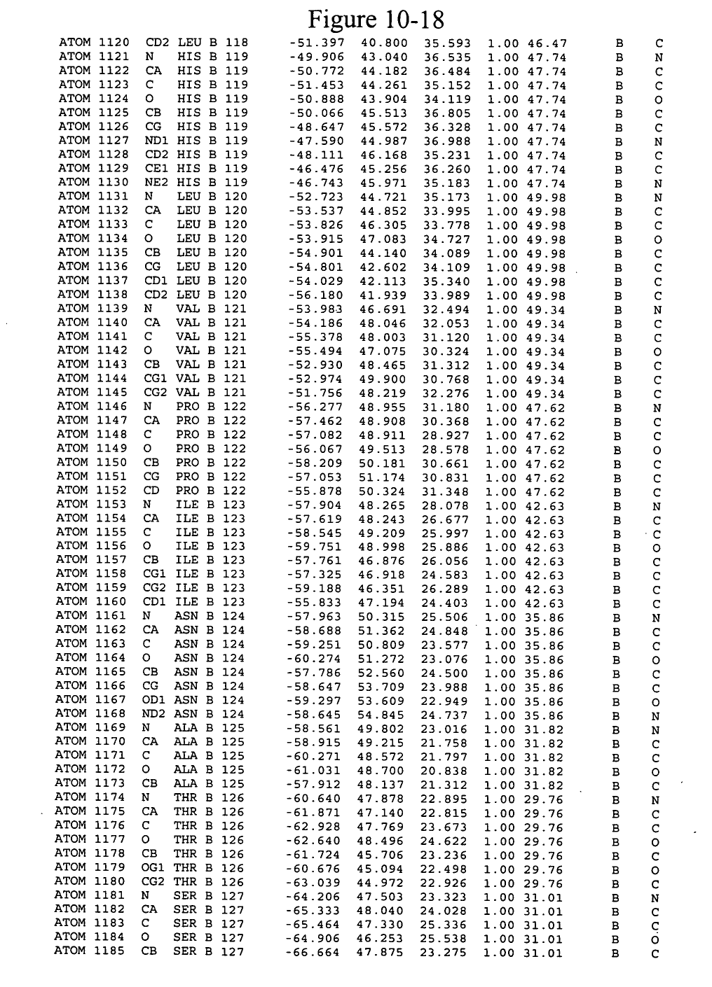

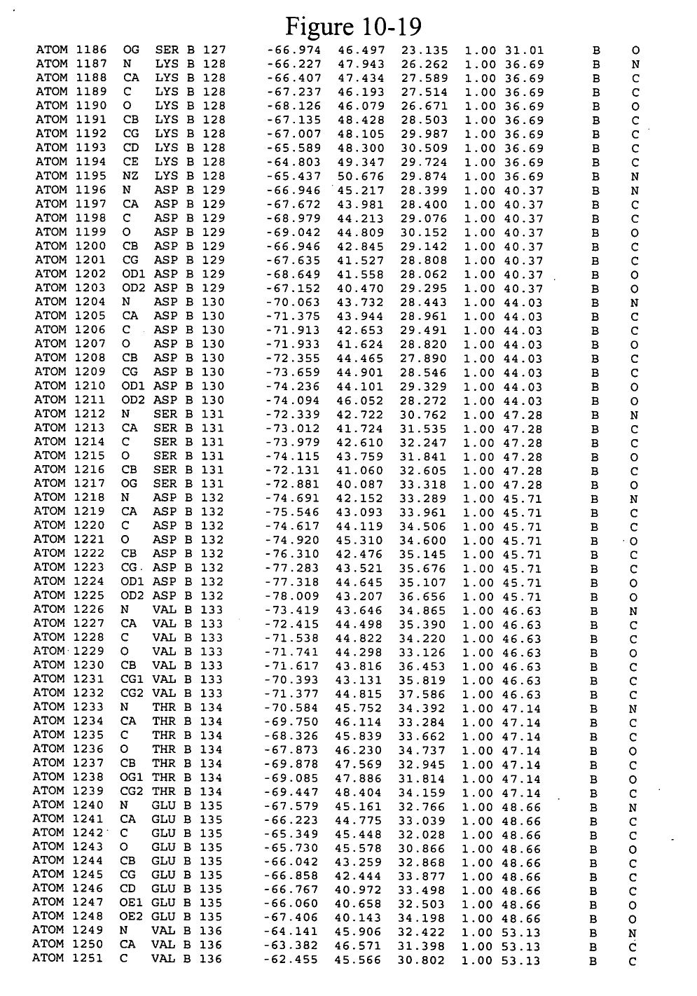

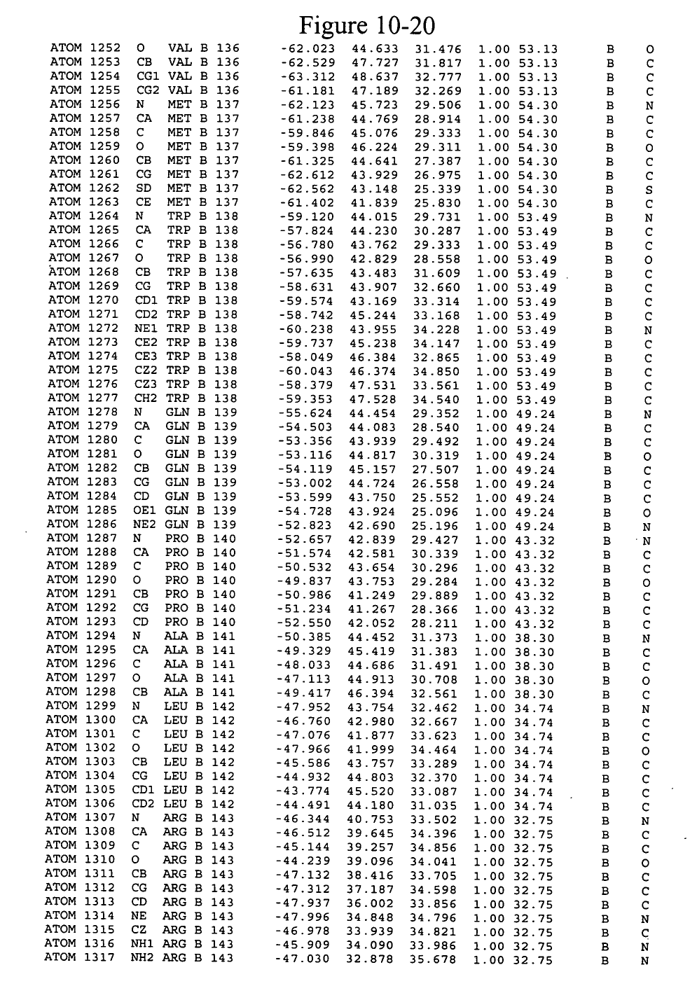

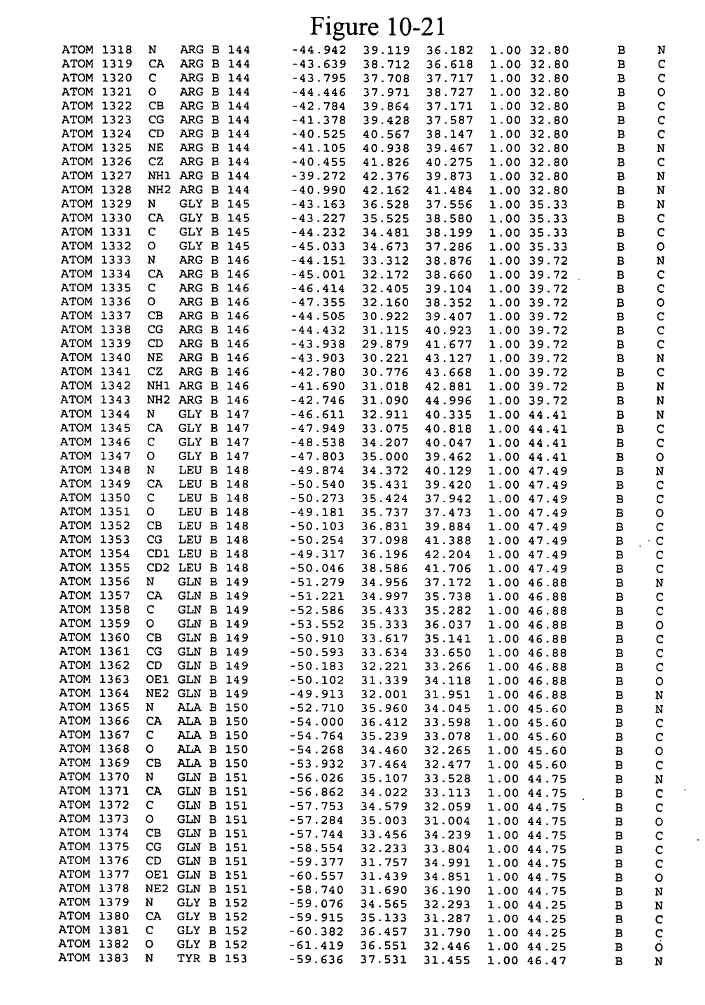

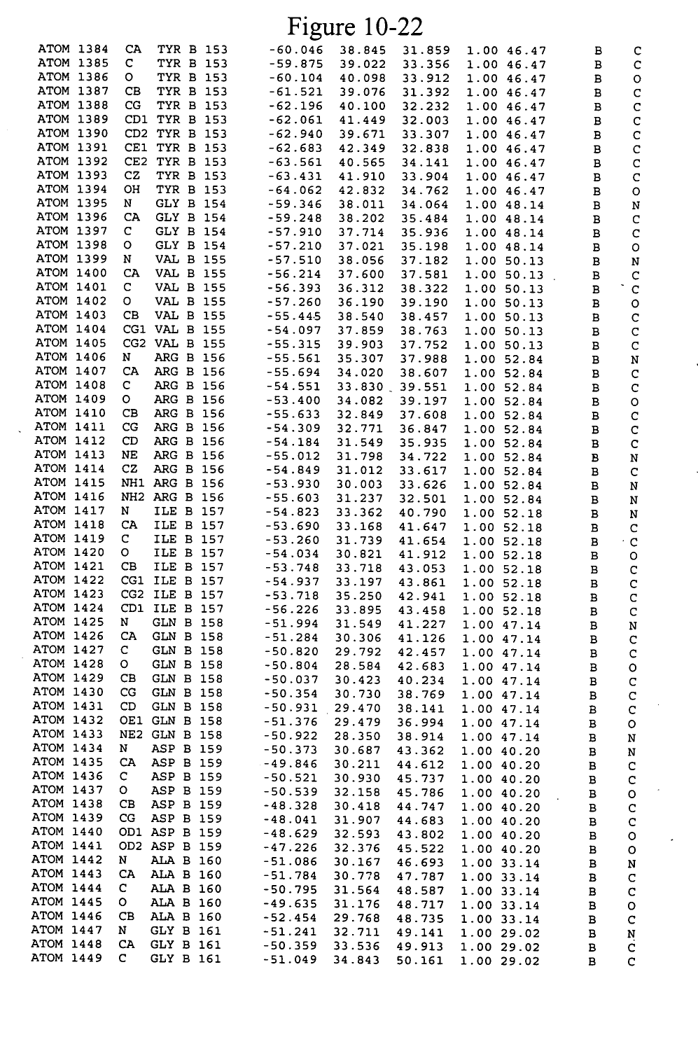

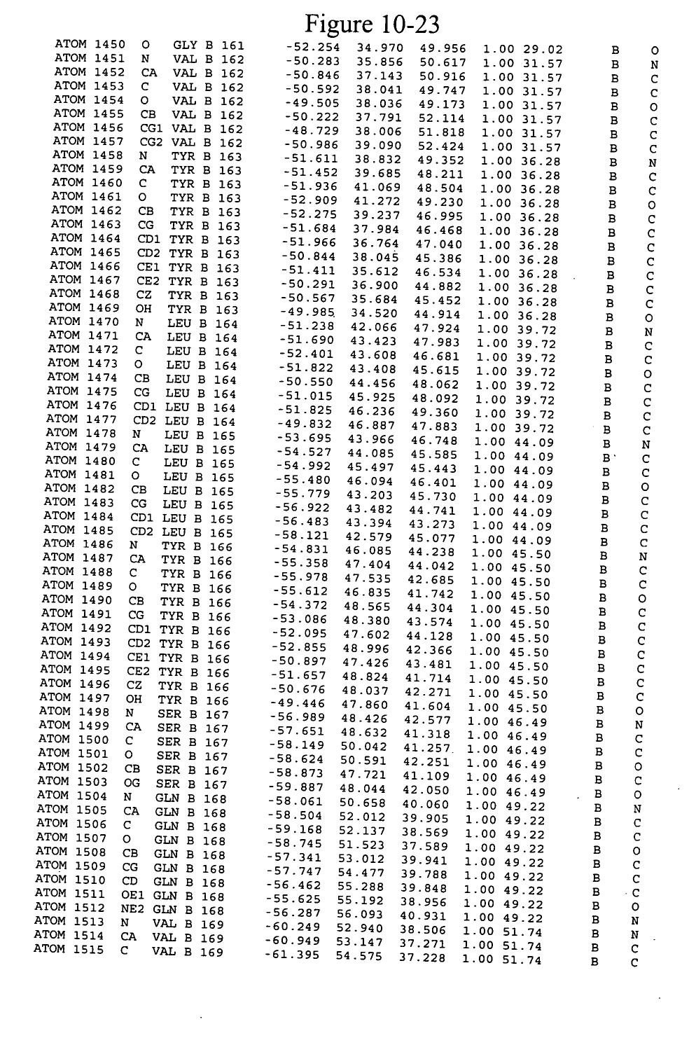

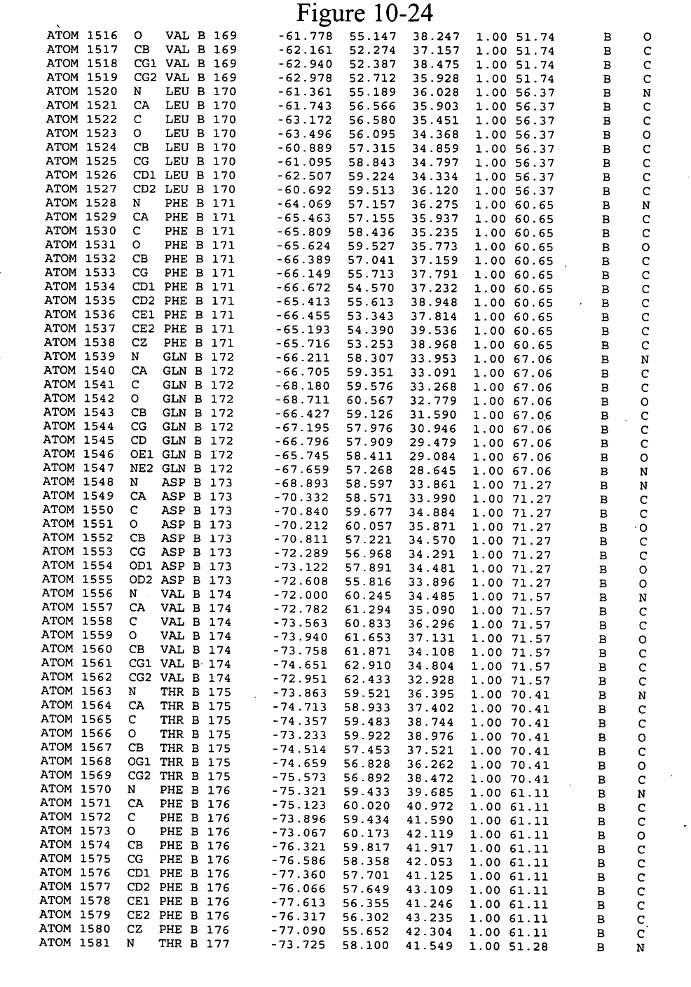

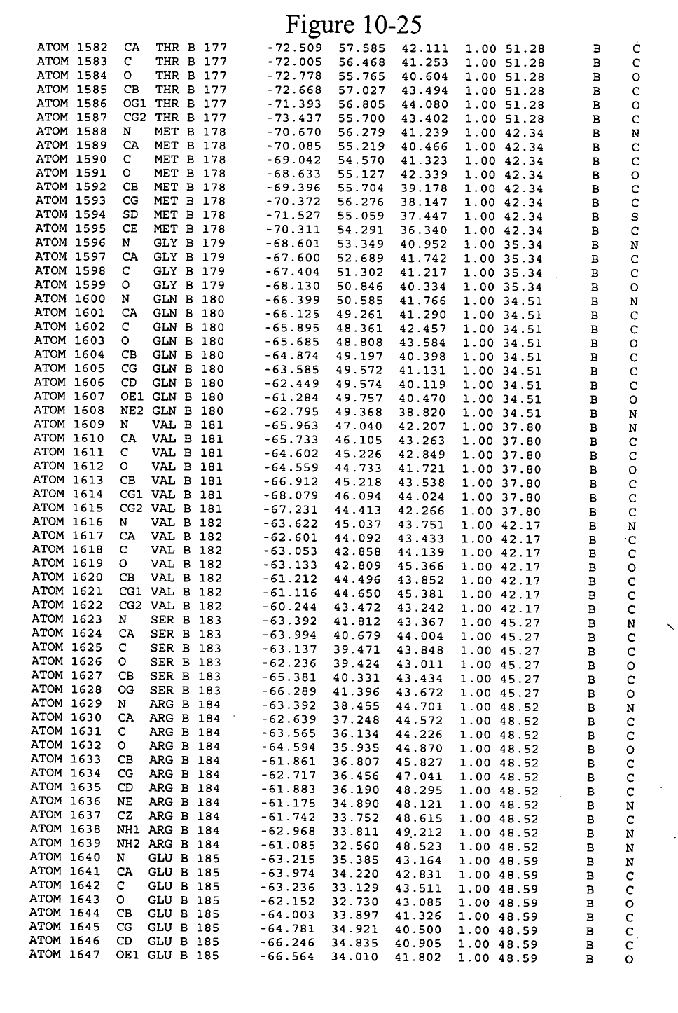

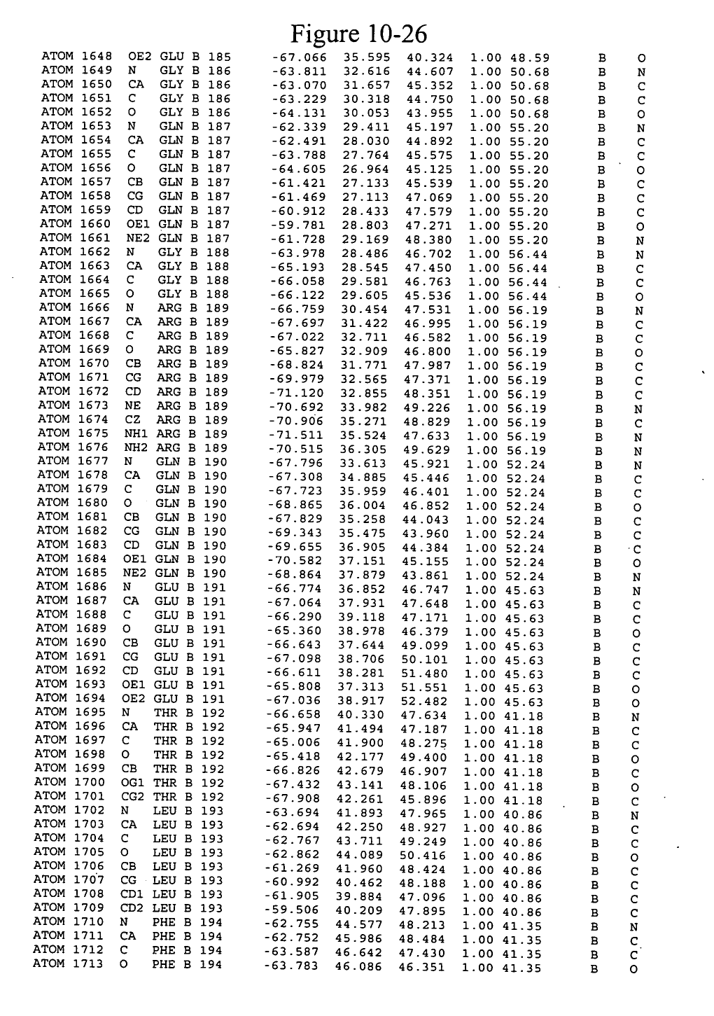

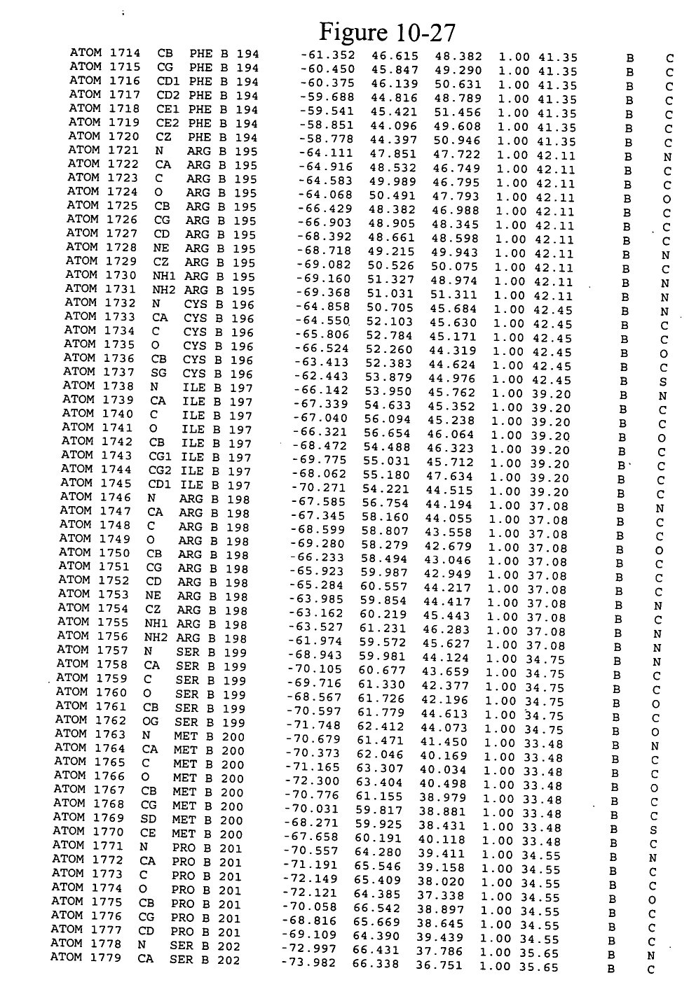

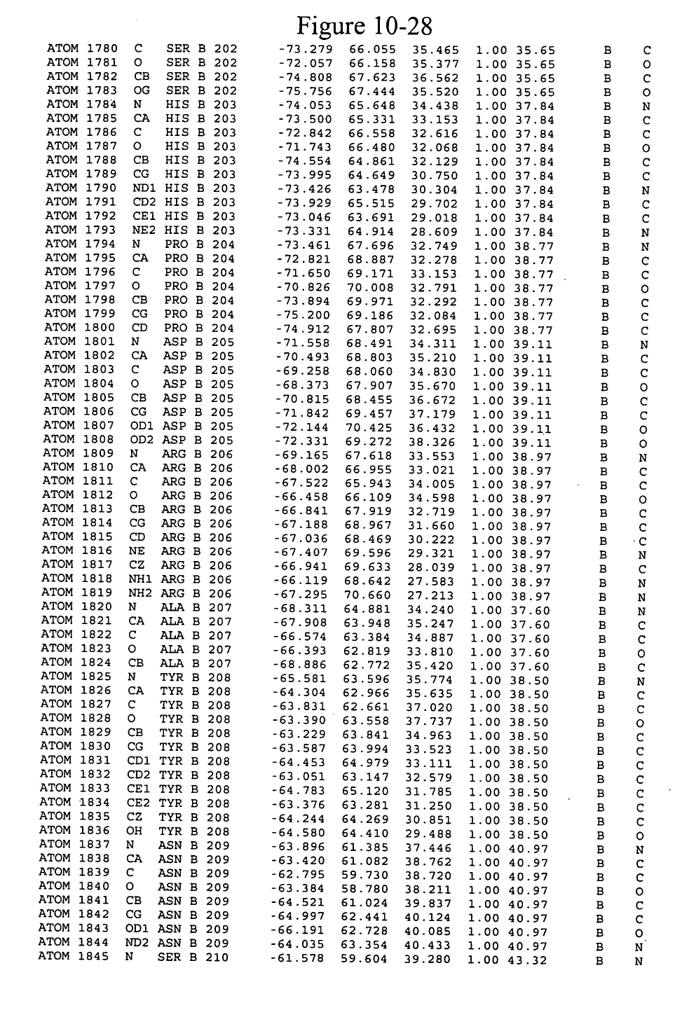

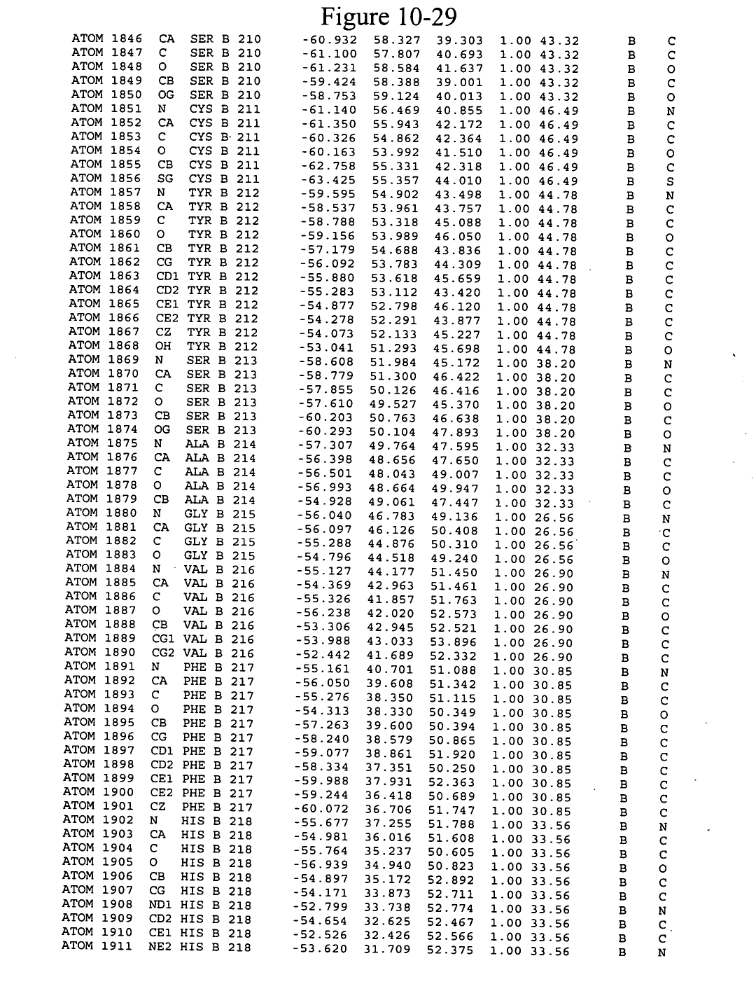

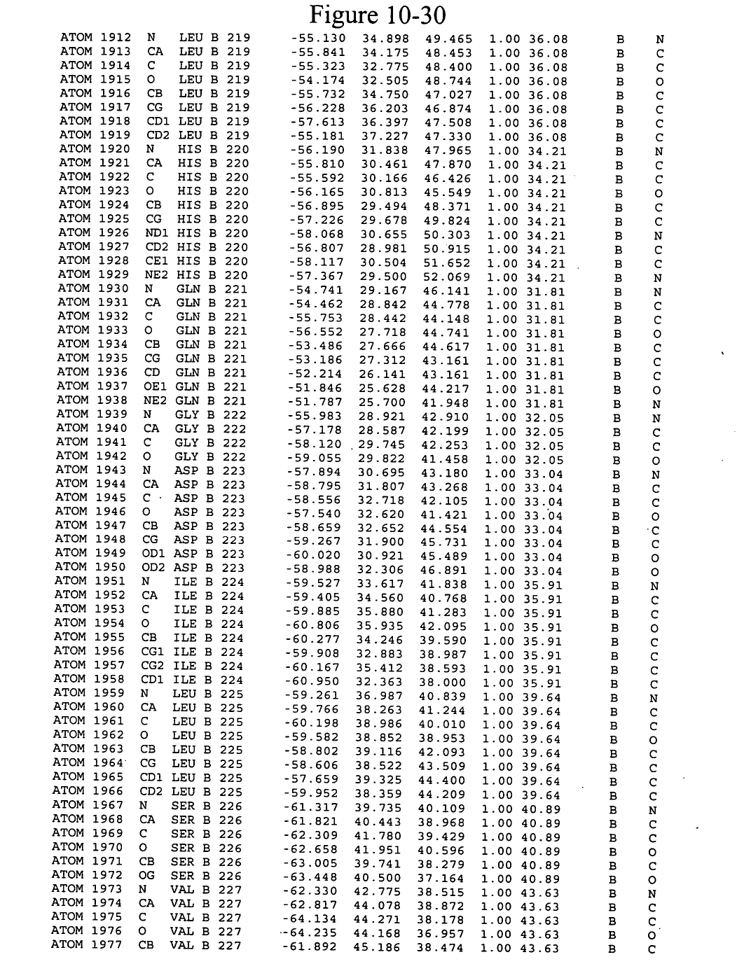

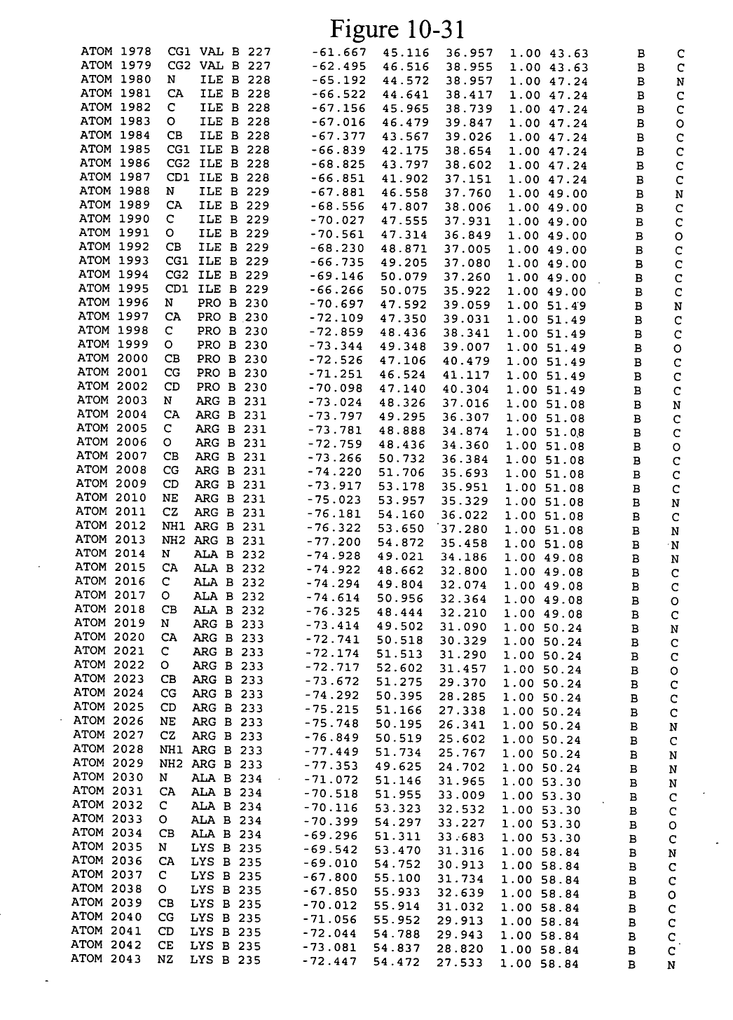

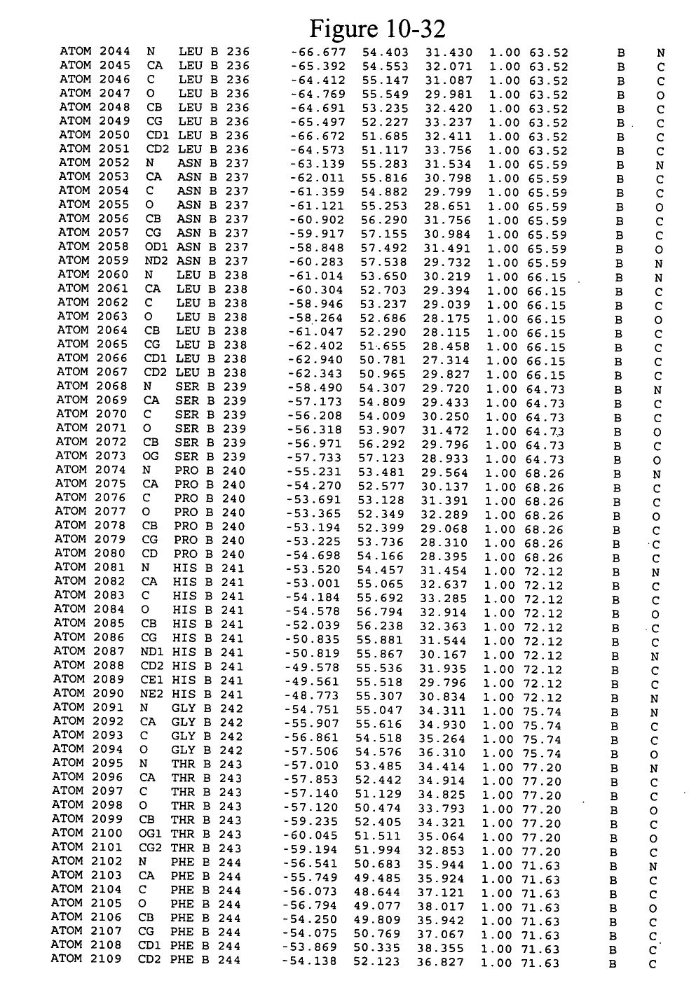

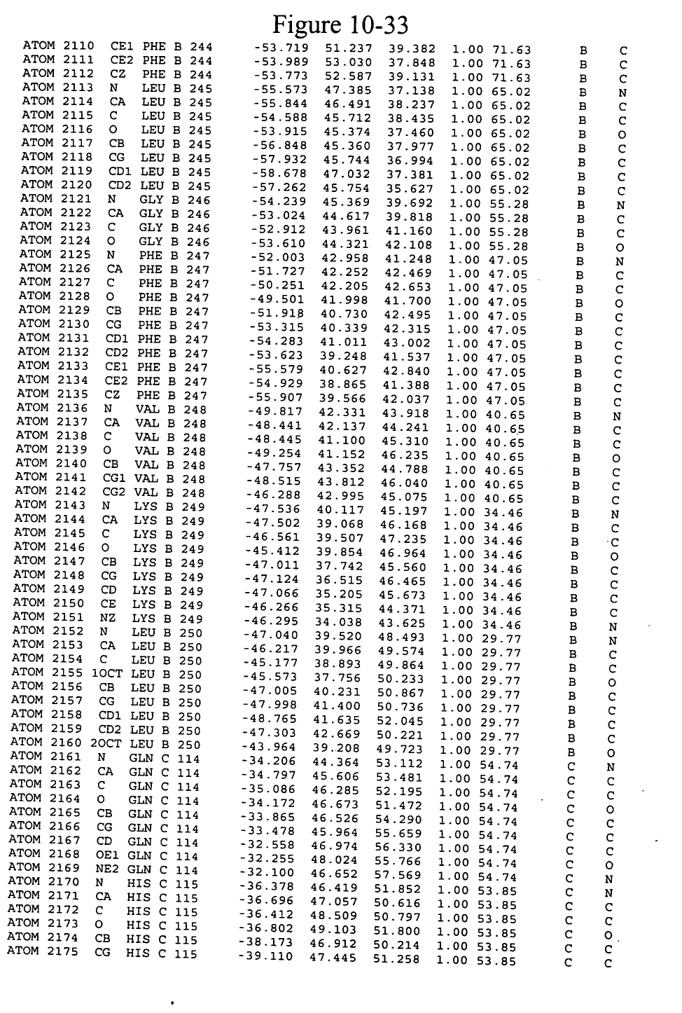

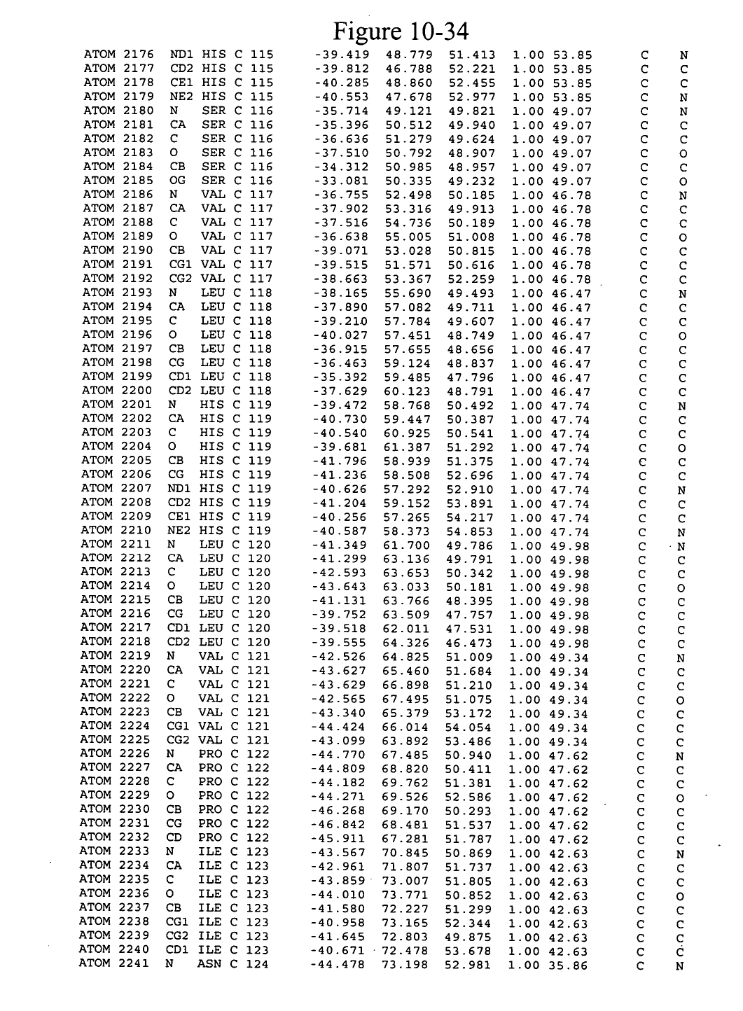

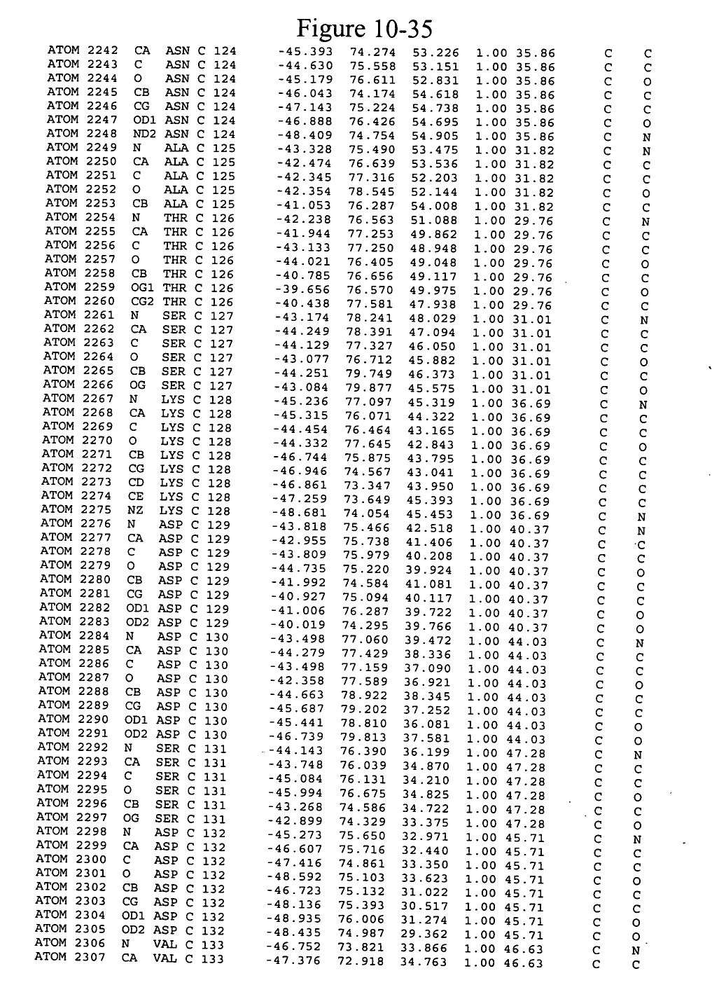

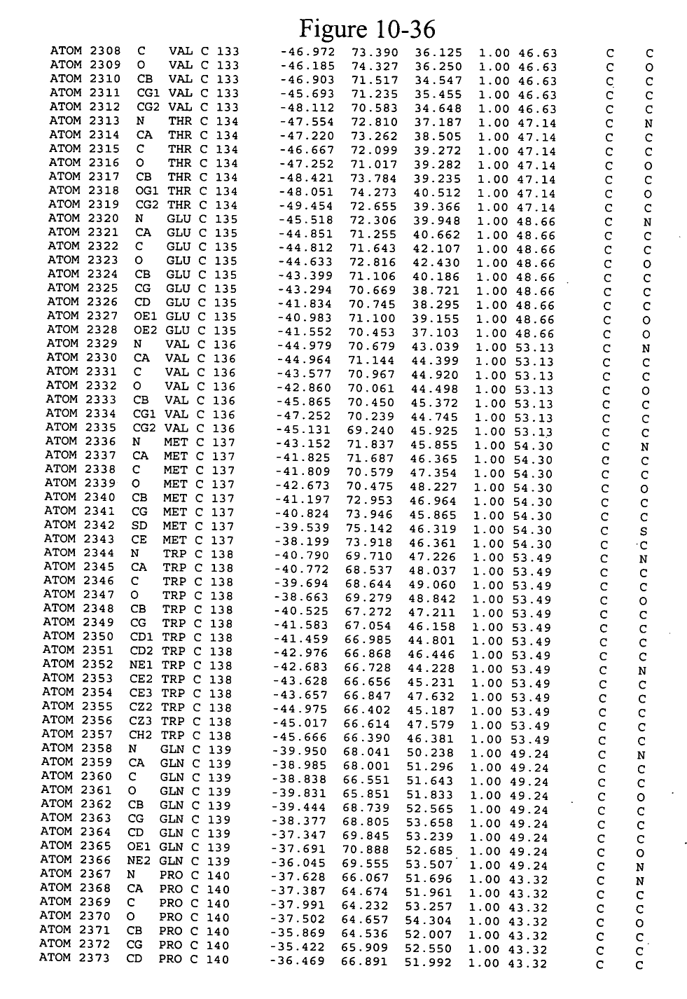

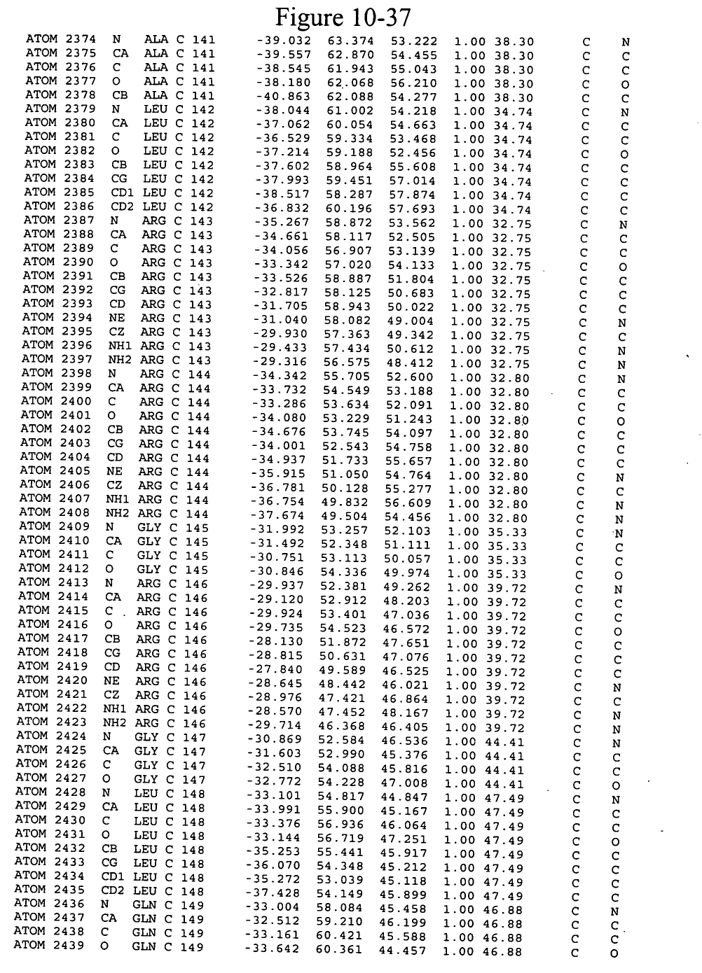

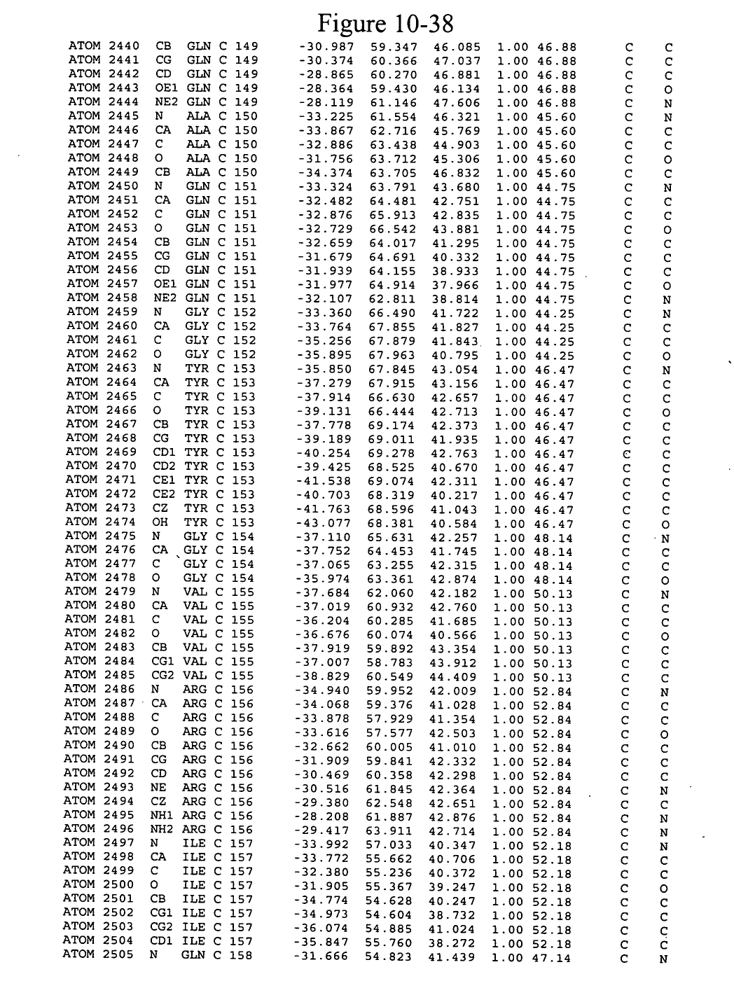

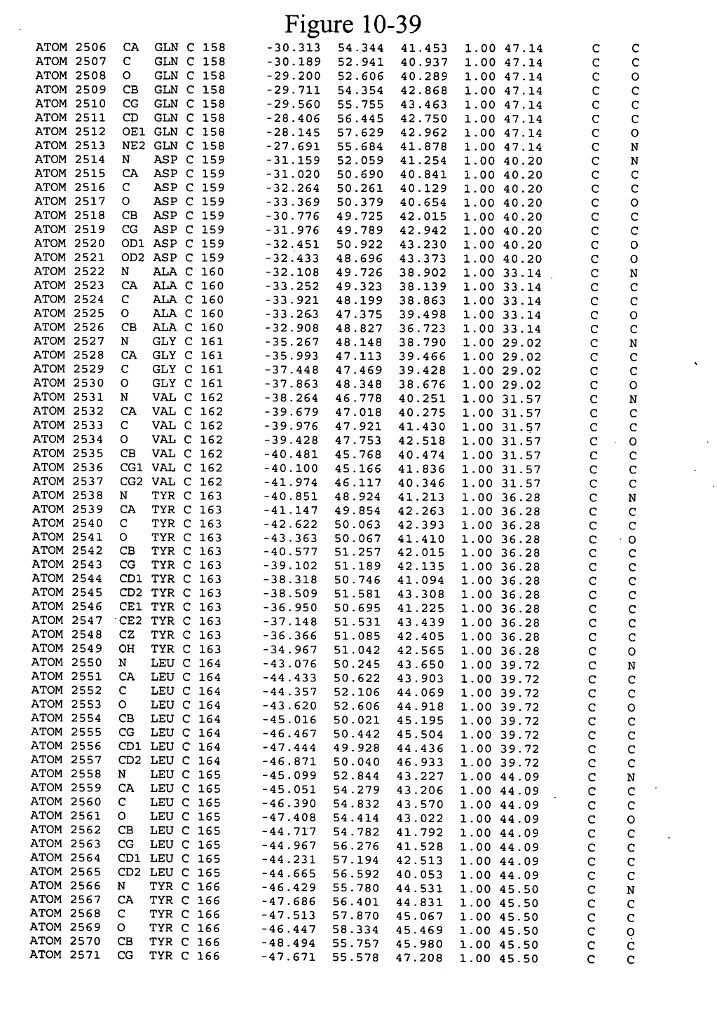

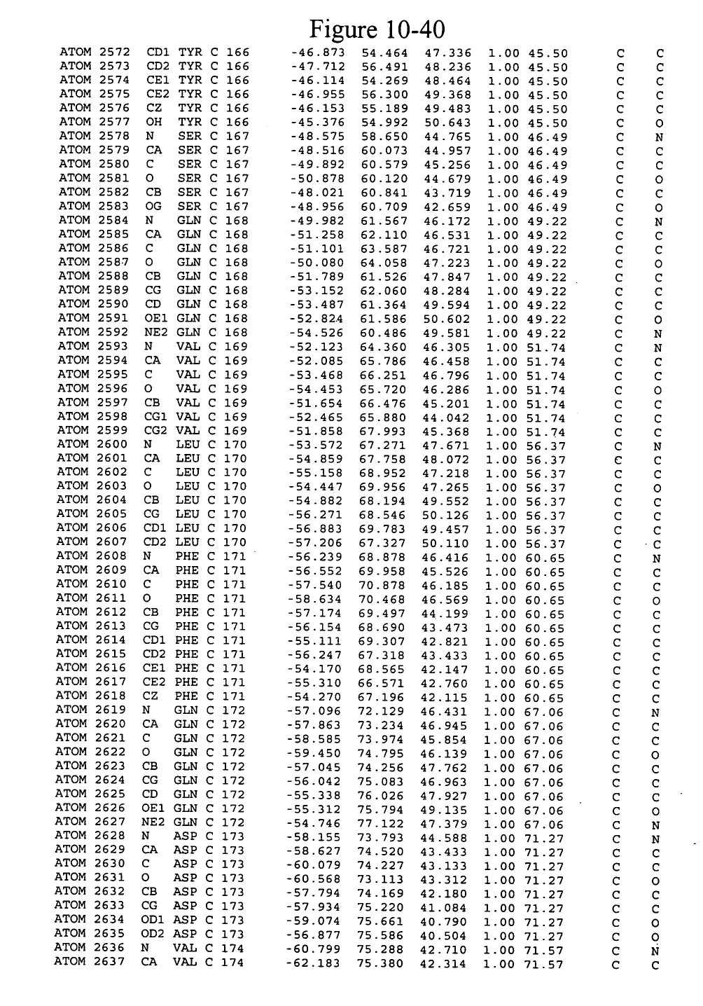

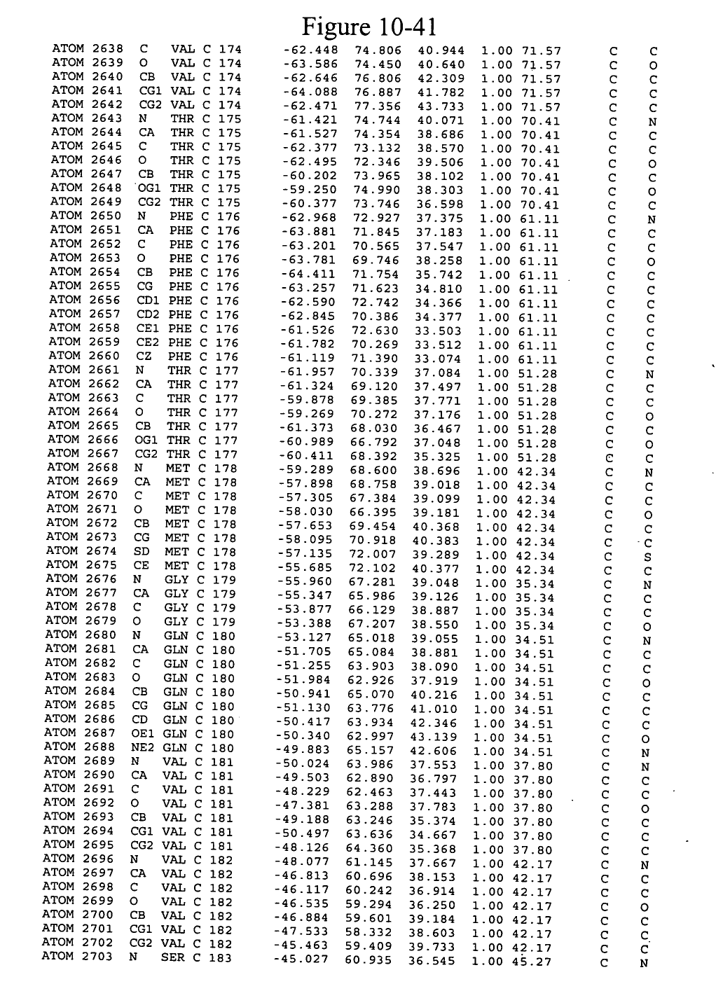

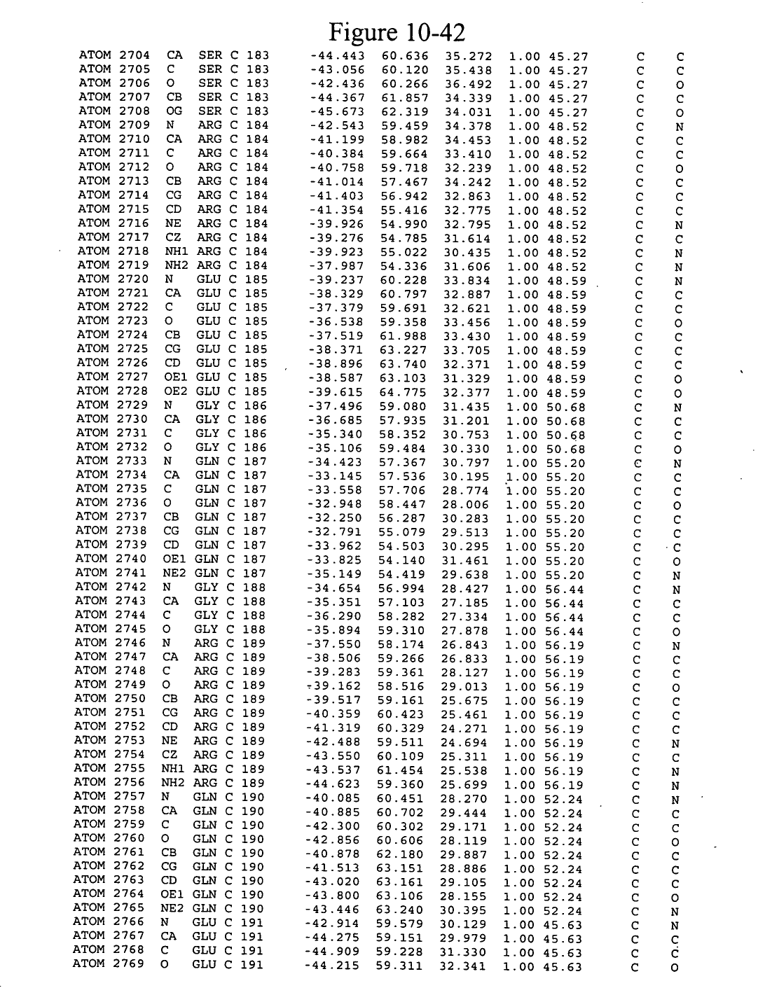

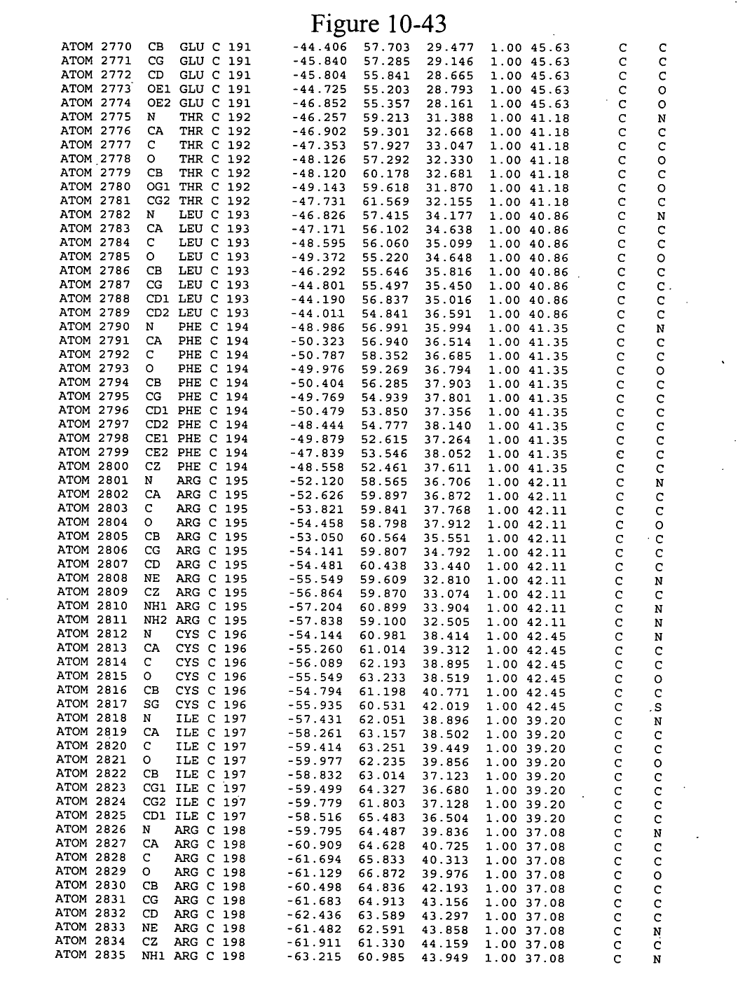

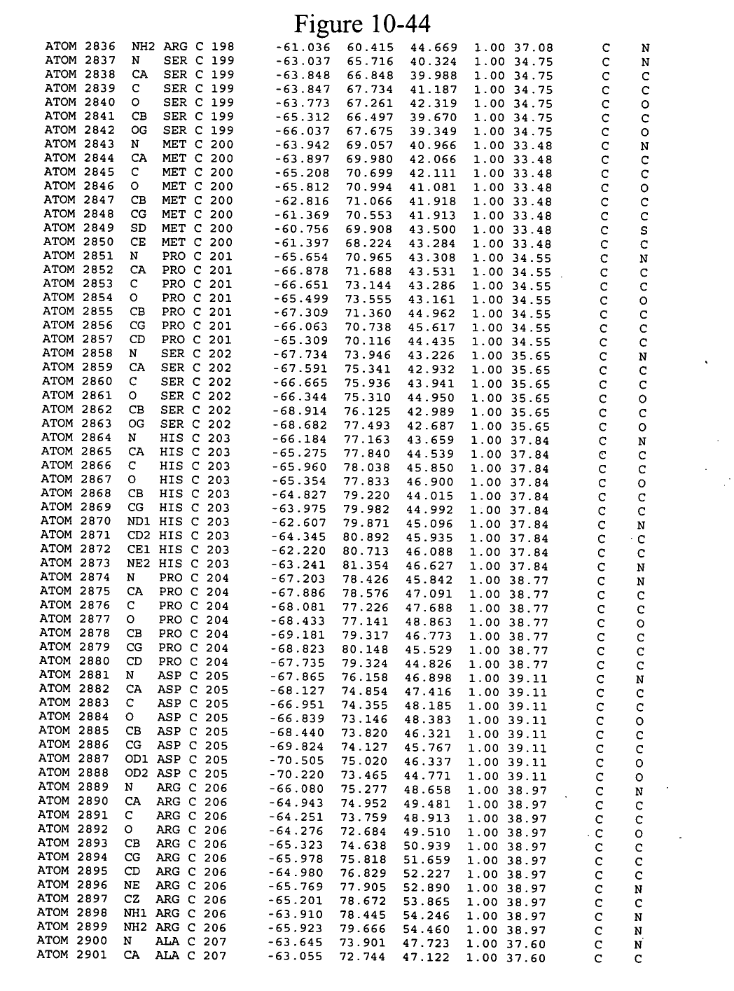

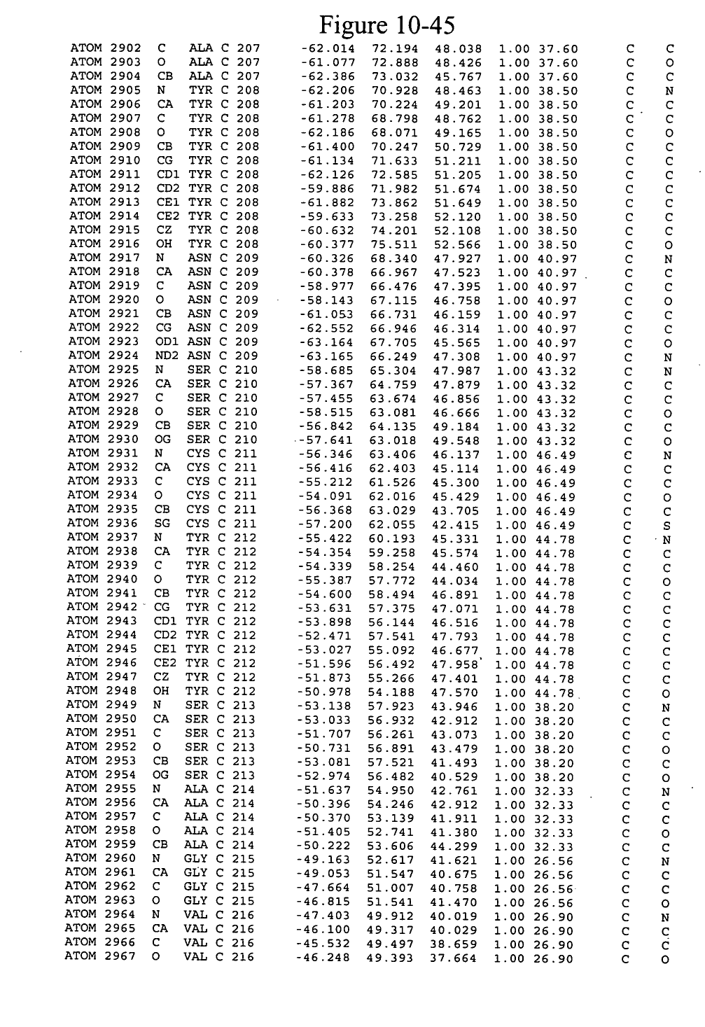

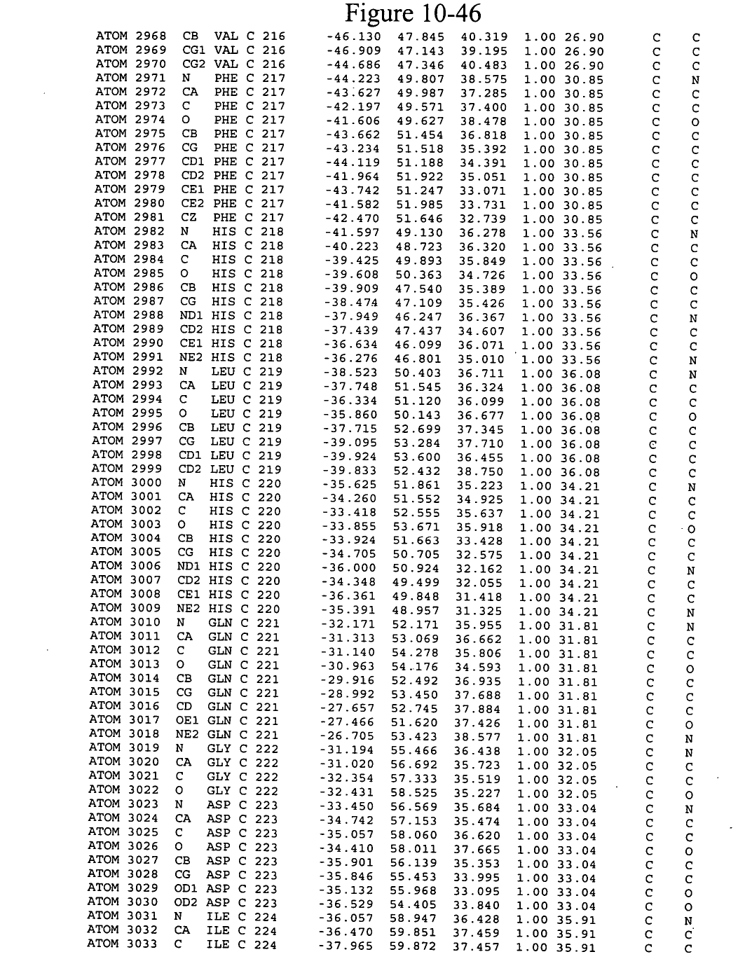

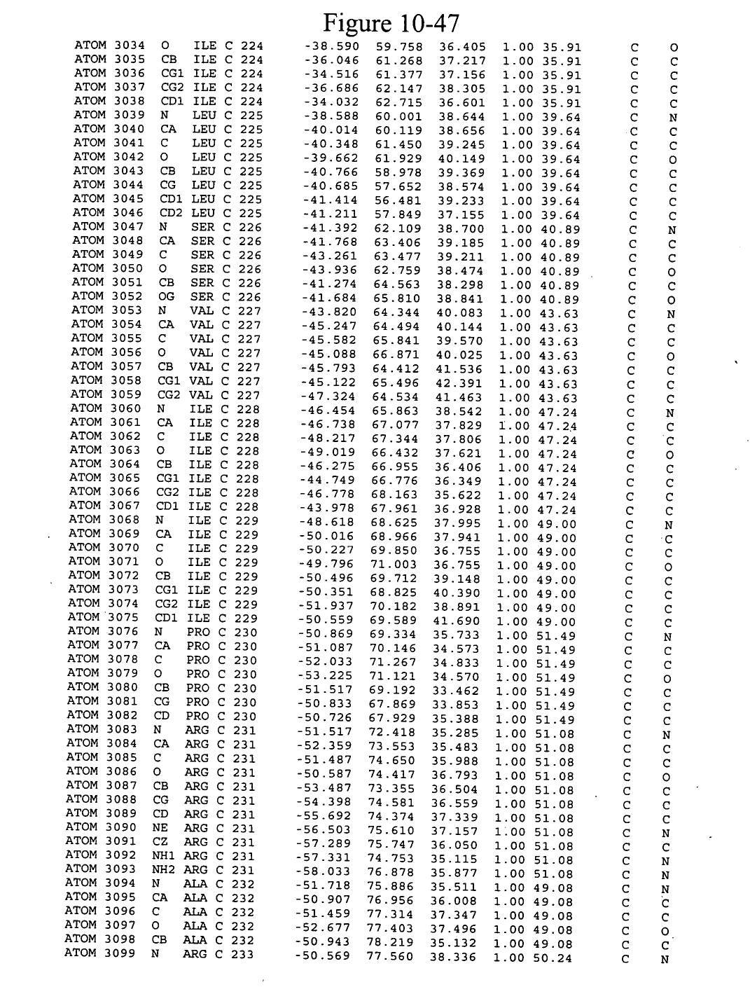

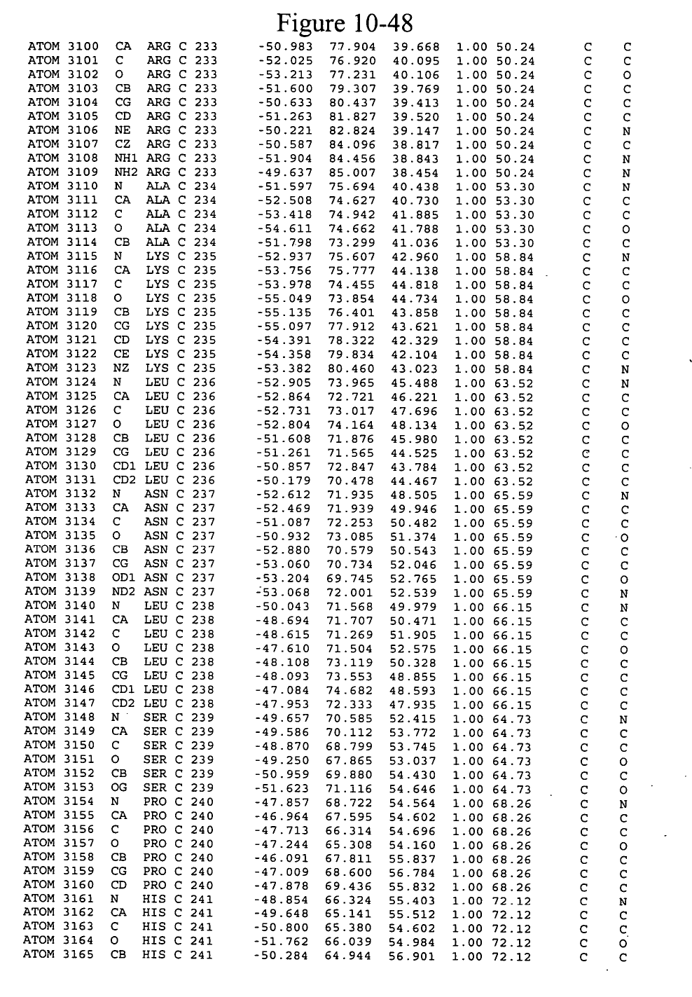

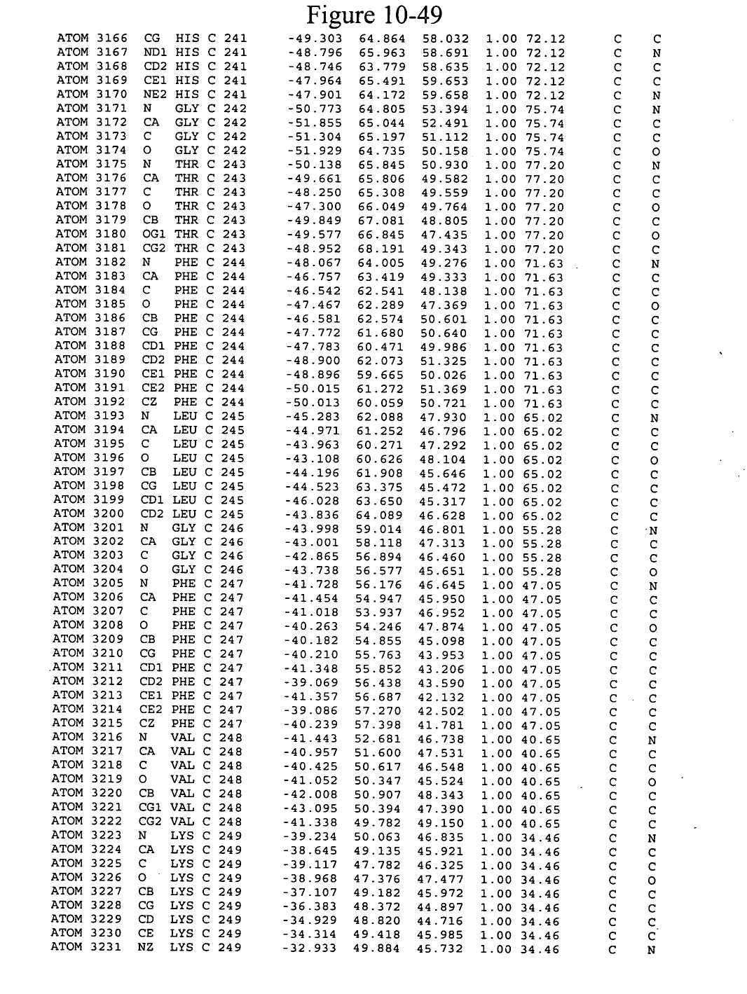

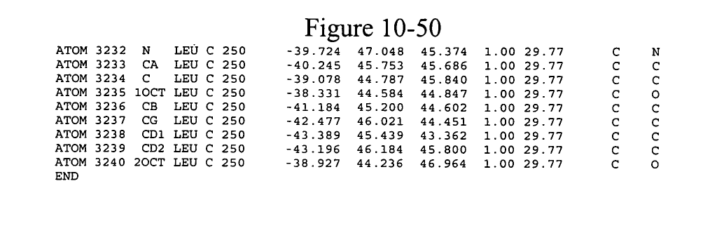

a) a homology model structure of at least a portion or all of a molecule or molecular complex whose structure is unknown and at least a portion of whose structure is similar to the structure of BAFF, wherein said homology model structure is defined by at least a portion of (or all) the homology model structure coordinates of all the APRIL amino acids set forth in FIG. 10;

wherein said computer comprises:

(i) a machine-readable data storage medium comprising a data storage material encoded with machine-readable data, wherein said data comprises at least a portion of the structure coordinates of all of the APRIL amino acids set forth in FIG. 10; and

(ii) instructions for processing said machine-readable data into said three-dimensional representation.

The computer described above can further comprise a display for displaying said homology model structure coordinates.

The structure coordinates set forth in FIG. 8 can also be used for determining at least a portion of the three-dimensional structure of molecules or molecular complexes which contain at least some structural features similar to at least a portion of BAFF. In particular, structural information about another crystallized molecule or molecular complex may be obtained. This may be achieved by any of a number of well-known techniques, including molecular replacement.

Therefore, another embodiment of this invention provides a method of utilizing molecular replacement to obtain structural information about a crystallized molecule or molecular complex whose structure is unknown comprising the steps of:

a) generating an X-ray diffraction pattern from said crystallized molecule or molecular complex; and

b) applying at least a portion of the structure coordinates set forth in FIG. 8 to the X-ray diffraction pattern to generate a three-dimensional electron density map of the molecule or molecular complex whose structure is unknown.

Preferably, the crystallized molecule or molecular complex comprises a BAFF polypeptide. In another preferred embodiment, the crystallized molecule or molecular complex comprises an APRIL polypeptide.

By using molecular replacement, all or part of the structure coordinates of the extracellular domain of BAFF provided by this invention (and set forth in FIG. 8) can be used to determine the structure of a crystallized molecule or molecular complex whose structure is unknown more rapidly and efficiently than attempting to determine such information ab initio. This method is especially useful in determining the structure of BAFF mutants and homologues, one such homologue being APRIL.

Molecular replacement provides an accurate estimation of the phases for an unknown structure. Phases are a factor in equations used to solve crystal structures that cannot be determined directly. Obtaining accurate values for the phases, by methods other than molecular replacement, is a time-consuming process that involves iterative cycles of approximations and refinements and greatly hinders the solution of crystal structures. However, when the crystal structure of a protein containing at least a homologous portion has been solved, the phases from the known structure provide a satisfactory estimate of the phases for the unknown structure.

Thus, molecular replacement involves generating a preliminary model of a molecule or molecular complex whose structure coordinates are unknown, by orienting and positioning the relevant portion of the extracellular domain of BAFF according to FIG. 8 within the unit cell of the crystal of the unknown molecule or molecular complex, so as best to account for the observed X-ray diffraction pattern of the crystal of the molecule or molecular complex whose structure is unknown. Phases can then be calculated from this model and combined with the observed X-ray diffraction pattern amplitudes to generate an electron density map of the structure whose coordinates are unknown. This, in turn, can be subjected to any well-known model building and structure refinement techniques to provide a final, accurate structure of the unknown crystallized molecule or molecular complex [E. Lattman, “Use of the Rotation and Translation Functions”, in Meth. Enzymol., 115, pp. 55-77 (1985); M. G. Rossmann, ed., “The Molecular Replacement Method”, Int. Sci. Rev. Ser., No. 13, Gordon & Breach, New York (1972)].

The structure of any portion of any crystallized molecule or molecular complex that is sufficiently homologous to any portion of the extracellular domain of BAFF can be solved by this method. The term “sufficiently homologous to any portion of the extracellular domain of BAFF” refers to a protein or section thereof that has a sequence homology of at least 25% compared to any portion of the extracellular domain of BAFF. In one embodiment, the sequence homology is at least 30%. In one embodiment, the sequence homology is at least 40%.

In a preferred embodiment, the method of molecular replacement is utilized to obtain structural information about a molecule or a molecular complex, wherein the molecule or molecular complex comprises a BAFF-like polypeptide. Preferably the BAFF-like polypeptide is BAFF, a mutant thereof or a homologue thereof, one such homologue being APRIL.

The structure coordinates of the extracellular domain of a BAFF as provided by this invention are particularly useful in solving the structure of other crystal forms of BAFF-like polypeptide, preferably other crystal forms of BAFF; BAFF-like polypeptide, preferably the extracellular domain of BAFF, or a BAFF-like polypeptide; or complexes comprising any of the above. APRIL is a BAFF-like polypeptide.

Such structure coordinates are also particularly useful to solve the structure of crystals of BAFF-like polypeptide, particularly the extracellular domain of BAFF co-complexed with one or more of its receptors, a variety of chemical entities, a compound, such as an agonist or antagonist of BAFF, or a variant of an agonist or antagonist of BAFF. This approach enables the determination of the optimal sites for interaction between chemical entities, interaction of candidate BAFF agonists or antagonists with BAFF, or interaction of a variant of a BAFF agonist or antagonist, and the extracellular domain of BAFF. For example, high resolution X-ray diffraction data collected from crystals exposed to different types of solvent allows determination of the location where each type of solvent molecule resides. Small molecules that bind tightly to these sites can then be designed and synthesized and tested for their BAFF agonist or antagonist activity.

All of the molecules or molecular complexes referred to above may be studied using well-known X-ray diffraction techniques and may be refined versus 1.5-3.5 Å resolution X-ray data to an R-working value of about 0.25 or less using computer software, such as X-PLOR (Yale University, ©1992, distributed by Molecular Simulations, Inc.; see, e.g., Blundell & Johnson, supra; Meth. Enzymol., vol. 114 & 115, H. W. Wyckoff et al., eds., Academic Press (1985)). This information may thus be used to optimize BAFF agonists or antagonists, and more importantly, to design new or improved BAFF agonists or antagonists. A BAFF agonist or antagonist may be an antibody, or an antigen binding fragment thereof.

A chemical entity or a compound (including an agonist or antagonist of BAFF), as well as variants of BAFF agonists or antagonists, which could be an antibody, or an antigen binding fragment thereof, or another protein, can be designed by computational means by performing fitting operations. A compound could be a macromolecule, such as a protein or a polypeptide.

The present invention also encompasses methods of evaluating the potential of a chemical entity to associate with a molecule or molecular complex of this invention, or a homologue of said molecule or molecular complex.

This invention provides a method for evaluating the potential of a chemical entity to associate with:

a) a molecule or molecular complex defined by at least a portion or all of the structure coordinates of the BAFF amino acids, set forth in FIG. 8; or

b) a homologue of said molecule or molecular complex having a root mean square deviation from the backbone atoms of said amino acids of between 0.00 Å and 1.50Å, preferably between 0.00 Å and 1.00 Å, more preferably between 0.00 Å and 0.50 Å;

comprising the steps of:

(i) employing computational means to perform a fitting operation between said chemical entity and said molecule or molecular complex or between said chemical entity and a homologue of said molecule or molecular complex; and

(ii) analyzing the results of said fitting operation to quantify the association between said chemical entity and said molecule or molecular complex or said homologue of said molecule or molecular complex. In this method, the molecule or molecular complex, or the homologue of the molecule or molecular complex, preferably comprises a binding site; said binding site could be a binding site of BAFF for one or more of its receptors. The fitting operation in (i) above could be between said chemical entity and the binding site of the molecule or molecular complex or the binding site of the homologue of the molecule or molecular complex. The association in (ii) could be the association between said chemical entity and the binding site of the molecule or molecular complex or the binding site of the homologue of the molecule or molecular complex.

This invention also provides a method for evaluating the potential of a chemical entity to associate with:

a) a molecule or molecular complex comprising a first binding site defined by at least one (preferably at least four) or a plurality of BAFF amino acids selected from the group consisting of His218, Val219, Phe220, Gly221, Asp222, Glu223, Leu224, Ser225, Leu226, Val227, Pro264, Arg265, Glu266, Gly161, Ser162, Tyr163, Ala151, Asp152, Ser153, Glu154, Thr155, Pro156, Leu240, Pro241, Asn242, Ser171 and Phe172 according to FIG. 8; or P b) a homologue of said molecule or molecular complex comprising a second binding site having a root mean square deviation from the backbone atoms of said at least one (preferably at least four) or a plurality of BAFF amino acids between 0.00 Å and 1.50Å, preferably between 0.00 Å and 1.00 Å, more preferably between 0.00 Å and 0.50 Å;

comprising the steps of:

(i) employing computational means to perform a fitting operation between said chemical entity and said first binding site or said second binding site; and

(ii) analyzing the results of said fitting operation to quantify the association between said chemical entity and said first binding site or said second binding site.

Preferably, the first binding site or the second binding site is a binding site of BAFF for one or more receptors of BAFF.

As determined by homology to other TNF family members (see Example 1), the binding site of BAFF for one or more of its receptors comprises a plurality of amino acid residues selected from the group consisting of His218, Val219, Phe220, Gly221, Asp222, Glu223, Leu224, Ser225, Leu226, Val227, Pro264, Arg265, Glu266, Gly161, Ser162, Tyr163, Ala151, Asp152, Ser153, Glu154, Thr155, Pro156, Leu240, Pro241, Asn242, Ser171 and Phe172.

Also, as determined by homology to other TNF family members (see Example 1), the binding site of BAFF for one or more of its receptors comprises at least one (preferably at least four) amino acid residues selected from the group consisting of His218, Val219, Phe220, Gly221, Asp222, Glu223, Leu224, Ser225, Leu226, Val227, Pro264, Arg265, Glu266, Gly161, Ser162, Tyr163, Ala151, Asp152, Ser153, Glu154, Thr155, Pro156, Leu240, Pro241, Asn242, Ser171 and Phe172.

The present invention also encompasses a method for identifying a potential agonist of BAFF comprising the steps of:

a) using at least a portion or all of the structure coordinates of the amino acids of BAFF according to FIG. 8 +a root mean square deviation from the backbone atoms of said BAFF amino acids between 0.00 Å and 1.50Å, preferably between 0.00 Å and 1.00 Å, more preferably between 0.00 Å and 0.50 Å, to generate a three-dimensional structure of a molecule or a molecular complex;

b) employing said three-dimensional structure to design or select said potential agonist;

c) synthesizing said potential agonist; and

d) contacting said potential agonist with BAFF to determine the ability of said potential agonist to interact with BAFF.

The present invention also encompasses a method for identifying a potential antagonist of BAFF comprising the steps of:

a) using at least a portion or all of the structure coordinates of the amino acids of BAFF according to FIG. 8 ± a root mean square deviation from the backbone atoms of said BAFF amino acids between 0.00 Å and 1.50Å, preferably between 0.00 Å and 1.00 Å, more preferably between 0.00 Å and 0.50 Å, to generate a three-dimensional structure of a molecule or a molecular complex;

b) employing said three-dimensional structure to design or select said potential antagonist;

c) synthesizing said potential antagonist; and

d) contacting said potential antagonist with BAFF to determine the ability of said potential antagonist to interact with BAFF.

The molecule or molecular complex preferably comprises a binding site; said binding site could be a binding site of BAFF for one or more of its receptors. This method could further comprise the step of:

e) determining whether said potential antagonist interrupts the interaction between BAFF and one of its receptors.

This invention also provides a method for identifying a potential antagonist of BAFF comprising the steps of:

a) using the structure coordinates of at least one (preferably at least four) or a plurality of BAFF amino acids selected from the group consisting of His218, Val219, Phe220, Gly221, Asp222, Glu223, Leu224, Ser225, Leu226, Val227, Pro264, Arg265, Glu266, Gly161, Ser162, Tyr163, Ala151, Asp152, Ser153, Glu154, Thr155, Pro156, Leu240, Pro241, Asn242, Ser171 and Phe172 according to FIG. 8 or ± a root mean square deviation from the backbone atoms of said at least one (preferably at least four) or a plurality of BAFF amino acids between 0.00 Å and 1.50Å, preferably between 0.00 Å and 1.00 Å, more preferably between 0.00 Å and 0.50 Å, to generate a three-dimensional structure of a molecule or a molecular complex comprising a binding site;

b) employing said three-dimensional structure to design or select said potential antagonist;

c) synthesizing said potential antagonist; and

d) contacting said potential antagonist with BAFF to determine the ability of said potential antagonist to interact with BAFF. The binding site in step a) could be a binding site of BAFF for one or more of its receptors.

This method could further comprise the step of:

e) determining whether said potential

antagonist interrupts the interaction between BAFF and one of its receptors or activates BAFF.

This invention also provides a method for identifying a potential agonist of BAFF comprising the steps of:

a) using the structure coordinates of at least one (preferably at least four) or a plurality of BAFF amino acids selected from the group consisting of His218, Val219, Phe220, Gly221, Asp222, Glu223, Leu224, Ser225, Leu226, Val227, Pro264, Arg265, Glu266, Gly161, Ser162, Tyr163, Ala151, Asp152, Ser153, Glu154, Thr155, Pro156, Leu240, Pro241, Asn242, Ser171 and Phe172 according to FIG. 8 or ± a root mean square deviation from the backbone atoms of said at least one (preferably at least four) or a plurality of BAFF amino acids between 0.00 Å and 1.50Å, preferably between 0.00 Å and 1.00 Å, more preferably between 0.00 Å and 0.50 Å, to generate a three-dimensional structure of a molecule or a molecular complex comprising a binding site;

b) employing said three-dimensional structure to design or select said potential agonist;

c) synthesizing said potential antagonist; and

d) contacting said potential agonist with BAFF to determine the ability of said potential agonist to interact with BAFF.

The binding site in step a) could be a binding site of BAFF for one or more of its receptors.

A potential agonist or a potential antagonist of BAFF is a compound. A compound could be a macromolecule, such as a protein or a polypeptide.

This invention also encompasses methods for evaluating the potential of a variant of an agonist or an antagonist of BAFF to associate with:

a) a molecule or a molecular complex defined by at least a portion or all of the structure coordinates of the BAFF amino acids, set forth in FIG. 8; or

b) a homologue of said molecule or molecular complex having a root mean square deviation from the backbone atoms of said amino acids of between 0.00 Å and 1.50 Å, preferably between 0.00 Å and 1.00 Å, more preferably between 0.00 Å and 0.50 Å;

comprising the steps of:

(i) employing computational means to perform a fitting operation between the variant and said molecule or molecular complex or said homologue of said molecule or molecular complex; and

(ii) analyzing the results of said fitting operation to quantify the association between said variant and said molecule or molecular complex or between said variant and said homologue of said molecule or molecular complex.

The molecule or molecular complex or the homologue of the molecule or molecular complex preferably comprises a binding site; said binding site could be a binding site of BAFF for one or more of its receptors.

This invention also provides a method for evaluating the potential of a variant of an agonist or an antagonist of BAFF, to associate with:

a) a first binding site of a molecule or a molecular complex defined by structure coordinates of at least one (preferably at least four) or a plurality of BAFF amino acids selected from the group consisting of His218, Val219, Phe220, Gly221, Asp222, Glu223, Leu224, Ser225, Leu226, Val227, Pro264, Arg265, Glu266, Gly161, Ser162, Tyr163, Ala151, Asp152, Ser153, Glu154, Thr155, Pro156, Leu240, Pro241, Asn242, Ser171 and Phe172, set forth in FIG. 8; or

b) a homologue of said molecule or molecular complex comprising a second binding site having a root mean square deviation from the backbone atoms of said at least one (preferably at least four) or a plurality of BAFF amino acids between 0.00 Å and 1.50Å, preferably between 0.00 Å and 1.00 Å, more preferably between 0.00 Å and 0.50 Å;

comprising the steps of:

(i) employing computational means to perform a fitting operation between the variant and said first binding site or said second binding site; and

(ii) analyzing the results of said fitting operation to quantify the association between said variant and said first binding site or said second binding site. Preferably, the first binding site in step a) or the second binding site in step b) is a binding site of BAFF for one or more of the receptors of BAFF. The fitting operation in (i) above could be between said variant and the binding site of the molecule or molecular complex or the binding site of the homologue of the molecule or molecular complex. The association in (ii) could be the association between said variant and the binding site of the molecule or molecular complex or the binding site of the homologue of the molecule or molecular complex.

Thus, the present invention provides BAFF agonist or antagonist variants with improved properties, such as increased or decreased binding affinity for BAFF.

The present invention also encompasses the chemical entities, agonists or antagonists of BAFF, variants of a BAFF agonist or antagonist, as well as compositions, such as pharmaceutical compositions, comprising the chemical entities, agonists or antagonists of BAFF, variants of a BAFF agonist or antagonist, identified by these methods.

For the first time, the present invention permits the use of molecular design techniques to design, select and synthesize chemical entities, compounds, including agonists or antagonists of BAFF, and variants of BAFF agonists or antagonists.

The design of chemical entities, compounds, including agonists or antagonists of BAFF, and variants of BAFF agonists or antagonists according to this invention generally involves consideration of two factors. First, chemical entities, compounds, including agonists or antagonists of BAFF, and variants of BAFF agonists or antagonists must be capable of physically and structurally associating with BAFF. Non-covalent molecular interactions important in the association of a protein, such as BAFF, with its binding partner include hydrogen bonding, van der Waals and hydrophobic interactions.

Second, the chemical entities, compounds, including agonists or antagonists of BAFF, and variants of BAFF agonists or antagonists must be able to assume a conformation that allows them to associate with BAFF directly. Although certain portions of chemical entities, compounds, including agonists or antagonists of BAFF, and variants of BAFF agonists or antagonists will not directly participate in these associations, those portions of chemical entities, compounds, including agonists or antagonists of BAFF, and variants of a BAFF agonist or antagonist may still influence the overall conformation of the molecule. This, in turn, may have a significant impact on potency. Such conformational requirements include the overall three-dimensional structure and orientation of the chemical entities, compounds, including agonists or antagonists of BAFF, and variants in relation to all or a portion of the binding site, e.g., active site or accessory binding site of BAFF, or the spacing between functional groups of a compound comprising several chemical entities that directly interact with BAFF.

The potential binding effect on BAFF of a chemical entity, a compound, including an agonist or antagonist of BAFF, and a variant of a BAFF agonist or antagonist can be analyzed prior to its actual synthesis or generation and testing by the use of computer modeling techniques. If the theoretical structure of the given entity or compound or variant suggests insufficient interaction and association with BAFF, synthesis and testing of the entity or compound or generation and testing that particular variant is obviated. However, if computer modeling indicates a strong interaction, the entity, compound, including an agonist or antagonist of BAFF, or variant may then be generated and tested for its ability to bind to BAFF and interrupt its association with one or more BAFF receptors using the assays described below. In this manner, generation of undesired or inoperative entities, compounds, including agonists or antagonists of BAFF, or variants may be avoided.

A BAFF-binding entity, compound, including an agonist or antagonist of BAFF, and variant of a BAFF agonist or antagonist can be computationally evaluated and designed by means of a series of steps in which chemical entities or fragments are screened and selected for their ability to associate with the binding sites of BAFF as defined in this invention. Likewise, a BAFF variant can be obtained. Examples of BAFF variants are: a BAFF variant that binds to only a subset of the receptors that bind BAFF; a BAFF variant that binds to a particular receptor of BAFF with higher or lower affinity than the native BAFF protein. For instance, BCMA and BAFF-R has only one cysteine-rich domain (“CRD”). Mutations of residues of BAFF involved in binding to the second CRD of TACI should result in a BAFF variant that can bind to BCMA and BAFF-R, but not to TACI.

One skilled in the art can use one of several methods to screen chemical entities for their ability to associate with BAFF and more particularly with a binding site of BAFF. This process may begin by visual inspection of, for example, the binding site for a receptor of BAFF on the computer screen, based on the BAFF structure coordinates in FIG. 8 generated from the machine-readable storage medium and the process of obtaining an exact binding site described herein. Selected chemical entities may then be positioned in a variety of orientations, or docked, within an individual binding site of BAFF, as defined supra (such as a binding site of BAFF for one of its receptors). Docking may be accomplished using software such as Quanta or Sybyl, followed by energy minimization and molecular dynamics with standard molecular mechanics forcefields, such as CHARMM and AMBER.

Specialized computer programs may also assist in the process of selecting chemical entities. These include, inter alia:
1. GRID (Goodford, P. J., “A Computational Procedure for Determining Energetically Favorable Binding Sites on Biologically Important Macromolecules”, J. Med. Chem., 28, pp. 849-857 (1985)). GRID is available from Oxford University, Oxford, UK.
2. MCSS (Miranker, A. and M. Karplus, “Functionality Maps of Binding Sites: A Multiple Copy Simultaneous Search Method.” Proteins: Structure, Function and Genetics, 11, pp. 29-34 (1991)). MCSS is available from Molecular Simulations, Burlington, Mass.
3. AUTODOCK (Goodsell, D. S. and A. J. Olsen, “Automated Docking of Substrates to Proteins by Simulated Annealing”, Proteins: Structure, Function, and Genetics, 8, pp. 195-202 (1990)). AUTODOCK is available from Scripps Research Institute, La Jolla, Calif.
4. DOCK (Kuntz, I. D. et al., “A Geometric Approach to Macromolecule-Ligand Interactions”, J. Mol. Biol., 161, pp. 269-288 (1982)). DOCK is available from University of California, San Francisco, Calif.

Once suitable chemical entities have been selected, they can be assembled into a single compound. Assembly may proceed by visual inspection of the relationship of the entities to each other on the three-dimensional image displayed on a computer screen in relation to the structure coordinates of BAFF. This is followed by manual model building using software such as Quanta or Sybyl.

The above-described evaluation process for chemical entities may be performed in a similar fashion for compounds that associate with BAFF or for variants of agonists and antagonists of BAFF.

Useful programs to aid one of skill in the art in connecting the individual chemical entities include:
1. CAVEAT (Bartlett, P. A. et al, “CAVEAT: A Program to Facilitate the Structure-Derived Design of Biologically Active Molecules”. In “Molecular Recognition in Chemical and Biological Problems”, Special Pub., Royal Chem. Soc., 78, pp. 182-196 (1989)). CAVEAT is available from the University of California, Berkeley, Calif.
2. 3D Database systems such as MACCS-3D (MDL Information Systems, San Leandro, Calif.). This area is reviewed in Martin, Y. C., “3D Database Searching in Drug Design”, J. Med. Chem., 35, pp. 2145-2154 (1992)).
3. HOOK (available from Molecular Simulations, Burlington, Mass.).

Instead of proceeding to build a BAFF agonist or antagonist or a BAFF binding compound in a step-wise fashion one chemical entity at a time, as described above, BAFF agonists or antagonists or other BAFF binding compounds, including variants of BAFF agonists or antagonists, may be designed as a whole or “de novo” using either an empty binding site (such as a binding site for one or more of the BAFF receptors) or optionally including some portion(s) of a known antagonist(s) of BAFF or a BAFF binding compound. These methods include:
1. LUDI (Bohm, H.-J., “The Computer Program LUDI: A New Method for the De Novo Design of Enzyme Inhibitors”, J. Comp. Aid. Molec. Design, 6, pp. 61-78 (1992)). LUDI is available from Biosym Technologies, San Diego, Calif.
2. LEGEND (Nishibata, Y. and A. Itai, Tetrahedron, 47, p. 8985 (1991)). LEGEND is available from Molecular Simulations, Burlington, Mass.
3. LeapFrog (available from Tripos Associates, St. Louis, Mo.).

Other molecular modeling techniques may also be employed in accordance with this invention. See, e.g., Cohen, N. C. et al., “Molecular Modeling Software and Methods for Medicinal Chemistry,” J. Med. Chem., 33, pp. 883-894 (1990). See also Navia, M. A. and M. A. Murcko, “The Use of Structural Information in Drug Design”, Current Opinions in Structural Biology, 2, pp. 202-210 (1992).

Once an entity, compound, including an agonist or antagonist of BAFF, or variant of agonists or antagonists of BAFF has been designed or selected by the above methods, the efficiency with which that entity, compound, including an agonist or antagonist of BAFF, or variant may bind to BAFF can be tested and optimized by computational evaluation. For example, a compound that has been designed or selected to function as a BAFF binding compound must also preferably traverse a volume not overlapping that occupied by the binding site when it is bound to the native BAFF. An effective BAFF binding compound must preferably demonstrate a relatively small difference in energy between its bound and free states (i.e., a small deformation energy of binding). Thus, the most efficient BAFF binding compound should preferably be designed with a deformation energy of binding of not greater than about 10 kcal/mole, preferably, not greater than 7 kcal/mole. BAFF binding compounds may interact with the BAFF in more than one conformation that is similar in overall binding energy. In those cases, the deformation energy of binding is taken to be the difference between the energy of the free compound and the average energy of the conformations observed when the compound binds to the protein.

A compound designed or selected as binding to BAFF may be further computationally optimized so that in its bound state it would preferably lack repulsive electrostatic interaction with the target protein. Such non-complementary (e.g., electrostatic) interactions include repulsive charge-charge, dipole-dipole and charge-dipole interactions. Specifically, the sum of all electrostatic interactions between the compound and the protein when the compound is bound to BAFF, preferably make a neutral or favorable contribution to the enthalpy of binding.

Specific computer software is available in the art to evaluate compound deformation energy and electrostatic interaction. Examples of programs designed for such uses include: Gaussian 92, revision C (M. J. Frisch, Gaussian, Inc., Pittsburgh, Pa. ©1992); AMBER, version 4.0 (P. A. Kollman, University of California at San Francisco, ©1994); QUANTA/CHARMM (Molecular Simulations, Inc., Burlington, Mass. ©1994); and Insight II/Discover (Biosysm Technologies Inc., San Diego, Calif. ©1994). These programs may be implemented, for instance, using a Silicon Graphics workstation, IRIS 4D/35 or IBM RISC/6000 workstation model 550. Other hardware systems and software packages will be known to those skilled in the art.

Once a BAFF-binding compound has been optimally selected or designed, as described above, substitutions may then be made in some of its atoms or side groups to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analyzed for efficiency of fit to BAFF by the same computer methods described in detail above.

Another approach made possible and enabled by this invention is computational screening of small molecule databases for chemical entities or compounds that can bind in whole, or in part, to BAFF; preferably to a binding site of BAFF for one or more of its receptors. In this screening, the quality of fit of such entities to the binding site may be judged either by shape complementarity or by estimated interaction energy. Meng, E. C. et al., J. Comp. Chem., 13, pp. 505-524 (1992).

The same methods described above for designing and obtaining chemical entities, compounds, including agonists or antagonists of BAFF, and variants of agonists or antagonists of BAFF can be employed to design and obtain BAFF variants.

Synthetic Compounds

The compounds of this invention can be synthetic compounds. In one embodiment, a synthetic compound designed by the methods of this invention has a molecular weight equal to or under about 1000 daltons. A synthetic compound designed by the methods of this invention preferably is soluble under physiological conditions. A synthetic compound designed by the methods of this invention preferably is bioavailable. A synthetic compound designed by the methods of this invention is preferably orally administrable. A synthetic compound designed by the methods of this invention preferably is able to bind its target (BAFF) when the target is present at physiological concentrations. A synthetic compound designed by methods of this invention preferably is non-toxic or has a medically acceptable toxicity.

Assays for Confirming that the Novel Compounds Bind and Interrupt Interaction Between BAFF and One or More of Its Receptors

A person skilled in the art is aware of conventional assays for assessing whether the entities, compounds, including agonists or antagonists of BAFF, or variants of BAFF agonists or antagonists designed according to the methods of this invention, once made, bind specifically to BAFF and whether they interrupt the interaction between BAFF and one of its receptors or act as agonists of BAFF.

Conditions Associated with Inappropriate BAFF Induced Activation in a Subject

The chemical entities, compounds, including agonists or antagonists of BAFF, and variants of BAFF agonists or antagonists designed according to methods of this invention, as well as a composition, such as a pharmaceutical composition, comprising one or more chemical entities, compounds, including agonists or antagonists of BAFF, or variants, or combinations thereof, designed by methods of this invention, can be used to treat or prevent subjects having conditions associated with inappropriate or abnormal BAFF expression or activation, possibly in conjunction with one or more agents.

Examples of conditions associated with inappropriate or abnormal BAFF expression or activation in a subject, include, inter alia: systemic lupus erythematosis, lupus nephritis, lupus neuritis, asthma, chronic obstructive pulmonary disease, bronchitis, emphysema, multiple sclerosis, uveitis, Alzheimer's disease, traumatic spinal cord injury, stroke, atherosclerosis, coronary restenosis, ischemic congestive heart failure, cirrhosis, hepatitis C, diabetic nephropathy, glomerulonephritis, osteoarthritis, rheumatoid arthritis, psoriasis, atopic dermatitis, systemic sclerosis, radiation-induced fibrosis, Crohn's disease, ulcerative colitis, multiple myeloma and cachexia.

Conditions associated with inappropriate or abnormal BAFF expression or activation in a subject, include, inter alia: cancer, autoimmune diseases, allergy, unwanted immune response, unwanted inflammatory response, rejection of donor tissue or organ, an inhibitor response to a therapeutic agent, such as a protein.

Subjects

The novel entities, compounds, including agonists or antagonists of BAFF, and variants of agonists or antagonists of BAFF designed according to this invention can be administered for treatment or prophylaxis of any mammalian subject suffering or about to suffer a condition associated with inappropriate BAFF expression or activation. Preferably, the subject is a primate, more preferably a higher primate, most preferably a human. In other embodiments, the subject may be a mammal of commercial importance, or a companion animal, or other animal of value, such as a member of an endangered species. Thus, a subject may be, inter alia, sheep, horses, cattle, goats, pigs, dogs, cats, rabbits, guinea pigs, hamsters, gerbils, rats and mice.

Route of Administration

The novel entities, compounds, including agonists or antagonists of BAFF, and variants of agonists or antagonists of BAFF designed according to this invention may be administered in any manner which is medically acceptable. Depending on the specific circumstances, local or systemic administration may be desirable. Local administration may be, for example, by subconjunctival administration. Preferably, the novel entities, compounds, including agonists or antagonists of BAFF, and variants of agonists or antagonists of BAFF is administered via an oral, an enteral, or a parenteral route such as by an intravenous, intraarterial, subcutaneous, intramuscular, intraorbital, intraventricular, intraperitoneal, subcapsular, intracranial, intraspinal, topical or intranasal injection, infusion or inhalation. The novel entities, compounds, including agonists or antagonists of BAFF, and variants of agonists or antagonists of BAFF also may be administered by implantation of an infusion pump, or a biocompatible or bioerodible sustained release implant, into the subject.

Dosages and Frequency of Treatment

Generally, the methods described herein involve administration of the novel entities, compounds, including agonists or antagonists of BAFF, and variants of agonists or antagonists of BAFF designed according to methods of this invention at desired intervals (e.g., daily, twice weekly, weekly, biweekly, monthly or at other intervals as deemed appropriate) over at least a two- or three-week period. The administration schedule is adjusted as needed to treat the condition associated with inappropriate or abnormal BAFF activation in the subject. The present treatment regime can be repeated in the event of a subsequent episode of illness.

The amount and frequency of dosing for any particular compound to be administered to a patient for inappropriate or abnormal BAFF expression or activation, or for a given immunological condition associated therewith, is within the skill and clinical judgment of ordinary practitioners of the medical and pharmaceutical arts. The general dosage and administration regime may be established by preclinical and clinical trials, which involve extensive but routine studies to determine the optimal administration parameters of the compound. Even after such recommendations are made, the practitioner will often vary these dosages for different subjects based on a variety of considerations, such as the individual's age, medical status, weight, sex, and concurrent treatment with other pharmaceuticals. Determining the optimal dosage and administration regime for each of the novel entity, compound, agonist or antagonist of BAFF used would be a routine matter for those of skill in the medical and pharmaceutical arts.

Generally, the frequency of dosing may be determined by an attending physician or similarly skilled practitioner, and might include periods of greater dosing frequency, such as at daily or weekly intervals, alternating with periods of less frequent dosing, such as at monthly or longer intervals.

For treatment, a novel entity, compound, including an agonist or antagonist of BAFF, or variant of an agonist or antagonist of BAFF designed by methods of this invention can be formulated in a pharmaceutical or prophylactic composition which includes, respectively, a pharmaceutically or prophylactically effective amount thereof dispersed in a pharmaceutically acceptable carrier. In some embodiments, the pharmaceutical or prophylactic composition can also include a pharmaceutically or prophylactically effective amount of another medically beneficial compound.

Formulation

In general, chemical entities, compounds, including agonists or antagonists of BAFF, or variants of BAFF agonists or antagonists of this invention are suspended, dissolved or dispersed in a pharmaceutically acceptable carrier or excipient. The resulting therapeutic composition does not adversely affect the recipient's homeostasis, particularly electrolyte balance. Thus, an exemplary carrier comprises normal physiologic saline (0.15M NaCl, pH 7.0 to 7.4). Other acceptable carriers are well known in the art and are described, for example, in Remington's Pharmaceutical Sciences, Gennaro, ed., Mack Publishing Co., 1990. Acceptable carriers can include biocompatible, inert or bioabsorbable salts, buffering agents, oligo- or polysaccharides, polymers, viscoelastic compound such as hyaluronic acid, viscosity-improving agents, preservatives, and the like.

A chemical entity, compound, including an agonist or antagonist of BAFF, or variant of a BAFF agonist or antagonist of this invention may be administered in a pharmaceutically effective, prophylactically effective or therapeutically effective amount, which is an amount sufficient to produce a detectable, preferably medically beneficial effect on a subject at risk or afflicted with a condition associated with inappropriate or abnormal BAFF expression or activation. Medically beneficial effects include preventing, inhibiting, reversing or attenuating deterioration of, or detectably improving, the subject's medical condition.

APRIL

As illustrated in Example 2 (in which a homology model of human APRIL amino acids 114 to 250 was built; see also FIG. 10), a homology model of APRIL may be built based on the crystal structure coordinates of a BAFF polypeptide. Furthermore, the structure coordinates of a BAFF polypeptide may be used to solve the crystal structure of APRIL, as discussed herein. The three-dimensional structure of a binding site of APRIL for one or more of the receptors of APRIL may be determined using the methods described herein for the determination of a binding site of BAFF for one or more receptors of BAFF. A putative binding site of APRIL for its receptors is determined herein by one embodiment of this invention. The putative receptor binding site residues on human APRIL for one or more of its receptors include Gly188, Arg189, Gln190, Glu191, Pro230, Arg231, Ala232, Ser131, Asp132, Val133, Pro122, Ile123, Asn124, Ala125, Thr126, Ser127, Arg206, Ala207, Tyr208, Ala14l and Leu142, and possibly other human APRIL amino acid residues. See Example 2. As in the case of BAFF, a computer for generating a three-dimensional representation of the binding site of APRIL for one or more of its receptors is included in this invention.

Once the three-dimensional structure of APRIL and a binding site of APRIL for one of the receptors of APRIL are obtained, they may be used to design and obtain chemical entities, compounds, such as agonists and antagonists of APRIL and variants of agonists or antagonists of APRIL, as well as compositions (including pharmaceutical compositions) comprising the entities, compounds, including agonists or antagonists of BAFF, or variants, by the same manner described herein for BAFF. Also, once the three-dimensional structure of a binding site of APRIL for one of the receptors of APRIL is obtained, it may be used to design and obtain novel APRIL proteins that can bind to a subset of its receptors, can bind to a receptor that it could not bind to before, and that can bind to a receptor of APRIL with higher or lower affinity than the native APRIL protein.

As in the case of BAFF, methods for generating APRIL variants and the variants themselves are included in this invention.

As in the case of BAFF, this invention includes molecules or molecular complexes defined by the structure coordinates of APRIL set forth in FIG. 10 and molecules or molecular complexes comprising a binding site of APRIL (as detailed in Example 2) for one of its receptors. This invention also includes computers for producing three-dimensional representations of molecules or molecular complexes defined by the structure coordinates of APRIL set forth in FIG. 10 and molecules or molecular complexes comprising a binding site of APRIL (as detailed in Example 2) for one of its receptors. This invention also includes methods of using the structures of molecules or molecular complexes defined by the structure coordinates of APRIL set forth in FIG. 10 and methods of using the structures of molecules or molecular complexes comprising a binding site of APRIL (as detailed in Example 2) for one of its receptors.

The following are EXAMPLES that illustrate the methods and compositions of this invention. These examples should not be construed as limiting: the examples are included for the purposes of illustration only.

All references cited herein are hereby incorporated by reference.

### EXAMPLE 1

### Determination of the Crystal Structure of a BAFF Polypeptide

The crystal structure of the TNF-homologous domain of human BAFF at 2.8 Å resolution is provided herein. The structure coordinates are shown in FIG. 8.

A myc-tagged extracellular domain fragment of human BAFF (hBAFF) (residues 136-285) (see FIG. 9b; SEQ ID NO: 2) was expressed in yeast cells, purified and crystallized.

Recombinant myc-hBAFF (residues Q136-L285 of BAFF with the myc sequence EQKLISEEDLNKEL (SEQ ID NO: 4) attached to the N-terminus) was cloned and expressed in yeast cells (Pichia pastoris). The protein was purified by anion exchange chromatography followed by gel filtration. Briefly, supernatant from Pichia cells was dialyzed; exchanged into a buffer of 10 mM Tris-HCl, pH 7.2; 50 mM NaCl; then loaded onto a Q column and eluted with a NaCl gradient (50 mM-500 mM). Further purification of myc-hBAFF was achieved by size exclusion chromatography using a Superdex 75 (26/70) column. The purified protein was analyzed by SDS-PAGE followed by Coomassie Blue staining, Western blot analysis using a mouse monoclonal 9E10 antibody (an anti-myc antibody), and N-terminal sequencing.

For crystallization, the protein was concentrated to 9 mg/ml in PBS buffer (150 mM sodium phosphate pH 7, 150 mM NaCl). Crystallization conditions were determined using the crystallization screening kits from Hampton Research (Liguna Niguel, Calif.). Crystals of optimal size were grown by vapor diffusion at 4° C. in hanging drops made by mixing 3 μl of protein solution with 3 μl of reservoir solution of 8% PEG 4000, 0.1 M sodium acetate pH 4.5. The crystals, which were rod-shaped with hexagonal cross-section, having maximal dimensions 0.2×0.2×1 mm, grew within 3 to 7 days.

The crystals were cryoprotected by gradually equilibrating them in a solution containing 25% ethylene glycol, 12% PEG 4000, 0.1 M Na acetate pH 4.5, and then flash frozen in liquid nitrogen. Native X-ray diffraction data up to 3.3 Å resolution were collected at −175° C. on an R-axis IV detector system (Molecular Structure Corporation, Woodlands, Tex.) using Cu Kα radiation. The unit cell was hexagonal, with cell dimensions a=b=122.12 Å, c=157.55 Å. Data processing with DENZO and SCALEPACK (Otwinowski, Z., Oscillation Data Reduction Program, Proceeding of the CCP4 study weekend: data collection and processing. Daresbury Laboratory, Warrington, UK: Sawer, L., Isaacs, N. & Bailey, S. eds. 56-62(1993)) indicated that the space group was P6₁ or its enantiomorph P6₅. The Matthews coefficient (Matthews, B. W., J. Mol. Biol., 33: p. 491-497 (1968)) was 2.83 Å ³Da⁻¹ with a solvent content of 56.1%, indicating that there are two trimers of BAFF in the asymmetric unit. Data statistics are shown in Table 1.

The crystal structure was solved by multiple isomorphous replacement (MIR) and refined to 2.8 Å resolution. Attempts to solve the structure by molecular replacement with the program AMoRe (Navaja, J., AMoRe: an Automated Package for Molecular Replacement, Acta Crystallogr. A, 50: p. 157-163 (1994)) from the CCP4 program package (Collaborative Computational Projects No. 4. The CCP4 Suite: Programs for Protein Crystallography, Acta Cryst. D50: p. 760-763) using a variety of TNF-related structures as search probes were not successful. No obvious peaks corresponding to the 3-fold axes of the two BAFF trimers were observed in the self rotation function, apparently due to a diffuse Patterson map, similar to the case of TNF (Jones, E. Y., et al., Acta Crystallogr A,. 47(Pt 6): p. 753-70 (1991)). A search for heavy atom derivatives was undertaken and useful phase information was obtained from Hg, Pt, Ir and Sm derivatives by using the program SOLVE (Terwilliger, T. C. and J. Berendzen, Acta Crystallogr D Biol Crystallogr, 55(Pt 4): p. 849-61 (1999)) (Table 1). The resulting electron density map (figure of merit 0.41) was considerably improved by density modification with the program RESOLVE (figure of merit 0.67) (Terwilliger, T. C., Acta Crystallogr D, 56: p. 965-972 (2000)). Inspection of maps revealed continuous density for the polypeptide chains and indicated that the correct space group is P6₅. A partial model of BAFF based on the human CD40L structure (Karpusas, M., et al., Structure, 3(12): p. 1426 (1995) and Karpusas, M., et al., Structure, 3: p. 1031-1039 (1995)) was manually fit on to the map and was rigid-body refined with XPLOR (Brunger, A. T., X-PLOR Version 3.0: a System for X-ray Crystallography and NMR, New Haven, USA, Yale University Press (1992)). The initial crystallographic R-factor was 50.5%. The fitted model was used to calculate non-crystallographic symmetry (NCS) operators. Phases calculated from the model were combined with experimental phases with SIGMAA from CCP4 package (Collaborative Computational Projects No. 4. The CCP4 Suite: Programs for Protein Crystallography, Acta Cryst. D50: p. 760-763) and were improved by solvent flattening, histogram matching and 6-fold averaging with the program DM from the CCP4 package (Collaborative Computational Projects No. 4. The CCP4 Suite: Programs for Protein Crystallography, Acta Cryst. D50: p. 760-763). The RESOLVE and DM maps as well as the 2Fo-Fc maps were used for iterative model building with the graphics program QUANTA (Molecular Simulations, Inc., San Diego, Calif.).

All subsequent refinement steps were carried out using the program CNX (Brunger, A. T., Crystallography & NMR System: a New Software for Macromolecular Structure Determination, Acta Crystallogr. D., 54: p. 905-921 (1998); and Molecular Simulations, Inc.). These included maximum likelihood positional refinement, torsion angle simulated annealing and grouped B-factor refinement with NCS restraints. 10% of the data were allocated for calculation of the R-free factor. A bulk-solvent correction was employed after the complete model was built. Simulated annealing omit maps were used to check validity of the model. NCS restrains were removed for certain regions of the molecule at the later stages of refinement. After completion of refinement, the R-working and R-free factors of the model were 22.6% and 26.8% respectively for the data (F>2σ) in the 35-3.3 Å resolution range. At that stage, synchrotron data to 2.8 Å resolution were collected at beamline X4A of National Syncrotron Light Source (NSLS) and the structure,was further refined against the new data (Table 1). The R-working and R-free factors of the final refined model were 21.7% and 25.0% respectively for the data (F>2σ) in the 30-2.8 Å resolution range (R-working and R-free are 22.2% and 25.4% respectively for the data (F>0σ) in the 35-2.8 Å resolution range). Stereochemistry statistics were calculated with PROCHECK (Laskowski, R. A., et al., J. Appl. Crystallogr., 26: p. 283-290 (1993)) and CNX (Brunger, A. T., Crystallography & NMR System: a New Software for Macromolecular Structure Determination, Acta Crystallogr. D., 54: p. 905-921 (1998)). Electrostatic potential surfaces were calculated with GRASP (Nicholls, A., GRASP: Graphical Representation and Analysis of Surface Properties (New York, Columbia University) (1992)). Additional data statistics are presented in Table 1.

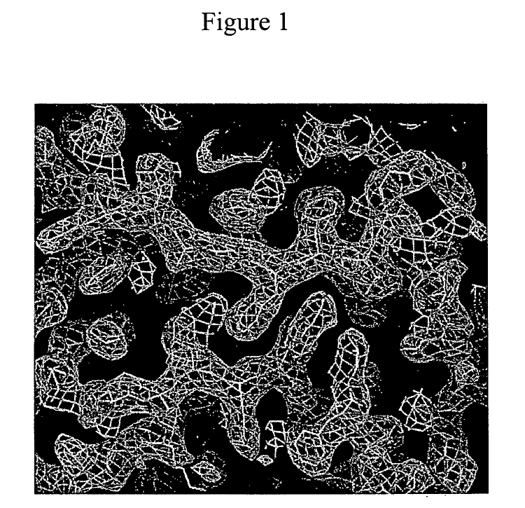

Despite the limited resolution range, all residues except the myc-tag and N-terminal residues 136-141 were uniformly well defined in the final 2Fo-Fc electron density map (FIG. 1). The asymmetric unit of the crystal contained two trimers of BAFF. The final crystallographic R-working and R-free were 21.7% and 25.0% respectively for the data (F>2σ) in the 30-2.8 Å resolution range. The model consists of 864 amino acid residues constituting 6 polypeptide chains. No water molecules were added to the model. The root mean square (r.m.s.) deviations on bond lengths were 0.008 Å and on bond angles were 1.4°. All non-glycine residues have φ/ψ angles in the allowed regions of the Ramachandran diagram and 84.8% of the residues had φ/ψ angles in the most favored regions. The average B-factor of the main chain atoms is 37.4 Å² Crystallographic statistics are summarized in Table 1.

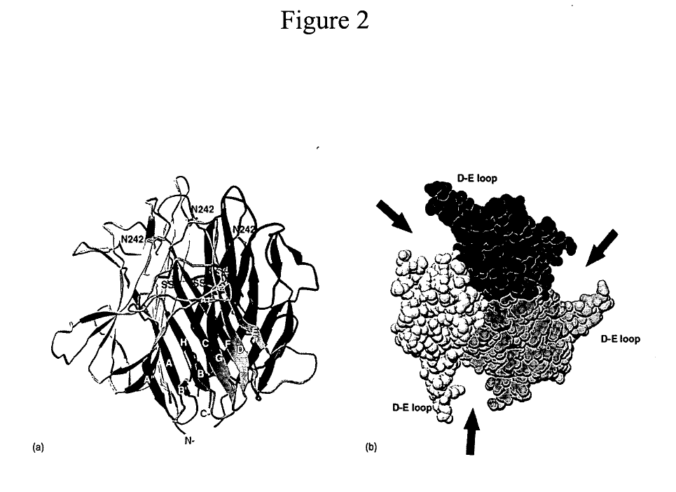

Like the other TNF family members, the crystallized BAFF fragment is a homotrimeric protein with an overall shape that resembled that of a truncated pyramid (FIGS. 2a and 2b). The dimensions of the molecular trimer were 58×58×54 Å. Each monomer folded as a sandwich of two antiparallel β-sheets with Greek key topology. In the description that follows, for the β-strands and other structural features, the notation introduced for TNF is used (Eck, M. J. and S. R. Sprang, J Biol Chem, 264(29): p. 17595-605 (1989)). The inner β-sheet is involved in monomer contacts and is composed of β-strands A″, A, H, C and F (FIG. 2a). The outer sheet contains most of the solvent-exposed residues and is composed of A1, A2, B′, B, G, D and E strands. The β-strands are connected by loops whose length varies considerably. The core of the protein is mostly hydrophobic but it also contains a few buried polar residues involved in interactions, such the one between the His210 and Tyr201 side chains. A disulfide bridge connecting Cys232 of β-strand E with Cys245 of β-strand F was observed. There is also a free cysteine (Cys146) at the N-terminal end of the A strand that is partially exposed to the solvent.

The three monomers were related to each other by a 3-fold axis that was aligned approximately with the β-strands of the monomers. The monomer interface was primarily hydrophobic, characterized by the participation of 5 aromatic side chain residues (Tyr192, Phe194, Tyr196, Tyr246, Phe278). A 3-residue cluster was formed by Gln234 from each monomer on the 3-fold axis near the top of the pyramid. Approximately 945 Å² of monomer solvent accessible surface was buried to form the trimer and 56% of that surface area was hydrophobic.

The r.m.s. positional deviation between equivalent residues from different BAFF monomers was small (0.66 Å). The deviation was mostly due to significant differences in the conformation for the D-E loop that include positional shifts as large as 3.5 Å for some atoms. Three different hairpin-like conformations were observed for the D-E loop. The first one was adopted by four out of the six monomers and was stabilized by an internal hydrogen bond between the amide group of Glu223 and the carbonyl group of Phe220 and by interactions with symmetry related molecules. The other two conformations were adopted by the other two monomers respectively and were characterized by the absence of any crystal contacts for the loop. In one of the monomers, the loop was stabilized by a hydrogen bond between side chain atom Oε1 of Glu223 and the amide nitrogen of Phe220. The side chain of Glu223 was also stabilized by an interaction of Oε2 with the Nζ of Lys216.

Weak electron density was observed for the biantennary complex-type carbohydrate attached to residue Asn242 on the F strand of BAFF. Mass spectrometry analysis of the protein material used for the crystallization indicated the presence of a BAFF component corresponding to the protein plus a high mannose glycan. Apparently, the crystallization process selected both the glycosylated and aglycosylated species of BAFF. The electron density was not clear enough to allow model building of the carbohydrate residues. However, it is obvious that residues Tyr206 and Arg231 make contacts with the carbohydrate. There are no crystal contacts close to that region and the rest of the carbohydrate is disordered within a large solvent channel in the crystal.

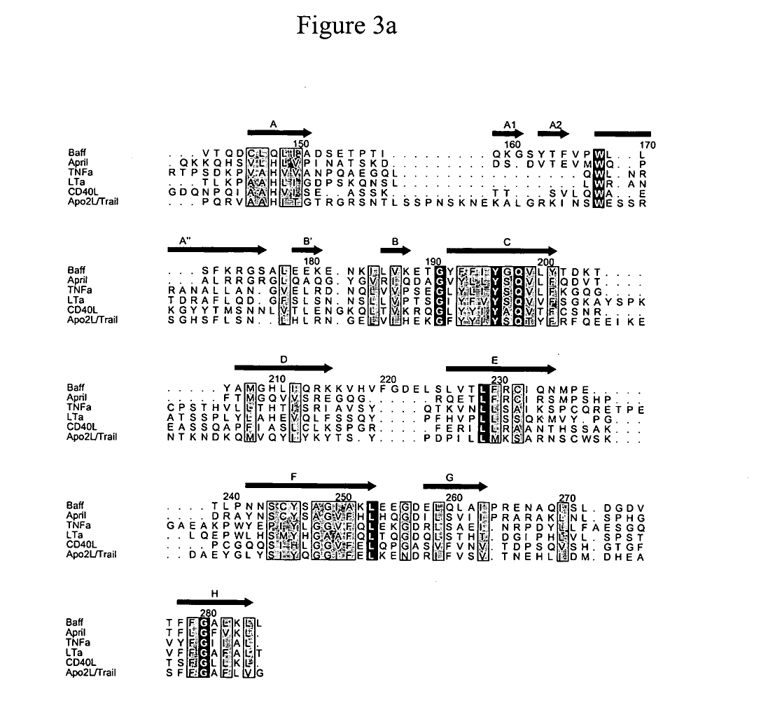

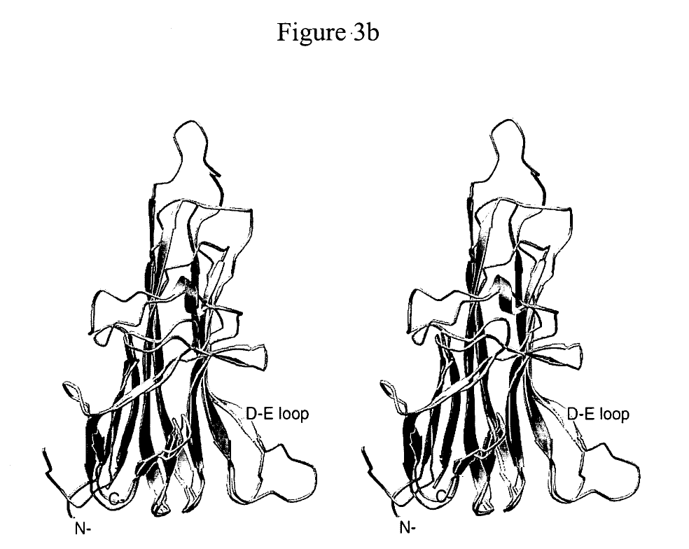

Although the overall structure of BAFF was similar to that of other TNF family members, the structure of the loops and certain β-strands varied considerably as compared to that of other TNF family members. BAFF has low sequence homology with other family members: the sequence identity of BAFF with each of TNF-α, LT-α, CD40L and TRAIL is 21.5%, 21.5%, 17.4% and 20.1%, respectively, based on structural alignments (FIGS. 3a and 3b); and the corresponding r.m.s. positional deviations between equivalent Cα atoms of BAFF with each of TNF-α, LT-α, CD40L and TRAIL is 2.1 Å, 2.0 Å, 2.0 Å and 2.4 Å, respectively. The BAFF crystal structure determined herein is the first available structure of a group of TNF family member proteins characterized by the presence of a disulfide bridge connecting β-strands E and F.

The BAFF structure showed conservation of hydrophobic residues that are important components of the protein core of TNF family members, such as Trp168 and Phe279. The size and position of the major β-strands is similar to that of other TNF family members, with the exception of strand F, which is markedly shorter. The structure showed that the first loop that connected strands A and A″ was rather long and contained two short β-strands (termed A1 and A2), which formed an extension of the external β-sheet. Analogous β-strands have not been observed in the previously determined members of the TNF family except for a strand, similar to A1, that has been observed in one of the available Apo2L/TRAIL structures (Mongkolsapaya, J., et al., Nat Struct Biol, 6(11): p. 1048-1053 (1999)). The conformation of the A-A″ loop was stabilized by the two small β-strands and a few other interactions with other parts of the molecule, as evidenced by the well defined electron density. Another unusual feature of the BAFF structure was the absence of the loop connecting A′ and A″ strands. In the case of BAFF, these two strands formed a single continuous A″ strand with a β-bulge in the middle.

The C-D and E-F loops of the BAFF structure that are located at the “top” of the pyramid were shorter than those of the other members of the TNF family. Their shorter length may account for the well-defined electron density, which is not common for that region of TNF ligands. The most notable feature of the BAFF structure was an unusually long and extended D-E loop (residues Lys216-Ser215) (FIGS. 2a and 2b). This D-E loop appeared to be the longest D-E loop of all the known members of the TNF family and corresponded to an insertion of 6-11 amino acid residues (depending to the TNF member it is compared with). This loop is flexible, as evidenced by the presence of three different conformations in the crystal.

Although the structure of the G-H loop is generally conserved in the four other known structures of TNF family members, it is significantly different in BAFF, particularly for the N-terminal part of the loop (Pro264-Asn267). That part of the loop was observed to extend further away from the core of the molecule and was stabilized by several interactions, including an H-bond between Oδ1 of Asp203 and the carbonyl oxygen of Ala268 and an H-bond between Oε1 of Gln159 and the amide nitrogen of Asn267.

The disulfide bridge connecting strands E and F may play a role in stabilizing the BAFF molecule. The bridge lay close to the 3-fold axis in a region of the molecule that frequently contain stabilizing elements, such as other disulfide bonds, as in TNFα and CD40L, or such as a Zn²⁺ binding site, as in Apo2L/TRAIL (Hymowitz, S. G., et al., Biochemistry, 39(4): p. 633-40 (2000)).

In the absence of structure-function data for BAFF and a co-crystal structure of a complex of BAFF and one of its receptors, the location of the binding site for the three known BAFF receptors (BCMA, TACI and BAFF-R) may only be inferred by analogy to what is known for other TNF family members. It is therefore expected to lie in the elongated cleft formed between adjacent monomers of a BAFF trimer. Three receptor binding sites are expected to exist per BAFF trimer; roughly consisting of residues of the D-E, A″-A, C-D and G-H loops, involving BAFF amino acid residues His218-Val227, Pro264-Glu266, Gly161-Tyr163, Ala151-Pro156, Leu240-Asn242,Ser171 and Phe172, and perhaps other residues. There is almost no conservation of any binding site residues of BAFF relative to LT-α and TRAIL. The character of the putative binding site surface is mixed and includes positively and negatively charged polar residues, uncharged polar residues and hydrophobic residues.

In the two cases of known TNF ligand-receptor complexes, the receptors are observed to be elongated molecules that bind along the whole length of ligand cleft, making a large number of contacts with the TNF ligand. Two consecutive CRDs from the receptors make contacts with the TNF ligand that can be grouped into two patches, the top patch (patch A) and the bottom patch (patch B) (Hymowitz, S. G., et al., Mol Cell, 4(4): p. 563-71 (1999)). Patch A corresponds mostly to contacts with the third receptor CRD and patch B corresponds mostly to contacts with the second CRD. In all the TNF family members with known structures, the binding site cleft is particularly shallow.

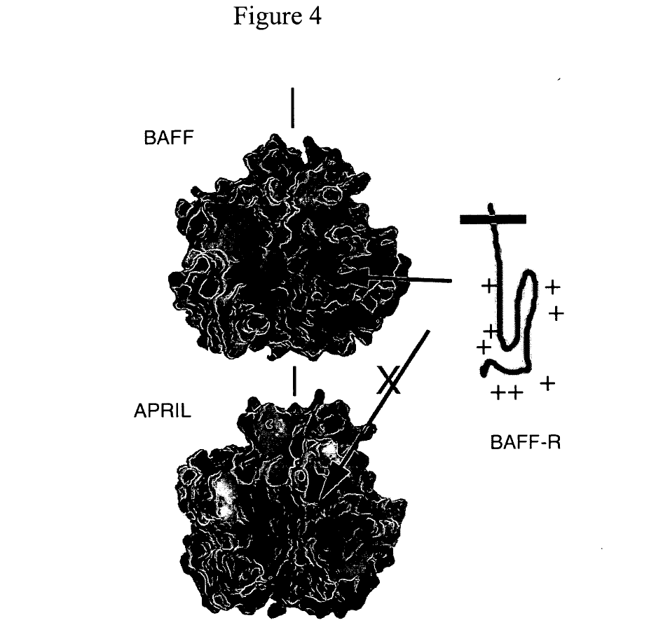

In the case of BAFF, however, the presence of the unusually extended D-E loop results in the formation of a relatively deep, concave site in the lower part of the cleft (bottom of the pyramid) that is likely to constitute an important part of the receptor binding site. This site corresponds to the patch B described. The observed flexibility of the D-E loop may be a feature necessary for structural adaptation for receptor binding. Calculation of electrostatic potential surface shows that there is a predominance of negative charges on the surface of patch B (FIG. 4). These charges are mostly localized on the “rim” of the cavity. The residues that are primarily responsible for these charges are Asp152, Asp222, Glu223, Glu254, Asp273 and Asp275. The rest of the putative receptor binding site has mostly neutral charges, with the exception of a small positively charged area due to residue Arg231. This arginine is conserved in CD40L (Arg2O7), where it was found to be an important contributor to binding and specificity (Singh, J., et al., Protein Sci, 7(5): p. 1124-35 (1998)). Arg231 of BAFF is positioned between patches A and B and makes contacts with the carbohydrate that is attached to residue Asn242.

The carbohydrate of BAFF appears to occupy part of the putative receptor binding site, near patch A. The carbohydrate would be in steric conflict with a bound receptor having the size and shape of TNFR (tumor necrosis factor receptor) unless it adopts a limited set of conformations.

Understanding the interaction of BAFF with its receptors at a structural level is of interest due to several unusual characteristics of its receptors. The extracellular domains of BCMA and BAFF-R are the smallest known of all TNF receptors: they have a size equivalent to one CRD. All the other TNF receptors have at least two CRDs (such as TACI) and usually around four (such as TNFR and CD40). Although the N-terminal CRD of several receptors, such as TNFR and CD40, is not involved in direct contacts with the ligand, it appears to be necessary for ligand binding and may have other important functions (Chan, K. F., et al., Immunity, 13(4): pp. 419-422 (2000)). Thus, it is of note that the smaller size of BAFF receptors is sufficient for the different aspects of the function of these molecules.

The existing structural information for other TNF family receptors is not sufficient for the modeling of the interactions of BAFF with its receptors. Analysis of the BCMA and TACI sequences suggests that these proteins may adopt the AlC2 motif (Thompson, J. S., et al., J Exp Med, 192(1): p. 129-35 (2000)). This motif has been observed in the fourth CRD of TNFR, which is not involved in ligand-receptor contacts (Naismith, J. H., et al., Structure, 4(11): p. 1251-62 (1996)) and therefore cannot be used to model a BAFF-receptor complex. In addition, a satisfactory alignment of the BAFF-R sequence with any of the other receptor sequences is not easy to generate; indicating that the BAFF-R extracellular domain may adopt a distantly related, or even new, folding motif relative to other TNFR family members (Thompson et al., BAFF-R, a Novel TNF Receptor That Specifically Interacts with BAFF, Sciencexpress (Aug. 16, 2001), at http://www.sciencexpress.org) and Thompson, J. S. et al., Science (Sep. 14, 2001); 293 (5537): 2108-2111.

The fact that BAFF has an unusually long D-E loop that facilitates the formation of a deep cleft may be related to the unusually small size of two of the BAFF receptors, BCMA and BAFF-R. The potential increase in ligand-receptor number of contacts due to the deep cleft may compensate for the reduction of contacts due the absence of a second CDR. Thus, it is likely that BCMA and BAFF-R bind to the cleft in a manner analogous to the second CRD of TNFR.

Also, the observation of the negatively charged region of the BAFF cleft is of interest because the BAFF-R receptor appears to be a protein with an unusually large number of positively charged residues, particularly close to the N-terminus (Thompson et al., BAFF-R, a Novel TNF Receptor That Specifically Interacts with BAFF, Sciencexpress (Aug. 16, 2001), at http://www.sciencexpress.org and Thompson, J. S. et al., Science (Sep. 14, 2001); 293 (5537): 2108-2111). The apparent presence of electrostatic complementarity may suggest that the binding energy of BAFF-R-BAFF association may have a significant electrostatic component. Homology modeling of the closely related APRIL molecule, as detailed in Example 2, indicates that APRIL does not have a similar negatively-charged area but, instead, it has an extensively positively charged area (FIG. 4). This observation is consistent with the fact that BAFF-R does not bind to APRIL (Thompson et al., BAFF-R, a Novel TNF Receptor That Specifically Interacts with BAFF, Sciencexpress (Aug. 16, 2001), at http://www.sciencexpress.org and Thompson, J. S. et al., Science (Sep. 14, 2001); 293 (5537): 2108-2111). In contrast, BCMA and TACI sequences indicate that these molecules contain mixed electrostatic charges, which is consistent with the fact that these molecules bind to both BAFF and APRIL. The above considerations suggest that predictions of specificity between different TNF family ligands and receptors based on electrostatic complementarities may be feasible in cases where sufficient structural information is available.

TABLE 1
Summary of Crystallographic Analysis Diffraction data

| | Native | Native | | | | |
|---|---|---|---|---|---|---|
| | 1 | 2 (X4A) | Hg^{§} | Pt^{§§} | Sm^{§§§} | Ir^{§§§§} |
| Soaking | — | — | 0.1 mM, | 1 mM, | 2 mM, | 1 mM, |
| conditions | | | 24 hr. | 24 hr. | 24 hr. | 24 hr. |
| Cell | | | | | | |
| dimensions | | | | | | |
| a (Å) | 122.12 | 121.72 | 122.62 | 122.72 | 122.61 | 122.75 |
| c (Å) | 157.55 | 160.74 | 158.35 | 156.71 | 159.2 | 156.49 |
| Space group | P6₅ | P6₅ | P6₅ | P6₅ | P6₅ | P6₅ |
| Resolution | 35-3.3 | 30-2.8 | 35-3.5 | 35-4.2 | 35-3.7 | 35-4.1 |
| (Å) | (3.42-3.3)† | (2.9-2.8)† | | | | |
| Unique | 20,072 | 31,743 | 16,814 | 9,127 | 14,417 | 10,227 |
| reflections | | | | | | |
| Completeness | 99.7 | 95.6 | 98.3 | 93.1 | 99.5 | 96.8 |
| (%) | (99.6)† | (81.8)† | | | | |
| Average I/σ | 19.1. | 14.2 | 12.6 | 10.8 | 14.3 | 8.1 |
| | (3.8)† | (2.9)† | | | | |
| R_{merge}* (%) | 9.0 | 7.9 | 12.4 | 15.3 | 14.9 | 16.5 |
| | (38.4)† | (26.7)† | | | | |
| # of sites | — | — | 4 | 3 | 1 | 5 |
| Rᵢₛₒ | — | — | 22.9 | 24.8 | 26.8 | 30.9 |

Phasing:

| | | | | |
|---|---|---|---|---|
| Figure of Merit: | centric | 0.73 | accentric | 0.67 |

Refinement:

| | |
|---|---|
| Resolution range used (F > 2σ) (Å) | 30-2.8 |
| R-factor (%) | 21.7 |
| R-free (%) | 25.0 |

Model:

| | |
|---|---|
| No. of non-H atoms | 6,858 |
| No. of protein residues | 864 |
| Contents of asymmetric unit | 2 BAFF trimers |
| Average B-factor, main chain (Å²) | 37.4 |
| Average B-factor, side chain (Å²) | 45.2 |

Stereochemistry:

RMS deviations

| | |
|---|---|
| Bond lengths (Å) | 0.008 |
| Bond angles (Å) | 1.44 |
| Dihedrals (°) | 26.6 |
| Improper (°) | 0.82 |

^{§}Hg = (C₂H₅HgO)HPO₂
^{§§}Pt = K₂Pt(NO₂)₄
^{§§§}Sm = SmCl₃
^{§§§§}Ir = (NH₄)IrCl₆
*R_{merge} = ΣₕΣᵢ|Iₕᵢ − Iₕ|/ΣₕᵢIₕᵢ
†Values for the highest resolution shell given in parenthesis

### EXAMPLE 2

### Homology Model Structure of APRIL

According to Martin, A. C., et al., Proteins Suppl., 1: p. 14-28 (1997) and Sanchez, R. and A. Sali, Proteins, Suppl(1): p. 50-8 (1997), sequence homology of 30% or higher is sufficient for the generation of homology models of significant level of accuracy. APRIL has 34% sequence identity with BAFF (FIG. 3a). Therefore, a homology model of the TNF-homologous domain of a human APRIL trimer was built by using the crystal structure of BAFF as a template. The homology model structure coordinates of human APRIL are shown in FIG. 10.

A sequence alignment of APRIL and BAFF TNF-homologous domain sequences was generated with the program QUANTA and refined manually (FIG. 3a). The alignment and the crystal structure of BAFF were used for the generation of a homology model of human APRIL (residues 114-250 (see FIG. 9c)) with the program MODELER (Sali, A. and T. L. Blundell, J Mol Biol, 212(2): p. 403-28 (1990)). This is an automated program that used spatial restraints such as inter-Cα distances and dihedral angles from the BAFF structure and generated the model by minimizing the violations of the restraints. A trimer of APRIL was generated from the monomer by applying the same transformations that relate the three BAFF monomers.

Based on the modeling, the overall structures of APRIL and BAFF are predicted to be very similar. The only major differences in APRIL, relative to BAFF, is a 6-residue deletion in the D-E loop, a 2-residue insertion in the top of the E-F loop and a one residue deletion in the A-A″ loop of APRIL. The aromatic residues that are involved in the formation of the trimerization interface are conserved, except for Phe194 of BAFF that is a leucine in APRIL. There are no significant steric conflicts between monomers of an APRIL trimer.

The shorter length of the D-E loop of APRIL is a significant structural difference that is likely to play a role in differences of specificity between APRIL and BAFF. As a consequence of the shorter loop size, the putative receptor binding site of APRIL for one or more of its receptors is much more shallow. The other structural differences that may account for specificity differences may be the different surface amino acid side chains. Most of the putative receptor binding site residues are different in APRIL when compared to BAFF except for those in a contiguous region that is located near patch A, which consists mostly of residues of the E strand. The putative receptor binding site residues of APRIL for one or more of its receptors include Gly188, Arg189, Gln190, Glu191, Pro230, Arg231, Ala232, Ser131, Asp132, Val133, Pro122, Ile123, Asn124, Ala125, Thr126, Ser127, Arg206, Ala207, Tyr208, Ala141 and Leu142, and possibly other residues. Calculation of the electrostatic potential surface of an APRIL trimer indicates that there is a predominance of positive charges on the putative receptor binding site (FIG. 4). This is opposite to the predominance of negative charges in BAFF. The dominant feature of positive potential is associated with Arg195 that is positioned in the middle of the binding site and is conserved in BAFF (Arg231).

Equivalents

The invention may be embodied in other specific forms without departing from the spirit or essential characteristics thereof. The foregoing embodiments are therefore to be considered in all respects illustrative of, rather than limiting on, the invention disclosed herein. All changes which come within the meaning and range of equivalency of the claims are intended to be embraced therein.

1. A crystallizable composition comprising a BAFF polypeptide.

2. The crystallizable composition according to claim 1, wherein said BAFF polypeptide is a polypeptide comprising the extracellular domain of BAFF.

3. The crystallizable composition according to claim 1, wherein said BAFF polypeptide comprises a polypeptide consisting of amino acid 136 to amino acid 285 of human BAFF (SEQ ID NO: 2).

4. A crystallizable composition comprising a trimer of BAFF polypeptides.

5. A crystal comprising a BAFF polypeptide.

6. The crystal according to claim 5, wherein said BAFF polypeptide comprises the extracellular domain of BAFF.

7. The crystal according to claim 5, wherein said BAFF polypeptide comprises a polypeptide consisting of amino acid 136 to amino acid 285 of human BAFF (SEQ ID NO: 2).

8. A crystal comprising a trimer of BAFF polypeptides.

9. A computer for producing a three-dimensional representation of:
a) a molecule or a molecular complex defined by the structure coordinates of the BAFF amino acids set forth in FIG. 8, or b) a homologue of said molecule or molecular complex, wherein said homologue has a root mean square deviation from the backbone atoms of said amino acids between 0.00 Å and 1.50 Å; and
wherein said computer comprises:
(i) a machine-readable data storage medium comprising a data storage material encoded with machine-readable data, wherein said data comprises the structure coordinates of the BAFF amino acids set forth in FIG. 8; and
(ii) instructions for processing said machine-readable data into said three-dimensional representation.

10. The computer for producing a three-dimensional representation according to claim 9, wherein said homologue has a root mean square deviation from the backbone atoms of said amino acids of between 0.00 Å and 1.00 Å.

11. The computer for producing a three-dimensional representation according to claim 9, wherein said homologue has a root mean square deviation from the backbone atoms of said amino acids of between 0.00 Å and 0.50 Å.

12. A computer for determining the structure coordinates corresponding to X-ray diffraction data obtained from a molecule or molecular complex, wherein said computer comprises:
a) a machine-readable data storage medium comprising a data storage material encoded with machine-readable data, wherein said data comprises the structure coordinates of BAFF according to FIG. 8;
b) a machine-readable data storage medium comprising a data storage material encoded with machine-readable data, wherein said data comprises X-ray diffraction data obtained from said molecule or molecular complex; and
c) instructions for performing a Fourier transform of the machine readable data of (a) and for processing said machine readable data of (b) into structure coordinates.

13. A computer for producing a three-dimensional representation of:
a) a molecule or molecular complex comprising a first binding site defined by structure coordinates of a plurality of BAFF amino acids selected from the group consisting of His218, Val219, Phe220, Gly221, Asp222, Glu223, Leu224, Ser225, Leu226, Val227, Pro264, Arg265, Glu266, Gly161, Ser162, Tyr163, Ala151, Asp152, Ser153, Glu154, Thr155, Pro156, Leu240, Pro241, Asn242, Ser171 and Phe172 according to FIG. 8; or
b) a homologue of said molecule or molecular complex, wherein said homologue comprises a second binding site that has a root mean square deviation from the backbone atoms of a plurality of BAFF amino acids between 0.00 Å and 1.50 Å;
wherein said computer comprises:
(i) a machine-readable data storage medium comprising a data storage material encoded with machine-readable data, wherein said data comprises the structure coordinates of said plurality of BAFF amino acids selected from the group consisting of His218, Val219, Phe220, Gly221, Asp222, Glu223, Leu224, Ser225, Leu226, Val227, Pro264, Arg265, Glu266, Gly161, Ser162, Tyr163, Ala151, Asp152, Ser153, Glu154, Thr155, Pro156, Leu240, Pro241, Asn242, Ser171 and Phe172 according to FIG. 8; and
(ii) instructions for processing said machine-readable data into said three-dimensional representation.

14. The computer for producing a three-dimensional representation according to claim 13, wherein said homologue comprises a second binding site that has a root mean square deviation from the backbone atoms of said amino acids of between 0.00 Å and 1.00 Å.

15. The computer for producing a three-dimensional representation according to claim 13, wherein said homologue comprises a second binding site that has a root mean square deviation from the backbone atoms of said amino acids of between 0.00 Å and 0.50 Å.

16. The computer according to any one of claims 13-15, wherein said first binding site is a binding site of BAFF for one or more receptors of BAFF.

17. The computer according to any one of claims 13-15, wherein said second binding site is a binding site of BAFF for one or more receptors of BAFF.

18. A computer for producing a three-dimensional representation of:
a) a molecule or molecular complex comprising a first binding site defined by structure coordinates of at least four BAFF amino acids selected from the group consisting of His218, Val219, Phe220, Gly221, Asp222, Glu223, Leu224, Ser225, Leu226, Val227, Pro264, Arg265, Glu266, Gly161, Ser162, Tyr163, Ala151, Asp152, Ser153, Glu154, Thr155, Pro156, Leu240, Pro241, Asn242, Ser171 and Phe172 according to FIG. 8; or
b) a homologue of said molecule or molecular complex, wherein said homologue comprises a second binding site that has a root mean square deviation from the backbone atoms of said at least four BAFF amino acids of between 0.00 Å and 1.50 Å;
wherein said computer comprises:
(i) a machine-readable data storage medium comprising a data storage material encoded with machine-readable data, wherein said data comprises the structure coordinates of said at least four BAFF amino acids selected from the group consisting of His218, Val219, Phe220, Gly221, Asp222, Glu223, Leu224, Ser225, Leu226, Val227, Pro264, Arg265, Glu266, Gly161, Ser162, Tyr163, Ala151, Asp152, Ser153, Glu154, Thr155, Pro156, Leu240, Pro241, Asn242, Ser171 and Phe172 according to FIG. 8; and
(ii) instructions for processing said machine-readable data into said three-dimensional representation.

19. The computer for producing a three-dimensional representation according to claim 18, wherein said homologue comprises a second binding site that has a root mean square deviation from the backbone atoms of said amino acids of between 0.00 Å and 1.00 Å.

20. The computer for producing a three-dimensional representation according to claim 18, wherein said homologue comprises a second binding site that has a root mean square deviation from the backbone atoms of said amino acids of between 0.00 Å and 0.50 Å.

21. The computer according to any one of claims 18-20, wherein said first binding site is a binding site of BAFF for one or more receptors of BAFF.

22. The computer according to any one of claims 18-20, wherein said second binding site is a binding site of BAFF for one or more receptors of BAFF.

23. The computer according to any one of claims 9, 12, 13 or 18, further comprising a display for displaying said structure coordinates.

24. A computer for determining at least a portion of the structure coordinates corresponding to an X-ray diffraction pattern of a molecule or a molecular complex whose structure is unknown, wherein said computer comprises:
a) a machine-readable data storage medium comprising a data storage material encoded with machine-readable data, wherein said data comprises at least a portion of the structure coordinates according to FIG. 8;
b) a machine-readable data storage medium comprising a data storage material encoded with machine-readable data, wherein said data comprises an X-ray diffraction pattern of said molecule or molecular complex;
c) a working memory for storing instructions for processing said machine-readable data of a) and b);
d) a central processing unit coupled to said working memory and to said machine-readable data of a) and b) for performing a Fourier transform of the machine readable data of (a) and for processing said machine readable data of (b) into structure coordinates; and
e) a display coupled to said central processing unit for displaying said structure coordinates of said molecule of molecular complex.

25. The method according to claim 24, wherein said molecule or molecular complex whose structure is unknown comprises an APRIL polypeptide.

26. The method according to claim 25, wherein said APRIL polypeptide comprises the extracellular domain of APRIL.

27. The method according to claim 24, wherein said molecule or molecular complex whose structure is unknown comprises a trimer of APRIL polypeptides.

28. A method for evaluating the potential of a chemical entity to associate with:
a) a molecule or molecular complex defined by the structure coordinates of the BAFF amino acids set forth in FIG. 8; or b) a homologue of said molecule or molecular complex having a root mean square deviation from the backbone atoms of said amino acids between 0.00 Å and 1.50 Å;
comprising the steps of:
(i) employing computational means to perform a fitting operation between said chemical entity and the molecule or molecular complex or said homologue of said molecule or molecular complex; and
(ii) analyzing the results of said fitting operation to quantify the association between said chemical entity and said molecule or molecular complex or said homologue of said molecule or molecular complex.

29. The method according to claim 28, wherein said homologue has a root mean square deviation from the backbone atoms of said amino acids between 0.00 Å and 1.00 Å.

30. The method according to claim 28, wherein said homologue has a root mean square deviation from the backbone atoms of said amino acids between 0.00 Å and 0.50 Å.

31. A method for evaluating the potential of a chemical entity to associate with:
a) a molecule or molecular complex comprising a first binding site defined by a plurality of BAFF amino acids selected from the group consisting of His218, Val219, Phe220, Gly221, Asp222, Glu223, Leu224, Ser225, Leu226, Val227, Pro264, Arg265, Glu266, Gly161, Ser162, Tyr163, Ala151, Asp152, Ser153, Glu154, Thr155, Pro156, Leu240, Pro241, Asn242, Ser171 and Phe172 according to FIG. 8; or
b) a homologue of said molecule or molecular complex comprising a second binding site having a root mean square deviation from the backbone atoms of a plurality of BAFF amino acids between 0.00 Å and 1.50 Å;
comprising the steps of:
(i) employing computational means to perform a fitting operation between said chemical entity and said first binding site or said second binding site; and
(ii) analyzing the results of said fitting operation to quantify the association between said chemical entity and said first binding site or said second binding site.

32. The method according to claim 31, wherein said homologue has a root mean square deviation from the backbone atoms of said amino acids between 0.00 Å and 1.00 Å.

33. The method according to claim 31, wherein said homologue has a root mean square deviation from the backbone atoms of said amino acids between 0.00 Å and 0.50 Å.

34. The method according to any one of claims 31-33, wherein said first binding site is a binding site of BAFF for one or more receptors of BAFF.

35. The method according to any one of claims 31-33, wherein said second binding site is a binding site of BAFF for one or more receptors of BAFF.

36. A method for evaluating the potential of a chemical entity to associate with:
a) a molecule or molecular complex comprising a first binding site defined by at least four BAFF amino acids selected from the group consisting of His218, Val219, Phe220, Gly221, Asp222, Glu223, Leu224, Ser225, Leu226, Val227, Pro264, Arg265, Glu266, Gly161, Ser162, Tyr163, Ala151, Asp152, Ser153, Glu154, Thr155, Pro156, Leu240, Pro241, Asn242, Ser171 and Phe172 according to FIG. 8; or
b) a homologue of said molecule or molecular complex comprising a second binding site having a root mean square deviation from the backbone atoms of said at least four BAFF amino acids between 0.00 Å and 1.50 Å;
comprising the steps of:
(i) employing computational means to perform a fitting operation between said chemical entity and said first binding site or said second binding site; and
(ii) analyzing the results of said fitting operation to quantify the association between said chemical entity and said first binding site or said second binding site.

37. The method according to claim 36, wherein said homologue has a root mean square deviation from the backbone atoms of said amino acids between 0.00 Å and 1.00 Å.

38. The method according to claim 36, wherein said homologue has a root mean square deviation from the backbone atoms of said amino acids between 0.00 Å and 0.50 Å.

39. The method according to any one of claims 36-38, wherein said first binding site is a binding site of BAFF for one or more receptors of BAFF.

40. The method according to any one of claims 36-38, wherein said second binding site is a binding site of BAFF for one or more receptors of BAFF.

41. A chemical entity evaluated by the method according to any one of claims 28-40.

42. A compound assembled from one or more of a chemical entity according to claim 41.

43. A method for identifying a potential antagonist of BAFF comprising the steps of:
a) using structure coordinates of the amino acids of BAFF according to FIG. 8 ± a root mean square deviation from the backbone atoms of said amino acids between 0.00 Å and 1.50 Å, to generate a three-dimensional structure of a molecule or a molecular complex comprising a binding site;
b) employing said three-dimensional structure to design or select said potential antagonist;
c) synthesizing said potential antagonist; and
d) contacting said potential antagonist with BAFF to determine the ability of said potential antagonist to interact with BAFF.

44. The method according to claim 43, wherein said root mean square deviation from the backbone atoms of said amino acids is between 0.00 Å and 1.00 Å.

45. The method according to claim 43, wherein said root mean square deviation from the backbone atoms of said amino acids is between 0.00 Å and 0.50 Å.

46. The method according to claim 43, wherein said binding site is a binding site of BAFF for one or more of the receptors of BAFF.

47. A method for identifying a potential antagonist of BAFF comprising the steps of:
a) using the structure coordinates of a plurality of BAFF amino acids selected from the group consisting of His218, Val219, Phe220, Gly221, Asp222, Glu223, Leu224, Ser225, Leu226, Val227, Pro264, Arg265, Glu266, Gly161, Ser162, Tyr163, Ala151, Asp152, Ser153, Glu154, Thr155, Pro156, Leu240, Pro241, Asn242, Ser171 and Phe172 according to FIG. 8 or ± a root mean square deviation from the backbone atoms of said plurality of BAFF amino acids between 0.00 Å and 1.50Å, to generate a three-dimensional structure of a molecule or a molecular complex comprising a binding site;
b) employing said three-dimensional structure to design or select said potential antagonist;
c) synthesizing said potential antagonist; and
d) contacting said potential antagonist with BAFF to determine the ability of said potential antagonist to interact with BAFF.

48. The method according to claim 47, wherein said root mean square deviation from the backbone atoms of said amino acids is between 0.00 Å and 1.00 Å.

49. The method according to claim 47, wherein said root mean square deviation from the backbone atoms of said amino acids is between 0.00 Å and 0.50 Å.

50. The method according to claim 47, wherein said binding site is a binding site of BAFF for one or more of the receptors of BAFF.

51. A method for identifying a potential antagonist of BAFF comprising the steps of:
a) using the structure coordinates of at least four BAFF amino acids selected from the group consisting of His218, Val219, Phe220, Gly221, Asp222, Glu223, Leu224, Ser225, Leu226, Val227, Pro264, Arg265, Glu266, Gly161, Ser162, Tyr163, Ala151, Asp152, Ser153, Glu154, Thr155, Pro156, Leu240, Pro241, Asn242, Ser171 and Phe172 according to FIG. 8 or ± a root mean square deviation from the backbone atoms of said at least four BAFF amino acids between 0.00 Å and 1.50Å, to generate a three-dimensional structure of a molecule or a molecular complex comprising a binding site;
b) employing said three-dimensional structure to design or select said potential antagonist;
c) synthesizing said potential antagonist; and
d) contacting said potential antagonist with BAFF to determine the ability of said potential antagonist to interact with BAFF.

52. The method according to claim 51, wherein said root mean square deviation from the backbone atoms of said at least four BAFF amino acids is between 0.00 Å and 1.00 Å.

53. The method according to claim 51, wherein said root mean square deviation from the backbone atoms of said at least four BAFF amino acids is between 0.00 Å and 0.50 Å.

54. The method according to claim 51, wherein said binding site is a binding site of BAFF for one or more of the BAFF receptors.

55. The method according to any one of claims 43-54, further comprising the step of:
(e) determining whether said potential antagonist interrupts BAFF and a receptor of BAFF interaction.

56. A potential antagonist of BAFF identified by the method according to any one of claims 43-55.

57. A method for evaluating the potential of a variant of an antagonist of BAFF to associate with:
a) a molecule or a molecular complex defined by the structure coordinates of the BAFF amino acids, set forth in FIG. 8; or
b) a homologue of said molecule or molecular complex having a root mean square deviation from the backbone atoms of said amino acids between 0.00 Å and 1.50 Å;
comprising the steps of:
(i) employing computational means to perform a fitting operation between the variant and said molecule or molecular complex; and
(ii) analyzing the results of said fitting operation to quantify the association between said variant and said molecule or molecular complex.

58. The method according to claim 57, wherein said homologue has a root mean square deviation from the backbone atoms of said amino acids of between 0.00 Å and 1.00 Å.

59. The method according to claim 57, wherein said homologue has a root mean square deviation from the backbone atoms of said amino acids of between 0.00 Å and 0.50 Å.

60. A method for evaluating the potential of a variant of an antagonist of BAFF to associate with:
a) a first binding site of a molecule or a molecular complex defined by structure coordinates of a plurality of BAFF amino acids selected from the group consisting of His218, Val219, Phe220, Gly221, Asp222, Glu223, Leu224, Ser225, Leu226, Val227, Pro264, Arg265, Glu266, Gly161, Ser162, Tyr163, Ala151, Asp152, Ser153, Glu154, Thr155, Pro156, Leu240, Pro241, Asn242, Ser171 and Phe172, set forth in FIG. 8; or
b) a homologue of said molecule or molecular complex comprising a second binding site having a root mean square deviation from the backbone atoms of a plurality of BAFF amino acids between 0.00 Å and 1.50 Å;
comprising the steps of:
(i) employing computational means to perform a fitting operation between the variant and said first binding site or said second binding site; and
(ii) analyzing the results of said fitting operation to quantify the association between said variant and said first binding site or said second binding site.

61. The method according to claim 60, wherein said homologue has a root mean square deviation from the backbone atoms of a plurality of BAFF amino acids of between 0.00 Å and 1.00 Å.

62. The method according to claim 60, wherein said homologue has a root mean square deviation from the backbone atoms of a plurality of BAFF amino acids of between 0.00 Å and 0.50 Å.

63. The method according to any one of claims 60-62, wherein said first binding site is a binding site of BAFF for one or more receptors of BAFF.

64. The method according to any one of claims 60-62, wherein said second binding site is a binding site of BAFF for one or more receptors of BAFF.

65. A method for evaluating the potential of a variant of an antagonist of BAFF to associate with:
a) a first binding site of a molecule or a molecular complex defined by structure coordinates of at least four BAFF amino acids selected from the group consisting of His218, Val219, Phe220, Gly221, Asp222, Glu223, Leu224, Ser225, Leu226, Val227, Pro264, Arg265, Glu266, Gly161, Ser162, Tyr163, Ala151, Asp152, Ser153, Glu154, Thr155, Pro156, Leu240, Pro241, Asn242, Ser171 and Phe172, set forth in FIG. 8; or
b) a homologue of said molecule or molecular complex comprising a second binding site having a root mean square deviation from the backbone atoms of said at least four BAFF amino acids between 0.00 Å and 1.50 Å;
comprising the steps of:
(i) employing computational means to perform a fitting operation between the variant and said first binding site or said second binding site; and
(ii) analyzing the results of said fitting operation to quantify the association between said variant and said first binding site or said second binding site.

66. The method according to claim 65, wherein said homologue has a root mean square deviation from the backbone atoms of said at least four BAFF amino acids of between 0.00 Å and 1.00 Å.

67. The method according to claim 65, wherein said homologue has a root mean square deviation from the backbone atoms of said at least four BAFF amino acids of between 0.00 Å and 0.50 Å.

68. The method according to any one of claims 65-67, wherein said first binding site is a binding site of BAFF for one or more receptors of BAFF.

69. The method according to any one of claims 65-67, wherein said second binding site is a binding site of BAFF for one or more receptors of BAFF.

70. A variant of an antagonist of BAFF identified by the method according to any one of claims 57-69.

71. A pharmaceutical composition comprising a pharmaceutically suitable carrier and a chemical entity according to claim 41 or a compound according to claim 42, a potential antagonist of BAFF according to claim 56, or a variant of an antagonist of BAFF according to claim 70.

72. A method of treating a condition associated with inappropriate or abnormal BAFF induced activation in a subject, comprising the step of administering an effective amount of a pharmaceutical composition according to claim 71 to the subject.

73. A method of preventing a condition associated with inappropriate or abnormal BAFF induced activation in a subject, comprising the step of administering an effective amount of a pharmaceutical composition according to claim 71 to the subject.

74-83. (canceled)

84. A method of utilizing the structure coordinates of BAFF to obtain a homology model structure of at least a portion of a molecule whose structure is unknown and at least a portion of whose structure is similar to the structure of BAFF, comprising the step of:
applying at least a portion of the structure coordinates set forth in FIG. 8 to generate a three-dimensional molecular model of at least a portion of the molecule whose structure is unknown to generate a homology model structure of at least a portion of that molecule.

85. The method according to claim 84, wherein said molecule whose structure is unknown comprises an APRIL polypeptide.

86. The method according to claim 85, wherein said APRIL polypeptide comprises the extracellular domain of APRIL.

87. The method according to claim 84, wherein said molecule whose structure is unknown comprises a trimer of APRIL polypeptides.

88. A computer for producing a three-dimensional representation of:
a) a homology model structure of at least a portion of a molecule whose structure is unknown and at least a portion of whose structure is similar to the structure of BAFF, wherein said homology model structure is defined by at least a portion of the homology model structure coordinates of the APRIL amino acids set forth in FIG. 10;
wherein said computer comprises:
(i) a machine-readable data storage medium comprising a data storage material encoded with machine-readable data, wherein said data comprises at least a portion of the structure coordinates of all of the APRIL amino acids set forth in FIG. 10; and
(ii) instructions for processing said machine-readable data into said three-dimensional representation

89. The computer according to claim 88, further comprising a display for displaying said homology model structure coordinates.
